SEQUENCE LISTING (95 sequences)

SEQ ID NO: 1 - DNA, 1764 bp, synthetic construct
  source 1..1764: mol_type = other DNA
atggcggaggagagacgacaaaacgcacttgaaatcaggatccaagctgctaagctagctaagaagagggaccatcctacacacaaagccaatggagatgaagacagatatccagagacacttataggtagctttactaaaggactgccgcacgagaaggaaacaggtttattgtctaaccctgctgacttcgctgatttcgttcgagctattaataccggtgctatcaaggacatgaggaggttgaaattgggtatcgatgaagatcctcgttttatcagtggaatcgcgtccaaaggtgacaagcacccttttgctgatacacgagcttgggaatctatggcagcaggactgacttacgatcttgagggaccagatgcccaggcggtcactatgcctccagcgcctaagcttgatagcgacgaactggtgacggagatcacggaatcttattggatggctttattgagagatgttccttttaccgagtttgaacgagatgggaacactgctgccgctgcagctagcatatcccggacacgatgggttcaatataatgagcaaccttcgcaaagacctagcacacttacagacgaagagatagcaagactacgaggaccgtataccaaaaaaaacgtattcaggggtgtcaccaatggggagaatgtaggcccttatttatcacagtttcttctggttggaacaaagggcattggtgatgcccaacaagttagtgatggttatgtacagtatggaggaatgagaatggaccaaagagttcgagtggctgtgcctaaaagagattacatgaccacatgggcttcttggctcgatgttcaaaatgctggtgatcttagaggtagagaaatctatgacgacgacacaccattccgattcataacaacgccacgtgatctggctacatgggtgcattttgatgcattataccaggcttatttgaatgcttgtataatactattagatattaaggctccgttcgatcctcacatccctttccaagcggatgatgatgttgataaacagcaaggattcgcaacgtttggtggtccgcatattctttctctttgcactgaggtggctacaagagcgctgaaagcggttcgttttcagaagtacaacttacacaggaggttgagacctgaggcaattggtggtcttgtcgagaggtttaagaaaacaaatggcgacccaaaattcgcacctgttaaaaaattggttaatgacttagacggtgatatgttgcggcgagttgagcaacataactgtgaacaaaataagctctccgatgacggccatgctagaagggaagactatagcccagagggagagtcttcacaatcatatcttttacctatggcattccctgagggttcccctatgcatccttcatacggtgcgggacacgccacggtggccggtgcatgtgtcactgtgcttaaagcctttttcgatcatgaatatgaacttgatttttgctatgtccctacaaccgatggaaagaggcttgagaaagttaatattaatgaaaaacttactgttgagggcgaacttaataagctttgtgctaacataagtattggtcgtaactgggcgggcgttcattactactctgactatttcgagagtatcaaggttggagaggagatcgctattggtatcttgcaggaacaaaaattgacttatggagaggatttttttatgactctacctaagtttgacggtgagaaaataagaatataatga

SEQ ID NO: 2 - protein, 586 aa, synthetic construct
MAEERRQNALEIRIQAAKLAKKRDHPTHKANGDEDRYPETLIGSFTKGLPHEKETGLLSNPADFADFVRAINTGAIKDMRRLKLGIDEDPRFISGIASKGDKHPFADTRAWESMAAGLTYDLEGPDAQAVTMPPAPKLDSDELVTEITESYWMALLRDVPFTEFERDGNTAAAAASISRTRWVQYNEQPSQRPSTLTDEEIARLRGPYTKKNVFRGVTNGENVGPYLSQFLLVGTKGIGDAQQVSDGYVQYGGMRMDQRVRVAVPKRDYMTTWASWLDVQNAGDLRGREIYDDDTPFRFITTPRDLATWVHFDALYQAYLNACIILLDIKAPFDPHIPFQADDDVDKQQGFATFGGPHILSLCTEVATRALKAVRFQKYNLHRRLRPEAIGGLVERFKKTNGDPKFAPVKKLVNDLDGDMLRRVEQHNCEQNKLSDDGHARREDYSPEGESSQSYLLPMAFPEGSPMHPSYGAGHATVAGACVTVLKAFFDHEYELDFCYVPTTDGKRLEKVNINEKLTVEGELNKLCANISIGRNWAGVHYYSDYFESIKVGEEIAIGILQEQKLTYGEDFFMTLPKFDGEKIRI

SEQ ID NO: 3 - DNA, 1764 bp, synthetic construct
  source 1..1764: mol_type = other DNA
atggcagaagaaaggcggcagaacgcgctagagatccgcattcaagcggcaaagctcgcaaagaagcgggatcacccgacacataaggcaaacggcgatgaggaccgctatccagagacgcttatcggctcattcaccaaaggcttgccgcacgagaaggagacagggcttctctccaatcctgcagactttgctgatttcgttagggctattaatacaggcgccatcaaggacatgaggcggctcaagctgggtattgacgaagaccctcggttcatctccggtatagcctcaaaaggcgacaaacatcccttcgcggacaccagagcctgggaatctatggctgctggcttgacctatgatctggaggggcctgacgcgcaggccgttacgatgccccccgccccgaaacttgacagcgacgagttggtgactgaaattaccgaatcatactggatggcccttctccgagatgttccgtttactgagttcgagcgcgacggaaatactgcagctgctgcagcgtccataagcagaactcgatgggtacagtacaacgagcaaccttcacagcggccatcgactttgacggacgaggaaatagcccgtttgcgcgggccttatacgaagaagaatgttttcaggggggtgaccaacggtgagaacgtaggcccatacctctcccagttcctcctcgtgggcaccaaaggcatcggcgatgcacagcaagtgtcggatggatacgtccagtacggcggcatgaggatggatcagcgcgtaagggtagctgtgcctaagagggattacatgaccacttgggcgagctggctggacgttcagaatgcgggggacttgagaggacgcgaaatatacgacgacgacacgccattcaggttcataactaccccgcgtgatctggcaacttgggtgcacttcgacgccttgtaccaagcataccttaacgcgtgtattatcctgctcgatatcaaggctccgttcgatccgcacataccatttcaagccgacgacgacgtcgacaagcagcaaggattcgcaaccttcggaggtccgcatatcctcagtctctgcacagaggtcgccaccagggcactcaaagccgttcgcttccagaagtataacctacaccggcgtctccgccctgaagccatcggaggcctggttgagcggttcaagaagaccaatggcgacccaaagttcgctcctgtgaaaaagctcgtgaatgacctcgacggagacatgcttcgccgagtcgagcaacacaattgcgagcagaacaaactttccgacgatggccacgcgagaagagaggactacagcccggagggagagtcaagccagagctatcttcttcctatggcgttcccggagggttctcctatgcatccatcatatggtgctggccacgctaccgtcgccggggcgtgcgtgacagtgttgaaggcttttttcgaccacgaatacgagcttgacttctgctatgtccccaccaccgacggtaagcgtctcgagaaggtcaacatcaatgagaagttgaccgttgagggcgagctgaataagttgtgtgcaaacatcagcatagggcgcaactgggcaggggtccactactactctgactatttcgagtccattaaggtgggagaggagatagccatcggtattctgcaggagcagaagcttacatacggggaggacttctttatgacactaccgaaatttgatggcgagaagattaggatttaatga

SEQ ID NO: 4 - protein, 590 aa, synthetic construct
MTDTQNPNRAEVAFKVRVSAAELARARGSPAHLSNNSESRFRNPDGTRSLLANFTKGLPHIKETALVESAIDYDRFVRAIDSGDPRDFADLPLGPQGVEPRFTSGIASDPEVGTRAWESGGAGLVFDLEGPDAQAVTMPPAPELDSDELVAEVTECYWMSLLRDVPFPTFESNSHIQAAAESINNTQWIKFKNNPPAHLTAAERSRLRGPVTTANVFRGITPGDEVGPYLSQFLLVGTTGIANGNEVGDGFIQYGGMRMDQRVRVAKPHIDYMTTFGAYLDVQNAANVSGRELYKEEEPRFRFIHTPRDLATYVHFDALYQAYLNACIILLDIGAPFDSGIPFQLDNDIDKQQGFATFGGPHILSLVTEVATRALKAVRFQKFNVHRRLRPEAIGARVDRYCATKAPEFAGAAKLSEALDKELLQKVHDHNKKQNLLSDRGNPRANDFNPDGEVSEGNLLMPMAFPEGSPMHPAYGAGHATVAGACVTVLKAFFDGGYRLPFCYITDEDGTGLQAVEIDEPLTVDGELNKICSNISIGRNWAGVHYFTDYIESIRIGEEIAIGILQEQKLTFSENFSMTLNKFDGSTIRI

SEQ ID NO: 5 - protein, 611 aa, synthetic construct
MRERSQKSYSIRVDAAELARSRQHPIHEANGDEQRYGGDKYFMSFTKGLIHNPNTGLLEDPRDFVEFRRAIDNGFIDPFTDRVRHGAKYKVNDQGNIVEEDNPQDLDFRQWEAPTAGVVHELEGPDAQAVTIPPAPPLLDSTRNTNPELIFEMAEVYELAILRDVPLNNFDNKNTTDELTASIARLNNLDYVNNQNGRPRKVNQAGELDLQTVFRGSSPGVEVGPYLSQFLLIGNKGVNGVDNTTTGRIAYGVQQIDQRVPVASVQNYMTQWENYVRVQRGIKQERETYVEGNGSHRFIYTPRDLATYVHYDALYQAYLNACFILLGMGTPFDPSFDHLSGGGEAAKDPKTRPHAGGFALYAGPHILTLVTEVATRALKAIRFQKFNNHIRLRPEALAARIDLVRRKNNGEIQESLIPQALSQYVHGFIDKLGSNGYGSLTTSTLGRIAKIAESSYLLPMAFPEGSPMHPAYGAGHATVAGACVTILKAFFDTSTVLIKDSNNEYKFKHQEAQDQFIAFRTNSSGTDLITEDINNISEALTLEGELNKLAANISIGRNMAGVHYFSDYYDSIRMGEQIAIGILQEQALTYSTDPFVMSVPTFDGNIVRIGY

SEQ ID NO: 6 - protein, 622 aa, synthetic construct
MTDQRKLTAQRVREDANALAAGRIHPRHQANGDEQRYESANYAMSFTKGLDHNTTTGLIEQSGDFEAFRSAIDNGFAEDFTRHVAVPRAEPRRKWEAPTAGTVYELQGPDPQAVTIPPAPALCSDELTFEMAEVYELALLRDLPFNAFVAGGGSAALADSTARLNSLAYAQDGFNRRPRTTNSSNQLDAQTVFRGSSPGVDQGPYLSQFMLIGNASPSEGITPEQGFINFGAQRIDQRVLEARQQDDYMMKWDDWHRVQQGYEVRADRFDPCKSSGPGQAFTGQRRFIHTPRDLATYVHVDALYQAYLNACLLLLGNGTAFDPGFDLLSGGGEGLLHDPAGGQKVPLNAGGFALWGGPHVLSLVTEVATRGLKAVRYQKFNNHLRLRPEALAARIEKAQEIESRFPTICGCFSEMASDLQQVVDLIRNHNQSLAGEATALLPMAFAEGSPMHPAYGAGHATVAGACVTILKAFFNTSALFVKINDVAGFHSKQHILARLKCGDSVEAGAYQETDCGKRLEFERCGSFHLIEGKYATFKPDGKTNQSCCPLTLEGELNKLAANISIGRNMAGVHYFSDYYDSLRMGEEIAIGILEEQALCYKTDPFVLSVPTFDGDVRRIGQR

SEQ ID NO: 7 - protein, 625 aa, synthetic construct
MAGPTASGTARRNEAFDKRVAAATVARERVHPPHAANGEEYEYRATFDRHPAVAAAKAAAAGASREASNGDPGATDGGATAAGAPPSSQSQVQGPSHIGSFTKGLQHNLSTGLLEDPAAFRAFVRAIDSGDPRDFVDVPIADRFTRPPTRGVRSWESASAGLAFDLQGPDAQALTMPPAPRLGSAELVAEMIEVYAMALARDVPFATAEWAAAKPIAAAVRELNATRWHGAAATDRELGLSPAAAARRRVTHFTPATVFRGVGRGVADGPYLSQFLLLGNAGVGGSSGAPTDGFITYGANRVDLRVPVATPGVDYLTDWESFVDVQNGADTRGTETYVPAADGTKTRFIATPRDLATYVHYDALYQAYLNAALWLLAEGVPFDAGLPWTAPDEVDHQQGFATFGGPHLLTLVTEVATRALKAVRFQKFNVHRRLRPEAVGGLLDRLPVAPGPLAPVAPLAEEAAETLRRVADANAAAAAGGSGAGAPVTHSVLLPMAFPEGSPMHPSYGAGHATVAGACVTVLKGWFDAGHALGTTAYEATPDGRCLREVSTGGVALTVEGELNKLAANISIGRNWAGVHYYSDYIESVQLGEAVAIGILEEQKLTYGENWSMTVPLTDGTTVRI

SEQ ID NO: 8 - protein, 678 aa, synthetic construct
MKRVRPTQPRALYSAFSRRHVACALLCVVSCIFVSFNHQTFVGLTALLAAFPPCLGYDEPAEPTQPLLSGSVCRVRDSLDFLDPVPRAKVTLLKRLAIAKDEFSVGPTCHINNGDEENVPLFAGQFHKTLPHDKFGQVDEDAYKKLQECIFTSDINVCDDVPSGAKKNGAKLTNPLGGTAHQVAGADSDNIFIPTPDSLLSERLAAQQSEVYWMALARDIPFGEFATNDLIRLAAENLQGLPAFKGLNIPRSKGGKIDPVTDLFRTTWPGVTTGPVVSQFMLSDFLIDSINVTPKADPLTPFTDYMTSFQPWLDVQNGASDVDTTFDSENPRFIRNGRDLATISFRDLLYTEAFRAALILFTQGALGGSIGPYEEAERQQGFATFGAPHILTAMGSASSSTRHAWYQKWQVHRMLRPEAYGGLVHNTLMKNVITPLPDSILRNTELLNRVEVHNQKMNGDGEKTFLLPMASAQGSPTHPAYPSGHAINVGAYITSLKAFLGFEAGQRCFPDPVVSNNEGTERIPYVPSGREIVGECINDKGKKVDGLTYEGELNKVSANVLIGRSHLGVHWRMDGVYGALVGEVSAVRRLQQELPGLAEARPNDDLKRKEIPPATYNFRLYSGKMLELYGANLYKLDGKLCEGAFTGDDFCDEVQEDDYESFEHIVEDLAKFSLHTEL

SEQ ID NO: 9 - DNA, 1292 bp, synthetic construct
  source 1..1292: mol_type = other DNA
atggcccatacatcagaatctgtgaatcctagaggtaacaaaacactttcattagtaaactgaatttgaaggatgaattttttttttgatatatgaatgttgatgaatcagatgtttgcattgtgggtgttgcacgtactccaatgggtggctttctcggatctctttcatctttacctgccacaaagcttggatctttagctattgcagctgctttgaagagagcaaatgttgatccagctcttgttcaagaagttgtctttggcaatgttcttagtgctaatttgggtcaagctcctgctcgtcaagctgctttaggtgcaggaatccctaactctgttatctgtactacagttaacaaggtttgtgcatcaggcatgaaagcggtaatgattgctgctcaaagtatccagttagggatcaatgatgtagttgtggcgggtggtatggaaagcatgtctaatacaccaaaatatttggcagaagcaaggaagggatctcgttttggtcatgattctttagtagatggaatgttgaaggatggactatgggatgtctataacgactgtgggatgggaagctgtgcagaattatgcgctgagaagtttcagattacaagggagcagcaagatgactatgcagttcagagttttgagcgtggtattgctgcccaggaagctggcgccttcacatgggaaatcgtcccggttgaagtttctggaggaagaggtaggccatcaaccattgttgacaaggacgaaggtcttgggaagtttgatgctgcaaaattgaggaaactccgtcctagtttcaaagagaatggagggactgttacagctggaaatgcgtctagcataagtgatggtgcagctgcccttgtcctagtgagcggagagaaggctcttcagctaggacttctagtattagcaaaaattaaagggtatggtgacgcagctcaggaaccagagtttttcactactgctcctgctcttgctataccaaaagccattgcacatgctggtttggaatcttctcaagttgattactatgagatcaatgaagcatttgcagttgtagcacttgcaaatcaaaagctactcgggattgctccagagaaagtgaacgtaaatggaggagctgtctccttaggacaccctctaggctgcagtggcgcccgtattctaatcacgttgcttgggatactaaagaagagaaacggaaagtacggtgtgggaggagtgtgcaacggaggaggaggtgcttctgctctagttcttgagctcctttgataa

SEQ ID NO: 10 - DNA, 1215 bp, synthetic construct
  source 1..1215: mol_type = other DNA
atggcccatacatcagaatctgtgaatcctagagatgtttgcattgtgggtgttgcacgtactccaatgggtggctttctcggatctctttcatctttacctgccacaaagcttggatctttagctattgcagctgctttgaagagagcaaatgttgatccagctcttgttcaagaagttgtctttggcaatgttcttagtgctaatttgggtcaagctcctgctcgtcaagctgctttaggtgcaggaatccctaactctgttatctgtactacagttaacaaggtttgtgcatcaggcatgaaagcggtaatgattgctgctcaaagtatccagttagggatcaatgatgtagttgtggcgggtggtatggaaagcatgtctaatacaccaaaatatttggcagaagcaaggaagggatctcgttttggtcatgattctttagtagatggaatgttgaaggatggactatgggatgtctataacgactgtgggatgggaagctgtgcagaattatgcgctgagaagtttcagattacaagggagcagcaagatgactatgcagttcagagttttgagcgtggtattgctgcccaggaagctggcgccttcacatgggaaatcgtcccggttgaagtttctggaggaagaggtaggccatcaaccattgttgacaaggacgaaggtcttgggaagtttgatgctgcaaaattgaggaaactccgtcctagtttcaaagagaatggagggactgttacagctggaaatgcgtctagcataagtgatggtgcagctgcccttgtcctagtgagcggagagaaggctcttcagctaggacttctagtattagcaaaaattaaagggtatggtgacgcagctcaggaaccagagtttttcactactgctcctgctcttgctataccaaaagccattgcacatgctggtttggaatcttctcaagttgattactatgagatcaatgaagcatttgcagttgtagcacttgcaaatcaaaagctactcgggattgctccagagaaagtgaacgtaaatggaggagctgtctccttaggacaccctctaggctgcagtggcgcccgtattctaatcacgttgcttgggatactaaagaagagaaacggaaagtacggtgtgggaggagtgtgcaacggaggaggaggtgcttctgctctagttcttgagctcctttgataa

SEQ ID NO: 11 - protein, 403 aa, synthetic construct
MAHTSESVNPRDVCIVGVARTPMGGFLGSLSSLPATKLGSLAIAAALKRANVDPALVQEVVFGNVLSANLGQAPARQAALGAGIPNSVICTTVNKVCASGMKAVMIAAQSIQLGINDVVVAGGMESMSNTPKYLAEARKGSRFGHDSLVDGMLKDGLWDVYNDCGMGSCAELCAEKFQITREQQDDYAVQSFERGIAAQEAGAFTWEIVPVEVSGGRGRPSTIVDKDEGLGKFDAAKLRKLRPSFKENGGTVTAGNASSISDGAAALVLVSGEKALQLGLLVLAKIKGYGDAAQEPEFFTTAPALAIPKAIAHAGLESSQVDYYEINEAFAVVALANQKLLGIAPEKVNVNGGAVSLGHPLGCSGARILITLLGILKKRNGKYGVGGVCNGGGGASALVLELL

SEQ ID NO: 12 - DNA, 1215 bp, synthetic construct
  source 1..1215: mol_type = other DNA
atggctcatacgtcagagtcagtaaacccacgtgacgtttgcatcgtgggggtagcccgtaccccaatgggcggtttcctcggaagcctctcctccctgccagcgaccaagctcgggtcactggcgatcgctgcagcgctgaagcgagctaacgtggacccggccctggtgcaggaggtagtcttcggaaatgtcctgtcagccaatcttgggcaagctcccgctaggcaagctgccctcggcgcgggcattccgaacagcgtcatctgcactaccgtcaacaaggtgtgcgcgtccggaatgaaagcggttatgattgcggcccaatccatacaacttggcatcaatgatgttgtagtggccggcggtatggagtcaatgtctaacacgccgaagtatctagcagaggcaagaaagggaagcaggtttggccatgattcccttgtcgacggtatgctcaaagacgggctgtgggacgtttacaacgactgcggcatggggagctgcgcggagctgtgcgcagaaaagtttcagattacccgcgaacagcaggacgattacgccgtgcagtcattcgaacgcgggatcgccgcgcaggaggcaggtgccttcacgtgggaaatcgttcccgtcgaggtaagtggtggtcgcggccggcctagcactatcgttgataaggacgagggtctgggcaagttcgacgctgcgaagctccggaagctaaggccgtccttcaaggagaacggcgggacggtcacggccggcaatgcatctagcataagcgatggagcggcggcgctagtccttgtcagcggggaaaaggctctacagctaggcttgctcgtcctcgccaagattaagggctatggcgatgctgcgcaggaaccagagttctttacgacggcgccggcgctggctatccccaaggctatcgctcatgcgggactggaatcctctcaagtggattattacgaaattaatgaggcgttcgcagtggtcgccctggctaaccagaagcttctcgggatcgcgccggagaaggttaatgtgaatggtggtgccgtgtctctgggtcacccgctcggatgcagcggtgcacgtatcttgatcacgctactcggtatcctcaaaaaacggaacgggaagtacggggtcggcggtgtttgcaacggggggggaggagcgagcgccctggtcctcgagctcttgtgataa

SEQ ID NO: 13 - DNA, 1998 bp, synthetic construct
  source 1..1998: mol_type = other DNA
atcttctgatgagaataagcaaatccctattttttttgttgaagaaaagaaaagcgtgtccacgtggactggactctgaaattttcctcctcccttctggagtcttcctcatcagaaatcagaagaggagagggtgggcaactcgcagatcgccttctcgtcgcgctcgcgccgcaggggtcgcctgaggtatgcagcttcacctcccccaactttctagggttctaatcgcctctgctcgctcggattatgcgtggtgggtatggctccgattccgccggagtagctggatctgtgtgccccgtgctaatttaggtttggctttcgattcggccgcgcttcggtttgttctcgcgcgtgattgcttcgttcggccatagggctcatctcaggctcgacatgtggacggccacaaacataaaaaatccttgttaaatttacggttcatgtggtaggggggcttttcaggatcggaggtttaggtgatttggagtagcaaaacgattttgctgtgttagagaattcgagaatctgtgccgtcagatgctaattagctggttttgaacaaatgttaagacatcctgatattatttgctgcgtttaatgttcaaactttttgcacgggtgttgctgagctataaaggatatgaggatatagtaccaagttctatcgttttgttctgatacatacaatttgctgtagtttatggcaccatcatactgagatatatatacctgtgcacctttcggcttctgtgcagaacacatagacaatggtgaaggatggctcatacgtcagagtcagtaaacccacgtgacgtttgcatcgtgggggtagcccgtaccccaatgggcggtttcctcggaagcctctcctccctgccagcgaccaagctcgggtcactggcgatcgctgcagcgctgaagcgagctaacgtggacccggccctggtgcaggaggtagtcttcggaaatgtcctgtcagccaatcttgggcaagctcccgctaggcaagctgccctcggcgcgggcattccgaacagcgtcatctgcactaccgtcaacaaggtgtgcgcgtccggaatgaaagcggttatgattgcggcccaatccatacaacttggcatcaatgatgttgtagtggccggcggtatggagtcaatgtctaacacgccgaagtatctagcagaggcaagaaagggaagcaggtttggccatgattcccttgtcgacggtatgctcaaagacgggctgtgggacgtttacaacgactgcggcatggggagctgcgcggagctgtgcgcagaaaagtttcagattacccgcgaacagcaggacgattacgccgtgcagtcattcgaacgcgggatcgccgcgcaggaggcaggtgccttcacgtgggaaatcgttcccgtcgaggtaagtggtggtcgcggccggcctagcactatcgttgataaggacgagggtctgggcaagttcgacgctgcgaagctccggaagctaaggccgtccttcaaggagaacggcgggacggtcacggccggcaatgcatctagcataagcgatggagcggcggcgctagtccttgtcagcggggaaaaggctctacagctaggcttgctcgtcctcgccaagattaagggctatggcgatgctgcgcaggaaccagagttctttacgacggcgccggcgctggctatccccaaggctatcgctcatgcgggactggaatcctctcaagtggattattacgaaattaatgaggcgttcgcagtggtcgccctggctaaccagaagcttctcgggatcgcgccggagaaggttaatgtgaatggtggtgccgtgtctctgggtcacccgctcggatgcagcggtgcacgtatcttgatcacgctactcggtatcctcaaaaaacggaacgggaagtacggggtcggcggtgtttgcaacggggggggaggagcgagcgccctggtcctcgagctcttgtgataa

SEQ ID NO: 14 - DNA, 1402 bp, synthetic construct
  source 1..1402: mol_type = other DNA
atgaagaagcgcttaaccacttccacttgttcttcttctccatcttcctctgtttcttcttctactactacttcctctcctattcagtcggaggctccaaggcctaaacgagccaaaagggctaagaaatcttctccttctggtgataaatctcataacccgacaagccctgcttctacccgacgcagctctatctacagaggagtcactaggtttttatttttttggaaattaaatgattggttgttgagattggatttgggttttgtcttaaaactgcatttgtaagattgcatgttgttttgtgggattttgcagacatagatggactgggagattcgaggctcatctttgggacaaaagctcttggaattcgattcagaacaagaaaggcaaacaagtttatctgggagcatatgacagtgaagaagcagcagcacatacgtacgatctggctgctctcaagtactggggacccgacaccatcttgaattttccggcagagacgtacacaaaggaattggaagaaatgcagagagtgacaaaggaagaatatttggcttctctccgccgccagagcagtggtttctccagaggcgtctctaaatatcgcggcgtcgctaggcatcaccacaacggaagatgggaggctcggatcggaagagtgtttgggaacaagtacttgtacctcggcacctataatacgcaggaggaagctgctgcagcatatgacatggctgcgattgagtatcgaggcgcaaacgcggttactaatttcgacattagtaattacattgaccggttaaagaagaaaggtgttttcccgttccctgtgaaccaagctaaccatcaagagggtattcttgttgaagccaaacaagaagttgaaacgagagaagcgaaggaagagcctagagaagaagtgaaacaacagtacgtggaagaaccaccgcaagaagaagaagagaaggaagaagagaaagcagagcaacaagaagcagagattgtaggatattcagaagaagcagcagtggtcaattgctgcatagactcttcaaccataatggaaatggatcgttgtggggacaacaatgagctggcttggaacttctgtatgatggatacagggttttctccgtttttgactgatcagaatctcgcgaatgagaatcccatagagtatccggagctattcaatgagttagcatttgaggacaacatcgacttcatgttcgatgatgggaagcacgagtgcttgaacttggaaaatctggattgttgcgtggtgggaagagagagcccaccctcttcttcttcaccattgtcttgcttatctactgactctgcttcatcaacaacaacaacaacaacctcggtttcttgtaactatttggtctgataa

SEQ ID NO: 15 - DNA, 1296 bp, synthetic construct
  source 1..1296: mol_type = other DNA
atgaagaagcgcttaaccacttccacttgttcttcttctccatcttcctctgtttcttcttctactactacttcctctcctattcagtcggaggctccaaggcctaaacgagccaaaagggctaagaaatcttctccttctggtgataaatctcataacccgacaagccctgcttctacccgacgcagctctatctacagaggagtcactagacatagatggactgggagattcgaggctcatctttgggacaaaagctcttggaattcgattcagaacaagaaaggcaaacaagtttatctgggagcatatgacagtgaagaagcagcagcacatacgtacgatctggctgctctcaagtactggggacccgacaccatcttgaattttccggcagagacgtacacaaaggaattggaagaaatgcagagagtgacaaaggaagaatatttggcttctctccgccgccagagcagtggtttctccagaggcgtctctaaatatcgcggcgtcgctaggcatcaccacaacggaagatgggaggctcggatcggaagagtgtttgggaacaagtacttgtacctcggcacctataatacgcaggaggaagctgctgcagcatatgacatggctgcgattgagtatcgaggcgcaaacgcggttactaatttcgacattagtaattacattgaccggttaaagaagaaaggtgttttcccgttccctgtgaaccaagctaaccatcaagagggtattcttgttgaagccaaacaagaagttgaaacgagagaagcgaaggaagagcctagagaagaagtgaaacaacagtacgtggaagaaccaccgcaagaagaagaagagaaggaagaagagaaagcagagcaacaagaagcagagattgtaggatattcagaagaagcagcagtggtcaattgctgcatagactcttcaaccataatggaaatggatcgttgtggggacaacaatgagctggcttggaacttctgtatgatggatacagggttttctccgtttttgactgatcagaatctcgcgaatgagaatcccatagagtatccggagctattcaatgagttagcatttgaggacaacatcgacttcatgttcgatgatgggaagcacgagtgcttgaacttggaaaatctggattgttgcgtggtgggaagagagagcccaccctcttcttcttcaccattgtcttgcttatctactgactctgcttcatcaacaacaacaacaacaacctcggtttcttgtaactatttggtctgataa

SEQ ID NO: 16 - protein, 430 aa, synthetic construct
MKKRLTTSTCSSSPSSSVSSSTTTSSPIQSEAPRPKRAKRAKKSSPSGDKSHNPTSPASTRRSSIYRGVTRHRWTGRFEAHLWDKSSWNSIQNKKGKQVYLGAYDSEEAAAHTYDLAALKYWGPDTILNFPAETYTKELEEMQRVTKEEYLASLRRQSSGFSRGVSKYRGVARHHHNGRWEARIGRVFGNKYLYLGTYNTQEEAAAAYDMAAIEYRGANAVTNFDISNYIDRLKKKGVFPFPVNQANHQEGILVEAKQEVETREAKEEPREEVKQQYVEEPPQEEEEKEEEKAEQQEAEIVGYSEEAAVVNCCIDSSTIMEMDRCGDNNELAWNFCMMDTGFSPFLTDQNLANENPIEYPELFNELAFEDNIDFMFDDGKHECLNLENLDCCVVGRESPPSSSSPLSCLSTDSASSTTTTTTSVSCNYLV

SEQ ID NO: 17 - DNA, 1296 bp, synthetic construct
  source 1..1296: mol_type = other DNA
atgaaaaagcggctcaccacttccacatgctcatcctccccgagcagctcagtttcctcctctacaaccacatctagccccatccagtctgaggcgccccgaccaaagagggccaaacgggctaagaagtcctccccgtccggggataagtctcataatcccacgtcccccgcttctactcgccggtcttcaatataccgcggagttacccgccataggtggaccggtcgtttcgaggctcatttgtgggataagtcttcgtggaatagtattcagaataaaaaaggtaagcaagtgtacctcggtgcttatgattcggaggaagccgccgcccacacctacgacctggcagccctgaagtattggggccccgacaccatcctaaactttccagctgaaacgtacaccaaggagctcgaggagatgcaaagggtcacgaaggaggaatacctggcctctcttagacgtcagagcagtggcttctccaggggtgtttccaagtaccgcggagtcgcacggcatcaccacaatggtagatgggaggccaggatcgggcgggtgttcggcaataaatacctgtacctcgggacttacaacacccaggaggaagctgctgcagcttatgacatggcggctattgagtaccgcggcgccaacgcagtgacgaatttcgacatctccaactacatcgaccgtctcaagaaaaagggcgtcttcccgtttccagttaatcaagcgaaccaccaagaaggtatattggtcgaggcgaagcaggaggtcgagacccgggaggctaaggaggaacctcgcgaagaagtgaaacagcaatacgtggaggaaccgccacaagaagaggaggagaaggaggaggagaaggctgagcagcaagaggccgagatcgtaggctactcagaggaagccgcagtggtgaactgctgcattgactcgtcgacaatcatggagatggatcggtgcggtgacaataacgaattggcttggaacttctgtatgatggacacggggttcagtccattcctaactgaccagaacctagcgaacgagaaccccatagaataccccgaactattcaacgagttggcgttcgaagacaatatagactttatgttcgacgacggtaaacatgagtgtctgaacctcgaaaatctagactgctgcgtcgtcggcagggaatcacccccctccagttcctctccgctgtcgtgcctcagtacggactcggcgagctctaccacgaccactacaactagcgtttcatgcaactacttggtctgataa

SEQ ID NO: 18 - DNA, 2079 bp, synthetic construct
  source 1..2079: mol_type = other DNA
atcttctgatgagaataagcaaatccctattttttttgttgaagaaaagaaaagcgtgtccacgtggactggactctgaaattttcctcctcccttctggagtcttcctcatcagaaatcagaagaggagagggtgggcaactcgcagatcgccttctcgtcgcgctcgcgccgcaggggtcgcctgaggtatgcagcttcacctcccccaactttctagggttctaatcgcctctgctcgctcggattatgcgtggtgggtatggctccgattccgccggagtagctggatctgtgtgccccgtgctaatttaggtttggctttcgattcggccgcgcttcggtttgttctcgcgcgtgattgcttcgttcggccatagggctcatctcaggctcgacatgtggacggccacaaacataaaaaatccttgttaaatttacggttcatgtggtaggggggcttttcaggatcggaggtttaggtgatttggagtagcaaaacgattttgctgtgttagagaattcgagaatctgtgccgtcagatgctaattagctggttttgaacaaatgttaagacatcctgatattatttgctgcgtttaatgttcaaactttttgcacgggtgttgctgagctataaaggatatgaggatatagtaccaagttctatcgttttgttctgatacatacaatttgctgtagtttatggcaccatcatactgagatatatatacctgtgcacctttcggcttctgtgcagaacacatagacaatggtgaaggatgaaaaagcggctcaccacttccacatgctcatcctccccgagcagctcagtttcctcctctacaaccacatctagccccatccagtctgaggcgccccgaccaaagagggccaaacgggctaagaagtcctccccgtccggggataagtctcataatcccacgtcccccgcttctactcgccggtcttcaatataccgcggagttacccgccataggtggaccggtcgtttcgaggctcatttgtgggataagtcttcgtggaatagtattcagaataaaaaaggtaagcaagtgtacctcggtgcttatgattcggaggaagccgccgcccacacctacgacctggcagccctgaagtattggggccccgacaccatcctaaactttccagctgaaacgtacaccaaggagctcgaggagatgcaaagggtcacgaaggaggaatacctggcctctcttagacgtcagagcagtggcttctccaggggtgtttccaagtaccgcggagtcgcacggcatcaccacaatggtagatgggaggccaggatcgggcgggtgttcggcaataaatacctgtacctcgggacttacaacacccaggaggaagctgctgcagcttatgacatggcggctattgagtaccgcggcgccaacgcagtgacgaatttcgacatctccaactacatcgaccgtctcaagaaaaagggcgtcttcccgtttccagttaatcaagcgaaccaccaagaaggtatattggtcgaggcgaagcaggaggtcgagacccgggaggctaaggaggaacctcgcgaagaagtgaaacagcaatacgtggaggaaccgccacaagaagaggaggagaaggaggaggagaaggctgagcagcaagaggccgagatcgtaggctactcagaggaagccgcagtggtgaactgctgcattgactcgtcgacaatcatggagatggatcggtgcggtgacaataacgaattggcttggaacttctgtatgatggacacggggttcagtccattcctaactgaccagaacctagcgaacgagaaccccatagaataccccgaactattcaacgagttggcgttcgaagacaatatagactttatgttcgacgacggtaaacatgagtgtctgaacctcgaaaatctagactgctgcgtcgtcggcagggaatcacccccctccagttcctctccgctgtcgtgcctcagtacggactcggcgagctctaccacgaccactacaactagcgtttcatgcaactacttggtctgataa

SEQ ID NO: 19 - DNA, 1978 bp, synthetic construct
  source 1..1978: mol_type = other DNA
agtctagctcaacagagcttttaacccaaattggtacaatagaatacaactttagatcataattctcaaaagaaagagattccttagctattctatctgccactccatttccttctcggcttgtatgcacaagcataaaatcctcaaacttgctaagtagatactttatgtcttggataattggattgagacttgacaagcataactttcatgtaaccaaagacacaagttgctgagaatccacctcaaaaatgatcttcctataattgaatcgggataatgacagcacagcccatctaagagcctccacttctacttccagcacgcttcttacttttaccacagctcttgcacctaaccataacaccttccctgtatgatcgcgaagcacccaccctaagccacattttaatccttctgttggccatgccccatcaaagttgcacttaacccaagattgtggtggagcttcccatgtttctcgtctgtcccgacggtgttgtggttggtgctttccttacattctgagcctctttccttctaatccactcatctgcatcttcttgtgtccttactaatacctcattggttccaaattccctccctttaagcaccagctcgtttctgttcttccacagcctcccaagtatccaagggactaaagcctccacattcttcagatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtccccttcctctatataaggaa

SEQ ID NO: 20 - DNA, 1993 bp, synthetic construct
  source 1..1993: mol_type = other DNA
ctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcag

SEQ ID NO: 21 - DNA, 1701 bp, synthetic construct
  source 1..1701: mol_type = other DNA
gtggtcgagattgtgtattattctttagttattacaagacttttagctaaaatttgaaagaatttactttaagaaaatcttaacatctgagataatttcagcaatagattatatttttcattactctagcagtatttttgcagatcaatcgcaacatatatggttgttagaaaaaatgcactatatatatatatattattttttcaattaaaagtgcatgatatataatatatatatatatatatatatgtgtgtgtgtatatggtcaaagaaattcttatacaaatatacacgaacacatatatttgacaaaatcaaagtattacactaaacaatgagttggtgcatggccaaaacaaatatgtagattaaaaattccagcctccaaaaaaaaatccaagtgttgtaaagcattatatatatatagtagatcccaaatttttgtacaattccacactgatcgaatttttaaagttgaatatctgacgtaggatttttttaatgtcttacctgaccatttactaataacattcatacgttttcatttgaaatatcctctataattatattgaatttggcacataataagaaacctaattggtgatttattttactagtaaatttctggtgatgggctttctactagaaagctctcggaaaatcttggaccaaatccatattccatgacttcgattgttaaccctattagttttcacaaacatactatcaatatcattgcaacggaaaaggtacaagtaaaacattcaatccgatagggaagtgatgtaggaggttgggaagacaggcccagaaagagatttatctgacttgttttgtgtatagttttcaatgttcataaaggaagatggagacttgagaagttttttttggactttgtttagctttgttgggcgttttttttttttgatcaataactttgttgggcttatgatttgtaatattttcgtggactctttagtttatttagacgtgctaactttgttgggcttatgacttgttgtaacatattgtaacagatgacttgatgtgcgactaatctttacacattaaacatagttctgttttttgaaagttcttattttcatttttatttgaatgttatatatttttctatatttataattctagtaaaaggcaaattttgcttttaaatgaaaaaaatatatattccacagtttcacctaatcttatgcatttagcagtacaaattcaaaaatttcccatttttattcatgaatcataccattatatattaactaaatccaaggtaaaaaaaaggtatgaaagctctatagtaagtaaaatataaattccccataaggaaagggccaagtccaccaggcaagtaaaatgagcaagcaccactccaccatcacacaatttcactcatagataacgataagattcatggaattatcttccacgtggcattattccagcggttcaagccgataagggtctcaacacctctccttaggcctttgtggccgttaccaagtaaaattaacctcacacatatccacactcaaaatccaacggtgtagatcctagtccacttgaatctcatgtatcctagaccctccgatcactccaaagcttgttctcattgttgttatcattatatatagatgaccaaagcactagaccaaacctcagtcacacaaagagtaaagaagaaca

SEQ ID NO: 22 - DNA, 960 bp, synthetic construct
  source 1..960: mol_type = other DNA
gaattcatgtgtgagggcaattagtgattgtaaaaataaaattgtgttttgtaaaaaacttttactgtcgaaattatttagggtgatgaaaaaatcagtaaactacgaatgatagcttaaagagtttctatcaaagtgattgaggaatagtttgttgcaaattaaacctctaacaaaatgttttctgttgtggtttttcatctctacaaattttgaattttatgatgaattagaaagatagaatgagttactttagattttaaaaggttgttcaagtttacaaaacagattactagaatcatgattaaaaatttacaagctacatattgtctaaaccaatgatgttgaacataccagatgatagtttttcagtgtttgaacaatcaattggatagtttttatgtttctgcaaaatatgcaaataatcagtgtttttgagtctttgcattttgatttaaaagcaaaaacaactgagtttcaaggttaaattaattacattattcatgagatttatcaggttagtggataaactgacaatggaatcaatgttattgtaaattggtagtgatgttggacttctaatgttactctctatgatgtttcggtcatcaatatcacactatctttacttttatttaaaggaaagatcacacaaataagttatctctattcagaactattaagctgcttccaaaagacttgcaacatgtggtctcgaaatgctttggctgcaatgaaaaaatcatagcaaaagctagtggactagagactgccacataagaatagtaaacgttaaaaccaaaatctcaaaaatccaatgagtaaagagatatagattacttcatagataacaaacgttactcgcaattttcctatataatccaaccctacctaaccattttcaatcactctcactcacaagttagtcaccaaaaaaaaaaaaaaacacaaaaagtttca

SEQ ID NO: 23 - DNA, 991 bp, synthetic construct
  source 1..991: mol_type = other DNA
gatccttattcgtgagatgagtcgggtcgggtcaacgcaccatgcaaatgtcatacttatatactcaaatataacactaatcaaaaaaacaatttttgttacatataagagaaaaagtaatacatttttcataataagtaatgttgacaagtgccccttacttataagggcaaatataatacttttgaggaaaaatgtaatacttttaaatcgaaatgtaaaagtattgtattttcccttaaaagtattacatttacccttataagtaacaaaaattttattcttgattagtattgcatttgagcatataagtatgacatttgtgcatataagtgttacatttacatattgacccgaaccgacccgatccgtctcacggtgagacggtctcacaccaatttttgccaattgtatattatttccttaataacattgcaacttaacgtgcgcaataatgaattgagcaaaagaagcccgtacatgcatctgctccaaagtcccaaatactggtacccgtaaagatcgagcttaggatttgttaatcgggcttttatggatttgtgggtttgataagatattttgataccgggctgggttgaatgaaaaagcccaatatagtctagcagctgctgagaaatgggaatgggactactgacagaaaggatggcgaagggcaaaaataagcaaccatgatcacacagtcatatcctcttgaggatgagataagattaacgagggcctcccacgtggcaccccttttgtggtggcttatatcaatacattgccctgatgtggatattaaggttgtaatgtcatgcaccacataaatccaatggccctgcttcctcaaagattaggttgattaaacgtcgcccgttagattggactcatgtgacaccttatcgttatatataccaagcaatagcacactatcaccaccattacaaagtaaagtagtaagtaatacagagaagagcggcagctgctagca

SEQ ID NO: 24 - DNA, 421 bp, synthetic construct
  source 1..421: mol_type = other DNA
gacggtggcattcagctcccaatttatattcccaacggcactacctccaaaatttatagactctcatccccttttaaaccaacttagtaaacgtttttttttttaattttatgaagttaagtttttaccttgtttttaaaaagaatcgttcataagatgccatgccagaacattagctacacgttacacatagcatgcagccgcggagaattgtttttcttcgccacttgtcactcccttcaaacacctaagagcttctctctcacagcacacacatacaatcacatgcgtgcatgcattattacacgtgatcgccatgcaaatctcctttatagcctataaattaactcatccgcttcactctttactcaaaccaaaactcatcaatacaaacaagattaaaaacatacacctcgagatg

SEQ ID NO: 25 - DNA, 1022 bp, synthetic construct
  source 1..1022: mol_type = other DNA
gctcatgctaagctgcgcaaaatacttcctaatcaaaacagtaacaacgagtaattagcaaaatccgagcagaaaactctcacccacctccgaaattcacgtcttcactaaaattttcgaaaggaatcgatcaataccaacccattacacaaaatacataatcaaaatggcgagaatcgtacctggaaactttgcttcaagtcgcagagagaggaaaaggaagatcgtggagaaaggggtttagggtttaagctcagacttctattggagtaaatgggacggtgtcacattttccgttttggaaatgaactttgggctcacgttatgggctattagatatttgatgggctttctagtaaatacaatataagttattgggcttagtttaaataagcccatgttggaaatatttgacacatgtcttggctactagtgctaaacatgcaaccgaacagttgtcgagacaagtcgcagcatatacaatggatcaaacacgcctagtgtcgccgcgtctcgctcatgtgtcaccttgtttcctcgtttttttttaatttttcataagttcttttgttttatcttcaatacaaatttttggctgtatcttgcaaactcttcgatcatatcgccaatatacgtgaacactggtgatctaatttgttgtgttaattgttaaatttagattctattctccggtttaaaagtgaattatatgtatcatggttaaaacattgtaagtaagatgataataaaatgataaatttagttgatggataacgtgaagcaaaaaatgagatagatacatttgattttgtcgtattttgacatatgcggagagtgagctacgcgcatgaagatcaagagacacttgctcgagctcacagagtgacgtgtaaaaagcttagactgaagtccccatgcaaacctaatcctacgtggctcaaaccacgagctcacttgacaatatataaactcctcctaagtcccgttctcttcatccatctctcacaacaaacaaaaagaaa

SEQ ID NO: 26 - DNA, 250 bp, synthetic construct
  source 1..250: mol_type = other DNA
atatgaagatgaagatgaaatatttggtgtgtcaaataaaaagcttgtgtgcttaagtttgtgtttttttcttggcttgttgtgttatgaatttgtggctttttctaatattaaatgaatgtaagatctcattataatgaataaacaaatgtttctataatccattgtgaatgttttgttggatctcttctgcagcatataactactgtatgtgctatggtatggactatggaatatgattaaagataag

SEQ ID NO: 27 - DNA, 234 bp, synthetic construct
  source 1..234: mol_type = other DNA
aataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

SEQ ID NO: 28 - DNA, 4395 bp, synthetic construct
  source 1..4395: mol_type = other DNA
atggcggaggagagacgacaaaacgcacttgaaatcaggatccaagctgctaagctagctaagaagagggaccatcctacacacaaagccaatggagatgaagacagatatccagagacacttataggtagctttactaaaggactgccgcacgagaaggaaacaggtttattgtctaaccctgctgacttcgctgatttcgttcgagctattaataccggtgctatcaaggacatgaggaggttgaaattgggtatcgatgaagatcctcgttttatcagtggaatcgcgtccaaaggtgacaagcacccttttgctgatacacgagcttgggaatctatggcagcaggactgacttacgatcttgagggaccagatgcccaggcggtcactatgcctccagcgcctaagcttgatagcgacgaactggtgacggagatcacggaatcttattggatggctttattgagagatgttccttttaccgagtttgaacgagatgggaacactgctgccgctgcagctagcatatcccggacacgatgggttcaatataatgagcaaccttcgcaaagacctagcacacttacagacgaagagatagcaagactacgaggaccgtataccaaaaaaaacgtattcaggggtgtcaccaatggggagaatgtaggcccttatttatcacagtttcttctggttggaacaaagggcattggtgatgcccaacaagttagtgatggttatgtacagtatggaggaatgagaatggaccaaagagttcgagtggctgtgcctaaaagagattacatgaccacatgggcttcttggctcgatgttcaaaatgctggtgatcttagaggtagagaaatctatgacgacgacacaccattccgattcataacaacgccacgtgatctggctacatgggtgcattttgatgcattataccaggcttatttgaatgcttgtataatactattagatattaaggctccgttcgatcctcacatccctttccaagcggatgatgatgttgataaacagcaaggattcgcaacgtttggtggtccgcatattctttctctttgcactgaggtggctacaagagcgctgaaagcggttcgttttcagaagtacaacttacacaggaggttgagacctgaggcaattggtggtcttgtcgagaggtttaagaaaacaaatggcgacccaaaattcgcacctgttaaaaaattggttaatgacttagacggtgatatgttgcggcgagttgagcaacataactgtgaacaaaataagctctccgatgacggccatgctagaagggaagactatagcccagagggagagtcttcacaatcatatcttttacctatggcattccctgagggttcccctatgcatccttcatacggtgcgggacacgccacggtggccggtgcatgtgtcactgtgcttaaagcctttttcgatcatgaatatgaacttgatttttgctatgtccctacaaccgatggaaagaggcttgagaaagttaatattaatgaaaaacttactgttgagggcgaacttaataagctttgtgctaacataagtattggtcgtaactgggcgggcgttcattactactctgactatttcgagagtatcaaggttggagaggagatcgctattggtatcttgcaggaacaaaaattgacttatggagaggatttttttatgactctacctaagtttgacggtgagaaaataagaataggaagcggagctactaacttcagcctgctgaagcaggctggagacgtggaggagaaccctggacctatggcccatacatcagaatctgtgaatcctagagatgtttgcattgtgggtgttgcacgtactccaatgggtggctttctcggatctctttcatctttacctgccacaaagcttggatctttagctattgcagctgctttgaagagagcaaatgttgatccagctcttgttcaagaagttgtctttggcaatgttcttagtgctaatttgggtcaagctcctgctcgtcaagctgctttaggtgcaggaatccctaactctgttatctgtactacagttaacaaggtttgtgcatcaggcatgaaagcggtaatgattgctgctcaaagtatccagttagggatcaatgatgtagttgtggcgggtggtatggaaagcatgtctaatacaccaaaatatttggcagaagcaaggaagggatctcgttttggtcatgattctttagtagatggaatgttgaaggatggactatgggatgtctataacgactgtgggatgggaagctgtgcagaattatgcgctgagaagtttcagattacaagggagcagcaagatgactatgcagttcagagttttgagcgtggtattgctgcccaggaagctggcgccttcacatgggaaatcgtcccggttgaagtttctggaggaagaggtaggccatcaaccattgttgacaaggacgaaggtcttgggaagtttgatgctgcaaaattgaggaaactccgtcctagtttcaaagagaatggagggactgttacagctggaaatgcgtctagcataagtgatggtgcagctgcccttgtcctagtgagcggagagaaggctcttcagctaggacttctagtattagcaaaaattaaagggtatggtgacgcagctcaggaaccagagtttttcactactgctcctgctcttgctataccaaaagccattgcacatgctggtttggaatcttctcaagttgattactatgagatcaatgaagcatttgcagttgtagcacttgcaaatcaaaagctactcgggattgctccagagaaagtgaacgtaaatggaggagctgtctccttaggacaccctctaggctgcagtggcgcccgtattctaatcacgttgcttgggatactaaagaagagaaacggaaagtacggtgtgggaggagtgtgcaacggaggaggaggtgcttctgctctagttcttgagctccttggttcaggtgccactaatttttctctcttgaaacaggccggtgacgttgaagagaacccaggtccaatgaagaagcgcttaaccacttccacttgttcttcttctccatcttcctctgtttcttcttctactactacttcctctcctattcagtcggaggctccaaggcctaaacgagccaaaagggctaagaaatcttctccttctggtgataaatctcataacccgacaagccctgcttctacccgacgcagctctatctacagaggagtcactagacatagatggactgggagattcgaggctcatctttgggacaaaagctcttggaattcgattcagaacaagaaaggcaaacaagtttatctgggagcatatgacagtgaagaagcagcagcacatacgtacgatctggctgctctcaagtactggggacccgacaccatcttgaattttccggcagagacgtacacaaaggaattggaagaaatgcagagagtgacaaaggaagaatatttggcttctctccgccgccagagcagtggtttctccagaggcgtctctaaatatcgcggcgtcgctaggcatcaccacaacggaagatgggaggctcggatcggaagagtgtttgggaacaagtacttgtacctcggcacctataatacgcaggaggaagctgctgcagcatatgacatggctgcgattgagtatcgaggcgcaaacgcggttactaatttcgacattagtaattacattgaccggttaaagaagaaaggtgttttcccgttccctgtgaaccaagctaaccatcaagagggtattcttgttgaagccaaacaagaagttgaaacgagagaagcgaaggaagagcctagagaagaagtgaaacaacagtacgtggaagaaccaccgcaagaagaagaagagaaggaagaagagaaagcagagcaacaagaagcagagattgtaggatattcagaagaagcagcagtggtcaattgctgcatagactcttcaaccataatggaaatggatcgttgtggggacaacaatgagctggcttggaacttctgtatgatggatacagggttttctccgtttttgactgatcagaatctcgcgaatgagaatcccatagagtatccggagctattcaatgagttagcatttgaggacaacatcgacttcatgttcgatgatgggaagcacgagtgcttgaacttggaaaatctggattgttgcgtggtgggaagagagagcccaccctcttcttcttcaccattgtcttgcttatctactgactctgcttcatcaacaacaacaacaacaacctcggtttcttgtaactatttggtctgataa

SEQ ID NO: 29 - DNA, 57 bp, synthetic construct
  source 1..57: mol_type = other DNA
gctactaacttcagcctgctgaagcaggctggagacgtggaggagaaccctggacct

SEQ ID NO: 30 - protein, 19 aa, synthetic construct
ATNFSLLKQAGDVEENPGP

SEQ ID NO: 31 - DNA, 63 bp, synthetic construct
  source 1..63: mol_type = other DNA
caatgcactaattatgccttactgaagctagcaggcgatgttgaaagcaatcctggtccttaa

SEQ ID NO: 32 - protein, 20 aa, synthetic construct
QCTNYALLKLAGDVESNPGP

SEQ ID NO: 33 - DNA, 78 bp, synthetic construct
  source 1..78: mol_type = other DNA
ggttcaggtgttaagcaaactcttaattttgatttgctaaagctagcgggcgatgtagaaagcaaccccggaccataa

SEQ ID NO: 34 - protein, 25 aa, synthetic construct
GSGVKQTLNFDLLKLAGDVESNPGP

SEQ ID NO: 35 - DNA, 57 bp, synthetic construct
  source 1..57: mol_type = other DNA
gaaggccgggggtctctattgacttgtggagatgtcgaggagaatccgggaccttag

SEQ ID NO: 36 - protein, 18 aa, synthetic construct
EGRGSLLTCGDVEENPGP

SEQ ID NO: 37 - DNA, 69 bp, synthetic construct
  source 1..69: mol_type = other DNA
ggtagtggtgcgactaactttagtttgctcaaacaggcaggtgatgtagaagagaatccagggccttga

SEQ ID NO: 38 - protein, 22 aa, synthetic construct
GSGATNFSLLKQAGDVEENPGP

SEQ ID NO: 39 - DNA, 69 bp, synthetic construct
  source 1..69: mol_type = other DNA
gttaagcaaactctgaacttcgatttattaaagctggcgggtgacgtagaatcgaatcccggaccatag

SEQ ID NO: 40 - protein, 22 aa, synthetic construct
VKQTLNFDLLKLAGDVESNPGP

SEQ ID NO: 41 - DNA, 1353 bp, synthetic construct
  source 1..1353: mol_type = other DNA
atgaagaagattcttatcgcactaatatcgtttgcttttgcggtttcgtgcaaagctccacaaaaagaagaacctattaacattacccccgaagagcttgacgcttcaatagacagggtgacggaaattatgatccacgatatcttttcccctcccgttgcgagtaggatctttgcctatcccaacgttgccgcctacgaaattgtagccgccaccaatgacaactacaactccttggccggccaattgaacgggctgaccgccataccggaacccgataccactaagaccatcaactacgagcttgcagccgtcgtcgcccatatggagcttagcaaaaggttgattttctcagaagaccgaatggaatccctgcgcgatagcctatacatggtttgggaagggaaaaatcctgttctattctccgattccaaagcctacggcctacaagtggccgaccatataggcgaatggatgaacaaggacaattacgcccaaacccgcaccatgccgaaatttacggtagatgcggacgaccccggccgctggcaacccaccccacctgcctacatggacggtattgaaccccactggaataaaatcaggccatttgtattggattcggcagcacagttcaagcccgttccacctccggcatattcccttgaagaagactccgcgttttataaagaattaaaagaagtctatgacgtaaggaacaaaatcaccgaggaaggcgatagttccgaagaaattcagattgcccgcttttgggattgtaacccttatgtatcggttacccgtggccacttgatgttcgccaccaagaaaataaccccaggtgcgcattggatgggaattgccaaaattgccgcacgtaaaaccaacagtgattttgccaaaacccttttcgcctataccaaggcctcggtagccatggcggatgcctttatcagttgttgggacgaaaagtacagaagcaacctcatccgtccggaaaccgtaatcaaccaacatatagacgacagctggaaaccagtgctacaaacccctccgtttccagagtacaccagcggacatagtgtagtctcaggggcggcatcggttgtactgaccgaggtctttggtgacaatttctcctttgacgacgatacggaagtaccttacggcctacctatccgaagctttaaatcctttaagcaagctgccgacgaagcagcgatcagtcgcatgtacggaggcatacactaccgtgcagctattgaagtaggggtaaaacaaggcagggacctaggtacctttgtcgtaaacaaactacatatgctatccgataagaaagtagcccaaaactag

SEQ ID NO: 42 - protein, 453 aa, synthetic construct
MGSMKKILIALISFAFAVSCKAPQKEEPINITPEELDASIDRVTEIMIHDIFSPPVASRIFAYPNVAAYEIVAATNDNYNSLAGQLNGLTAIPEPDTTKTINYELAAVVAHMELSKRLIFSEDRMESLRDSLYMVWEGKNPVLFSDSKAYGLQVADHIGEWMNKDNYAQTRTMPKFTVDADDPGRWQPTPPAYMDGIEPHWNKIRPFVLDSAAQFKPVPPPAYSLEEDSAFYKELKEVYDVRNKITEEGDSSEEIQIARFWDCNPYVSVTRGHLMFATKKITPGAHWMGIAKIAARKTNSDFAKTLFAYTKASVAMADAFISCWDEKYRSNLIRPETVINQHIDDSWKPVLQTPPFPEYTSGHSVVSGAASVVLTEVFGDNFSFDDDTEVPYGLPIRSFKSFKQAADEAAISRMYGGIHYRAAIEVGVKQGRDLGTFVVNKLHMLSDKKVAQN

SEQ ID NO: 43 - DNA, 1875 bp, synthetic construct
  source 1..1875: mol_type = other DNA
atgaaggggcttgcaggaccagccggtgctatggccgttgtcgcgctcgggcttgtccccggcggagcaatcgggaaatctttgcgacaagagccctctgaaccccgcctaagtggcggcgtggatacggctgcatcgccatcgaaggacaccctgaaagggagtctttcgcgtaagctccaagtcgtcaacgatgatgccctcgatgttagtggcacgcccgcagaaagagctgccaacgcgctgaaccagcggatcgaatttgcggagacggagttcacggcatccgaaggcacgctccaccttaacaacggagaccgctcatccgccgccacgttccacaagtcgctgccgcacgacagcctaggacaggtgaacagcgaggactttgacctcctcatggagtgcatcgctcaaggtgatttcgacacatgcgagctggtgccggccggagacgacggcaggctgtccaaccccctcgggggtatcgccgtcgagatggcgggagccgccggccccgctttgaccctccctccggcctcagcgatcaactccgaggacttggctgctcaaatggcggaacagtactggatggccttgaccagggacgtacctttctctcagtacggcgaggatgaggcgacagtggctgcagcagacaacttggccaccatgcctggttttgccgacattgtcggggtggccgtcgatccggaaaccagaagagcggatccgcagtcgcagcttttccggtcctctgccttcggcgtcgagacagggcccttcatttcccagctactggtgaaggacttcacgattgattctatcactgtgacgcctatgcagaagacgtttgcgcccggagcagactacatgaccgactacgacgaatggctctccatacagaacggtggcagccctgactcggaagcggacctagacgacgaggaccggtacatccgcaactcccgcgacctctctaggctggtggctaccgacaccgtcaacacggaggcgtaccgagccgctctgattcttctcgacccggaccagggagccgatggccgggcagccatcagcgctcccggcttgaacggtccctacgcggacagcagccgccaggccggcttcgtcaactacggcgtgtctcaccttatgaggctcgtcggaaccgccgagctggcccagaagtccgcgtggtaccaaaagtggaacgtgcacatgttcgtacgtccggaggctttcggcggaagcatccacaacgtcctcttgggcaaactcgacgtagagatcgccccctcgcttctcaaaaacacggatttgctagacagggtggctgcacggaacggggaaatcaacgggcggccaggagtactcgaccgcacctacctcctctcccaggccctccccgaggggtcgccaactcacccatcgtaccccgctgggcacgccacccagaacggtgcattcgccacggtgctcaaggccctggtcgggctggagcgtggctctgtctgcttcaatgaccccgtgttccccgacgacgaagggctgacccttctgccctacaccggagacgacggaaacaactgcctaacattcgagggagaaatcaacaagctggccgtcaacgtggcattgggcaggaacatgttgggtgttcactggaggatcgacagcgagttgggtttgctcctcggcgagacggcagctgtgaggatcctgcagcaggaggccgtggcatacccagagaacgcgggatacgagttccgtctgatgtcaggcaagaccatcaggctcgaaaccgacgggacattcttcatcgacgacacgctgtgcagcggggacgcgttcatgggagctgacctgtgctaa

SEQ ID NO: 44 - protein, 624 aa, synthetic construct
MKGLAGPAGAMAVVALGLVPGGAIGKSLRQEPSEPRLSGGVDTAASPSKDTLKGSLSRKLQVVNDDALDVSGTPAERAANALNQRIEFAETEFTASEGTLHLNNGDRSSAATFHKSLPHDSLGQVNSEDFDLLMECIAQGDFDTCELVPAGDDGRLSNPLGGIAVEMAGAAGPALTLPPASAINSEDLAAQMAEQYWMALTRDVPFSQYGEDEATVAAADNLATMPGFADIVGVAVDPETRRADPQSQLFRSSAFGVETGPFISQLLVKDFTIDSITVTPMQKTFAPGADYMTDYDEWLSIQNGGSPDSEADLDDEDRYIRNSRDLSRLVATDTVNTEAYRAALILLDPDQGADGRAAISAPGLNGPYADSSRQAGFVNYGVSHLMRLVGTAELAQKSAWYQKWNVHMFVRPEAFGGSIHNVLLGKLDVEIAPSLLKNTDLLDRVAARNGEINGRPGVLDRTYLLSQALPEGSPTHPSYPAGHATQNGAFATVLKALVGLERGSVCFNDPVFPDDEGLTLLPYTGDDGNNCLTFEGEINKLAVNVALGRNMLGVHWRIDSELGLLLGETAAVRILQQEAVAYPENAGYEFRLMSGKTIRLETDGTFFIDDTLCSGDAFMGADLC

SEQ ID NO: 45 - DNA, 1347 bp, synthetic construct
  source 1..1347: mol_type = other DNA
atgaatatgaatagtaagtcgctactacttctttgtgcgctcgttgtgggcacggcttcctttgctgctgaaaaacaagagccttggagtagtatctataaatctcaactttttcacattacagaggtcatggtgacagatgttgccagcccacctgtcgcagctagaatctatgcctattcctgtcttgccagctatttggtcatgaagcagcatggagctgactttagagaaaaggactttacccaagcttccattcttgactttccagaacttgattccaatcaagagttgagctctcccgaatttgctgcaatctatgccatgcttcgtgttggggaaaatgtaatgccttccggctatcttttaaaagaaaagcagcaaaactggatagatcaagccatcaaatcaaaactcatcaaaaagtctaaagttgatttgcacattcaggaagcagaaaaagtcgtgagtcaagtcatgaaattagcgagtacggatggctaccgcgaattaccagccttgacccgctacagtcccacagaggcagaaggtcgctggtatcctactcctccagcctacatcgaggcgatagaacctgaatggcgtaccattcaaccctttttcctagtctccctcgatgattatgatcccagcccgatggcacctttcagcctggaaccggaaagttcatttcatcagcaaatgatagaggtctacgagactactaagtccttggatgaagagcaaaaactaattgccaatttctgggattgcaatccatttatggtagagttttcgggtcatatggctattggggtaaagaaaatttcaccaggtggacactggatggggattaccggaattgctgctgaaaaagcagaattgaatttggcagaaactgcttatatccatgcccttgtcgggatgacgctacacgatgcatttatttcttgctggaaaaccaaatacgaaaccgatcggattcgccctgaaaccatcatcaacaaaaccattgaccaacgctggcgaccactgcttcagacgccaccttttcctgagtacacttcaggtcattcggtgatttcaagggcagctgccattgttttgacgggttactttggagataatttctcctacatcgatgactcggaaacttattttggtcttcctgagcgtgcatttgattcatttttacaagcttcagaagaggctgccatttccaggctttacgggggtattcacttcagagacgctattgaagagggagttcgccaaggagaaaaaatcggaaaaatgatctggcaagctatcgcagaaaaagaactactgaccaggcaaaatcaataa

SEQ ID NO: 46 - protein, 448 aa, synthetic construct
MNMNSKSLLLLCALVVGTASFAAEKQEPWSSIYKSQLFHITEVMVTDVASPPVAARIYAYSCLASYLVMKQHGADFREKDFTQASILDFPELDSNQELSSPEFAAIYAMLRVGENVMPSGYLLKEKQQNWIDQAIKSKLIKKSKVDLHIQEAEKVVSQVMKLASTDGYRELPALTRYSPTEAEGRWYPTPPAYIEAIEPEWRTIQPFFLVSLDDYDPSPMAPFSLEPESSFHQQMIEVYETTKSLDEEQKLIANFWDCNPFMVEFSGHMAIGVKKISPGGHWMGITGIAAEKAELNLAETAYIHALVGMTLHDAFISCWKTKYETDRIRPETIINKTIDQRWRPLLQTPPFPEYTSGHSVISRAAAIVLTGYFGDNFSYIDDSETYFGLPERAFDSFLQASEEAAISRLYGGIHFRDAIEEGVRQGEKIGKMIWQAIAEKELLTRQNQ

SEQ ID NO: 47 - DNA, 1953 bp, synthetic construct
  source 1..1953: mol_type = other DNA
atgaagggtaccgcaggatccgcccttggcgtggtcgtcatcgccttcgggcttttacccggagctatcggggaatcgctgacacgagagcatgctgaacactacccagccggtggtgtagaatcgagcgccttagacggcagtgctctgcgtcgtctccagcctgtatccgatgatgccttcgacgtcaacggcacgcccgcggaaagagctgccaacgcgctgatggagcggaccgattgggcggagaccgagttcttggcatcggaagacgtgttccacgaaaaccttggagatttttcctcggccgccacgttccacaagtcgcttccacacgacgaactcggacaggtgttcagcttcgatttcgaagaactcatcgagtgtgttgcttcaggagatttcgacacgtgccaggaggtgccggccggaaacgtcgaaggcttcctggtcaaccctcttggtggtctcgccaatgacatggcaggacccgcggggcatgctttaaccatccctgccgctccagcactcgactccgaggacctggctgctcaaattgcggaggtgtactggatggccctgacaagggaggtgccgttttcacagtacggcgaagacccgacaactgtggaggccgcagccagcttggccgccatgccgggtttcgcggatctcaactttgtggcggtcgggccggatggcagccccgacccactgacgcagcttttccggtcttcggccgtcggtgtagagataggaccccatgtttcacagctactggtgaataacttcacgattgactccatcactgtggaggctaagcaaaatacgtttgcgcccggagaagactacatggccgagttcgacgaatggctatccatacagaacggtgaatttcctgtagaaccggagatcctcgaccctgtgccgcggtatatccgcaacggacgcgacctttcgacaatggcagcaaccgacaccatcaatacggaggcgtaccgatccgccctgatccttatcgagcaagacgccatcagccgtttcggtatcaacggcccgtacgtggcagacggtcgtcagagcggtttcgtcaactacggcatctcccatactatgagacttgttggaagcggcgagctgtccatgaggtcctcatggtaccagaagtggaacgttcacctgtatgcccgtccggaggccatcggtggaaccatccacaacgtcctcaatggcgatctcgacataacgttcgccgactcgattatcaacaacgacgagttgttcagtaaggtggaggctaggaacatggagatcaccgggataccatccaccttccttctaccccagaccgtcagggaggggtcaccaactcacccatcgtacccctctggacacgccgtccaaaacggtgccttttcaacgatactcaaggctcttgtcgggctggagaggggattggagtgcttcaacgaccccgtggtcccctctgatgatggattgacccttctgcccttcgacggctgcctgacgttcgagggagaaatcaacaaatatgccgccaacgtcgctcttggcaggaattggatgggtgttcactggaggatggacgctcaagaagggttgctgctcggagaaacggtcgccgtgcggatcctgatgcaggagctcgcggggttcccagagttggagccctacgagttccgtgtcatgtcaggcgagaggaacgtgacggaggcggtgacggatagacgtatcgccaacatgtacctcaagtacgccgggttgcgggattggtggacgcatgatgttcgatccggagaatatcttctccggggggcgctggatatattttcgagccccagccaatcagtgattgaccggttgggctacggtgtccataccgtgtgcggcggtgtcgacgctcggtgtcgacgctcgggccctcgtttttaa

SEQ ID NO: 48 - protein, 650 aa, synthetic construct
MKGTAGSALGVVVIAFGLLPGAIGESLTREHAEHYPAGGVESSALDGSALRRLQPVSDDAFDVNGTPAERAANALMERTDWAETEFLASEDVFHENLGDFSSAATFHKSLPHDELGQVFSFDFEELIECVASGDFDTCQEVPAGNVEGFLVNPLGGLANDMAGPAGHALTIPAAPALDSEDLAAQIAEVYWMALTREVPFSQYGEDPTTVEAAASLAAMPGFADLNFVAVGPDGSPDPLTQLFRSSAVGVEIGPHVSQLLVNNFTIDSITVEAKQNTFAPGEDYMAEFDEWLSIQNGEFPVEPEILDPVPRYIRNGRDLSTMAATDTINTEAYRSALILIEQDAISRFGINGPYVADGRQSGFVNYGISHTMRLVGSGELSMRSSWYQKWNVHLYARPEAIGGTIHNVLNGDLDITFADSIINNDELFSKVEARNMEITGIPSTFLLPQTVREGSPTHPSYPSGHAVQNGAFSTILKALVGLERGLECFNDPVVPSDDGLTLLPFDGCLTFEGEINKYAANVALGRNWMGVHWRMDAQEGLLLGETVAVRILMQELAGFPELEPYEFRVMSGERNVTEAVTDRRIANMYLKYAGLRDWWTHDVRSGEYLLRGALDIFSSPSQSVIDRLGYGVHTVCGGVDARCRRSGPRF

SEQ ID NO: 49 - DNA, 1843 bp, synthetic construct
  source 1..1843: mol_type = other DNA
atgaaggggcttgcaggaccagccggtgctatggtcgttgtcgcgctcgggcttgtccccggcggagcaatcgggaaatctttgcgacaagagccctctgaaccccgcctcagtggcggcgtggatacggctgtatcgccatcgaaggacaccctgaaagggagtctttcgcgtaatcttcaagtcgccaacgatgatgccctcgatgttaatggcacgcccgcagaaagagctgccaacgcgctggcccagcggatcgaattggcggagacggagttcgcggcatccgaaggcttgttccacgtcaacaacggagaccgctcatccgccgccacgttccacaagtcgctgccgcacgacagcctaggacaggtggacagcgcggcctttgaagccctcacagagtgcatcgctcaaggtgatttcgacatatgcgagctggtgccggccggagacgttggcaggctgtccaaccccctcgcgggtatcaccgtcgagatggccggagccgccggctccgccttgaccctccctccggcctcagcgctcgactccgaggacttggctgctcaaatggcagaactgcactggatggccttgaccagggacgtacctttctctcagtacggcgaagatgaggcgactgtggctgcagcagacaacttggccaccatgcctggttttcaaaacatggtcggggtggccgtcgatcgggatggcagagcggatccgcagtcgcagcttttccggacctctgccttcggcgtcgagacagggcccttcatctcccagctactggttcaggacttcacgattgattccatcactgtggcgcctatccagaagacgtttgaacccggagcagactacatggccgactacgacgaatggctcttcatacagaacggtggcgtgcctgaccatgacgatgtgctctttgacgacgtgaaccggtacatccgcaactcccgcgacctctctaggctggtggctgccgacaccgtcaacacggaggcgtaccgagccgctctgattcttctcgagcagggagccatcagcggtcccggctccaacggtccctacgcgggcagcagccgccaggccggcttcgtcaactacggcgtgtcccaccttatgaggctcgtcggaaccgccgagctgtcccagaagtccgcgtggtaccagaagtggaacgtgcacatgttcgtacgtccggaggctttcggcggaaccatccacaatgtcctcttgggcaaactcaacgtagacataaacccctcgcttctcaaaaacacggagttgctagagagggtggctgaacggaacggggtaatcaacgggcggccaggagtactcgaccgcacctacctcctctcccaggccgtcatcgaggggtcgccaactcacccatcgtaccccgctgggcacgccacccagaacggtgcattcgccacggtgctcaaggccctggtcgggctggagcgtggctctgactgcttcagagaccccaaggtccccgacgacgaagggctgacccttctggacttcaccggggactgcctaacattcgagggagaaatcaacaagctggccgtcaacgtggcattcggcaggaacatgtgcggtgttcactggaggatcgacagcgagcagggtttgctcctcggcgagatggcagctgtgaggatcctgcagcaggaggccgtgacattcccagagaacgcgggatacgagttcaatctgatgtcaggcgagaccatcaggctcgaaaccgacgggacattcttcatcaacgacaggctatgcagcggggacgcgttcatgggagctgacctgtgctaat

SEQ ID NO: 50 - protein, 613 aa, synthetic construct
MKGLAGPAGAMVVVALGLVPGGAIGKSLRQEPSEPRLSGGVDTAVSPSKDTLKGSLSRNLQVANDDALDVNGTPAERAANALAQRIELAETEFAASEGLFHVNNGDRSSAATFHKSLPHDSLGQVDSAAFEALTECIAQGDFDICELVPAGDVGRLSNPLAGITVEMAGAAGSALTLPPASALDSEDLAAQMAELHWMALTRDVPFSQYGEDEATVAAADNLATMPGFQNMVGVAVDRDGRADPQSQLFRTSAFGVETGPFISQLLVQDFTIDSITVAPIQKTFEPGADYMADYDEWLFIQNGGVPDHDDVLFDDVNRYIRNSRDLSRLVAADTVNTEAYRAALILLEQGAISGPGSNGPYAGSSRQAGFVNYGVSHLMRLVGTAELSQKSAWYQKWNVHMFVRPEAFGGTIHNVLLGKLNVDINPSLLKNTELLERVAERNGVINGRPGVLDRTYLLSQAVIEGSPTHPSYPAGHATQNGAFATVLKALVGLERGSDCFRDPKVPDDEGLTLLDFTGDCLTFEGEINKLAVNVAFGRNMCGVHWRIDSEQGLLLGEMAAVRILQQEAVTFPENAGYEFNLMSGETIRLETDGTFFINDRLCSGDAFMGADLC

SEQ ID NO: 51 - DNA, 1365 bp, synthetic construct
  source 1..1365: mol_type = other DNA
atgggttctatgaaaaagatcctgatcgcgttgatctctttcgcgtttgcagtatcctgcaaagcacctcaaaaagaggaaccgataaacatcactccggaggaattggatgcgtctattgaccgggtgacagagatcatgatccatgatatcttcagcccacctgttgcttcccgaattttcgcatatccaaacgtggcggcgtacgaaattgtcgctgcaactaatgataattacaactcacttgctggtcaacttaacggactaacggcaataccagagccagatacaaccaaaaccatcaattacgagctggctgctgtcgttgctcatatggaactctctaagcgtctcattttctcagaggaccgtatggaatcccttagagatagcttgtacatggtttgggagggcaaaaatcctgttctgttcagcgactccaaggcctatgggttacaagtcgctgaccacatcggtgagtggatgaacaaggacaactatgcacaaacccgtacaatgccgaaattcacggtggatgccgatgatccaggtaggtggcagccaaccccgcctgcttatatggatgggattgaaccacattggaacaaaatcagaccatttgtacttgattctgccgcgcagttcaagccagtcccaccgcccgcttattcattggaagaagatagtgctttctacaaggaattgaaggaggtttatgatgtgcggaacaagattactgaggagggagattcatctgaggagatacagattgctagattctgggattgtaatccatacgtgtcagtgactagaggacacttgatgttcgcaactaaaaaaatcacaccaggtgctcattggatgggaatcgctaaaattgcggccagaaagacaaattcggacttcgctaaaactctctttgcatacactaaagcctctgtggcaatggctgatgcatttatctcttgttgggatgagaagtacagaagtaacttgatcaggccagagacagtaatcaaccaacacattgatgatagctggaaacctgttttgcagacaccaccattccccgaatacacgtctggccatagcgtcgtgtcgggtgccgctagtgtcgttctgacagaggtgtttggagataatttcagtttcgatgatgacacggaggtcccatatgggctgccgattagatcgtttaaaagctttaagcaggctgctgacgaagccgccattagcagaatgtacgggggaatccactatcgtgcagctattgaggttggcgttaaacagggaagggatttggggaccttcgtagtgaataaattacatatgttatctgataagaaggtcgcgcagaattaatag

SEQ ID NO: 52 - DNA, 1365 bp, synthetic construct
  source 1..1365: mol_type = other DNA
atggggtctatgaagaagatccttatagctctgatctcgttcgcgttcgctgtctcgtgcaaggccccgcaaaaggaggagccgataaacataactccagaagagctggatgcttccattgatagggtgacggagatcatgatccatgacatattctccccgccggtggcttccaggattttcgcgtatccaaacgtggcagcgtacgagatagtggcagcaacaaatgataactacaactcgctcgctgggcaactgaacggccttacagcaattccagagccggataccaccaaaactattaattacgagctggcagctgttgtcgcccacatggagctgagcaaaagactgatcttctcggaggatcgcatggagtcactacgcgacagtctctacatggtctgggaaggcaagaacccggtgctgttcagtgactccaaggcatacggccttcaagtcgccgaccatataggtgagtggatgaacaaagacaattacgcgcagacgagaacgatgcccaagttcactgttgacgcggatgatcctggccggtggcagccaaccccgcctgcgtacatggatggcatcgagccccattggaataaaattagaccgttcgtgctggactcggcggcacagtttaaacccgttccgccgcctgcgtacagcctggaggaggattccgcgttctacaaagagctgaaggaggtctacgacgtgcgcaataagataacagaggagggagattccagcgaggagatccaaatagccagattttgggattgcaatccttacgtgtcggttaccagggggcacctcatgttcgcgacgaagaagattacgccaggcgcgcattggatgggaatcgcgaagatagcggcgaggaagacgaactcggatttcgccaagacgctgttcgcgtacacaaaggcgtcggttgccatggcagatgcttttatatcctgttgggatgagaagtacaggtcaaatctgattaggcccgagacagtcatcaatcaacacattgacgactcatggaaaccggtcctccagacacccccattccccgagtacacctctggacattctgtagtatctggtgctgcgtcagttgtcctcacagaggtgttcggtgacaatttctcgttcgatgatgacaccgaggtgccgtacggcctgcctattcggtcttttaagtctttcaagcaagctgccgatgaagccgccatatcgcgtatgtacggtggaatacactacagagccgcaattgaagtgggcgtgaagcagggcagggatttgggtaccttcgtcgtaaacaagctccacatgttgagcgacaagaaggtagcccagaactaatag

SEQ ID NO: 53 - DNA, 1878 bp, synthetic construct
  source 1..1878: mol_type = other DNA
atgaaagggcttgcgggacccgctggtgctatggcagtcgttgcactaggcttggttcctggcggagcgatagggaagtcgcttcgtcaggagcctagtgagccaagactttctggaggagttgataccgcagcatctccaagcaaagacactctcaaagggtcacttagtcgtaagctacaggtagtgaacgatgatgctctagacgtttcaggtaccccagcagaaagggccgcgaatgctttaaaccagcgtattgaattcgccgaaacagaattcacagcatctgaaggtactttacatcttaataatggcgacagatcttcagctgctacatttcataagtcactcccacatgatagtttaggccaagtgaatagtgaagatttcgatttgcttatggagtgcattgctcagggagattttgatacttgcgaacttgttccggcgggagacgatgggcgactatctaatccgttggggggtatcgctgtcgaaatggcaggtgcggctggacctgcactcacgctacctcctgcatccgcaatcaatagtgaggatctagcagctcaaatggctgaacagtactggatggccctaacaagagatgtgccatttagtcaatacggtgaagatgaggcaaccgtcgctgcagctgataacttggcaactatgccaggttttgctgatattgttggtgtggctgtggatccagaaactaggcgggctgatcctcagtctcaacttttcagatccagcgctttcggagttgagactggccctttcatctcacaacttttggtgaaagattttactatagatagtattacggttacccctatgcaaaagacctttgctcctggagcggattacatgactgattacgatgagtggctttccatccaaaatggaggatccccagactccgaagcggatttggatgatgaggacagatatatacgaaatagccgagatctcagtagactcgttgcgaccgacactgttaatacagaggcttatagggctgctttgatactcttagatcccgatcaaggagccgatggacgtgctgccatcagcgcaccaggccttaacggaccttacgctgattcgtctagacaggccggttttgtcaactacggagtctcccatctgatgcggttggtaggtactgccgagcttgctcaaaaatctgcgtggtatcagaaatggaatgtgcatatgtttgttcggcctgaggcatttggaggaagtatacataacgttttattgggaaaactcgatgttgagatagcaccttccctacttaaaaatactgatttgcttgaccgtgttgctgcacgtaacggagagatcaacggaaggcctggggtcctagatcgaacatatcttttaagtcaagctctgcctgagggtagcccaactcatccatcttatcctgcaggacacgccactcagaacggtgctttcgctaccgttcttaaggctttggttggattggaaagaggctccgtttgctttaacgaccccgttttccctgacgatgaaggactgacactccttccttatacgggagatgatggaaataattgtcttacatttgagggagaaattaataaactcgctgtgaacgttgctttgggaaggaacatgcttggggtccattggagaatagatagtgagttgggtcttctattgggtgaaaccgccgctgttaggattttacagcaagaagcagttgcttatccagaaaatgctggatatgaatttagattgatgtctggaaaaactataagactagaaacagatggtacgttttttattgatgacactttatgcagtggcgatgcttttatgggcgctgatctttgctaatag

SEQ ID NO: 54 - DNA, 1878 bp, synthetic construct
  source 1..1878: mol_type = other DNA
atgaagggcctggctggtccagcaggcgccatggccgtggtcgcgcttggtctggttccaggcggggcaattgggaagagcctccgccaggagccttctgagcctcgcctcagtgggggtgtcgacaccgcggcttcaccttccaaagatacgctcaaaggatcactgtcccgcaagcttcaggtggtcaacgatgacgctctggatgtgagcggcacacccgccgagagggcagccaatgcgttgaaccagagaatagagttcgccgaaaccgagttcacggcgtctgaggggacgctacacctgaataacggcgaccggtcttccgcggctacgttccacaagagtctcccacacgactcgctgggacaagtgaactccgaagattttgaccttttgatggagtgtatcgcccaaggagattttgacacttgcgagctggttccggccggggacgatggcaggttgagcaatcctctcggcgggatcgccgtagagatggctggggcggcaggtcctgccctcacgctgccccctgcaagcgcgattaattccgaagatctcgccgcccagatggctgagcaatactggatggcgctgacacgggacgtgccgtttagtcagtacggggaagatgaggccacagttgccgcggccgacaatcttgcaaccatgccgggcttcgcagatattgtcggagttgctgtcgacccggaaactaggcgcgccgacccacaatcgcaactgtttaggtcctcagcattcggtgtcgaaaccgggcccttcatcagccaactcctcgtaaaggacttcaccatcgatagcatcaccgtcacccccatgcagaagacttttgcccctggcgccgactacatgaccgactacgacgaatggctgagtatccagaacggcgggagcccagattccgaagccgatctcgatgatgaggatcgctacatcaggaattcccgcgacttgagtcgcttggtggcgacagatacggttaatacagaggcctatagggcggctctcattttgctggaccccgaccaaggtgctgacggccgtgccgctattagtgcgcctggcttgaatggcccctacgccgacagctccaggcaggccgggtttgtcaattacggcgtctcgcatttgatgcgactcgttggaaccgcggagctggctcagaagtcagcgtggtatcagaagtggaacgtccacatgttcgtcaggccggaggcctttggaggcagtatccacaacgtgctcctcggcaagctcgacgtggaaatcgctccatctcttcttaagaatacagatctcctcgatcgcgtggccgcgcgcaatggcgagatcaacggtaggcccggcgtcttggatcgaacctacttgctcagtcaagcgctgccggaaggctcacccacacatccgtcgtacccggctggccacgcgacccagaatggggcgttcgccaccgtgctgaaggccctagtaggcctagagcgaggtagcgtgtgctttaacgatccagtcttcccggatgatgaaggcctgaccctcttgccatacacgggcgacgatggtaacaactgcctgacgttcgagggcgagataaataagctcgccgtcaacgtcgcactgggtcggaacatgctaggcgttcactggcgcatcgatagtgaactgggactgctgctcggcgaaaccgccgccgtgaggatccttcagcaggaagccgtggcttatccagagaatgctggctacgagttccgcctgatgagcggaaagaccattagactggagacggacggcacattcttcatagatgacacgttgtgctccggagacgcctttatgggtgcggacctgtgctaatag

SEQ ID NO: 55 - DNA, 1350 bp, synthetic construct
  source 1..1350: mol_type = other DNA
atgaacatgaattccaaatcactgttattgctatgcgcactagtagtcggtactgcttcttttgctgcagaaaaacaagagccatggtcttcgatttataagtctcaactcttccatataacggaagtcatggtgactgatgttgcctcacctccagtagcggcgaggatttacgcttattcgtgtcttgcatcgtacttagttatgaaacaacatggagcagattttagagaaaaggatttcacgcaggccagcatattggattttccagagttagactctaaccaagaactaagttcacctgagtttgcggcaatttatgctatgttaagagttggtgaaaacgtgatgccttccggatacctgctcaaggaaaaacaacaaaattggatagaccaggcaatcaagagcaaactgattaaaaaatcaaaagtggatttgcatattcaagaagctgaaaaggtggtatctcaggtgatgaagctcgctagtaccgacggctatcgtgaactccctgcattgaccagatattcacctacagaagctgagggcaggtggtatcctacaccgccagcctacatagaagctatagagcccgagtggcgtactatccaaccatttttcttggtctcgttggatgattacgatcctagtcctatggcaccgttctctctagagcctgagtcatcattccatcagcagatgattgaagtctacgagacgacaaagtcattggatgaggagcaaaaactcatagctaatttctgggactgtaaccccttcatggtggaattttctggacacatggccattggagttaaaaagattagtccaggggggcactggatgggaatcacaggaatcgcggctgaaaaggcagaattaaatttagcagagaccgcttatattcatgctctggttggtatgactttacatgatgcgtttatttcgtgctggaagactaagtatgaaacggatcgtattcgtccggagactattatcaataaaaccatcgatcaaagatggagacctcttctgcagactccgccattccctgaatatacctcaggccattcagtcatttctagagctgcagcaatagttttgaccggctacttcggtgataattttagttacatagatgattctgagacctactttgggttaccagaacgggcattcgactcattccttcaggcgagcgaggaggcggcgatttccaggctatacgggggtattcattttagggacgctatagaggaaggtgttcgtcaaggtgaaaagatcggtaagatgatatggcaagcgatcgcagagaaggagctgctaactcgtcagaaccaataatag

SEQ ID NO: 56 - DNA, 1350 bp, synthetic construct
  source 1..1350: mol_type = other DNA
atgaatatgaactctaagagtctgctgctcttgtgcgccctggtggtgggcacagcaagctttgcggcggagaagcaggagccctggtcgtcaatttacaaatcgcagctgttccatatcacggaggtcatggttactgacgtcgcgtctccccctgtggctgcgcggatctacgcatacagctgtctcgcctcctatcttgttatgaagcagcacggggctgactttcgcgagaaggatttcacgcaagcctccattctggacttcccagaacttgacagtaaccaagagttgtcttcaccagagttcgccgcgatatacgctatgcttcgcgtgggcgagaatgtcatgccgtccgggtacctcttgaaagaaaagcaacaaaattggatcgaccaagccattaagtcaaagctgatcaagaaatcaaaagtcgacctacacatccaggaggcggagaaggttgtgagccaagtgatgaagctcgcctcgactgatggctaccgtgagcttccggccctaactaggtatagcccgacagaggccgagggtcgctggtaccctacaccaccggcctacattgaggcgatcgaacccgagtggagaacaattcaaccgtttttcctcgtgtctcttgatgactacgacccttctccgatggcaccattttcgctggaaccagagtcaagctttcatcagcagatgatagaggtctacgagactaccaagtcgcttgacgaagagcaaaaactcatcgcaaacttttgggactgcaatcctttcatggtcgagttctcgggccatatggcaatcggtgttaaaaagatctctccggggggtcactggatgggcataactggtatcgcggccgagaaggcggaattgaatctggccgagacagcgtacatacatgcgctcgtaggtatgacccttcatgacgctttcatctcttgttggaagaccaagtacgaaacagaccggatcaggccggaaacgatcataaataagaccatcgaccagcgctggcgcccgctgttgcagactcctccgtttccagaatacacgagtggccactccgtcatctcccgcgctgccgccatcgtgcttaccggctactttggagataatttcagctatattgacgattcggagacgtacttcggcttgccggagcgagctttcgactcattcttgcaagcatccgaggaagcagccatcagtcgcttgtacggcgggattcacttccgcgacgccatcgaggaaggtgttcgccaaggcgagaagatcggaaaaatgatctggcaggcgatcgccgaaaaggaactccttacccgacagaaccagtaatag

SEQ ID NO: 57 - DNA, 1956 bp, synthetic construct
  source 1..1956: mol_type = other DNA
atgaaaggaactgctggatccgctttgggggtagtggtcatcgcattcggacttctccctggagcaatcggggaaagccttaccagggaacatgctgaacattatcccgcaggaggagttgagtcaagcgctttggacgggtctgctctccgaagacttcagccagtcagtgatgacgcatttgatgtgaatggaactccggctgagagagctgctaatgctcttatggagcggaccgattgggcagagaccgaattcctggcttcggaggatgtgtttcacgaaaacctgggtgatttcagctccgctgctacattccataagagcctaccacacgatgaacttggtcaggttttttcgtttgatttcgaagaactaattgaatgtgttgcttctggagattttgatacatgtcaggaggtccccgcagggaacgttgagggatttttggtcaacccattagggggtctggctaatgatatggccggtcctgcaggtcatgcccttactattcctgcagcgccggcactggactcagaagatctggcggctcagatagctgaggtttactggatggctctgacacgtgaagttccattctcacagtacggtgaggatccgacaaccgtcgaagcagctgcgagtcttgcggctatgcctggatttgcagatttgaattttgttgcagtaggacctgatggctcgcctgatccactaacacaactatttagaagttctgcagtaggtgttgagattggccctcatgtcagtcaactccttgtgaataactttacgatcgattctattactgtagaagcgaagcaaaacacttttgcccctggagaagattacatggctgaatttgatgaatggttgtcgatccaaaacggtgagttccctgttgaacctgaaattctagaccctgttcctagatacatcaggaacggtcgagatcttagtactatggctgcgactgatacaattaatactgaagcctacaggtcagcactcattttaatagagcaggacgctatctcacggttcggaataaacgggccttacgtggctgatggaagacagagtgggtttgtcaactacggtatctctcataccatgaggttggttggtagtggagaactttccatgcggtcatcttggtaccaaaagtggaatgtgcatctttatgcacggcccgaagccatcggaggaaccatacacaacgtccttaacggggacttggatattaccttcgctgactcgattattaataatgatgagttattctctaaagtcgaggcgcgtaatatggagatcactggtatcccttctacattccttttgcctcaaactgtgagagaaggcagcccgactcacccgtcctatccttctggtcatgctgtgcaaaacggtgccttttccacaattctaaaagccttggtcggattggaacgtggattagaatgctttaacgatcctgttgttccatcagatgatggattaaccttgcttccgttcgatggatgcttaacgtttgagggtgaaataaacaagtacgccgccaacgtcgcgctaggacggaattggatgggtgttcattggagaatggatgctcaggagggtctcttgctaggagagacagttgctgtcagaatattaatgcaggagttagctggttttccggaattagaaccatacgaattcagggttatgagcggagaaaggaatgtgaccgaggctgtgaccgacaggaggatcgcgaacatgtatctgaaatacgctggattgagagactggtggacacatgatgtccgttccggagagtaccttctgagaggggcactcgacattttctcgagtccgtcacaatcagtcatcgaccgacttggttacggtgttcataccgtttgcggtggtgtcgacgctcgatgcaggaggtcaggtccacgtttctaatag

SEQ ID NO: 58 - DNA, 1956 bp, synthetic construct
  source 1..1956: mol_type = other DNA
atgaaggggaccgccgggtcagcgctgggggtcgtagtaattgcgtttggcctgctacctggggcgatcggtgaatcactgactcgcgagcacgccgagcactaccccgccggcggcgtcgaaagttctgcactggacgggtccgcgctccggagactccagccagtctccgatgacgccttcgatgttaacggtacgcccgccgaacgggcggccaatgctcttatggagcgcacagactgggctgaaacggagttcctcgcctccgaggacgtctttcacgaaaatctgggcgactttagcagcgcagccacgttccacaagagcttgccccacgatgagctcggtcaagtgttctcgttcgatttcgaagaactgatcgaatgcgttgcctccggtgacttcgacacgtgtcaggaggttccggccggcaatgtggagggcttcctggttaaccctttggggggcttggccaacgatatggcaggaccagcaggccatgcacttacgatccccgccgcgccagcgctagattccgaggacctcgccgctcagattgccgaggtctactggatggccctgacccgcgaagtaccgttctcacaatacggggaagacccgacaacagttgaagcagcagccagtctcgccgctatgccggggtttgcagatttgaacttcgttgccgtcggcccggacggcagcccggacccactcacccagctatttcggtctagtgctgtaggagttgagatcggccctcacgtaagccagctgctcgttaataacttcacaattgactcaataacggtggaggcaaagcagaacacattcgcccccggcgaggactatatggcggagttcgacgagtggctaagcatccagaacggtgaattccctgttgagccggagatccttgatcccgtaccaagatatatccggaatggccgggacctttctaccatggcggccaccgatacgatcaacacggaggcctaccgctctgccctcatcctcattgaacaagatgccatctcaaggtttggcattaatgggccatatgttgcggacgggcgccagagtggattcgtcaactacggcatctctcacacaatgcgtctggtgggatccggcgagctgagtatgcggtcctcatggtatcagaagtggaacgtccatctctacgccaggcctgaggctattggcggtactattcacaacgtactcaacggcgatttggacatcaccttcgcggactccatcatcaacaacgacgaacttttctccaaagtggaagccaggaacatggagattactggcattccctccactttcctgctcccgcagacggttcgtgagggaagccctacacatcctagctatccgtccggccacgcagttcagaacggcgcgttcagtaccattttgaaagcactggtgggcttggagaggggactcgaatgcttcaatgatccagttgtccctagcgatgatggcctgaccctcctgcctttcgacggctgcctcaccttcgaaggcgaaattaataagtatgccgccaacgtggctctcggccgcaactggatgggtgtccactggcgaatggatgcccaagaagggctcctcctgggggagacggtggccgttcggatcctaatgcaggaactggcaggatttcctgagctggagccctatgagttccgggtcatgtccggggaacgcaacgtgactgaggcggtcactgaccgtcggattgccaatatgtatctcaagtacgccgggctccgggactggtggactcacgatgtgaggtccggagagtacctcctcaggggcgcgctggatattttttcaagtcccagccagtccgtcatcgacagacttggatacggcgtccacactgtctgtgggggggtggatgccagatgccgaagaagtggcccgcgcttttaatag

SEQ ID NO: 59 - DNA, 1845 bp, synthetic construct
  source 1..1845: mol_type = other DNA
atgaagggtttggccggaccagcgggagctatggtcgtggttgcgctaggacttgttccgggaggagcaatcggaaaaagcttaagacaagagccaagcgagccacggctatcaggcggagtcgatactgctgtaagcccttccaaagatactctgaagggttcactttcaagaaacttgcaggtcgcaaacgatgatgctttggacgttaacggaacaccggcagaaagggcggcaaatgctctagctcaaaggatcgagcttgctgaaaccgagttcgctgcctccgagggactcttccacgtcaataacggcgacaggtctagcgccgctacttttcataaatccttaccacacgattctttgggtcaggtcgattctgctgctttcgaggctttgactgagtgcattgcccagggtgattttgatatctgcgaactcgtacctgccggggatgtaggtaggttgtcgaatcccctagcaggtataactgttgagatggctggtgcggctggttctgcccttacactccctcccgcaagtgctctggattccgaggatttagcggctcagatggctgagctacactggatggctcttacaagagatgtacctttttcacagtatggggaggatgaagccacagtcgctgctgcagataatcttgcgacgatgccaggattccaaaacatggttggcgtggcagtcgatcgggatggacgagcggacccccaatcacaactattcagaacttccgcttttggtgtcgagacgggtcctttcatctcacaacttttggtccaagatttcactatagacagcattactgttgctcctattcaaaaaacctttgaaccaggggctgattatatggctgactacgatgaatggctgttcatacagaacggaggagtaccggaccatgatgacgttcttttcgacgacgttaatagatatattaggaactctagagatcttagtagacttgtcgctgctgatacggtgaatacggaggcttatagagccgcacttattcttcttgagcaaggagcaatatctggtccaggatcgaatggtccttacgcaggatcttcaagacaagccggttttgtgaactacggagtttctcatcttatgcgattggtgggtaccgcggagttatcacaaaaaagtgcgtggtatcaaaaatggaatgtgcacatgttcgtaagacctgaggcgtttggtggtaccattcacaacgtgctccttggaaaacttaatgttgatataaatccctcattgctaaaaaatacagaactgttggaaagagttgcagagcggaatggtgttataaacgggcggccaggtgtgctcgatcgaacatacctcttatctcaagccgtaattgagggatcacctactcacccaagttatccggcgggccacgcaacccaaaatggagcttttgctactgtgctgaaagcattggtaggcctagagaggggctccgattgttttagagatcctaaggttccagatgatgaaggacttacactccttgactttactggagattgtctgacatttgagggagaaattaacaaactcgcggttaatgttgcatttggtcgaaatatgtgtggagtgcactggagaatagattcagaacaaggcctcctcttgggtgaaatggctgccgttcgtatcttacaacaagaagctgtaacatttccagaaaatgccggctatgaattcaatcttatgagcggtgagactatcaggcttgagaccgacggaactttcttcatcaatgatcgtctgtgcagtggtgatgctttcatgggagccgatttatgctaatag

SEQ ID NO: 60 - DNA, 1839 bp, synthetic construct
  source 1..1839: mol_type = other DNA
atgaaaggcctagcaggccccgccggggcgatggttgtggtggccttgggtctcgtgccgggcggcgcgatcgggaaatccctccggcaggaacctagtgagccacggcttagcggaggcgtggacaccgcagtcagtccatcaaaggacaccctaaagggctccctgtccagaaatctgcaagtcgcgaacgatgacgccctcgatgttaacggtacaccggcggaacgggccgccaacgctctggcacagcgtatagagcttgcagaaactgagttcgcggcgagcgagggtcttttccatgtgaacaacggcgacaggtcttctgccgctacctttcacaaatctctccctcacgattccctcggtcaggtcgactcggcggcgttcgaggctctaaccgagtgtattgcacagggcgacttcgacatatgtgagttggtaccagccggggacgttgggaggctcagcaacccactggccggtataacggtcgagatggccggcgccgcaggcagcgctcttactctgcccccagccagcgccctcgactcagaagacctcgcagctcaaatggctgaactgcactggatggctctcacaagggacgtgcctttctcgcagtatggcgaggacgaggcgacggtcgctgcagcagacaacctggcaacgatgccaggctttcagaacatggtcggcgttgcggtcgatagagatggtagggccgatccgcaatctcaactcttcaggacttcagcgttcggcgttgaaacaggcccttttatctcccagctgcttgtgcaggacttcactatagactccatcacggtcgcgcctattcagaaaaccttcgagccgggagccgattacatggccgactacgacgagtggctcttcatacagaacgggggggttccagaccacgatgacgtattgtttgatgacgttaaccgctacattcgcaactcgagggatctctcgcgcctggtcgctgccgatacggtcaataccgaggcatacagggccgcgctgatcctcctggaacagggcgccatttctgggcctggctccaacggaccttacgcgggctctagcaggcaagccggattcgtcaactatggtgtctcacacctcatgagactggttgggacggctgagctttcccagaaatcggcctggtatcagaagtggaacgttcacatgttcgtcagaccagaggcttttggtgggacgatacacaacgtgctgctaggcaaacttaatgtggacattaaccctagcctgctgaaaaacaccgagctgcttgagcgggtcgccgagcgcaatggagttattaacgggagaccaggtgtgctggaccggacttacctgctgagtcaggcggtcattgagggttcccccacacacccatcctacccggcggggcatgccacacagaatggggcatttgcgactgttctgaaggcgctcgtcgggctggagcgagggagcgactgcttccgggaccccaaggtgcctgacgatgagggtctcacactgctcgacttcacaggcgactgcctgaccttcgagggtgagatcaacaagttggccgtcaatgtggcattcggtcgtaatatgtgtggagtgcactggcgcatagactcggagcagggccttttgctgggtgaaatggctgcggtcagaattctccagcaggaggcagtgacgttcccggagaatgcggggtacgagttcaacctaatgtccggggagactattcgactcgaaactgatgggaccttcttcataaatgatagattgtgcagtggcgacgcattcatgggcgccgatctttgc

SEQ ID NO: 61 - DNA, 1842 bp, synthetic construct
  source 1..1842: mol_type = other DNA
atgacgattgacttcactcctgtcgagcttcctgtggtcgaggaagacgccgagtacaactggaactacatactcttctggaacaatgtcggcttggaactcaatcgcgttacacacacctttggggccctcaaggccggtccaccactttcgcccagagctctaggcatgcttcagttggctgtgcacgatgcctactttgccatccatccctctgctggcttcacgaccttcttgacaccaggcgctgaagacggtgcataccgtctgcctgatccaagttatgcaaaggacgctcgccaggcagtagctggagcagcaatcgcgatgctgtccaagctttacatgaagcccaaggtcgtcccccgcagccctatctctcacaacgcctatgcccagctccagcacgtcctggacatctcggtaaccaaagcccctgcggcttgcgatccggcatctagcagtttcatcttcggcaaagcagtcgctactgctgtcttcgacctgcttttccacaaggaaggtgccgaccaatctggctacagtcccaagcccggccctttcaagttcaacgatgagcccactcaccctgttgaactcattcccgttgatgccaacatacccgatggtgataagatgccgcgccgccagtaccacgccccctactatggcgagacggccaagcgctttggcacccagactgagcacatgcttgccgacccaccaggcatacgttgcgctggcgaggttgccgaatatgacgatgctatccgtgaagtctacgccatgggcggagcacccggtctcaacaccaccaagcgcactcctcaccagacagtccaaggcatgttttgggcctacgacggcccgaaactgatcggtacgccaccaaggctctacaaccagatcgttcgcaaaatcgcagtgacgtacaagaaggacaatgacctggtcaacagcgaggtcaacaacgccgacttcgcccgtctccttgctctggtgaacgtggctatgaccgacgcaggcatcttcgcctggaaagagaagtgggagtttgagttctggcgcccgctttctggtgtccgtgacgatgttctccgtgaccctgagggtaaagcgtcgaccgcggcgatccattctggcttagcctcggcgccccagctacaaaactcagacgaagctcctttcaagccccccttccccgcctacccctccggccacgctacattcggcgctgcggccttccagatggtccgcaagtactacaacgggcgtcttggaaaatgggcgacgacgagccgagacaccatcgccgtcgaaatgtttgtatctgaggagctgaacggagtgagcagggacctcagcaacccctacgaccccaaacgccccatcacagatcaacctggtatcgtgccgacccggatgccacgccgattctcttcctgttgggagatgatgttcgaaaacgccgtctcacgtatcttcctcggtgtgcactggcgcttcgacgccgcagccggacaggacattttgatccctacgacgaagaaggacgtctatgctgtcgacgacaagggcgctgctctgttcaagaatgttgaggatattcgttataagacgaagggcacgaggaagggacacaagggtttgttgcccattggcggtgtgccccttggtattgaaattgcgaatgagatttacaacaacaaactcagccctacgccaccgggggagcagccaatgccgcagcctccgcagcaccaggggccaccaaggaagaagggggagttggcagaggcgaaggatgaggaacaggctcctatgatggatgtagcgccctag

SEQ ID NO: 62 - protein, 613 aa, synthetic construct
MTIDFTPVELPVVEEDAEYNWNYILFWNNVGLELNRVTHTFGALKAGPPLSPRALGMLQLAVHDAYFAIHPSAGFTTFLTPGAEDGAYRLPDPSYAKDARQAVAGAAIAMLSKLYMKPKVVPRSPISHNAYAQLQHVLDISVTKAPAACDPASSSFIFGKAVATAVFDLLFHKEGADQSGYSPKPGPFKFNDEPTHPVELIPVDANIPDGDKMPRRQYHAPYYGETAKRFGTQTEHMLADPPGIRCAGEVAEYDDAIREVYAMGGAPGLNTTKRTPHQTVQGMFWAYDGPKLIGTPPRLYNQIVRKIAVTYKKDNDLVNSEVNNADFARLLALVNVAMTDAGIFAWKEKWEFEFWRPLSGVRDDVLRDPEGKASTAAIHSGLASAPQLQNSDEAPFKPPFPAYPSGHATFGAAAFQMVRKYYNGRLGKWATTSRDTIAVEMFVSEELNGVSRDLSNPYDPKRPITDQPGIVPTRMPRRFSSCWEMMFENAVSRIFLGVHWRFDAAAGQDILIPTTKKDVYAVDDKGAALFKNVEDIRYKTKGTRKGHKGLLPIGGVPLGIEIANEIYNNKLSPTPPGEQPMPQPPQHQGPPRKKGELAEAKDEEQAPMMDVAP

SEQ ID NO: 63 - DNA, 1830 bp, synthetic construct
  source 1..1830: mol_type = other DNA
atggggtccgttacacccatcccactccctaagatcgatgaacccgaagagtacaacaccaactacatactattctggaaccatgtcggtttggaactcaaccgcgtaactcacactgttggaggccccctgacgggaccacctctctctgccagggctctgggtatgctgcacttggctattcacgacgcctacttttctatctgccctccgaccgacttcaccaccttcctctcacctgatactgagaatgccgcgtaccgtctacctagccctaatggtgcaaatgatgctcgccaagcagtcgctggagctgccctcaagatgctgtcttcactgtacatgaagcccgtcgagcagcctaaccctaaccccggcgccaacatctccgacaacgcttatgctcagcttggcttggttctcgaccgatcagttctggaggcacctggtggcgtggaccgagagtcagccagtttcatgtttggtgaggatgtagccgatgtcttcttcgcactcctcaacgatcctcgaggtgcttcgcaggagggctaccaccctacacccggccgctataaatttgacgatgaacctactcaccctgtcgtcctcattccagtagaccccaacaaccctaatggtcccaagatgcctttccgtcagtaccacgccccattctacggcaagaccacgaagcgttttgctacgcagagcgagcacttcctggccgacccaccgggcctgcgttctaatgcggacgagaccgcggagtatgacgacgccgtccgcgtcgctatcgccatgggtggtgctcaggctctcaactccaccaagcgtagcccatggcagacagcacagggcctatactgggcctacgatgggtcaaacctcattggcacaccacctcgcttttacaaccagatcgtacgtcgcatcgcagttacgtacaagaaggaagaggaccttgccaacagcgaagtcaacaatgcggatttcgcccgcctcttcgccctcgtcgacgtcgcttgcacagacgctggtatcttttcctggaaggagaaatgggagttcgaattctggcgcccactatctggtgtgcgagacgacggccgtccagaccatggagatcctttctggctcactctcggtgccccagctactaacaccaacgacattccattcaagcctcctttcccagcttacccatctggtcacgcgacctttggcggtgctgtgttccaaatggtgcgtcgatactacaacggccgcgtaggtacatggaaggacgacgaacccgacaacattgccatcgatatgatgatctcggaggagctcaacggcgtgaaccgcgacctacgccagccttatgaccccacggccccaatcgaagaccaacccggtatcgtgcgcacccgcattgttcgccacttcgactcggcctgggaactcatgttcgaaaacgccatttcgcgcatcttcctcggtgtccactggcgtttcgatgccgccgccgcccgcgacattctcattcccacgacgacaaaggacgtctacgctgtcgacaacaatggcgccaccgtgttccagaacgtagaggacattaggtacacaaccaggggtacgcgtgaggaccccgagggcctcttccctatcggtggtgtgccactgggtatcgagattgcggatgagatttttaataatggacttaagcctacgcccccggagatccagcctatgccgcaggagacaccggtgcagaagccggtgggacagcagccggttaagggcatgtgggaggaagagcaggcgccggtagtcaaggaggcgccgtag

SEQ ID NO: 64 - protein, 609 aa, synthetic construct
MGSVTPIPLPKIDEPEEYNTNYILFWNHVGLELNRVTHTVGGPLTGPPLSARALGMLHLAIHDAYFSICPPTDFTTFLSPDTENAAYRLPSPNGANDARQAVAGAALKMLSSLYMKPVEQPNPNPGANISDNAYAQLGLVLDRSVLEAPGGVDRESASFMFGEDVADVFFALLNDPRGASQEGYHPTPGRYKFDDEPTHPVVLIPVDPNNPNGPKMPFRQYHAPFYGKTTKRFATQSEHFLADPPGLRSNADETAEYDDAVRVAIAMGGAQALNSTKRSPWQTAQGLYWAYDGSNLIGTPPRFYNQIVRRIAVTYKKEEDLANSEVNNADFARLFALVDVACTDAGIFSWKEKWEFEFWRPLSGVRDDGRPDHGDPFWLTLGAPATNTNDIPFKPPFPAYPSGHATFGGAVFQMVRRYYNGRVGTWKDDEPDNIAIDMMISEELNGVNRDLRQPYDPTAPIEDQPGIVRTRIVRHFDSAWELMFENAISRIFLGVHWRFDAAAARDILIPTTTKDVYAVDNNGATVFQNVEDIRYTTRGTREDPEGLFPIGGVPLGIEIADEIFNNGLKPTPPEIQPMPQETPVQKPVGQQPVKGMWEEEQAPVVKEAP

SEQ ID NO: 65 - DNA, 1866 bp, synthetic construct
  source 1..1866: mol_type = other DNA
atggcaaatttcactcctgttcggcttcctaaggtcgaggaagatgaggtgtacaataagaactacatcctctactggaataatgtcggcttagacctcaaccgtgttacacacaccttgggaggccctcagaccggtccacccatctccgccagagctctaggcatgcttcaattggctatccacgatgcctactttactatcaagccctctgccgacttcacaaccttcttgacacccaacgccgaaatcgacgcataccgtctgcccgatccgactcattcggacgatgcccgccaggcggtagcgggagcagcagtcacgatgctgtcgatgctttacatgaggcctgcggaaatgcccagaccctcccccatatccaatgacacctatgcccagctcgagtatattatagaatcctcgatgaccaacgcacccggcggctgcaatacggtatcctacagtttcaacttcggcaaggcagtagccactaaattcttcgacctgcttttccacgaggaaggtgccaatcaacgtggctacacccccacattcgctcctttcaagttcaacgatgaacccactcatcctgttgaccttgtccccgaggacgccaacgagcccggtggtaacatagttcctagacgccagtatcatgcccccttctatggtacgagagccaagcgctttgcgacacagactgatcacattattgccgatccaccaggcattcgttccggcgctggtgaggtcaccgaatatgacgacgctatccgtgaagtttacgccatgggtggagcagccagtctcaacactaccaagcgcactccccaccagacagtccaaggcatgttctgggcttacgatggcgcaaagttgatcggtacaccaccgcgactatacaaccagatcatacgcaaaatcgcagtggcgtacaagcaagaagataacctggccgaaagcgagatcaacaacgccgactttgctcgtctcctcgctctggtgaacgtggctatggctgatgcaggcatcttctcctggaaggaaaagtggcagtttgagttctggcgcccgctttccggtgtccgcgccgatagtcttcgtgaccctaaacttgtagaccgtggcgacccattctggcttacgctcggggctccagctacaaactcagattcacttcccttcaagccgcccttccccgcctacccctctggtcacgccacgttcggtggtgcagccttccagatggtacgcaagtactacaacgggcgtcctggacttggttcctgggcagacgacgagccagacaacatcgccgtcgaatttgtctctgaggaactgaatggaataagcagagatctccgccaaccccacgaccccaaacgcgacattaccgaccaacccggtaccgtgcgtacccgtctcccccgccacttctcttcgtgctgggagatgatgttcgaaaacgccgtctcacgcatcttcctaggtgtgcactggcgcttcgacgctgcggccgccaaggacatcttggttcccacgacgaagaaggatgtctacgctgtagacgacaagggcgcttctctgttcaagaatgttgaggatattcgttataggacgaagagtacaagggagggatttgagggtaaataccccattggtggtgtacccctgggtattgagatcgcgaatgagattttcgacaacggactcgtgcctacgccgccggaattgcagcctgtggtgcagggtatgccgcagcctacaccgcagcctccgcagcatcaggggccaccaaggaagatggagaagttgccaaagccgaaggatgaggatcaggttcctatgatggatgtggagccctag

SEQ ID NO: 66 - protein, 621 aa, synthetic construct
MANFTPVRLPKVEEDEVYNKNYILYWNNVGLDLNRVTHTLGGPQTGPPISARALGMLQLAIHDAYFTIKPSADFTTFLTPNAEIDAYRLPDPTHSDDARQAVAGAAVTMLSMLYMRPAEMPRPSPISNDTYAQLEYIIESSMTNAPGGCNTVSYSFNFGKAVATKFFDLLFHEEGANQRGYTPTFAPFKFNDEPTHPVDLVPEDANEPGGNIVPRRQYHAPFYGTRAKRFATQTDHIIADPPGIRSGAGEVTEYDDAIREVYAMGGAASLNTTKRTPHQTVQGMFWAYDGAKLIGTPPRLYNQIIRKIAVAYKQEDNLAESEINNADFARLLALVNVAMADAGIFSWKEKWQFEFWRPLSGVRADSLRDPKLVDRGDPFWLTLGAPATNSDSLPFKPPFPAYPSGHATFGGAAFQMVRKYYNGRPGLGSWADDEPDNIAVEFVSEELNGISRDLRQPHDPKRDITDQPGTVRTRLPRHFSSCWEMMFENAVSRIFLGVHWRFDAAAAKDILVPTTKKDVYAVDDKGASLFKNVEDIRYRTKSTREGFEGKYPIGGVPLGIEIANEIFDNGLVPTPPELQPVVQGMPQPTPQPPQHQGPPRKMEKLPKPKDEDQVPMMDVEP

SEQ ID NO: 67 - DNA, 1710 bp, synthetic construct
  source 1..1710: mol_type = other DNA
atggtcgcttcattttccattctgctcctgctttctgccgtgcttctccacgtccacgcaatctctctcagcaacccatctttgcaaacgcttccaaatgtgtcccgtcaaacaacctgttcatcctctgaagactgtccagacacaaaccccttcgtaagatgcgttcgcaatcagtgcatcgatcttcgagacacagttccagatttaccaacccgcccacccacaacaacatcagccacacaacccaacgcacccaacacaccagcagagggcggttcctgtctaaccataacagaatgtccatccggtcttatctgcgtcgccaccaatcccggtggcagatgtgaactgtccagaggaatgttcggagttgcaaccttcgtcgttaacttgattcagcaagactatacacccgcacctgaactatcaccggatcaaggtggccctacagtcacttctagagcaatggccatcatctttctcgccgctcatgacacctttgcagtcgtctctggagagttctcggctaacacaaatctcggagtgcttcgatccatcccgattcgagtcgccgcacgccgtttagacgacgaggaaagggagcgacaagcggaggctgccatgcaggccgccgctttaaccgcagcaaaaagactgtacccgtctttaacacgaattatagaccccgtgttcgaactcgttacggcccgtgctcaatcgcaatttgttgagtttggagaggatgttggaaatgacttgttcgagtctagactcaatgatggatctcaacgcgatcaacttgacgatgagtttgagattggggagcttttgagacatcaacccgaccctaatttccccatcactgcaggcgatccgactgccgtgcagaggaactttggtcgattttggggtgaagtcacaccgtttgccattcgtgacgtcgagcgtgatgcattcttaggtccattcccggaacttccctctgccgaatatcgtgcaaatcttgcggaggttgatgtaatgggcgaatgtaacgactttgtcaatgacgatggtatccttcttcaggacattggaatcttttggggctatgacggggcatccgaaataggagttcctcctcgtctgttcttgcagactgtgcttgctgtcgatgaagttcaggagctttctttgccagatagtgttcgagctctcactggtgtgggggttgccatggctgacgccggtattgcagcttggtactggaagtttttctacgatctttggaggcctactatcggggtgcgaaacgacacaatctctcctgatccagagtggaatccgcgtggaattccgttaagtaaccgcaatgacataccgagaccaccggagtgtgtcggagtcaatcctaacttccctgcatatccatccggacacgcttcattcggggcagcttgttttgtcacccttgccaatatccttggcaaagaacccaaggatgtggttgccagaattacttcggatgaatttaacggggtcactacggagggcactacgggagaggtgcgaagagtgttcacccaaactatcaactttcaggaagctattgaacagaataatgcaggccgcgtgtacttgggcgttcattggagatttgattccgacggtggggaggatgtcggtagacagattgctgaaattgcagctaaccaattcgacatttaa

SEQ ID NO: 68 - protein, 569 aa, synthetic construct
MVASFSILLLLSAVLLHVHAISLSNPSLQTLPNVSRQTTCSSSEDCPDTNPFVRCVRNQCIDLRDTVPDLPTRPPTTTSATQPNAPNTPAEGGSCLTITECPSGLICVATNPGGRCELSRGMFGVATFVVNLIQQDYTPAPELSPDQGGPTVTSRAMAIIFLAAHDTFAVVSGEFSANTNLGVLRSIPIRVAARRLDDEERERQAEAAMQAAALTAAKRLYPSLTRIIDPVFELVTARAQSQFVEFGEDVGNDLFESRLNDGSQRDQLDDEFEIGELLRHQPDPNFPITAGDPTAVQRNFGRFWGEVTPFAIRDVERDAFLGPFPELPSAEYRANLAEVDVMGECNDFVNDDGILLQDIGIFWGYDGASEIGVPPRLFLQTVLAVDEVQELSLPDSVRALTGVGVAMADAGIAAWYWKFFYDLWRPTIGVRNDTISPDPEWNPRGIPLSNRNDIPRPPECVGVNPNFPAYPSGHASFGAACFVTLANILGKEPKDVVARITSDEFNGVTTEGTTGEVRRVFTQTINFQEAIEQNNAGRVYLGVHWRFDSDGGEDVGRQIAEIAANQFDI

SEQ ID NO: 69 - DNA, 2121 bp, synthetic construct
  source 1..2121: mol_type = other DNA
atgtttcgtcgcgatttgcaacgattcgaaaggcccgtcgcttctccttttctcaatcgccgcgcggggaataccatgccactcagttggaatcgttggatcggaacgtccaagtcagctcgatctcgcaaacaacaacgaaaatcgactcgccgcaagttgatgtcagagaatctggagaccaggaaccttctcgccgccaatctgttccacaacgaggcgatgccggaagacgtcaatgaagatgatcaggtcacggctttggatgctctcaccatcatcaatcggatgaaccgcgaacaagctggtgaatccgcgggtgatgtcagacgcggacagatgacggacgtcaacaacgacggtcgcaacagtgccctggatgcgttgatggtgatcaaccgtttgaaccgcgaccaaggtggaccacgagattcaggatcgcccgacgcgggatcgggaaaccaagaccaaacagattccgaaacgagcggagacaattcggatgttgagggcaccaatggcgatgatccatccaccgacggaacaacgtcgggcgatcaaacagatgctgattcagcgattgagttgaccggggatgctgtgcttgattggaacaacctgttcaacgagttgacctccaacagcgaggattatcaaaacccggggtacgcttctcgagcgatggcgatgttgaacgtggcgatctatgactccgtcgcgatagcttctggcgattctgaaattgcattctacgaatacgattcatcattgacgaactcttccggtgacgtttcatcgcaagctgccgcatcgagtgccgcctactccgtcctgagttccctgtactcggaccagcaagaaacgatcgattcgttctatggagatgtgctagcaacttacagccaagatccagaaaccgaggcggggttgatcctcggcgcggaagttaacagtaccgtgatcgcgaatcgtgcaaatgatggatcggactcggtcgtggagtacacctacaccgatgagataggttcttttcattcggatccgctgaatccggacgtcccagtatggggaccgggttggggtgatgtggacacgttcgcaatctccgatgcggacgccttcacaccggagtcgcctccggatttgaccagcgaagaatacgcggcgtcttacaacgaagtgaaggaattgggggcggtcgacagcaccacacgaacggccgaccaaaccgaagcggggatcttttgggcttacgaccgagatggtttgggcacgccgctcacgctgttcaacgacattctagaaacagtcgccgtgcaagaaggcaatacgtttgaggagaatgcggctttgttcgcccaggcatcagtcgcgatggctgatgcgggggttgtggcgtggacgaccaagttcggagaagaactttggcgtccggtcaccgcaatccaagaaggcgatttcgatggaaacacgctgaccgaaggcgatgctgattggaccgctttgggggcgcctgatggaggtgatgacattgttggcttcacgccgcaatttccgacctacatctccggccacgcgacctttggcggtgccttgtttgggaccctgcaagaattctatggcaccgatgacatctcgttcactgtcgcgtcggaagaacttgagattctgttggacaatcctgaattgcaggaagcctacggtttgaatcttgatgacgccgagcgaactttcagttccttcagtgaggccatggccgagaatggacgcagccgagtttacttgggcattcacttcgacttcgatgatttggtcggtcaggaagtcgggcaatccatcgccgcagcggtcgcatcggaattcagcgtggcgacgcccgaagatgactccactggcaaaggcgtcgacgatggccaactcgcaacaatggaccgtgacgaccagccaaaccgagacggcgtcgacggttcgaggcagcggaatcgcgaaccggtcgctcgggacacgactcaagtgcctcagcaacaaacttcatcgcgggatgagtcgaatcgacgatcagaggacgatcagcgaatcgcagccattgaccagattttcgcagagaaccgcttgatttga

SEQ ID NO: 70 - protein, 706 aa, synthetic construct
MFRRDLQRFERPVASPFLNRRAGNTMPLSWNRWIGTSKSARSRKQQRKSTRRKLMSENLETRNLLAANLFHNEAMPEDVNEDDQVTALDALTIINRMNREQAGESAGDVRRGQMTDVNNDGRNSALDALMVINRLNRDQGGPRDSGSPDAGSGNQDQTDSETSGDNSDVEGTNGDDPSTDGTTSGDQTDADSAIELTGDAVLDWNNLFNELTSNSEDYQNPGYASRAMAMLNVAIYDSVAIASGDSEIAFYEYDSSLTNSSGDVSSQAAASSAAYSVLSSLYSDQQETIDSFYGDVLATYSQDPETEAGLILGAEVNSTVIANRANDGSDSVVEYTYTDEIGSFHSDPLNPDVPVWGPGWGDVDTFAISDADAFTPESPPDLTSEEYAASYNEVKELGAVDSTTRTADQTEAGIFWAYDRDGLGTPLTLFNDILETVAVQEGNTFEENAALFAQASVAMADAGVVAWTTKFGEELWRPVTAIQEGDFDGNTLTEGDADWTALGAPDGGDDIVGFTPQFPTYISGHATFGGALFGTLQEFYGTDDISFTVASEELEILLDNPELQEAYGLNLDDAERTFSSFSEAMAENGRSRVYLGIHFDFDDLVGQEVGQSIAAAVASEFSVATPEDDSTGKGVDDGQLATMDRDDQPNRDGVDGSRQRNREPVARDTTQVPQQQTSSRDESNRRSEDDQRIAAIDQIFAENRLI

SEQ ID NO: 71 - DNA, 1473 bp, synthetic construct
  source 1..1473: mol_type = other DNA
atggatcacatcttatattggaataatgtcgcactcgaggccaatcgccgcgacttcagtaatgtacctggcaccgataatcccaatcctaaacctgaacagggcggcccgacactcagttcccgtgccttggccattgtacatttggcaatgtatgacgcgcatgctggtgtcatcagcaatccggccacgctgccacgctacctcgcttcaccatccgcaccggttggtggcgccagcgcggccgcatcagtagcagctgcagcacatgcttgcttgagcgctttgtacccgcgccagaagcatcatttcgatgcagcgcaccaagctgcaggcctatccggtgcgggacttgctgagggtcatggcttcggactggccgtagctacagcgatactggccgaccgtgccaaagatccaggtgcaggcgatgacggctatgctgcttcgatgcatcccggcggccaccgtcccgacccggccaatccgaagcagggttttcatgcacctttctacggcgcacgcagcaagggtttcgcaatcacgaaacggcatgagcttgacaaaccacctgctcttggaagtggggagtataacaaggcacttaaacaggttcgaggcaagggcattgcgcctgaatggataggcacactgccagaccacctcgagaaacgcacaccggaagaaacattaatcggcctatactgggcctacgacggtccacgcgaaatcggaacaccaccacgtttcttcaaccaaatcattcgcaaacttgtgacaactgcaataaatccggctacgggcatgatcaacaccgtcgatgacaatgcgcgcttattcgcgttcgtaaacgtgacgatgggtgatgccggcattttagcttgggatcagaagtatattcacgacttctggcgaccagtgctgggtatccgtgaacacagtgcttcgatgggacctgccgcacccacaccttcgcacgccatcggtgatgactgcgatccctgctggctgccgctgggtgcccccgcaagcaacagcaacgatcgcaatttcacgcccccgttcccggcttacccatcgggacacgctaccttcggagctgctgcgctgcatgtaactcggttattctatggtattgctcacaacaatcgcgccaccgacaagctattcaagcgttccttggtttccgatgaactcaacggtataacacacgacaatgctggtaatgttcgatcgcgacatgcgcgacactttccgggtggactctggcagatgatcgtggagaatggcttttctcgcgtgttccttggcgtgcactggtcgtttgatgcgttcgcactcgacaacgccaacaagcccgatctgacgcgcaatatcggcggtgtgccgctgggattagcaattgcagaagatatttttgctgcaggtggaggcaaggcgcccgctaaatcttgtgttgaaccacgaccctaa

SEQ ID NO: 72 - protein, 490 aa, synthetic construct
MDHILYWNNVALEANRRDFSNVPGTDNPNPKPEQGGPTLSSRALAIVHLAMYDAHAGVISNPATLPRYLASPSAPVGGASAAASVAAAAHACLSALYPRQKHHFDAAHQAAGLSGAGLAEGHGFGLAVATAILADRAKDPGAGDDGYAASMHPGGHRPDPANPKQGFHAPFYGARSKGFAITKRHELDKPPALGSGEYNKALKQVRGKGIAPEWIGTLPDHLEKRTPEETLIGLYWAYDGPREIGTPPRFFNQIIRKLVTTAINPATGMINTVDDNARLFAFVNVTMGDAGILAWDQKYIHDFWRPVLGIREHSASMGPAAPTPSHAIGDDCDPCWLPLGAPASNSNDRNFTPPFPAYPSGHATFGAAALHVTRLFYGIAHNNRATDKLFKRSLVSDELNGITHDNAGNVRSRHARHFPGGLWQMIVENGFSRVFLGVHWSFDAFALDNANKPDLTRNIGGVPLGLAIAEDIFAAGGGKAPAKSCVEPRP

SEQ ID NO: 73 - DNA, 1752 bp, synthetic construct
  source 1..1752: mol_type = other DNA
atgcttgttctacctgatgtcgacgaacctgccgggcttaacgcatttccggtcctgtactggaatcatgccggcctccaaatgaaccgtatcacgcattcgcttcgcggcccgcaaggtggccctaccatgagctctagagctctgggacttcttcatcttgccatgcacgacgccttcttcggcgctctcggtcatgacgaacactcgtctcccgcaacctggttgcgagacgtcaatcgtccgccactcgatccttccatccaggcaacggaagcaaatgccaatgcagccctcaccgctgccgccattacaatccttgagcgactatattgtgctccgggcgaggtctgtgacgcggcaagcaatgcgctgcgcgacgcgatgcgcaacttcaagagcccattcggtgtggcaattgcagcaggatcgccggcgcatcactatggcgtgaccattgccgaaagagtccacgcagcacttgcggtattgccaggcgagccgggcgccgatcagggcgactaccggccccggggaggtcgctatcacttcgatgacgaaccactcaaccctgtccgccgcgtggacattgatcctcgagacccttcaaaaggacaaaaggcacagcgtgtctatcacgcgcccttttacggtgagaccgtgagaacctttgcagcgaccgatccgaatggccatcggatcgccgactggccaagaccaccggagccagaatacgtggcagcacttgaggaggtccagcggcttggcggcgcaccgctcctgaacacaacgcatcggacggcagacgagacggtggcggcctatttctgggcttatgatggagccaatctcattggaacacctccgcgtctttacaatcagatcctgcgcaatatcgcctggtcgaggagcgatgcctcgctgagcgacttcgacagaagcaaagagttcgtccggctgtttgcgcttgccaatgtggcgatggcggatgcaggcaaattcgcctgggccgaaaaataccggcacgatctctggcgcccattgtctggcattcgccaacacgacgcaagcgcattcgcctccgagaccggaagcacaatggtgacgccgcctgccgatccgttctggctcgcacttggtgcgccagaaacaaactccaaccgtctgagcttcaaaccacccttccccgcctatccctcaggccatgcaacctttggcgcagcatgctttcagatggcaaggctctactacgcctcgaacgggacggccgctgttgacagaaatggggtcgacgacatcggtttcacatttgtgtcggaggagatgaatggcgtaagccgcgacttgcatcagccttacgatccttcatcgccgatcgaggaccagccagggcttgttcggacctatgtgaagcgcaacttcccgtccctctggcatgcaatatgggaaaatgcgttcagccggatttggctgggtgtccactggcgtttcgacgcctttgactatcgcgataccggaagcggtcgcgatgccgacgggcgcgagcgctacaaggaccctgccgatgtcggctattcgcatgtctggactgcaaagcggaacgccggcgacccactgccgatcggcggcgtccccttgggtctcggcatcgccaatgacatttttgacagcggcatgcgacagcaagccgacgaaatgccgaaagcgcggtcgacacctcagcaaagcaagatcacggacacgacctactaa

SEQ ID NO: 74 - protein, 583 aa, synthetic construct
MLVLPDVDEPAGLNAFPVLYWNHAGLQMNRITHSLRGPQGGPTMSSRALGLLHLAMHDAFFGALGHDEHSSPATWLRDVNRPPLDPSIQATEANANAALTAAAITILERLYCAPGEVCDAASNALRDAMRNFKSPFGVAIAAGSPAHHYGVTIAERVHAALAVLPGEPGADQGDYRPRGGRYHFDDEPLNPVRRVDIDPRDPSKGQKAQRVYHAPFYGETVRTFAATDPNGHRIADWPRPPEPEYVAALEEVQRLGGAPLLNTTHRTADETVAAYFWAYDGANLIGTPPRLYNQILRNIAWSRSDASLSDFDRSKEFVRLFALANVAMADAGKFAWAEKYRHDLWRPLSGIRQHDASAFASETGSTMVTPPADPFWLALGAPETNSNRLSFKPPFPAYPSGHATFGAACFQMARLYYASNGTAAVDRNGVDDIGFTFVSEEMNGVSRDLHQPYDPSSPIEDQPGLVRTYVKRNFPSLWHAIWENAFSRIWLGVHWRFDAFDYRDTGSGRDADGRERYKDPADVGYSHVWTAKRNAGDPLPIGGVPLGLGIANDIFDSGMRQQADEMPKARSTPQQSKITDTTY

SEQ ID NO: 75 - DNA, 780 bp, synthetic construct
  source 1..780: mol_type = other DNA
atgcagtggcaggtcaacggagcgtcgcaaatcggtgttccgccgcggctgtataatcaaatcgttcgtgagataattgggaacaatgtgaacggagctgagcgggcggcggcctcggcgcgtcttttagcgctcgtcaacgtcgcgatggcggacgcaggtatcgcttcttggtactacaagtacacgtaccagctttggcgccctgtattaggaattagagaatacgacgatagttattggtacaacggcaccgcggtttctcacgcgcttcacaaacggtgcgatccttggtggatacccctaggctccccaagaacgaacgagtctggtcgacatagctttactcctcccttccccgcctatccttcggggcacgctacgttcggtgccgctgccttcgaaataactcggcgttttttcggggtcgcacccggtgcccaagacaatctattctttaacacaatatcggatgagtgcgacggccgagccattgccgaagatggctctttcagggggcgtcagcggcggcaccacgattccttgctccgtggaatgttcgacaatgcggtgagtcgcgtgtatctcggtgttcaccggcgctttgacggcattggagacaacgttaccactcaccaagatatcttgaatgataactcgaatattggcggcgtacccttgggacgcgctctagcccacgatattttcaacaatggccttgcaaagagcgctgcagcgagagcggtaatcactcccaaaaatctcgctccggtaccttga

SEQ ID NO: 76 - protein, 259 aa, synthetic construct
MQWQVNGASQIGVPPRLYNQIVREIIGNNVNGAERAAASARLLALVNVAMADAGIASWYYKYTYQLWRPVLGIREYDDSYWYNGTAVSHALHKRCDPWWIPLGSPRTNESGRHSFTPPFPAYPSGHATFGAAAFEITRRFFGVAPGAQDNLFFNTISDECDGRAIAEDGSFRGRQRRHHDSLLRGMFDNAVSRVYLGVHRRFDGIGDNVTTHQDILNDNSNIGGVPLGRALAHDIFNNGLAKSAAARAVITPKNLAPVP

SEQ ID NO: 77 - DNA, 1680 bp, synthetic construct
  source 1..1680: mol_type = other DNA
atgcaaacttgctcaacctcagatgatgctgatgaccctacaccacctaacgagcgggatgacgaggcattcgctagcagagtagcagctgccaagagagagctcgagggtactggcacagtctgccagatcaataatggagaaactgacctggctgcgaagttccacaagtcactccctcacgatgatttgggacaggttgacgctgatgcttttgccgctctggaagattgtatccttaatggagatttgtctatctgtgaggatgtgccggtggggaactctgagggagatcctgtaggtagattggtaaaccctacagctgcatttgcaattgacatttcagggccggcattcagcgccaccacaatcccccctgttcctacccttccgtcgcctgaattggcggctcaactggctgaagtctattggatggcactggcaagagatgtaccgttcatgcaatatggaacagatgatattaccgtgaccgccgctgctaacttggctggaatggagggtttcccaaacctcgatgctgtttctattggttcggacggcactgtcgaccccctctcccaattattccgagctacctttgtaggtgtggagactggaccttttattagtcaattgctggtcaattcatttacgatagatagtattactgttgaacctaagcaggagacctttgctccagatgtgaactacatggttgacttcgatgaatggctcaacattcagaacggtggccccccagccggaccggagcttctcgatgacgagctccggttcgtccggaacgcccgggatttggcaagagttacctttaccgataacatcaacactgaagcttaccgtggggctctcattctgcttgggttggatgctttcaacagggcgggtgttaacggacctttcattgacattgaccggcaggctggcttcgtgaatttcgggatttctcactactttcgtctgatcggagcagccgagttagcacaaaggtcttcttggtaccaaaagtggcaagtacacaggtttgctaggcctgaagcgttaggaggtactctccatctaaccataaaaggagagctcaatgcggactttgacttgagtttgttggagaacgctgagctgttaaaacgtgttgctgcgattaacgcagcacaaaatccaaacaacgaagtcacgtatcttttacctcaggcgattcaggagggttctcccactcatccatcttatccttctggacacgcaactcaaaacggagcatttgctactgttcttaaggccctcattggcttagataggggtggcgattgttaccctgatcctgtttacccagacgatgacggtttgaaactaatcgattttagggggagttgtcttactttcgagggtgagataaacaaattagccgttaacgtagcatttggcagacagatgctcgggatccattatcgtttcgatgggatccaaggacttttgttaggggagacgatcacggttcgtactctccaccaggaattaatgacatttgctgaggagtcaacgttcgaatttagactcttcactggtgaggtcattaagttgttccaggatggaacatttaccatcgatggctttaagtgccctggccttgtgtatactggggtcgagaactgtgttagctaatag

SEQ ID NO: 78 - protein, 558 aa, synthetic construct
MQTCSTSDDADDPTPPNERDDEAFASRVAAAKRELEGTGTVCQINNGETDLAAKFHKSLPHDDLGQVDADAFAALEDCILNGDLSICEDVPVGNSEGDPVGRLVNPTAAFAIDISGPAFSATTIPPVPTLPSPELAAQLAEVYWMALARDVPFMQYGTDDITVTAAANLAGMEGFPNLDAVSIGSDGTVDPLSQLFRATFVGVETGPFISQLLVNSFTIDSITVEPKQETFAPDVNYMVDFDEWLNIQNGGPPAGPELLDDELRFVRNARDLARVTFTDNINTEAYRGALILLGLDAFNRAGVNGPFIDIDRQAGFVNFGISHYFRLIGAAELAQRSSWYQKWQVHRFARPEALGGTLHLTIKGELNADFDLSLLENAELLKRVAAINAAQNPNNEVTYLLPQAIQEGSPTHPSYPSGHATQNGAFATVLKALIGLDRGGDCYPDPVYPDDDGLKLIDFRGSCLTFEGEINKLAVNVAFGRQMLGIHYRFDGIQGLLLGETITVRTLHQELMTFAEESTFEFRLFTGEVIKLFQDGTFTIDGFKCPGLVYTGVENCVS

SEQ ID NO: 79 - DNA, 1521 bp, synthetic construct
  source 1..1521: mol_type = other DNA
atgagctacggacttcggcacgtccaactggcagcttgcgccgactatagccgactgcacaatgcggcgcgaacggtaagccaagcctacccattcgggacaccactgttctgccggccgagaaaaatctgcttctcggttgaaccgcgtgcggcaagtgaagcagttcacaatcaagccacatcaagatcgctagagtccgcgggcagcgcgacgcgacgtcggccagagccgcctcgcgaaacgtccttcagatcgaaagggcacgccatgaaatgctcctcgtggaccatctcccgcttatcgctgattgtagcgctgatcgcggcctcggcgtcgctggcacgcgccgacgtgatcatggactggaatgcaaaggccgacgcgatcgctgccgagaagcaaattcttccagcgccccacagccgcgtcttgtccatgatgcacgtcgcgatgttcgaggcggtcaacgcgatcgaccgccggtacgcgccctacaagctctccttgcccgcggatcgttcgacgtcgcgggaagctgcggcggcagttgccgcccacgacgtgctgctgtcggtctatccggacctcaagccggacctggatgcgacgctgacgaattctctggcgccgattgccgacggcgagtccaagatcgccggcatcagcctcgggaaagaggccgcaatgcagatcatcgagcttcgtgcgaacgatggcagcgccgctccggagacctaccgccccctgacgacaccgggcgcgtacgttccgaccaccgttccgctgttctcaaccacgggcgcgacgacgccctgggtcatggtttccggatcgcagtttcgccccggcccgccgccggcgcttggttcagaggtgtggacccgggatgtcaacgaaatccgcgaggtcggcagccgaagcagtacaacccggacgccggagcaaacgacgatcggccgattctggttctttgtcggtgcccgcacctacaatccgatcgtgaggcaagctgcaatggccaagggcatggatctcatcgactgcgcccggctgttcgcgctgacgtcaattgccggcaacgacgccctcgttgcggtgttcgatgcaaaataccattacaatctctggcgaccgatcaccgccatacgcaacgccgacctgacgtcgaatccggcgacgccacgcgacccgtcctggctgccactcggcgaaacgccgatgcatccggaatatccctgtgctcactgcatcacgtcggcggcgatttcgaccgtgctccagagcgtcgttggagatttcggcgagttctcgctgaccagctccacggcaccaggtgtcacgcgcaaatggtcgcggctccaggactacagcgacgaagtctccaacgcccgcatctgggccggctttcactaccggttctcgaccgaggtcggcaaggacatgggtcggaagatcggcgcactgaccgtcgcgacccagcttcgcggtgtggaagcgatggcggagccgaagcgttaa

SEQ ID NO: 80 - protein, 506 aa, synthetic construct
MSYGLRHVQLAACADYSRLHNAARTVSQAYPFGTPLFCRPRKICFSVEPRAASEAVHNQATSRSLESAGSATRRRPEPPRETSFRSKGHAMKCSSWTISRLSLIVALIAASASLARADVIMDWNAKADAIAAEKQILPAPHSRVLSMMHVAMFEAVNAIDRRYAPYKLSLPADRSTSREAAAAVAAHDVLLSVYPDLKPDLDATLTNSLAPIADGESKIAGISLGKEAAMQIIELRANDGSAAPETYRPLTTPGAYVPTTVPLFSTTGATTPWVMVSGSQFRPGPPPALGSEVWTRDVNEIREVGSRSSTTRTPEQTTIGRFWFFVGARTYNPIVRQAAMAKGMDLIDCARLFALTSIAGNDALVAVFDAKYHYNLWRPITAIRNADLTSNPATPRDPSWLPLGETPMHPEYPCAHCITSAAISTVLQSVVGDFGEFSLTSSTAPGVTRKWSRLQDYSDEVSNARIWAGFHYRFSTEVGKDMGRKIGALTVATQLRGVEAMAEPKR

SEQ ID NO: 81 - DNA, 780 bp, synthetic construct; identical to SEQ ID NO: 75
  source 1..780: mol_type = other DNA

SEQ ID NO: 82 - protein, 676 aa, synthetic construct
MLCHAADTTRGSPMPDTGVLRLLTSEQRAKGWRRQLEGEKSLGFHPSETPYIKYLEGSETWKKVKLPTDGISASKILGKIMARVRIATALAVVLAAPCLAFDEVTASGVFPEEHKHTGEGRHLQTCTNSDDALDPTAPNRRDNVAFASRRDAARRERDGTGTVCQITNGETDLATMFHKSLPHDELGQVTADDFAILEDCILNGDFSICEDVPAGDPAGRLVNPTAAFAIDISGPAFSATTIPPVPTLSSPELAAQLAELYWMALARDVPFMQYGTDEITTTAAANLAGMGGFPNLDAVSIGSDGTVDPFSQLFRATFVGVETGPFVSQLLVNSFTIDAITVEPKQETFAPDLNYMVDFDEWLNIQNGGPPAGPEELDEELRFIRNARDLARVSFVDNINTEAYRGSLILLELGAFSRPGINGPFIDSDRQAGFVNFGTSHYFRLIGAAELAQRASCYQKWQVHRFARPEALGGTLHNTIAGDLDADFDISLLENDELLKRVAEINAAQNPNNEVTYLLPQAIQVGSPTHPSYPSGHATQNGAFATVLKALIGLDRGGECFPNPVFPSDDGLELINFEGACLTYEGEINKLAVNVAFGRQMLGIHYRFDGIQGLLLGETITVRTLHQELMTFAEEATFEFRLFTGEVIKLFQDGTFSIDGDMCSGLVYTGVADCQA

SEQ ID NO: 83 - DNA, 1203 bp, synthetic construct
  source 1..1203: mol_type = other DNA
atgcacaagacgtcccgcaggcgactcatcgtcgccgccgccctgaccccggcggcgctcgcgctgcccacccccgcccacgccgacacgatcgacccggtcgccgagtggtacgacctgaccgccgacgcggtcaccacgtccggaccgtccgcgcaggtcagcggcagccggatctgggccctggcctggctttcggccgcccgcgcgctgcacggcgactcgcaccccgaccggcagagcgccgcgctggtctccgccgtccacgccgtgctgcgcgaccagatcccgtcccgcgcgacggatctcgatgccgcgctggaggcgtcgctggcccgcgttcccgacggctccccgaaacgacgcggaacggcggcgggccgtgccgaagcccggtcgctcctcaccggccgggccggtgacggtctcgaccccgcgtcggtcaacgcgccctacacccccgacccgccgacaccgggacgatggcagccgacgccaccggccttcggtcctggacagcagtccggaacccgtcacgccaggccgttcctgctcggccgcgccgaccgcttccgcccaccaccgccgcccgcgctcgactccgcccgcgatgcccgcgacctcgccgaggtccgtgccttcggcgcgctggacagcacggtgcgaacccaggcccagaccgacaccgcgcagttctggtacggctcctccctggtgctgtacaacgggatcctgcgggccgcgctactccagtcccggcggtcggcgtaccggcgcgcgcggctggtcgcgctgttccacgtcgccctcgtcgacacgcagatcgcgacctccgacgccaagtaccaccaccgctcgtggcggccggtcacggcgatccgggcgctgcacgatccacagtggacgccgtaccacgtcacgccctcgcaccccgactacgtctcgggccacaacacctactccggcgccgccgaggcggtgctcgccgaactggcgggccccgcgcggccttatacgatcggcagccccagcgcgccgggtgtcacccgcacctacaccgactggtcgaccccgtcgcgcgagaacgtcgacgctcgcgtctggtcggggatccacaccaggaccgccgacgaggccgggatcatcctcggccggaaggtcgcccgccacgccctgagccgcgccgaacggctcttcgactag

SEQ ID NO: 84 - protein, 400 aa, synthetic construct
MHKTSRRRLIVAAALTPAALALPTPAHADTIDPVAEWYDLTADAVTTSGPSAQVSGSRIWALAWLSAARALHGDSHPDRQSAALVSAVHAVLRDQIPSRATDLDAALEASLARVPDGSPKRRGTAAGRAEARSLLTGRAGDGLDPASVNAPYTPDPPTPGRWQPTPPAFGPGQQSGTRHARPFLLGRADRFRPPPPPALDSARDARDLAEVRAFGALDSTVRTQAQTDTAQFWYGSSLVLYNGILRAALLQSRRSAYRRARLVALFHVALVDTQIATSDAKYHHRSWRPVTAIRALHDPQWTPYHVTPSHPDYVSGHNTYSGAAEAVLAELAGPARPYTIGSPSAPGVTRTYTDWSTPSRENVDARVWSGIHTRTADEAGIILGRKVARHALSRAERLFD

SEQ ID NO: 85 - DNA, 1209 bp, synthetic construct
  source 1..1209: mol_type = other DNA
atgcactcacgtcgtcgcatcgtcgcggctgccgctgcggttctcctggcacccatgatcgcggtgccggtcgtcggatcggcggcgagcgccaaaccgctcggatcatcgatcgccgtcgactggcagcggacggcgaaccgcacgatctacaccgaggccggcagtgcgccgccgctcggcgcgctctacctcgccttcacgtcgctcgctgtccacgacgctgcgacgaaggcacagcggcacggcatgcacgcagccgcggcagcggtggcgacggccgcgcacgacgtgctcgtcgagtacttcccggcatccggggcggcactcgacaccgacctcgccaacagcctggcgagggtccgcgacggcgcgaagcaggacgccggtgtggccatcggtgccgccgccgcggacaggatgatcgcctcccgcgtcgacgacggccgcttcgaccccgcgtacgtctacagcaagccgcccgctcctggggtgtggcagcctgcgctggggggcgcgatggccctgccctggctgggctacgtcgacccggtcgtgcacatccgccccgttttcctcgacgggccggaccccatcaccagtgccgcctacgcgcgggactacgacgaggtccgcgcggtcggcgaggtcggctcgacgacccgcaccagcgaccagacggcgatcgcgcagttcttcgcgaacaacccgatggtcatgtaccgcacggcagtgtgcgacctgctcgccgccgagccgctgggtctgctgccgacgacccgcctcttcgcccggatcgacgcggccgtcgtgacctcgttcatcgcgagctggcggctcaagtacgagatcgggtattggcggcccttccaggccatcgctggcgcagacacggacgacaactccgcgaccacgccccaggacaactgggtgcccttggtgaccaacccggcgtacgccgactacacgagcgggcacgccaacgcgacgtcgccgtttgcacaggtgctgcgccgaacgctcggtgacgacaccgggctggtcctcagggccgggcccatcacccgcagctatgccaccttgaccgcgctggagcacgacgccctcaacgcccggatctggggtggcctccacttccgggacgccatggacgacggctactacctcggccacacgactgcggaccgcgtgatggcggccgtccactga

SEQ ID NO: 86 - protein, 402 aa, synthetic construct
MHSRRRIVAAAAAVLLAPMIAVPVVGSAASAKPLGSSIAVDWQRTANRTIYTEAGSAPPLGALYLAFTSLAVHDAATKAQRHGMHAAAAAVATAAHDVLVEYFPASGAALDTDLANSLARVRDGAKQDAGVAIGAAAADRMIASRVDDGRFDPAYVYSKPPAPGVWQPALGGAMALPWLGYVDPVVHIRPVFLDGPDPITSAAYARDYDEVRAVGEVGSTTRTSDQTAIAQFFANNPMVMYRTAVCDLLAAEPLGLLPTTRLFARIDAAVVTSFIASWRLKYEIGYWRPFQAIAGADTDDNSATTPQDNWVPLVTNPAYADYTSGHANATSPFAQVLRRTLGDDTGLVLRAGPITRSYATLTALEHDALNARIWGGLHFRDAMDDGYYLGHTTADRVMAAVH

SEQ ID NO: 87 - DNA, 1848 bp, synthetic construct
  source 1..1848: mol_type = other DNA
atgcaagtagatcctaattctgatcaaggtttcgagcaaaatgctcaggctaataacgaaatagataaactgaaacaaccccgtgtagctagcagacgtttttttggtactcacgccagtagacgctcatttctcggtcgcgctggtttgtttagtgccgctagcgttgttgcaggcgttttagattcacctttctcctcaaaaaagggagaagatattgtacaagcccaagacatcaacaggctccctaatcgacctttcgactacaagagctttgttaatagagcttatcgagtacgtgttgaagcagccagacttcaacagagtattccgattccgccgcatccgaccaacggggatgaggcgaagtatgctaacaaaattgcgacggatacacgaggattaccccataacaagctgggcgaagtggatctgaaggcttatcaatcttttattaatgcgttgaactccgaagatcccaatgagtatgaaaagatcatcctgggtggcaaacggaagctgctacaaccgctcggtccattagcggttagcttgagtgggctgaatgcgacccaattgacaattccagtgccacctaccctcgatagtgcagagcaggcggctgaattcctcgaaattgcttggcaacaactgttgcaggatataccctttagcgagttccacaataatacaactaacccgctgattctggcagcagttcaggatctgaatcggttgtcagcattcaaagggcctaaacagaacggacgggttacacccgaactcctatttcgaggcaccgcgatttacgttaatgcctctggtgcaacgagttactacacgttaccaggggtagatgtgggcccatacgtctcccagtttttactgcgtcctgtgcccgcaggaacacaaagttatcttcagcttaaccgtgttcccctagctattcccgaaaacagttttcaaaccaactataacgagtggttgctggtgcaaaatggtggcgattctggtcgaacgattaagtttgacccaacccctcgctactttatcaacgggcgcgaccagtcagagattgcccacacgccaccacccgtctatacaaatgctgctctaattctactcgccaagccagcccttgataacctcctagctggtggcgttggctcaccttacaatcccggtaatccttataacaactcgaagacccaagcgagtggttctgcgacatttggtccagggtattcacaatctatcatctcttgggttagtccacatgcaatcagagcagcttattggcagaagtactatgttcaccgtcgtctgcgagcagaggcttatggtgggttggttcacaacaacaaggtgaataaaacaaactatccgattcaccccgatgttttccagtcagaagcacttgatcgcttcttcagcaaaaacaaatcctatctgctaccgcaagcttttcctgaaggggcaccattccactcttcttatccagggggcgcttcggtgtccgcaggtgccagtgtcacgattttgaaagcactatttgatgagaattatgtgattccgaatccggtggtacccaatcccaaagatcccacccagttgattccctatcaaggcccactgctgaccgtgggcggcgaattgaacaaactggcggcaaatattggcttgggtcgtgatagtgctgggattcactggcgcacagatgcagcagcgtcgttggcccttggcgaagcgatcgccatcagcattcttaaggatgaaaagctaacgttccgcgaaaaatttcaaggtttcacgttcacaaaatttaatggtacaagaatcaccatttga

SEQ ID NO: 88 - protein, 615 aa, synthetic construct
MQVDPNSDQGFEQNAQANNEIDKLKQPRVASRRFFGTHASRRSFLGRAGLFSAASVVAGVLDSPFSSKKGEDIVQAQDINRLPNRPFDYKSFVNRAYRVRVEAARLQQSIPIPPHPTNGDEAKYANKIATDTRGLPHNKLGEVDLKAYQSFINALNSEDPNEYEKIILGGKRKLLQPLGPLAVSLSGLNATQLTIPVPPTLDSAEQAAEFLEIAWQQLLQDIPFSEFHNNTTNPLILAAVQDLNRLSAFKGPKQNGRVTPELLFRGTAIYVNASGATSYYTLPGVDVGPYVSQFLLRPVPAGTQSYLQLNRVPLAIPENSFQTNYNEWLLVQNGGDSGRTIKFDPTPRYFINGRDQSEIAHTPPPVYTNAALILLAKPALDNLLAGGVGSPYNPGNPYNNSKTQASGSATFGPGYSQSIISWVSPHAIRAAYWQKYYVHRRLRAEAYGGLVHNNKVNKTNYPIHPDVFQSEALDRFFSKNKSYLLPQAFPEGAPFHSSYPGGASVSAGASVTILKALFDENYVIPNPVVPNPKDPTQLIPYQGPLLTVGGELNKLAANIGLGRDSAGIHWRTDAAASLALGEAIAISILKDEKLTFREKFQGFTFTKFNGTRITI

SEQ ID NO: 89 - DNA, 2040 bp, synthetic construct
  source 1..2040: mol_type = other DNA
atgaccgacccaccaccaccaccaccactgtcaacgtctcccttcacattctccaccactgacaacagcgatggacgcaccaactttgaacgcccagtcgcgtatgccgaggcatatgtcgaggaaatagagacgtattatcatgtgaacaaccgcaatgttgattccgattatgcaccaccagacgtgaacaatctatcatccaatgatgatgatgatgatgtgaactcgaactcgaagccgaagaaagtttactatcaaatagcagggatggtgctgctgccgttgcttctttacgccggatacacacttggtgctaaaaccagcagtcaaaagattgccagcgccgtcgctgtagcaaatgttacaataagcaatgatcccatgtcaaaaacacccaaggcaaaaacacccaagacaacaggagcaggaggacccatagccaagcaagataagaaaaggcgcgatagcaacgatcgggatgtttgtctagagtggaatgctatcgcaatcacatccaacatcttcgatcacaaaggaaaggcaaagtcagcgcaagacctttccttgacgatggggccagttgcatcagctcgcgcatttgcaatgatgcacacggccatgtttgaggcatacaattgtgaaaaaaagactcatgataactcttacccagctggtcatcccattcataaaatcaattgcactggagtttcgacattatgtacagttgcgcaagccgtccatgacgtaatgtgtggagtaaaaggtgaagatgacatcattggccttttcgaggaagatcaggatgaccaggatatttgtgacatggccgacaccgctcttcgaaatgcactctcaagagaagaagacaaagataagtgtaaaaaaggcaccgaggcaggccgtgatatcgctaaatatgtacttatggaaagggcaaatgatggctacggaacagaaaatcggaccttgaaagatgaaatgtatgtccccccaagtggaaacaaccctggatgtcacatgccagatgccgagaaccccgaacagggcgtccttggggcggggtttggtaaggtgaaagcattctttctcacgagggaggagctcatgcaattctcaggggaggatgcgcctggtgtcaccgtcaatgaaaggacaggtgaggtcaaatttgacttaaatgatacagagtatctcgccgctttgatggaagtaagagagttgggagtattccgtggtggaacatctggagaatatgctcccacggatgacgaaacagtggttattggacagtattggtctgctgacggaagtccacatataggtacgccaatcgctttgttaaatgacatgacacggaaaattgtcatggaggtcgatattgacgagaaacaaagctgttacttatttccattgctaagtgttgttatgggtaatgttgcaattgctgtgtggcataagaagttcaataacaatctttggcggccacttttggcaatcagctcatcgtttcctgaatcagcatgggcgagaatgggggcctcacgagcgaatccgttcgacaacgaaactaattttgaccctcccttcccatcattcccatctggtcatgcagccttttgctgtgcgacgttccagacgattgctaacgtctaccaaacgcataacatcacattcaattacacatcacccgaggtgaatggtgaaacttatgaccagttcaaccgaaaacgcaagtgtatcatacgtgaatttcccacgctgaagcatgcaatggctgagtgcgccgcctctcgtgttttcaatggagttcattactcctttgatggggatgggggttgtaaagttggcatcgatattgctaactacgtgtacgagaacaagttcctgcctctggacggaagccaaaagacttcgatgccggatatgagggaagaaatttttgatgaaatgaagaaatacttgaacaatactgcaacaagtggctattctccaaacttttgcagtaaagctcctgaatatcccttgccccaaccttaa

SEQ ID NO: 90 - protein, 679 aa, synthetic construct
MTDPPPPPPLSTSPFTFSTTDNSDGRTNFERPVAYAEAYVEEIETYYHVNNRNVDSDYAPPDVNNLSSNDDDDDVNSNSKPKKVYYQIAGMVLLPLLLYAGYTLGAKTSSQKIASAVAVANVTISNDPMSKTPKAKTPKTTGAGGPIAKQDKKRRDSNDRDVCLEWNAIAITSNIFDHKGKAKSAQDLSLTMGPVASARAFAMMHTAMFEAYNCEKKTHDNSYPAGHPIHKINCTGVSTLCTVAQAVHDVMCGVKGEDDIIGLFEEDQDDQDICDMADTALRNALSREEDKDKCKKGTEAGRDIAKYVLMERANDGYGTENRTLKDEMYVPPSGNNPGCHMPDAENPEQGVLGAGFGKVKAFFLTREELMQFSGEDAPGVTVNERTGEVKFDLNDTEYLAALMEVRELGVFRGGTSGEYAPTDDETVVIGQYWSADGSPHIGTPIALLNDMTRKIVMEVDIDEKQSCYLFPLLSVVMGNVAIAVWHKKFNNNLWRPLLAISSSFPESAWARMGASRANPFDNETNFDPPFPSFPSGHAAFCCATFQTIANVYQTHNITFNYTSPEVNGETYDQFNRKRKCIIREFPTLKHAMAECAASRVFNGVHYSFDGDGGCKVGIDIANYVYENKFLPLDGSQKTSMPDMREEIFDEMKKYLNNTATSGYSPNFCSKAPEYPLPQP

SEQ ID NO: 91 - DNA, 1851 bp, synthetic construct
  source 1..1851: mol_type = other DNA
atgagcagagaaaacgatacaactcgatcagatccagatctatcgtcaccggaagagtcgaaatcgaacgtgccgatgatcgaccgacggaagctgctacgcgggatcgcagtcgcgggtgggttcgctgctggtggcccacagttgttcgcccaggaggcggatgcctatgatggtgagtacgacgacgtgtatgagctccaggatcgcctcgaagaggcaattgccagtcggcaggagacgattcagcagttgctcgtcaatctaacgacaatcgaatcggattacgacagcacgtatttcgatcttacgaagaagatgcacggaatcgaaatgtatccatcgttgttcatgaagggaatgtcgatggagacgaacagtaacgatgacccatatccagaaggcttcgtttccgtcggggcccacgaggccgtcatgagtccgattcttcggaatcgggatgaatataataatatcccacagggtgacgaacgccggtttattagtccccagtcagtccattctttcccaacggaggggatggattcgtggtccggaacgatgccacccgcaccatctcccggcagcgaccatatggccggggagatggtcgatctctattggatggcggcccttcgtgatcagccgttcgatgcctatagtgggacgctcgacggtgcgatcacgaaatcggatatcaatgcagatatcaccaccatagcggacgcgatacaaaccccgtggtggcacaatactggctatctgttcgtggaagcagcgaccgacgaacttgattatggaccgtacctctctcagtttctgattcaggatgtaatgttcggtgcattcccgattagtccagagatcaagacgttccaacagggtcaagattatcgtacaacacgccaggaatggttacgactgctagcaggagaaggccagggagcagagtcctcaccgcctgcatcaaggttgactgaagggccacgctacattgctacaggacgcgatctcgcggcgttggtgaatttcgatccgtcataccacgtttatgtcatggccgcactcacgctcctggatcaggggatctcaatgaacccagcactccaatacatgacagacgcaggaaacaagatattcgattacatcgacggtggacctgtagcgctgttggatctcattggtcgagctgcacgtaacgctcttctcacagcgtggtatcacaagtggcgcgtccatctccgccttcgtcccgaaacgttcgccggtcgaattcactcccaacggatagatagtcgatattatggcatttcagatctcgttttcgaatcgaatgcgctcaccgaaatcgaggagaacaccgggactgcctttctcccgctggcgtacaaggagggggcaccggtacatccggcgtatcccagtggacactcaactatcgcaggagcctgtggaaccgttctgaaaacctttttcaagaacgaagactggcctggagagctgttcgttccagtcgatcatggagccagccgaacgaccgttcccgtcccagcgaaccatcagggggtccatcaggagattgataaactgatgtcgaatatgggtctcggacgcttattcgctggcgttcactactacagcgatcactactggggaatcaaactcggagaacaaacagccgttgccatgttacgagacgtcctcgatcaagcatacactgacggtcgagatgtcactccaacctttaccgaatatttcggagattacagcaacccactggagatctcgatcgaggaattagagaatcttcgcgagagtgccacacacggtcttacatatccaaaatcgtaa

SEQ ID NO: 92 - protein, 616 aa, synthetic construct
MSRENDTTRSDPDLSSPEESKSNVPMIDRRKLLRGIAVAGGFAAGGPQLFAQEADAYDGEYDDVYELQDRLEEAIASRQETIQQLLVNLTTIESDYDSTYFDLTKKMHGIEMYPSLFMKGMSMETNSNDDPYPEGFVSVGAHEAVMSPILRNRDEYNNIPQGDERRFISPQSVHSFPTEGMDSWSGTMPPAPSPGSDHMAGEMVDLYWMAALRDQPFDAYSGTLDGAITKSDINADITTIADAIQTPWWHNTGYLFVEAATDELDYGPYLSQFLIQDVMFGAFPISPEIKTFQQGQDYRTTRQEWLRLLAGEGQGAESSPPASRLTEGPRYIATGRDLAALVNFDPSYHVYVMAALTLLDQGISMNPALQYMTDAGNKIFDYIDGGPVALLDLIGRAARNALLTAWYHKWRVHLRLRPETFAGRIHSQRIDSRYYGISDLVFESNALTEIEENTGTAFLPLAYKEGAPVHPAYPSGHSTIAGACGTVLKTFFKNEDWPGELFVPVDHGASRTTVPVPANHQGVHQEIDKLMSNMGLGRLFAGVHYYSDHYWGIKLGEQTAVAMLRDVLDQAYTDGRDVTPTFTEYFGDYSNPLEISIEELENLRESATHGLTYPKS

SEQ ID NO: 93 - DNA, 760 bp, synthetic construct
  source 1..760: mol_type = other DNA
tcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctgaaatcaccagtctctctctacaaatctatctctctcgagctttcgcagatctgtcgatcgacc

SEQ ID NO: 94 - DNA, 2385 bp, synthetic construct
  source 1..2385: mol_type = other DNA
tgccaagtgcccgataaaaagcacttggcaaagcgccgagcactcggaaagagccggattccggtgtggttgtttcatggaagttgtaagtagaccatcttaacaaggtcaacaatagaagtacaactcacaacattagatactgccaaagttaatgttgagaggcttcgtgaactccttatggatttactagtagttgaaaaaccaatactggttatctatatgagttataataattaaacattggtaatcaaagtgaacaattcttaggttaacatgaagactacaaagcatgtcaagaacggttgaaactgttaaaacactaagaaactctagattaattgtgttggactatgtccatacttctaagaatttggtatgccagtttatagaagatttgtcacataatgtgatagatggtgcatcgagaaagatggacttgagacccacttaatgccattccatagtagtagcattttctatgtgattggagatctcatgaagtaaaatggtgaaataagctagagaatgactgatggagagactccttaaataaaatctaggtaatttcatatgaacatttctccttcgatgtacgataggctaactaaaatcttaatatgatttgagtgacatatctaagcagggatgttatcttatagaacatcctataaggaacacacctatatgactttgactactaatcacagtctatgatatgtgggaaatatttttttactcatgaaaggcctcgaagtttgacttatatgcttcaaaccaatggataactgattgaatctgtttcagacagcagcatagttaacttcaaaattctatatctcccaaacttctcaaccaaaaattattaaattatgctgacagtaagaacttttatccctctacaaaagtctccatagttggaagagccaattcggccatttactagatgaaacctaccagttaacagacccgctcatttttgtcaggcaaacaagaacccttatatatcaaagagtagtggatttgaatttgtctaatatgaagtttatgggcttggatcatttaaagtattttataattctgaaagacaagattaaactaactatgtatgatcctttagcaccgtacagatggtcgatgggacagttacatggtttatatagatggacctatctatattccttcagtggttgagaacaaggtaaaggtgtgtcacactcacgcgtgcgccgcctccgccgagacgaggcatggcgttgcagggtgaggcatggcatggtgcagcacgatcgcagtcatggtcgtggcacagtgtagcactgttgtggatagatagataaactattgttgtagttagagtgcatgagtttttgaatgcaacactagaattttcacaatttaacttaattaagaagttatatcttgatggcacagagttacctgaccattgatctcacatgcccttttagctctctcgtactctagacttaccctgactatttaggtctcctgactcgccaatataaacaccatcctatcatttgctgtcgaatacttctgtgtcacaactcaactgtcagatgagtactccattacagcactgagcaggcctccgaaatgaatatctactcagatgaaggacgacaatcgtgttctagggaaatatcagcgacacaactccttacttcctccgcttcttcctagtgttttttgttgtgattgagtcgacatagcaacaacactgcactattacaaccagtacgactatatcaactaggcaatgtcttccttatatgttactatttattttgctcatattcattatgtttaaatcacataggcgcctttatgttggcatcaaaaaatcagtatcaactttatagattaaaatgaaactaaaagtacctaaatttctatcggtggggatcgagtgattctttaaaccgattattacacaagttaaccacactaaaattaacaatggtgaatcgtgccatgatttttttctagtggaaaatagccaaaccaagcaacacatatgtggctatcgttacacatgtgtaaaggtattgcatcacaccattgtcacccatgtatgtggacaataccgagaggaaaaaccacttatttattgtattttatcaagtttgtcttgcttacgtataaattataacccaacaaagtaatcactaaatgtcaaaaccaactagataccatgtcatctctaccttatcttactaatattctttttgcaaaatccaaaattaatcttgcacaagcacaaggactgagatgtgtataaatatctcttaaattagtagctaatatatcgcacatattattgagaccaactagcgacatagaaagcacaatagtgtaccaacc

SEQ ID NO: 95 - DNA, 7288 bp, synthetic construct
  source 1..7288: mol_type = other DNA
tgccaagtgcccgataaaaagcacttggcaaagcgccgagcactcggaaagagccggattccggtgtggttgtttcatggaagttgtaagtagaccatcttaacaaggtcaacaatagaagtacaactcacaacattagatactgccaaagttaatgttgagaggcttcgtgaactccttatggatttactagtagttgaaaaaccaatactggttatctatatgagttataataattaaacattggtaatcaaagtgaacaattcttaggttaacatgaagactacaaagcatgtcaagaacggttgaaactgttaaaacactaagaaactctagattaattgtgttggactatgtccatacttctaagaatttggtatgccagtttatagaagatttgtcacataatgtgatagatggtgcatcgagaaagatggacttgagacccacttaatgccattccatagtagtagcattttctatgtgattggagatctcatgaagtaaaatggtgaaataagctagagaatgactgatggagagactccttaaataaaatctaggtaatttcatatgaacatttctccttcgatgtacgataggctaactaaaatcttaatatgatttgagtgacatatctaagcagggatgttatcttatagaacatcctataaggaacacacctatatgactttgactactaatcacagtctatgatatgtgggaaatatttttttactcatgaaaggcctcgaagtttgacttatatgcttcaaaccaatggataactgattgaatctgtttcagacagcagcatagttaacttcaaaattctatatctcccaaacttctcaaccaaaaattattaaattatgctgacagtaagaacttttatccctctacaaaagtctccatagttggaagagccaattcggccatttactagatgaaacctaccagttaacagacccgctcatttttgtcaggcaaacaagaacccttatatatcaaagagtagtggatttgaatttgtctaatatgaagtttatgggcttggatcatttaaagtattttataattctgaaagacaagattaaactaactatgtatgatcctttagcaccgtacagatggtcgatgggacagttacatggtttatatagatggacctatctatattccttcagtggttgagaacaaggtaaaggtgtgtcacactcacgcgtgcgccgcctccgccgagacgaggcatggcgttgcagggtgaggcatggcatggtgcagcacgatcgcagtcatggtcgtggcacagtgtagcactgttgtggatagatagataaactattgttgtagttagagtgcatgagtttttgaatgcaacactagaattttcacaatttaacttaattaagaagttatatcttgatggcacagagttacctgaccattgatctcacatgcccttttagctctctcgtactctagacttaccctgactatttaggtctcctgactcgccaatataaacaccatcctatcatttgctgtcgaatacttctgtgtcacaactcaactgtcagatgagtactccattacagcactgagcaggcctccgaaatgaatatctactcagatgaaggacgacaatcgtgttctagggaaatatcagcgacacaactccttacttcctccgcttcttcctagtgttttttgttgtgattgagtcgacatagcaacaacactgcactattacaaccagtacgactatatcaactaggcaatgtcttccttatatgttactatttattttgctcatattcattatgtttaaatcacataggcgcctttatgttggcatcaaaaaatcagtatcaactttatagattaaaatgaaactaaaagtacctaaatttctatcggtggggatcgagtgattctttaaaccgattattacacaagttaaccacactaaaattaacaatggtgaatcgtgccatgatttttttctagtggaaaatagccaaaccaagcaacacatatgtggctatcgttacacatgtgtaaaggtattgcatcacaccattgtcacccatgtatgtggacaataccgagaggaaaaaccacttatttattgtattttatcaagtttgtcttgcttacgtataaattataacccaacaaagtaatcactaaatgtcaaaaccaactagataccatgtcatctctaccttatcttactaatattctttttgcaaaatccaaaattaatcttgcacaagcacaaggactgagatgtgtataaatatctcttaaattagtagctaatatatcgcacatattattgagaccaactagcgacatagaaagcacaatagtgtaccaaccatggcagaagaaaggcggcagaacgcgctagagatccgcattcaagcggcaaagctcgcaaagaagcgggatcacccgacacataaggcaaacggcgatgaggaccgctatccagagacgcttatcggctcattcaccaaaggcttgccgcacgagaaggagacagggcttctctccaatcctgcagactttgctgatttcgttagggctattaatacaggcgccatcaaggacatgaggcggctcaagctgggtattgacgaagaccctcggttcatctccggtatagcctcaaaaggcgacaaacatcccttcgcggacaccagagcctgggaatctatggctgctggcttgacctatgatctggaggggcctgacgcgcaggccgttacgatgccccccgccccgaaacttgacagcgacgagttggtgactgaaattaccgaatcatactggatggcccttctccgagatgttccgtttactgagttcgagcgcgacggaaatactgcagctgctgcagcgtccataagcagaactcgatgggtacagtacaacgagcaaccttcacagcggccatcgactttgacggacgaggaaatagcccgtttgcgcgggccttatacgaagaagaatgttttcaggggggtgaccaacggtgagaacgtaggcccatacctctcccagttcctcctcgtgggcaccaaaggcatcggcgatgcacagcaagtgtcggatggatacgtccagtacggcggcatgaggatggatcagcgcgtaagggtagctgtgcctaagagggattacatgaccacttgggcgagctggctggacgttcagaatgcgggggacttgagaggacgcgaaatatacgacgacgacacgccattcaggttcataactaccccgcgtgatctggcaacttgggtgcacttcgacgccttgtaccaagcataccttaacgcgtgtattatcctgctcgatatcaaggctccgttcgatccgcacataccatttcaagccgacgacgacgtcgacaagcagcaaggattcgcaaccttcggaggtccgcatatcctcagtctctgcacagaggtcgccaccagggcactcaaagccgttcgcttccagaagtataacctacaccggcgtctccgccctgaagccatcggaggcctggttgagcggttcaagaagaccaatggcgacccaaagttcgctcctgtgaaaaagctcgtgaatgacctcgacggagacatgcttcgccgagtcgagcaacacaattgcgagcagaacaaactttccgacgatggccacgcgagaagagaggactacagcccggagggagagtcaagccagagctatcttcttcctatggcgttcccggagggttctcctatgcatccatcatatggtgctggccacgctaccgtcgccggggcgtgcgtgacagtgttgaaggcttttttcgaccacgaatacgagcttgacttctgctatgtccccaccaccgacggtaagcgtctcgagaaggtcaacatcaatgagaagttgaccgttgagggcgagctgaataagttgtgtgcaaacatcagcatagggcgcaactgggcaggggtccactactactctgactatttcgagtccattaaggtgggagaggagatagccatcggtattctgcaggagcagaagcttacatacggggaggacttctttatgacactaccgaaatttgatggcgagaagattaggattggaagcggagctactaacttcagcctgctgaagcaggctggagacgtggaggagaaccctggacctatggcccatacatcagaatctgtgaatcctagagatgtttgcattgtgggtgttgcacgtactccaatgggtggctttctcggatctctttcatctttacctgccacaaagcttggatctttagctattgcagctgctttgaagagagcaaatgttgatccagctcttgttcaagaagttgtctttggcaatgttcttagtgctaatttgggtcaagctcctgctcgtcaagctgctttaggtgcaggaatccctaactctgttatctgtactacagttaacaaggtttgtgcatcaggcatgaaagcggtaatgattgctgctcaaagtatccagttagggatcaatgatgtagttgtggcgggtggtatggaaagcatgtctaatacaccaaaatatttggcagaagcaaggaagggatctcgttttggtcatgattctttagtagatggaatgttgaaggatggactatgggatgtctataacgactgtgggatgggaagctgtgcagaattatgcgctgagaagtttcagattacaagggagcagcaagatgactatgcagttcagagttttgagcgtggtattgctgcccaggaagctggcgccttcacatgggaaatcgtcccggttgaagtttctggaggaagaggtaggccatcaaccattgttgacaaggacgaaggtcttgggaagtttgatgctgcaaaattgaggaaactccgtcctagtttcaaagagaatggagggactgttacagctggaaatgcgtctagcataagtgatggtgcagctgcccttgtcctagtgagcggagagaaggctcttcagctaggacttctagtattagcaaaaattaaagggtatggtgacgcagctcaggaaccagagtttttcactactgctcctgctcttgctataccaaaagccattgcacatgctggtttggaatcttctcaagttgattactatgagatcaatgaagcatttgcagttgtagcacttgcaaatcaaaagctactcgggattgctccagagaaagtgaacgtaaatggaggagctgtctccttaggacaccctctaggctgcagtggcgcccgtattctaatcacgttgcttgggatactaaagaagagaaacggaaagtacggtgtgggaggagtgtgcaacggaggaggaggtgcttctgctctagttcttgagctccttggttcaggtgccactaatttttctctcttgaaacaggccggtgacgttgaagagaacccaggtccaatgaagaagcgcttaaccacttccacttgttcttcttctccatcttcctctgtttcttcttctactactacttcctctcctattcagtcggaggctccaaggcctaaacgagccaaaagggctaagaaatcttctccttctggtgataaatctcataacccgacaagccctgcttctacccgacgcagctctatctacagaggagtcactagacatagatggactgggagattcgaggctcatctttgggacaaaagctcttggaattcgattcagaacaagaaaggcaaacaagtttatctgggagcatatgacagtgaagaagcagcagcacatacgtacgatctggctgctctcaagtactggggacccgacaccatcttgaattttccggcagagacgtacacaaaggaattggaagaaatgcagagagtgacaaaggaagaatatttggcttctctccgccgccagagcagtggtttctccagaggcgtctctaaatatcgcggcgtcgctaggcatcaccacaacggaagatgggaggctcggatcggaagagtgtttgggaacaagtacttgtacctcggcacctataatacgcaggaggaagctgctgcagcatatgacatggctgcgattgagtatcgaggcgcaaacgcggttactaatttcgacattagtaattacattgaccggttaaagaagaaaggtgttttcccgttccctgtgaaccaagctaaccatcaagagggtattcttgttgaagccaaacaagaagttgaaacgagagaagcgaaggaagagcctagagaagaagtgaaacaacagtacgtggaagaaccaccgcaagaagaagaagagaaggaagaagagaaagcagagcaacaagaagcagagattgtaggatattcagaagaagcagcagtggtcaattgctgcatagactcttcaaccataatggaaatggatcgttgtggggacaacaatgagctggcttggaacttctgtatgatggatacagggttttctccgtttttgactgatcagaatctcgcgaatgagaatcccatagagtatccggagctattcaatgagttagcatttgaggacaacatcgacttcatgttcgatgatgggaagcacgagtgcttgaacttggaaaatctggattgttgcgtggtgggaagagagagcccaccctcttcttcttcaccattgtcttgcttatctactgactctgcttcatcaacaacaacaacaacaacctcggtttcttgtaactatttggtctgataacgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcatatgaagatgaagatgaaatatttggtgtgtcaaataaaaagcttgtgtgcttaagtttgtgtttttttcttggcttgttgtgttatgaatttgtggctttttctaatattaaatgaatgtaagatctcattataatgaataaacaaatgtttctataatccattgtgaatgttttgttggatctcttctgcagcatataactactgtatgtgctatggtatggactatggaatatgattaaagataagggaa

(19) United States
(12) Patent Application Publication
(10) Pub. No.: US 2024/0384284 A1
(43) Pub. Date: Nov. 21, 2024

(54) FEED CROPS FOR REDUCED METHANOGENESIS IN LIVESTOCK

(71) Applicant: Elysia Creative Biology, LLC, Raleigh, NC (US)

(72) Inventor: Eli David HORNSTEIN, Raleigh, NC (US)

(21) Appl. No.: 18/664,124

(22) Filed: May 14, 2024

Related U.S. Application Data
(60) Provisional application No. 63/503,222, filed on May 19, 2023.
(60) Provisional application No. 63/471,102, filed on Jun. 5, 2023.
(60) Provisional application No. 63/512,776, filed on Jul. 10, 2023.

(51) Int. Cl. C12N 15/82; C07K 14/415; C12N 9/08; C12N 9/10

(52) U.S. Cl. CPC C12N 15/8243; C07K 14/415; C12N 9/0065; C12N 9/1029; C12N 15/8234; C12Y 111/01018; C12Y 111/01802; C12Y 111/01806; C12Y 203/01009

(57) ABSTRACT

Described herein are methods and plants for the reduction of harmful byproducts of animal agriculture by biotechnological means. More particularly, the methods described herein use genetic engineering to enhance plant biosynthesis of small, halogenated molecules, including bromoform (CHBr₃), iodoform (CHI₃), chloroform (CHCL₃), or combinations thereof that inhibit enteric methanogenesis in livestock. The plants may include transgenic elements that further relate to targeting engineered metabolism to specific plant tissues and stabilizing specific end products. Such plants can be used as feedstock for ruminant animals.

### CROSS-REFERENCE TO RELATED APPLICATIONS

This application claims priority to U.S. Provisional Patent Application No. 63/503,222, filed May 19, 2023; U.S. Provisional Patent Application No. 63/471,102, filed Jun. 5, 2023; and U.S. Provisional Patent Application No. 63/512,776, filed Jul. 10, 2023, each of which are incorporated by reference herein in its entirety.

### REFERENCE TO SEQUENCE LISTING

This application was filed with a Sequence Listing XML in ST.26 XML format accordance with 37 C.F.R. § 1.831 and PCT Rule 13ter. The Sequence Listing XML file submitted in the USPTO Patent Center, “888689-0002-US02_sequence_listing_xml_14 May 2024.xml,” was created on May 14, 2024, contains 95 sequences, has a file size of 188 kilobytes (192,512 bytes), and is incorporated by reference in its entirety into the specification.

### BACKGROUND

Ruminant animals, including cattle and sheep, have a unique digestive system involving a multichambered stomach where microbes perform essential steps in digestion via a process known as enteric fermentation. The animal provides its ruminal microbes with a specialized environment and a constant supply of plant matter, from which the microbes convert structural carbohydrates such as cellulose to simple sugars that the animal can use for energy. This symbiosis makes ruminants an important part of the agricultural system due to their unique ability to digest food sources that most other animals cannot. However, enteric fermentation also results in production of methane (CH₄), a greenhouse gas (GHG), many times more powerful than CO₂. Ruminant enteric fermentation is the largest single source of human-generated methane, making up about 5% of worldwide GHG emissions in recent years (GWP₁₀₀). This is larger than the effect of worldwide deforestation, aviation, and shipping combined.

Leading scientists and conservation organizations have called for urgent focus on methane as a means to achieve immediate results in the transition to climate sustainability, and 150 countries have signed a pledge to reduce their methane emissions 30% by 2030. Elimination of enteric methane supports such worldwide efforts and offers large climate benefits, including a reduction in the GHG footprint of beef, dairy, and sheep products by 46-65%, and avoidance of up to 0.3° C. of warming within the next 20 years.

Production of methane is not an intrinsic requirement of enteric fermentation or ruminant agriculture, and the productivity of livestock is unchanged or even improved when methane emission is reduced. The proximate source of methane is a relatively small contingent of methanogenic microbe species in the rumen microbiome that use the Wolfe cycle to reduce CO₂ to methane across a multistep process that produces necessary precursors for DNA and protein and drives the flow of cellular energy via ferredoxin. In the second-to-last step of this cycle, methyl-coenzyme M reductase (MCR) reduces methyl-coenzyme M (CH₃—S-COM) with coenzyme B-thiol (CoB—SH) to the disulfide COM-S—S—CoB, while releasing CH₄ as a byproduct, which subsequently makes its way into the atmosphere. When ruminants' feed is supplemented with a compound that blocks the activity of MCR or precursor enzymes, methane production drops dramatically, as does the population of methanogens in the rumen.

Chemicals exist that can impede methanogenesis in vivo with varying mechanisms and efficiency. 3-nitrooxypropanol is a commercially available feed supplement that inactivates MCR by oxidation of its active site. Bromoform (CHBr₃) competitively inhibits methanogenesis by binding to both MCR and the enzyme that provides its substrate in the prior step of the Wolfe cycle, coenzyme M methyltransferase (CMM). Bromoform is one of the most effective antimethanogens characterized to date, effecting in vivo methane reduction of up to 98%. Bromoform and related haloalkanes occur naturally in marine organisms, most prominently the red macroalgae genus Asparagopsis which is farmed expressly for use as a bromoform-rich feed supplement to reduce enteric methane emissions from livestock.

Harnessing bromoform to reduce methane in form of the pure chemical or seaweed additives presents several challenges that impose additional costs and logistical requirements on farmers and the broader supply chain. Pure bromoform is expensive to synthesize, hard to formulate for delivery on farms, and presents health and environmental concerns when present as a concentrated point source. If relying on native biosynthesis in Asparagopsis or similar species, significant areas of ocean would need to be converted to aquaculture, or resource-intensive facilities built on land, to produce sufficient amounts of bromoform-bearing material to meaningfully reduce enteric methane emissions. This results in environmental impacts that infringe the benefits of any emission reduction achieved. Bromoform also volatilizes from seaweeds to the atmosphere, where it contributes to ionic degradation of ozone, and a lack of containment of this flux during the pre-harvest growth period in large-scale aquaculture is of environmental concern. Bromoform in the form of chemical additives or seaweed must also be transported long distances from production sites and specially formulated and packaged to manage volatilization of bromoform in storage, bioavailability, and practical use, adding further environmental costs and barriers to adoption.

A need exists for a system of integrating bromoform and similar molecules into animal feed without the barrier imposed by producing and delivering this chemical separately from the primary feed supply. There is also a need for the system to avoid the high land and resource demands associated with existing means of bromoform production via seaweed aquaculture. There is a need for the system to limit loss of bromoform from harvested plant material. Finally, there is a need for the system to contain or prevent bromoform leakage during the pre-harvest growth period and from unharvested material.

### SUMMARY

One embodiment described herein is a genetically modified feed crop for reducing methanogenesis in a livestock animal, the genetically modified feed crop comprising: a transgenic polynucleotide construct comprising a haloperoxidase (HPO) gene sequence encoding a HPO protein, wherein the HPO protein enables biosynthesis of bromoform (CHBr₃), chloroform (CHCl₃), iodoform (CHI₃), or a combination thereof in the genetically modified feed crop. In another aspect, the HPO gene sequence encoding a HPO protein is a vanadium-dependent bromoperoxidase (VBPO), a vanadium-dependent iodoperoxidase (VIPO), vanadium-dependent chloroperoxidase (VCPO), or a nonspecific vanadium-dependent haloperoxidase (VHPO). In another aspect, the genetically modified feed crop is a monocot plant or a dicot plant. In another aspect, the HPO gene sequence has at least 90-99% identity to any one of SEQ ID NO: 1, 3, 41, 43, 45, 47, 49, 51, 52, 53, 54, 55, 56, 57, 58, 59, 60, 61, 63, 65, 67, 69, 71, 73, 75, 77, 79, 81, 83, 85, 87, 89, or 91. In another aspect, the HPO gene sequence is selected from any one of SEQ ID NO: 1, 3, 41, 43, 45, 47, 49, 51, 52, 53, 54, 55, 56, 57, 58, 59, 60, 61, 63, 65, 67, 69, 71, 73, 75, 77, 79, 81, 83, 85, 87, 89, or 91. In another aspect, the HPO protein comprises an amino acid sequence having at least 90-99% identity to any one of SEQ ID NO: 2, 4-8, 42, 44, 46, 48, 50, 62, 64, 66, 68, 70, 72, 74, 76, 78, 80, 82, 84, 86, 88, 90, or 92. In another aspect, the HPO protein comprises an amino acid sequence selected from any one of SEQ ID NO: 2, 4-8, 42, 44, 46, 48, 50, 62, 64, 66, 68, 70, 72, 74, 76, 78, 80, 82, 84, 86, 88, 90, or 92. In another aspect, the HPO gene sequence comprises a whole-plant constitutive promoter and the HPO gene sequence is expressed under the whole-plant constitutive promoter. In another aspect, the whole-plant constitutive promoter comprises a polynucleotide sequence having at least 90-99% identity to any one of SEQ ID NO: 19-20, or 93. In another aspect, the whole-plant constitutive promoter comprises a polynucleotide sequence selected from any one of SEQ ID NO: 19-20, or 93. In another aspect, the HPO gene sequence comprises an endosperm-specific promoter or photosynthetic-tissue promoter and is expressed under the endosperm-specific promoter or photosynthetic-tissue promoter. In another aspect, the endosperm-specific promoter comprises a polynucleotide sequence having at least 90-99% identity to any one of SEQ ID NO: 24-25, or 94; and the photosynthetic-tissue promoter comprises a polynucleotide sequence having at least 90-99% identity to any one of SEQ ID NO: 21-23. In another aspect, the endosperm-specific promoter comprises a polynucleotide sequence selected from any one of SEQ ID NO: 24-25, or 94; and the photosynthetic-tissue promoter comprises a polynucleotide sequence selected from any one of SEQ ID NO: 21-23. The genetically modified feed crop of claim 1, further comprising: a second transgenic polynucleotide construct comprising: an acetoacetyl-CoA thiolase 2 (AACT2) gene sequence encoding an AACT2 protein. In another aspect, the AACT2 gene sequence has at least 90-99% identity to any one of SEQ ID NO: 9-10 or 12-13. In another aspect, the AACT2 gene sequence is selected from any one of SEQ ID NO: 9-10 or 12-13. In another aspect, the AACT2 protein comprises an amino acid sequence having at least 90-99% identity to SEQ ID NO: 11. In another aspect, the AACT2 protein comprises an amino acid sequence selected from SEQ ID NO: 11. In another aspect, the AACT2 gene sequence comprises a whole-plant constitutive promoter and the AACT2 gene sequence is expressed under the whole-plant constitutive promoter. In another aspect, the whole-plant constitutive promoter comprises a polynucleotide sequence having at least 90-99% identity to any one of SEQ ID NO: 19-20, or 93. In another aspect, the whole-plant constitutive promoter comprises a polynucleotide sequence selected from any one of SEQ ID NO: 19-20, or 93. In another aspect, the HPO gene sequence and AACT2 gene sequence are separated by a self-cleaving peptide gene sequence encoding for a self-cleaving peptide. In another aspect, the self-cleaving peptide gene sequence has at least 90-99% identity to any one of SEQ ID NO: 30, 32, 34, 36, 38, or 40. In another aspect, the self-cleaving peptide gene sequence is selected from any one of SEQ ID NO: 29, 31, 33, 35, 37, or 39. In another aspect, the self-cleaving peptide comprises an amino acid sequence having at least 90-99% identity to any one of SEQ ID NO: 30, 32, 34, 36, 38, or 40. In another aspect, the self-cleaving peptide comprises an amino acid sequence selected from any one of SEQ ID NO: 30, 32, 34, 36, 38, or 40. In another aspect, the HPO is a Vanadium-dependent bromoperoxidase (VBPO) gene sequence having at least 90-99% identity to any one of SEQ. ID NO: 1 or 3. In another aspect, the HPO is a Vanadium-dependent bromoperoxidase (VBPO) gene sequence selected from any one of SEQ ID NO: 1 or 3. In another aspect, the HPO is a Vanadium-dependent bromoperoxidase (VBPO) protein comprising an amino acid sequence having at least 90-99% identity to any one of SEQ ID NO: 2 or 4-8. In another aspect, the HPO is a Vanadium-dependent bromoperoxidase (VBPO) protein comprising an amino acid sequence selected from any one of SEQ ID NO: 2 or 4-8. In another aspect, the HPO is a Vanadium-dependent iodoperoxidase (VIPO) gene sequence having at least 90-99% identity to any one of SEQ. ID NO: 41, 43, 45, 47, 49 or 51-60. In another aspect, the HPO is a Vanadium-dependent iodoperoxidase (VIPO) gene sequence selected from any one of SEQ ID NO: 41, 43, 45, 47, 49 or 51-60. In another aspect, the HPO is a Vanadium-dependent iodoperoxidase (VIPO) protein comprising an amino acid sequence having at least 90-99% identity to any one of SEQ ID NO: 42, 44, 46, 48, or 50. In another aspect, the HPO is a Vanadium-dependent iodoperoxidase (VIPO) protein comprising an amino acid sequence selected from any one of SEQ ID NO: 42, 44, 46, 48, or 50. In another aspect, the HPO is a Vanadium-dependent chloroperoxidase (VCPO) gene sequence having at least 90-99% identity to any one of SEQ. ID NO: 61, 63, 65, 67, 69, 71, 73, or 75. In another aspect, the HPO is a Vanadium-dependent chloroperoxidase (VCPO) gene sequence selected from any one of SEQ ID NO: 61, 63, 65, 67, 69, 71, 73, or 75. In another aspect, the HPO is a Vanadium-dependent chloroperoxidase (VCPO) protein comprising an amino acid sequence having at least 90-99% identity to any one of SEQ ID NO: 62, 64, 66, 68, 70, 72, 74, or 76. In another aspect, the HPO is a Vanadium-dependent chloroperoxidase (VCPO) protein comprising an amino acid sequence selected from any one of SEQ ID NO: 62, 64, 66, 68, 70, 72, 74, or 76. In another aspect, the HPO is a nonspecific vanadium-dependent haloperoxidase (VHPO) gene sequence having at least 90-99% identity to any one of SEQ. ID NO: 77, 79, 81, 83, 85, 87, 89, or 91. In another aspect, the HPO is a nonspecific vanadium-dependent haloperoxidase (VHPO) gene sequence selected from any one of SEQ ID NO: 77, 79, 81, 83, 85, 87, 89, or 91. In another aspect, the HPO is a nonspecific vanadium-dependent haloperoxidase (VHPO) protein comprising an amino acid sequence having at least 90-99% identity to any one of SEQ ID NO: 78, 80, 82, 84, 86, 88, 90, or 92. In another aspect, the HPO is a nonspecific vanadium-dependent haloperoxidase (VHPO) protein comprising an amino acid sequence selected from any one of SEQ ID NO: 78, 80, 82, 84, 86, 88, 90, or 92.

In another aspect, the genetically modified feed crop further comprises: a third transgenic polynucleotide construct comprising: a Wrinkled 1 (WRI1) gene sequence encoding a WRI1 protein. In another aspect, the WRI1 gene sequence has at least 90-99% identity to any one of SEQ ID NO: 14-15 or 17-18. In another aspect, the WRI1 gene sequence is selected from any one of SEQ ID NO: 14-15 or 17-18. In another aspect, the WRI1 protein comprises an amino acid sequence having at least 90-99% identity to SEQ ID NO: 16. In another aspect, the WRI1 protein comprises an amino acid sequence selected from SEQ ID NO: 16. In another aspect, the HPO gene sequence and WRI1 gene sequence are separated by a self-cleaving peptide gene sequence encoding for a self-cleaving peptide. In another aspect, the self-cleaving peptide gene sequence has at least 90-99% identity to any one of SEQ ID NO: 30, 32, 34, 36, 38, or 40. In another aspect, the self-cleaving peptide gene sequence is selected from any one of SEQ ID NO: 29, 31, 33, 35, 37, or 39. In another aspect, the self-cleaving peptide comprises an amino acid sequence having at least 90-99% identity to any one of SEQ ID NO: 30, 32, 34, 36, 38, or 40. In another aspect, the self-cleaving peptide comprises an amino acid sequence selected from any one of SEQ ID NO: 30, 32, 34, 36, 38, or 40.

Another embodiment described herein is a method for reducing methanogenesis in a livestock animal, the method comprising: feeding a livestock animal a genetically modified feed crop comprising: a transgenic polynucleotide construct comprising a haloperoxidase (HPO) gene sequence encoding a HPO protein; wherein the HPO protein enables biosynthesis of bromoform (CHBr₃), chloroform (CHCl₃), iodoform (CHI₃), or a combination thereof in the genetically modified feed crop. In another aspect, the HPO gene sequence encoding a HPO protein is a vanadium-dependent bromoperoxidase (VBPO), a vanadium-dependent iodoperoxidase (VIPO), vanadium-dependent chloroperoxidase (VCPO), or a nonspecific vanadium-dependent haloperoxidase (VHPO). In another aspect, the method reduces the level of methane produced and released from the livestock animal as compared to a livestock animal that is not fed the genetically modified feed crop. In another aspect, the livestock animal is a cow, a sheep, a goat, a buffalo, a camel, a deer, an elk, an antelope, a gazelle, or a giraffe.

Another embodiment described herein is a method of making a genetically modified feed crop for reducing methanogenesis in a livestock animal, the method comprising: transforming a monocot plant or a dicot plant with a transgenic polynucleotide construct comprising a haloperoxidase (HPO) gene sequence encoding a HPO protein, wherein the HPO protein enables biosynthesis of bromoform (CHBr₃), chloroform (CHCl₃), iodoform (CHI₃), or a combination thereof in the genetically modified feed crop; and growing the transformed monocot plant or dicot plant comprising the transgenic polynucleotide construct to generate the genetically modified feed crop. In another aspect, the HPO gene sequence encoding a HPO protein is a vanadium-dependent bromoperoxidase (VBPO), a vanadium-dependent iodoperoxidase (VIPO), vanadium-dependent chloroperoxidase (VCPO), or a nonspecific vanadium-dependent haloperoxidase (VHPO). In another aspect, the genetically modified feed crop comprises an increased level of HPO protein as compared to a plant that is not transformed with the transgenic polynucleotide construct. In another aspect, the livestock animal is a cow, a sheep, a goat, a buffalo, a camel, a deer, an elk, an antelope, a gazelle, or a giraffe.

Another embodiment described herein is the use of the genetically modified feed crop as described herein for reducing methanogenesis in a livestock animal. In another aspect, the livestock animal is a ruminant animal. In another aspect, the livestock animal is a cow, a sheep, a goat, a buffalo, a camel, a deer, an elk, an antelope, a gazelle, or a giraffe.

### DESCRIPTION OF THE DRAWINGS

FIG. 1A-C show halogen (bromine, iodide, chlorine) uptake, metabolism, and release by red seaweeds (FIG. 1A), land plants (FIG. 1B), and engineered land plants as described herein (FIG. 1C). FIG. 1A shows that seaweeds such as Asparagopsis (i) take up ionic halogens that occur naturally in seawater (ii) at relatively high concentration. Once inside seaweed tissue, halogens such as bromine, iodine, chlorine, are metabolized to haloform, i.e., bromoform (CHBr₃), iodoform (CHI₃), or chloroform (CHCl₃), respectively, by VHPO enzymes and accumulate as a large pool of haloform in specialized cells (iii). A large amount of the haloform produced by seaweeds is released to the atmosphere (iv). Seaweeds and other marine organisms such as cyanobacteria and phytoplankton are the source of a large, naturally occurring flux of halogens from the ocean to the atmosphere in the form of haloform. FIG. 1B shows that unmodified land plants take up halogens from the soil via their roots (v). Bromine and other halogens may occur at lower levels in soil, freshwater, or groundwater than in seawater, but undergo bioaccumulation in land plant tissues (vi). Following uptake into plant tissues, halogens are metabolized to methyl halogens (CH₃X, where X=Br, I, or Cl) by HOL enzymes and released to the atmosphere (vii). Land plants are the source of a large, naturally occurring flux of halogens from the soil to the atmosphere in the form of methyl halides. FIG. 1C shows that in land plants modified as described herein, halogens are up from soil according to normal processes (viii). Once in plant tissues, however, activity of transgenic VHPO enzymes metabolizes halogens to haloforms, e.g., bromoform (CHBr₃), iodoform (CHI₃), or chloroform (CHCl₃) (ix). Haloform synthesis competitively inhibits native methyl halide biosynthesis and halogen bioaccumulation, resulting in reduced emission of methyl halides to the atmosphere and reduced ionic tissue halogens alongside novel bioaccumulation of haloform (ix, x). Additional aspects described herein enhance the holding capacity for bromoform, iodoform, or chloroform and metabolic favorability of biosynthesis, and direct bromoform, iodoform, or chloroform to target tissues.

FIG. 2 shows metabolic alterations and interactions with native plant metabolism of the haloform biosynthesis pathway encoded as described herein. Novel metabolic functions conferred as described herein are shown in thick lines. Native metabolic functions are shown in thin lines. Transgenic expression of Vanadium-dependent haloperoxidase(s) (VHPO) enables synthesis of haloform from naturally present halogen ions and organic substrates including acetoacetyl-S-CoA (AcAc-COA) (i). For example, vanadium-dependent bromoperoxidase (VBPO), vanadium-dependent chloroperoxidase (VCPO), vanadium-dependent iodoperoxidase (VIPO) or nonspecific vanadium-dependent haloperoxidase (VHPO) can be used to create bromoform, chloroform, or iodoform from bromine, chlorine, or iodine, respectively. AcAc-CoA is naturally ubiquitous due to its role as an intermediate in the mevalonate pathway (ii), but concentration is enhanced by transgenic expression of acetoacetyl-CoA thiolase 2 (AACT2) which synthesizes AcAc—Co from another, even more ubiquitous metabolite, acetyl-S-CoA (Ac-CoA) (iii). The Ac-COA substrate of AACT2 may originate from the upstream mevalonate pathway but also multiple other cellular sources including glycolysis, branched-chain amino acid degradation, fatty acid degradation, and activity of alcohol dehydrogenase (iv, v). Transgenic expression of Wrinkled 1 (WRI1) broadly upregulates lipid metabolism resulting in increased storage oil (triacylglycerol) synthesis in target tissues as well as increased metabolic sink strength for upstream intermediates involved in bromoform synthesis (vi, vii). This results in land plants with accumulated bromoform in oil-bearing tissues.

FIG. 3A-B show examples of gene and regulatory sequence combinations providing tissue-targeted haloform accumulation in monocots and dicots. FIG. 3A shows corn, a monocot, is made to synthesize haloform in endosperm. Expression of Wrinkled 1 (WRI1) under the Zein promoter causes starchy endosperm tissues to increase oil synthesis, providing an enhanced environment for bromoform accumulation. Inclusion of the rice SODCc2 intron prevents silencing of the transgene. For example, a particular VHPO such as Vanadium Dependent of the transgene. Bromoperoxidase (VBPO) of Asparagopsis taxiformis is also expressed under the Zein promoter, limiting the expression of this protein and the direct bromoform biosynthesis reaction to endosperm tissue. Bromoform biosynthesis takes place across different subtypes of endosperm across developmental stages of seed filling. Acetoacetyl-CoA thiolase 2 (AACT2) is expressed under the whole-plant constitutive promoter of Ubiquitin 1. Acetoacetyl-CoA (AcAc-COA), the organic substrate for bromoform synthesis by VBPO, is produced at increased levels throughout the plant and delivered to endosperm by both local biosynthesis and long-distance transport during seed provisioning. FIG. 3B shows Arabidopsis, a dicot oilseed, is made to synthesize bromoform in endosperm throughout developmental stages. Expression of Wrinkled 1 is not used because native oil biosynthesis in seeds may be sufficient for some applications. VBPO is expressed under the endosperm-specific Napin promoter, limiting biosynthesis of bromoform to endosperm tissue. AACT2 is expressed under the whole-plant constitutive promoter of Ubiquitin 10, and AcAc-CoA is synthesized within endosperm as well as delivered by seed-filling processes.

FIG. 4A-C show the percent change in methane relative to the starting value based on bromoform administration. FIG. 4A shows the methane reduction from 25 micrograms per gram bromoform delivered as transgenic plant seeds or synthetic bromoform. FIG. 4B shows the methane reduction from 50 micrograms per gram bromoform delivered as transgenic plant seeds or synthetic bromoform. FIG. 4C shows the methane reduction from 100 micrograms per gram bromoform delivered as transgenic plant seeds or synthetic bromoform.

### DETAILED DESCRIPTION

Unless otherwise defined, all technical and scientific terms used herein have the same meaning as commonly understood by one of ordinary skill in the art. For example, any nomenclatures used in connection with, and techniques of biochemistry, molecular biology, immunology, microbiology, genetics, cell and tissue culture, and protein and nucleic acid chemistry described herein are well known and commonly used in the art. In case of conflict, the present disclosure, including definitions, will control. Exemplary methods and materials are described below, although methods and materials similar or equivalent to those described herein can be used in practice or testing of the embodiments and aspects described herein.

As used herein, the terms “amino acid,” “nucleotide,” “polynucleotide,” “vector,” “polypeptide,” and “protein” have their common meanings as would be understood by a biochemist of ordinary skill in the art. Standard single letter nucleotides (A, C, G, T, U) and standard single letter amino acids (A, C, D, E, F, G, H, I, K, L, M, N, P, Q, R, S, T, V, W, or Y) are used herein.

As used herein, the terms such as “include,” “including,” “contain,” “containing,” “having,” and the like mean “comprising.” The present disclosure also contemplates other embodiments “comprising,” “consisting essentially of,” and “consisting of” the embodiments or elements presented herein, whether explicitly set forth or not.

As used herein, the term “a,” “an,” “the” and similar terms used in the context of the disclosure (especially in the context of the claims) are to be construed to cover both the singular and plural unless otherwise indicated herein or clearly contradicted by the context. In addition, “a,” “an,” or “the” means “one or more” unless otherwise specified.

As used herein, the term “or” can be conjunctive or disjunctive.

As used herein, the term “and/or” refers to both the conjuctive and disjunctive.

As used herein, the term “substantially” means to a great or significant extent, but not completely.

As used herein, the term “about” or “approximately” as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In one aspect, the term “about” refers to any values, including both integers and fractional components that are within a variation of up to +10% of the value modified by the term “about.” Alternatively, “about” can mean within 3 or more standard deviations, per the practice in the art. Alternatively, such as with respect to biological systems or processes, the term “about” can mean within an order of magnitude, in some embodiments within 5-fold, and in some embodiments within 2-fold, of a value. As used herein, the symbol “˜” means “about” or “approximately.”

All ranges disclosed herein include both end points as discrete values as well as all integers and fractions specified within the range. For example, a range of 0.1-2.0 includes 0.1, 0.2, 0.3, 0.4 . . . 2.0. If the end points are modified by the term “about,” the range specified is expanded by a variation of up to +10% of any value within the range or within 3 or more standard deviations, including the end points.

As used herein, the terms “active ingredient” or “active pharmaceutical ingredient” refer to a pharmaceutical agent, active ingredient, compound, or substance, compositions, or mixtures thereof, that provide a pharmacological, often beneficial, effect.

As used herein, the terms “control,” or “reference” are used herein interchangeably. A “reference” or “control” level may be a predetermined value or range, which is employed as a baseline or benchmark against which to assess a measured result. “Control” also refers to control experiments or control cells.

As used herein, the term “dose” denotes any form of an active ingredient formulation or composition, including cells, that contains an amount sufficient to initiate or produce a therapeutic effect with at least one or more administrations. “Formulation” and “composition” are used interchangeably herein.

As used herein, the term “prophylaxis” refers to preventing or reducing the progression of a disorder, either to a statistically significant degree or to a degree detectable by a person of ordinary skill in the art.

As used herein, the terms “effective amount” or “therapeutically effective amount,” refers to a substantially non-toxic, but sufficient amount of an action, agent, composition, or cell(s) being administered to a subject that will prevent, treat, or ameliorate to some extent one or more of the symptoms of the disease or condition being experienced or that the subject is susceptible to contracting. The result can be the reduction or alleviation of the signs, symptoms, or causes of a disease, or any other desired alteration of a biological system. An effective amount may be based on factors individual to each subject, including, but not limited to, the subject's age, size, type or extent of disease, stage of the disease, route of administration, the type or extent of supplemental therapy used, ongoing disease process, and type of treatment desired.

As used herein, the term “subject” refers to an animal. Typically, the subject is a mammal. A subject also refers to primates (e.g., humans, male or female; infant, adolescent, or adult), non-human primates, rats, mice, rabbits, pigs, cows, sheep, goats, horses, dogs, cats, fish, birds, and the like. In one embodiment, the subject is a ruminant animal. In another embodiment, the subject is a cow, goat, sheep, buffalo, camel, deer, moose, elk, antelope, gazelle, or giraffe.

As used herein, the terms “inhibit,” “inhibition,” or “inhibiting” refer to the reduction or suppression of a given biological process, condition, symptom, disorder, or disease, or a significant decrease in the baseline activity of a biological activity or process.

As used herein, “treatment” or “treating” refers to prophylaxis of, preventing, suppressing, repressing, reversing, alleviating, ameliorating, or inhibiting the progress of biological process including a disorder or disease, or completely eliminating a disease. A treatment may be either performed in an acute or chronic way. The term “treatment” also refers to reducing the severity of a disease or symptoms associated with such disease prior to affliction with the disease. “Repressing” or “ameliorating” a disease, disorder, or the symptoms thereof involves administering a cell, composition, or compound described herein to a subject after clinical appearance of such disease, disorder, or its symptoms. “Prophylaxis of” or “preventing” a disease, disorder, or the symptoms thereof involves administering a cell, composition, or compound described herein to a subject prior to onset of the disease, disorder, or the symptoms thereof. “Suppressing” a disease or disorder involves administering a cell, composition, or compound described herein to a subject after induction of the disease or disorder thereof but before its clinical appearance or symptoms thereof have manifest.

For the purposes of this description, the names of genes and proteins given from a particular species are understood to refer to a representative homolog of the named gene. A homolog is any genomic DNA sequence having 70% or greater identity to the genomic region corresponding to the representative gene, including introns, untranscribed regions, and regulatory regions; any protein-coding RNA or cDNA sequence having 70% or greater identity to the protein-coding RNA or cDNA sequence of the representative gene; or any protein having 70% or greater identity to the representative protein or protein product of the representative gene. Percent identity for DNA and RNA homology is determined over any nucleotide sequence window of 500 base pairs or greater, and protein homology is determined over any amino acid sequence window of 100 residues or greater.

As used herein, the terms “land plant,” “land plants,” “plant,” “plants,” “feed crop,” “feed crops,” “crop,” or “crops” shall be understood to refer to any member of the phylogenetic plant grouping known in the art as the Embryophyta, comprising hornworts, liverworts, mosses, lycophytes, ferns, and other pteridophytes, gymnosperms, and angiosperms. The terms shall be understood to include plant species having aquatic or semiaquatic growth habits but originating in this phylogenetic grouping. For example, duckweeds, reeds, rushes, seagrasses, mangroves, water lilies, domestic rice, wild rice, and water lettuces are land plants.

Haloform is the common name of a compound having the formula CHX₃, where X is a halogen atom, halogens comprising bromine, chlorine, iodine, or fluorine. A haloform may contain more than one type of halogen, for example, bromoiodochloroform is the common name of the compound CHBrICl, which is a haloform. Bromoform is the common name of the compound CHBr₃. Iodoform is the common name of the compound CHI₃. Chloroform is the common name of the compound CHCl₃. Nevertheless, the plants described herein may result in production of additional organohalide compounds having antimethanogenic (e.g., methane-reducing) effects, in combination with or separately from one or more haloforms. An organohalide is any compound containing at least one carbon atom and at least one halogen atom. As used herein, the term “haloform” is understood to represent CHX₃ and any other compounds containing carbon and a halogen whose formation results from activity of a haloperoxidase enzyme or that has the effect of impeding methanogenesis by a similar mechanism to a haloform. As used herein “haloform” refers to CHX₃ where X is a halogen atom, as well as any small organohalide molecules comprising up to 9 halogen atoms and up to 30 carbon atoms and including nitrogen, oxygen, bromine, chlorine, fluorine, or iodine atoms formed by activity of a VHPO on a halogen and an organic substrate, as either an end product or an intermediate, particularly including the organohalides CH₃Br, CH₂Br₂, CHBr₃, CH₃I, CH₂I₂, CHI₃, CH₃Cl, CH₂Cl₂, CHCl₃; other methyl halides in which bromine, iodine, chlorine, or fluorine occur in any combination within the structure described for methyl halides, primary alkyl halides, meaning any molecule of the structure R—CH₂X, R—CHX₂, or R—CX₃, where X is a halogen and R is an alkyl group or haloalkane (including haloalkanes of bromine, chlorine, fluorine, and iodine); secondary alkyl bromides, including molecules having the structure: R¹—(CHX)—R² or R¹—(CX₂)—R², where X is a halogen and R¹ and R² are alkyl groups or haloalkanes; and haloketones, such as molecules containing at least one carbon-oxygen double bond and at least one halogen atom, including 1,1 dihaloacetones, X₂(CH)(CO)CH₂, where X is a halogen; 1,5-halogenated 2,4-diones; 1-dihalogenated ketones; or 1-trihalogenated ketones. As used herein, the term “bromoform” is understood to represent CHBr₃ and any other compounds containing carbon and bromine whose formation results from activity of a bromoperoxidase enzyme or that has the effect of impeding methanogenesis by a similar mechanism to CHBr₃. As used herein, the term “iodoform” is understood to represent CHI₃ and any other compounds containing carbon and iodine whose formation results from activity of an iodoperoxidase enzyme or that has the effect of impeding methanogenesis by a similar mechanism to CHI₃. As used herein, the term “chloroform” is understood to represent CHCl₃ and any other compounds containing carbon and chlorine whose formation results from activity of a chloroperoxidase enzyme or that has the effect of impeding methanogenesis by a similar mechanism to CHCl₃.

Biological processes and molecular activities of chemicals, enzymes, proteins, and nucleic acids are faithfully described as understood in the art at the time of the description. Nevertheless, this description is not intended to be limited by a particular model of molecular or metabolic mechanism of its components. Alternative modes of action, molecular substrates, and interactions by the components different from those described here but having the same or similar effect should be understood to remain within the scope of the embodiments described herein.

Described herein are systems enabling haloform biosynthesis in land plants that are commonly used as animal feed, including grains like corn, soybean, and canola, and pasture crops like alfalfa and ryegrass. By synthesizing haloform directly in these crops, the benefits of methane reduction are achieved with no additional land, energy, or resources beyond what is already needed for growing the crops themselves. The engineered plants make haloform in situ from soil halogens and native plant metabolites and do not require special facilities or supplementation, with the further benefit of enabling methane reduction in otherwise difficult-to-serve systems including smallholders in the developing world. Further, methods and composition described herein uniquely integrates this molecule with a common method of stabilizing and storing haloform—immersion in vegetable oil—by biosynthesizing the active molecule directly within oil-bearing cells.

The methods and composition described herein use several genetic components that work alone or in various permutations. In one embodiment, the transgenic expression of a Vanadium-dependent Bromoperoxidase (VBPO) gene derived from Asparagopsis red algae (seaweed) or marine cyanobacteria is used to produce haloform by catalyzing the reaction of bromide with native plant carbonyl compounds including acetoacetyl-S-CoA (AcAc-COA). In another embodiment, the transgenic expression of acetoacetyl-CoA thiolase 2 (AACT2) of Arabidopsis thaliana, increases the availability of the AcAc-COA precursors. In another embodiment, the transgenic expression of the Wrinkled 1 (WRI1) transcription factor of Arabidopsis thaliana upregulates fatty acid biosynthesis, increasing the availability of precursors for both AcAc-COA and cuticle polymers, and increasing accumulation of storage oil and cuticular wax, which stabilize accumulated haloform and prevent its evaporation. In another embodiment, specific promoters direct the previously listed metabolic functions to the portion of feed plants consumed by animals, which avoids unnecessary tradeoffs with plant growth and limits unwanted emission of haloform into the atmosphere.

Another embodiment is a polynucleotide sequence described herein. In one aspect, the polynucleotide has at least 85% to 99% identity, including all percentages within the specified range, to SEQ ID NO: 1, 3, 9, 10, 12, 13, 14, 15, 17, 18, 19, 20, 21, 22, 23, 24, 25, 26, 27, 28, 29, 31, 33, 35, 37, 39, 41, 43, 45, 47, 49, 51, 52, 53, 54, 55, 56, 57, 58, 59, 60, 61, 63, 65, 67, 69, 71, 73, 75, 77, 79, 81, 83, 85, 87, 89, 91, 93, 94, or 95. In another aspect, the polynucleotide is selected from SEQ ID NO: 1, 3, 9, 10, 12, 13, 14, 15, 17, 18, 19, 20, 21, 22, 23, 24, 25, 26, 27, 28, 29, 31, 33, 35, 37, 39, 41, 43, 45, 47, 49, 51, 52, 53, 54, 55, 56, 57, 58, 59, 60, 61, 63, 65, 67, 69, 71, 73, 75, 77, 79, 81, 83, 85, 87, 89, 91, 93, 94, or 95.

Another embodiment described herein is a polynucleotide vector comprising one or more nucleotide sequences described herein.

Another embodiment described herein is a cell comprising one or more nucleotide sequences described herein or a polynucleotide vector described herein.

Another embodiment is a polypeptide encoded by a nucleotide sequence described herein. In one aspect, the polypeptide has at least 85% to 99% identity, including all percentages within the specified range, to SEQ ID NO: 2, 4, 5, 6, 7, 8, 11, 16, 30, 32, 34, 36, 38, 40, 42, 44, 46, 48, 50, 62, 64, 66, 68, 70, 72, 74, 76, 78, 80, 82, 84, 86, 88, 90, or 92. In another aspect, the polypeptide is selected from SEQ ID NO: 2, 4, 5, 6, 7, 8, 11, 16, 30, 32, 34, 36, 38, 40, 42, 44, 46, 48, 50, 62, 64, 66, 68, 70, 72, 74, 76, 78, 80, 82, 84, 86, 88, 90, or 92.

Another embodiment described herein is a process for manufacturing one or more of the nucleotide sequence described herein or a polypeptide encoded by the nucleotide sequence described herein, the process comprising: transforming or transfecting a cell with a nucleic acid comprising a nucleotide sequence described herein; growing the cells; optionally isolating additional quantities of a nucleotide sequence described herein; inducing expression of a polypeptide encoded by a nucleotide sequence of described herein; isolating the polypeptide encoded by a nucleotide described herein.

Another embodiment described herein is a means for manufacturing one or more of the nucleotide sequences described herein or a polypeptide encoded by a nucleotide sequence described herein, the process comprising: transforming or transfecting a cell with a nucleic acid comprising a nucleotide sequence described herein; growing the cells; optionally isolating additional quantities of a nucleotide sequence described herein; inducing expression of a polypeptide encoded by a nucleotide sequence of described herein; isolating the polypeptide encoded by a nucleotide described herein.

Another embodiment described herein is a nucleotide sequence or a polypeptide encoded by the nucleotide sequence produced by a method or means described herein Another embodiment described herein is the use of an effective amount of a polypeptide encoded by one or more of the nucleotide sequences described herein in SEQ ID NO: 1, 3, 9, 10, 12, 13, 14, 15, 17, 18, 28, 29, 31, 33, 35, 37, 39, 41, 43, 45, 47, 49, 51, 52, 53, 54, 55, 56, 57, 58, 59, 60, 61, 63, 65, 67, 69, 71, 73, 75, 77, 79, 81, 83, 85, 87, 89, or 91.

Another embodiment described herein is a research tool comprising a polypeptide encoded by a nucleotide sequence described herein.

Another embodiment described herein is a reagent comprising a polypeptide encoded by a nucleotide sequence described herein.

The polynucleotides described herein include variants that have substitutions, deletions, and/or additions that can involve one or more nucleotides. The variants can be altered in coding regions, non-coding regions, or both. Alterations in the coding regions can produce conservative or non-conservative amino acid substitutions, deletions, or additions. Especially preferred among these are silent substitutions, additions, and deletions, which do not alter the properties and activities of the binding.

Further embodiments described herein include nucleic acid molecules comprising polynucleotides having at least about 50%, 55%, 60%, 65%, 70%, 75%, 80%, 85%, 90%, 91%, 92%, 93%, 94%, 95%, 96%, 97%, 98%, or 99% identity, and more preferably at least about 90-99% or 100% identity to (a) nucleotide sequences, or degenerate, homologous, or codon-optimized variants thereof, encoding polypeptides having the amino acid sequences in SEQ ID NO: 2, 4, 5, 6, 7, 8, 11, 16, 30, 32, 34, 36, 38, 40, 42, 44, 46, 48, 50, 62, 64, 66, 68, 70, 72, 74, 76, 78, 80, 82, 84, 86, 88, 90, or 92; (b) nucleotide sequences, or degenerate, homologous, or codon-optimized variants thereof, encoding polypeptides having the amino acid sequences in SEQ ID NO: 2, 4, 5, 6, 7, 8, 11, 16, 30, 32, 34, 36, 38, 40, 42, 44, 46, 48, 50, 62, 64, 66, 68, 70, 72, 74, 76, 78, 80, 82, 84, 86, 88, 90, or 92; and (c) nucleotide sequences capable of hybridizing to the complement of any of the nucleotide sequences in (a) or (b) above and capable of expressing functional polypeptides of amino acid sequences in SEQ ID NO: 2, 4, 5, 6, 7, 8, 11, 16, 30, 32, 34, 36, 38, 40, 42, 44, 46, 48, 50, 62, 64, 66, 68, 70, 72, 74, 76, 78, 80, 82, 84, 86, 88, 90, or 92.

Further embodiments described herein include nucleic acid molecules comprising polynucleotides having at least about 50%, 55%, 60%, 65%, 70%, 75%, 80%, 85%, 90%, 91%, 92%, 93%, 94%, 95%, 96%, 97%, 98%, or 99% identity, and more preferably at least about 90-99% or 100% identity to (a) nucleotide sequences that are polycistronic sequences described herein in SEQ ID NO: 28 or 95.

Further embodiments described herein include nucleic acid molecules comprising polynucleotides having at least about 50%, 55%, 60%, 65%, 70%, 75%, 80%, 85%, 90%, 91%, 92%, 93%, 94%, 95%, 96%, 97%, 98%, or 99% identity, and more preferably at least about 90-99% or 100% identity to (a) nucleotide sequences that are promoter or terminator sequences described herein in SEQ ID NO: 20, 21, 22, 23, 24, 25, 26, 27, 93, or 94.

By a polynucleotide having a nucleotide sequence, for example, at least 90-99% “identical” to a reference nucleotide sequence encoding a polypeptide is intended that the nucleotide sequence encoding the polynucleotide be identical to the reference sequence except that the polynucleotide sequence can include up to about 10-to-1 point mutations, additions, or deletions per each 100 nucleotides of the reference nucleotide sequence encoding the polypeptide.

In other words, to obtain a polynucleotide having a nucleotide sequence about at least 90-99% identical to a reference nucleotide sequence, up to 10% of the nucleotides in the reference sequence can be deleted, added, or substituted, with another nucleotide, or a number of nucleotides up to 10% of the total nucleotides in the reference sequence can be inserted into the reference sequence. These mutations of the reference sequence can occur at the 5′- or 3′-terminal positions of the reference nucleotide sequence or anywhere between those terminal positions, interspersed either individually among nucleotides in the reference sequence or in one or more contiguous groups within the reference sequence. The same is applicable to polypeptide sequences about at least 90-99% identical to a reference polypeptide sequence.

As noted above, two or more polynucleotide sequences can be compared by determining their percent identity. Two or more amino acid sequences likewise can be compared by determining their percent identity. The percent identity of two sequences, whether nucleic acid or peptide sequences, is generally described as the number of exact matches between two aligned sequences divided by the length of the shorter sequence and multiplied by 100. An approximate alignment for nucleic acid sequences is provided by the local homology algorithm of Smith and Waterman, Advances in Applied Mathematics 2:4 82-489 (1981). This algorithm can be extended to use with peptide sequences using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14 (6): 6745-6763 (1986).

For example, due to the degeneracy of the genetic code, one having ordinary skill in the art will recognize that a large number of the nucleic acid molecules having a sequence at least 50%, 55%, 60%, 65%, 70%, 75%, 80%, 85%, 90%, 91%, 92%, 93%, 94%, 95%, 96%, 97%, 98%, 99%, or 100% identical to the nucleic acid sequence shown in SEQ ID NO: 1, 3, 9, 10, 12, 13, 14, 15, 17, 18, 28, 29, 31, 33, 35, 37, 39, 41, 43, 45, 47, 49, 51, 52, 53, 54, 55, 56, 57, 58, 59, 60, 61, 63, 65, 67, 69, 71, 73, 75, 77, 79, 81, 83, 85, 87, 89, or 91, or degenerate, homologous, or codon-optimized variants thereof, will encode a polypeptide.

The polynucleotides described herein include those encoding mutations, variations, substitutions, additions, deletions, and particular examples of the polypeptides described herein. For example, guidance concerning how to make phenotypically silent amino acid substitutions is provided in Bowie et al., “Deciphering the Message in Protein Sequences: Tolerance to Amino Acid Substitutions,” Science 247:1306-1310 (1990), wherein the authors indicate that proteins are surprisingly tolerant of amino acid substitutions.

Thus, fragments, derivatives, or analogs of the polypeptides of SEQ ID NO: 2, 4, 5, 6, 7, 8, 11, 16, 30, 32, 34, 36, 38, 40, 42, 44, 46, 48, 50, 62, 64, 66, 68, 70, 72, 74, 76, 78, 80, 82, 84, 86, 88, 90, or 92 can be (i) ones in which one or more of the amino acid residues (e.g., 1, 2, 3, 4, 5, 7, 8, 9, 10, 11, 12, 13, 14, 15, 16, 17, 18, 19, 20, 25, 30, 35, 40, 45, or 50 residues, or even more) are substituted with a conserved or non-conserved amino acid residue (preferably a conserved amino acid residue). Such substituted amino acid residues may or may not be one encoded by the genetic code, or (ii) ones in which one or more of the amino acid residues includes a substituent group (e.g., 1, 2, 3, 4, 5, 7, 8, 9, 10, 15, 20, 25, 30, 35, 40, 45, or 50 residues or even more), or (iii) ones in which the mature polypeptide is fused with another polypeptide or compound, such as a compound to increase the half-life of the polypeptide (for example, polyethylene glycol), or (iv) ones in which the additional amino acids are fused to the mature polypeptide, such as an IgG Fc fusion region peptide or leader or secretory sequence or a sequence which is employed for purification of the mature polypeptide or a proprotein sequence. Such fragments, derivatives, and analogs are deemed to be within the scope of those skilled in the art from the teachings herein.

In addition, fragments, derivatives, or analogs of the polypeptides of SEQ ID NO: 2, 4, 5, 6, 7, 8, 11, 16, 30, 32, 34, 36, 38, 40, 42, 44, 46, 48, 50, 62, 64, 66, 68, 70, 72, 74, 76, 78, 80, 82, 84, 86, 88, 90, or 92 can be substituted with one or more conserved or non-conserved amino acid residue (preferably a conserved amino acid residue). In some cases, these polypeptides, fragments, derivatives, or analogs thereof will have a polypeptide sequence at least 50%, 55%, 60%, 65%, 70%, 75%, 80%, 85%, 90%, 91%, 92%, 93%, 94%, 95%, 96%, 97%, 98%, 99% or 100% identical to the polypeptide sequence shown in SEQ ID NO: 2, 4, 5, 6, 7, 8, 11, 16, 30, 32, 34, 36, 38, 40, 42, 44, 46, 48, 50, 62, 64, 66, 68, 70, 72, 74, 76, 78, 80, 82, 84, 86, 88, 90, or 92 and will comprise functional or non-functional proteins or enzymes. Similarly, additions or deletions to the polypeptides can be made either at the N- or C-termini or within non-conserved regions of the polypeptide (which are assumed to be non-critical because they have not been photogenically conserved).

As described herein, in many cases the amino acid substitutions, mutations, additions, or deletions are preferably of a minor nature, such as conservative amino acid substitutions that do not significantly affect the folding or activity of the protein or additions or deletions to the N- or C-termini. Of course, the number of amino acid substitutions, additions, or deletions a skilled artisan would make depends on many factors, including those described herein. Generally, the number of substitutions, additions, or deletions for any given polypeptide will not be more than about 100, 90, 80, 70, 60, 50, 40, 30, 25, 20, 19, 18, 17, 16, 15, 14, 13, 12, 11, 10, 9, 8, 7, 5, 6, 4, 3, 2, or 1.

Described herein are modified land plants containing haloform at a concentration of at least about 10 μg per gram (parts per million) dry matter in the entire plant, or in any sub-part of the plant such as leaves, seeds, roots, tubers, or stems.

Also described herein are methods of producing plants containing desired levels of haloform by transgenic expression of a vanadium-dependent bromoperoxidase gene, with or without the use of additional complementary genetic modifications.

Also described herein are methods of adjusting haloform concentration in particular target plant species and tissues via altered biosynthesis of acetoacetyl as an organic precursor, altered biosynthesis of lipids to increase haloform stability, and the use of regulatory elements including codon optimization, introns, and constitutive promoters, as well as seed, endosperm, leaf, and photosynthetic tissue-specific promoters.

Also described herein are methods of reducing the enteric methane emissions of livestock animals, especially ruminants such as cows, by incorporation of plants having the desired characteristics (e.g., haloform levels present) into animal diets in a manner to provide a total haloform inclusion level of at least 2 μg haloform per gram total dry matter.

One embodiment described herein is a genetically modified feed crop for reducing methanogenesis in a livestock animal, the genetically modified feed crop comprising: a transgenic polynucleotide construct comprising a haloperoxidase (HPO) gene sequence encoding a HPO protein, wherein the HPO protein enables biosynthesis of bromoform (CHBr₃), chloroform (CHCl₃), iodoform (CHI₃), or a combination thereof in the genetically modified feed crop. In another aspect, the HPO gene sequence encoding a HPO protein is a vanadium-dependent bromoperoxidase (VBPO), a vanadium-dependent iodoperoxidase (VIPO), vanadium-dependent chloroperoxidase (VCPO), or a nonspecific vanadium-dependent haloperoxidase (VHPO). In another aspect, the genetically modified feed crop is a monocot plant or a dicot plant. In another aspect, the HPO gene sequence has at least 90-99% identity to any one of SEQ ID NO: 1, 3, 41, 43, 45, 47, 49, 51, 52, 53, 54, 55, 56, 57, 58, 59, 60, 61, 63, 65, 67, 69, 71, 73, 75, 77, 79, 81, 83, 85, 87, 89, or 91. In another aspect, the HPO gene sequence is selected from any one of SEQ ID NO: 1, 3, 41, 43, 45, 47, 49, 51, 52, 53, 54, 55, 56, 57, 58, 59, 60, 61, 63, 65, 67, 69, 71, 73, 75, 77, 79, 81, 83, 85, 87, 89, or 91. In another aspect, the HPO protein comprises an amino acid sequence having at least 90-99% identity to any one of SEQ ID NO: 2, 4-8, 42, 44, 46, 48, 50, 62, 64, 66, 68, 70, 72, 74, 76, 78, 80, 82, 84, 86, 88, 90, or 92. In another aspect, the HPO protein comprises an amino acid sequence selected from any one of SEQ ID NO: 2, 4-8, 42, 44, 46, 48, 50, 62, 64, 66, 68, 70, 72, 74, 76, 78, 80, 82, 84, 86, 88, 90, or 92. In another aspect, the HPO gene sequence comprises a whole-plant constitutive promoter and the HPO gene sequence is expressed under the whole-plant constitutive promoter. In another aspect, the whole-plant constitutive promoter comprises a polynucleotide sequence having at least 90-99% identity to any one of SEQ ID NO: 19-20, or 93. In another aspect, the whole-plant constitutive promoter comprises a polynucleotide sequence selected from any one of SEQ ID NO: 19-20, or 93. In another aspect, the HPO gene sequence comprises an endosperm-specific promoter or photosynthetic-tissue promoter and is expressed under the endosperm-specific promoter or photosynthetic-tissue promoter. In another aspect, the endosperm-specific promoter comprises a polynucleotide sequence having at least 90-99% identity to any one of SEQ ID NO: 24-25, or 94; and the photosynthetic-tissue promoter comprises a polynucleotide sequence having at least 90-99% identity to any one of SEQ ID NO: 21-23. In another aspect, the endosperm-specific promoter comprises a polynucleotide sequence selected from any one of SEQ ID NO: 24-25, or 94; and the photosynthetic-tissue promoter comprises a polynucleotide sequence selected from any one of SEQ ID NO: 21-23. The genetically modified feed crop of claim 1, further comprising: a second transgenic polynucleotide construct comprising: an acetoacetyl-CoA thiolase 2 (AACT2) gene sequence encoding an AACT2 protein. In another aspect, the AACT2 gene sequence has at least 90-99% identity to any one of SEQ ID NO: 9-10 or 12-13. In another aspect, the AACT2 gene sequence is selected from any one of SEQ ID NO: 9-10 or 12-13. In another aspect, the AACT2 protein comprises an amino acid sequence having at least 90-99% identity to SEQ ID NO: 11. In another aspect, the AACT2 protein comprises an amino acid sequence selected from SEQ ID NO: 11. In another aspect, the AACT2 gene sequence comprises a whole-plant constitutive promoter and the AACT2 gene sequence is expressed under the whole-plant constitutive promoter. In another aspect, the whole-plant constitutive promoter comprises a polynucleotide sequence having at least 90-99% identity to any one of SEQ ID NO: 19-20, or 93. In another aspect, the whole-plant constitutive promoter comprises a polynucleotide sequence selected from any one of SEQ ID NO: 19-20, or 93. In another aspect, the HPO gene sequence and AACT2 gene sequence are separated by a self-cleaving peptide gene sequence encoding for a self-cleaving peptide. In another aspect, the self-cleaving peptide gene sequence has at least 90-99% identity to any one of SEQ ID NO: 30, 32, 34, 36, 38, or 40. In another aspect, the self-cleaving peptide gene sequence is selected from any one of SEQ ID NO: 29, 31, 33, 35, 37, or 39. In another aspect, the self-cleaving peptide comprises an amino acid sequence having at least 90-99% identity to any one of SEQ ID NO: 30, 32, 34, 36, 38, or 40. In another aspect, the self-cleaving peptide comprises an amino acid sequence selected from any one of SEQ ID NO: 30, 32, 34, 36, 38, or 40. In another aspect, the HPO is a Vanadium-dependent bromoperoxidase (VBPO) gene sequence having at least 90-99% identity to any one of SEQ. ID NO: 1 or 3. In another aspect, the HPO is a Vanadium-dependent bromoperoxidase (VBPO) gene sequence selected from any one of SEQ ID NO: 1 or 3. In another aspect, the HPO is a Vanadium-dependent bromoperoxidase (VBPO) protein comprising an amino acid sequence having at least 90-99% identity to any one of SEQ ID NO: 2 or 4-8. In another aspect, the HPO is a Vanadium-dependent bromoperoxidase (VBPO) protein comprising an amino acid sequence selected from any one of SEQ ID NO: 2 or 4-8. In another aspect, the HPO is a Vanadium-dependent iodoperoxidase (VIPO) gene sequence having at least 90-99% identity to any one of SEQ. ID NO: 41, 43, 45, 47, 49 or 51-60. In another aspect, the HPO is a Vanadium-dependent iodoperoxidase (VIPO) gene sequence selected from any one of SEQ ID NO: 41, 43, 45, 47, 49 or 51-60. In another aspect, the HPO is a Vanadium-dependent iodoperoxidase (VIPO) protein comprising an amino acid sequence having at least 90-99% identity to any one of SEQ ID NO: 42, 44, 46, 48, or 50. In another aspect, the HPO is a Vanadium-dependent iodoperoxidase (VIPO) protein comprising an amino acid sequence selected from any one of SEQ ID NO: 42, 44, 46, 48, or 50. In another aspect, the HPO is a Vanadium-dependent chloroperoxidase (VCPO) gene sequence having at least 90-99% identity to any one of SEQ. ID NO: 61, 63, 65, 67, 69, 71, 73, or 75. In another aspect, the HPO is a Vanadium-dependent chloroperoxidase (VCPO) gene sequence selected from any one of SEQ ID NO: 61, 63, 65, 67, 69, 71, 73, or 75. In another aspect, the HPO is a Vanadium-dependent chloroperoxidase (VCPO) protein comprising an amino acid sequence having at least 90-99% identity to any one of SEQ ID NO: 62, 64, 66, 68, 70, 72, 74, or 76. In another aspect, the HPO is a Vanadium-dependent chloroperoxidase (VCPO) protein comprising an amino acid sequence selected from any one of SEQ ID NO: 62, 64, 66, 68, 70, 72, 74, or 76. In another aspect, the HPO is a nonspecific vanadium-dependent haloperoxidase (VHPO) gene sequence having at least 90-99% identity to any one of SEQ. ID NO: 77, 79, 81, 83, 85, 87, 89, or 91. In another aspect, the HPO is a nonspecific vanadium-dependent haloperoxidase (VHPO) gene sequence selected from any one of SEQ ID NO: 77, 79, 81, 83, 85, 87, 89, or 91. In another aspect, the HPO is a nonspecific vanadium-dependent haloperoxidase (VHPO) protein comprising an amino acid sequence having at least 90-99% identity to any one of SEQ ID NO: 78, 80, 82, 84, 86, 88, 90, or 92. In another aspect, the HPO is a nonspecific vanadium-dependent haloperoxidase (VHPO) protein comprising an amino acid sequence selected from any one of SEQ ID NO: 78, 80, 82, 84, 86, 88, 90, or 92.

In another aspect, the genetically modified feed crop further comprises: a third transgenic polynucleotide construct comprising: a Wrinkled 1 (WRI1) gene sequence encoding a WRI1 protein. In another aspect, the WRI1 gene sequence has at least 90-99% identity to any one of SEQ ID NO: 14-15 or 17-18. In another aspect, the WRI1 gene sequence is selected from any one of SEQ ID NO: 14-15 or 17-18. In another aspect, the WRI1 protein comprises an amino acid sequence having at least 90-99% identity to SEQ ID NO: 16. In another aspect, the WRI1 protein comprises an amino acid sequence selected from SEQ ID NO: 16. In another aspect, the HPO gene sequence and WRI1 gene sequence are separated by a self-cleaving peptide gene sequence encoding for a self-cleaving peptide. In another aspect, the self-cleaving peptide gene sequence has at least 90-99% identity to any one of SEQ ID NO: 30, 32, 34, 36, 38, or 40. In another aspect, the self-cleaving peptide gene sequence is selected from any one of SEQ ID NO: 29, 31, 33, 35, 37, or 39. In another aspect, the self-cleaving peptide comprises an amino acid sequence having at least 90-99% identity to any one of SEQ ID NO: 30, 32, 34, 36, 38, or 40. In another aspect, the self-cleaving peptide comprises an amino acid sequence selected from any one of SEQ ID NO: 30, 32, 34, 36, 38, or 40.

Another embodiment described herein is a method for reducing methanogenesis in a livestock animal, the method comprising: feeding a livestock animal a genetically modified feed crop comprising: a transgenic polynucleotide construct comprising a haloperoxidase (HPO) gene sequence encoding a HPO protein; wherein the HPO protein enables biosynthesis of bromoform (CHBr₃), chloroform (CHCl₃), iodoform (CHI₃), or a combination thereof in the genetically modified feed crop. In another aspect, the HPO gene sequence encoding a HPO protein is a vanadium-dependent bromoperoxidase (VBPO), a vanadium-dependent iodoperoxidase (VIPO), vanadium-dependent chloroperoxidase (VCPO), or a nonspecific vanadium-dependent haloperoxidase (VHPO). In another aspect, the method reduces the level of methane produced and released from the livestock animal as compared to a livestock animal that is not fed the genetically modified feed crop. In another aspect, the livestock animal is a cow, a sheep, a goat, a buffalo, a camel, a deer, an elk, an antelope, a gazelle, or a giraffe.

Another embodiment described herein is a method of making a genetically modified feed crop for reducing methanogenesis in a livestock animal, the method comprising: transforming a monocot plant or a dicot plant with a transgenic polynucleotide construct comprising a haloperoxidase (HPO) gene sequence encoding a HPO protein, wherein the HPO protein enables biosynthesis of bromoform (CHBr₃), chloroform (CHCl₃), iodoform (CHI₃), or a combination thereof in the genetically modified feed crop; and growing the transformed monocot plant or dicot plant comprising the transgenic polynucleotide construct to generate the genetically modified feed crop. In another aspect, the HPO gene sequence encoding a HPO protein is a vanadium-dependent bromoperoxidase (VBPO), a vanadium-dependent iodoperoxidase (VIPO), vanadium-dependent chloroperoxidase (VCPO), or a nonspecific vanadium-dependent haloperoxidase (VHPO). In another aspect, the genetically modified feed crop comprises an increased level of HPO protein as compared to a plant that is not transformed with the transgenic polynucleotide construct. In another aspect, the livestock animal is a cow, a sheep, a goat, a buffalo, a camel, a deer, an elk, an antelope, a gazelle, or a giraffe.

Another embodiment described herein is the use of the genetically modified feed crop as described herein for reducing methanogenesis in a livestock animal. In another aspect, the livestock animal is a ruminant animal. In another aspect, the livestock animal is a cow, a sheep, a goat, a buffalo, a camel, a deer, an elk, an antelope, a gazelle, or a giraffe.

It will be apparent to one of ordinary skill in the relevant art that suitable modifications and adaptations to the compositions, formulations, methods, processes, and applications described herein can be made without departing from the scope of any embodiments or aspects thereof. The compositions and methods provided are exemplary and are not intended to limit the scope of any of the specified embodiments. All of the various embodiments, aspects, and options disclosed herein can be combined in any variations or iterations. The scope of the compositions, formulations, methods, and processes described herein include all actual or potential combinations of embodiments, aspects, options, examples, and preferences herein described. The exemplary compositions and formulations described herein may omit any component, substitute any component disclosed herein, or include any component disclosed elsewhere herein. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. Furthermore, the foregoing discussion discloses and describes merely exemplary embodiments. All patents and publications cited herein are incorporated by reference herein for the specific teachings thereof.

Various embodiments and aspects of the inventions described herein are summarized by the following clauses:
Clause 1. A genetically modified feed crop for reducing methanogenesis in a livestock animal, the genetically modified feed crop comprising:
   a transgenic polynucleotide construct comprising a haloperoxidase (HPO) gene sequence encoding a HPO protein,
   wherein the HPO protein enables biosynthesis of bromoform (CHBr₃), chloroform (CHCl₃), iodoform (CHI₃), or a combination thereof in the genetically modified feed crop.
Clause 2. The genetically modified feed crop of clause 1, wherein the HPO gene sequence encoding a HPO protein is a vanadium-dependent bromoperoxidase (VBPO), a vanadium-dependent iodoperoxidase (VIPO), vanadium-dependent chloroperoxidase (VCPO), or a nonspecific vanadium-dependent haloperoxidase (VHPO).
Clause 3. The genetically modified feed crop of clause 1 or 2, wherein the genetically modified feed crop is a monocot plant or a dicot plant.
Clause 4. The genetically modified feed crop of any one of clauses 1-4, wherein the HPO gene sequence has at least 90-99% identity to any one of SEQ ID NO: 1, 3, 41, 43, 45, 47, 49, 51, 52, 53, 54, 55, 56, 57, 58, 59, 60, 61, 63, 65, 67, 69, 71, 73, 75, 77, 79, 81, 83, 85, 87, 89, or 91.
Clause 5. The genetically modified feed crop of any one of clauses 1-4, wherein the HPO gene sequence is selected from any one of SEQ ID NO: 1, 3, 41, 43, 45, 47, 49, 51, 52, 53, 54, 55, 56, 57, 58, 59, 60, 61, 63, 65, 67, 69, 71, 73, 75, 77, 79, 81, 83, 85, 87, 89, or 91.
Clause 6. The genetically modified feed crop of any one of clauses 1-5, wherein the HPO protein comprises an amino acid sequence having at least 90-99% identity to any one of SEQ ID NO: 2, 4-8, 42, 44, 46, 48, 50, 62, 64, 66, 68, 70, 72, 74, 76, 78, 80, 82, 84, 86, 88, 90, or 92.
Clause 7. The genetically modified feed crop of any one of clauses 1-6, wherein the HPO protein comprises an amino acid sequence selected from any one of SEQ ID NO: 2, 4-8, 42, 44, 46, 48, 50, 62, 64, 66, 68, 70, 72, 74, 76, 78, 80, 82, 84, 86, 88, 90, or 92.
Clause 8. The genetically modified feed crop of any one of clauses 1-7, wherein the HPO gene sequence comprises a whole-plant constitutive promoter and the HPO gene sequence is expressed under the whole-plant constitutive promoter.
Clause 9. The genetically modified feed crop of any one of clauses 1-8, wherein the whole-plant constitutive promoter comprises a polynucleotide sequence having at least 90-99% identity to any one of SEQ ID NO: 19-20, or 93.
Clause 10. The genetically modified feed crop of any one of clauses 1-9, wherein the whole-plant constitutive promoter comprises a polynucleotide sequence selected from any one of SEQ ID NO: 19-20, or 93.
Clause 11. The genetically modified feed crop of any one of clauses 1-10, wherein the HPO gene sequence comprises an endosperm-specific promoter or photosynthetic-tissue promoter and is expressed under the endosperm-specific promoter or photosynthetic-tissue promoter.
Clause 12. The genetically modified feed crop of any one of clauses 1-11, wherein the endosperm-specific promoter comprises a polynucleotide sequence having at least 90-99% identity to any one of SEQ ID NO: 24-25, or 94; and the photosynthetic-tissue promoter comprises a polynucleotide sequence having at least 90-99% identity to any one of SEQ ID NO: 21-23.
Clause 13. The genetically modified feed crop any one of clauses 1-12, wherein the endosperm-specific promoter comprises a polynucleotide sequence selected from any one of SEQ ID NO: 24-25, or 94; and the photosynthetic-tissue promoter comprises a polynucleotide sequence selected from any one of SEQ ID NO: 21-23.
Clause 14. The genetically modified feed crop any one of clauses 1-13, further comprising:
   a second transgenic polynucleotide construct comprising:
      an acetoacetyl-CoA thiolase 2 (AACT2) gene sequence encoding an AACT2 protein.
Clause 15. The genetically modified feed crop of any one of clauses 1-14, wherein the AACT2 gene sequence has at least 90-99% identity to any one of SEQ ID NO: 9-10 or 12-13.
Clause 16. The genetically modified feed crop of any one of clauses 1-15, wherein the AACT2 gene sequence is selected from any one of SEQ ID NO: 9-10 or 12-13.
Clause 17. The genetically modified feed crop of any one of clauses 1-, wherein the AACT2 protein comprises an amino acid sequence having at least 90-99% identity to SEQ ID NO: 11.
Clause 18. The genetically modified feed crop of any one of clauses 1-, wherein the AACT2 protein comprises an amino acid sequence selected from SEQ ID NO: 11.
Clause 19. The genetically modified feed crop of any one of clauses 1-, wherein the AACT2 gene sequence comprises a whole-plant constitutive promoter and the AACT2 gene sequence is expressed under the whole-plant constitutive promoter.
Clause 20. The genetically modified feed crop of clause 19, wherein the whole-plant constitutive promoter comprises a polynucleotide sequence having at least 90-99% identity to any one of SEQ ID NO: 19-20, or 93.
Clause 21. The genetically modified feed crop of clause 19, wherein the whole-plant constitutive promoter comprises a polynucleotide sequence selected from any one of SEQ ID NO: 19-20, or 93.
Clause 22. The genetically modified feed crop of any one of clauses 1-, wherein the HPO gene sequence and AACT2 gene sequence are separated by a self-cleaving peptide gene sequence encoding for a self-cleaving peptide.
Clause 23. The genetically modified feed crop of any one of clauses 1-22, wherein the self-cleaving peptide gene sequence has at least 90-99% identity to any one of SEQ ID NO: 30, 32, 34, 36, 38, or 40.
Clause 24. The genetically modified feed crop of any one of clauses 1-23, wherein the self-cleaving peptide gene sequence is selected from any one of SEQ ID NO: 29, 31, 33, 35, 37, or 39.
Clause 25. The genetically modified feed crop of any one of clauses 1-24, wherein the self-cleaving peptide comprises an amino acid sequence having at least 90-99% identity to any one of SEQ ID NO: 30, 32, 34, 36, 38, or 40.
Clause 26. The genetically modified feed crop of any one of clauses 1-25, wherein the self-cleaving peptide comprises an amino acid sequence selected from any one of SEQ ID NO: 30, 32, 34, 36, 38, or 40.
Clause 27. The genetically modified feed crop of any one of clauses 1-26, wherein the HPO is a Vanadium-dependent bromoperoxidase (VBPO) gene sequence having at least 90-99% identity to any one of SEQ. ID NO: 1 or 3.
Clause 28. The genetically modified feed crop of any one of clauses 1-27, wherein the HPO is a Vanadium-dependent bromoperoxidase (VBPO) gene sequence selected from any one of SEQ ID NO: 1 or 3.
Clause 29. The genetically modified feed crop of any one of clauses 1-28, wherein the HPO is a Vanadium-dependent bromoperoxidase (VBPO) protein comprising an amino acid sequence having at least 90-99% identity to any one of SEQ ID NO: 2 or 4-8.
Clause 30. The genetically modified feed crop of any one of clauses 1-29, wherein the HPO is a Vanadium-dependent bromoperoxidase (VBPO) protein comprising an amino acid sequence selected from any one of SEQ ID NO: 2 or 4-8.
Clause 31. The genetically modified feed crop of any one of clauses 1-30, wherein the HPO is a Vanadium-dependent iodoperoxidase (VIPO) gene sequence having at least 90-99% identity to any one of SEQ. ID NO: 41, 43, 45, 47, 49 or 51-60.
Clause 32. The genetically modified feed crop of any one of clauses 1-31, wherein the HPO is a Vanadium-dependent iodoperoxidase (VIPO) gene sequence selected from any one of SEQ ID NO: 41, 43, 45, 47, 49 or 51-60.
Clause 33. The genetically modified feed crop of any one of clauses 1-32, wherein the HPO is a Vanadium-dependent iodoperoxidase (VIPO) protein comprising an amino acid sequence having at least 90-99% identity to any one of SEQ ID NO: 42, 44, 46, 48, or 50.
Clause 34. The genetically modified feed crop of any one of clauses 1-33, wherein the HPO is a Vanadium-dependent iodoperoxidase (VIPO) protein comprising an amino acid sequence selected from any one of SEQ ID NO: 42, 44, 46, 48, or 50.
Clause 35. The genetically modified feed crop of any one of clauses 1-34, wherein the HPO is a Vanadium-dependent chloroperoxidase (VCPO) gene sequence having at least 90-99% identity to any one of SEQ. ID NO: 61, 63, 65, 67, 69, 71, 73, or 75.
Clause 36. The genetically modified feed crop of any one of clauses 1-35, wherein the HPO is a Vanadium-dependent chloroperoxidase (VCPO) gene sequence selected from any one of SEQ ID NO: 61, 63, 65, 67, 69, 71, 73, or 75.
Clause 37. The genetically modified feed crop of any one of clauses 1-36, wherein the HPO is a Vanadium-dependent chloroperoxidase (VCPO) protein comprising an amino acid sequence having at least 90-99% identity to any one of SEQ ID NO: 62, 64, 66, 68, 70, 72, 74, or 76.
Clause 38. The genetically modified feed crop of any one of clauses 1-37, wherein the HPO is a Vanadium-dependent chloroperoxidase (VCPO) protein comprising an amino acid sequence selected from any one of SEQ ID NO: 62, 64, 66, 68, 70, 72, 74, or 76.
Clause 39. The genetically modified feed crop of any one of clauses 1-38, wherein the HPO is a nonspecific vanadium-dependent haloperoxidase (VHPO) gene sequence having at least 90-99% identity to any one of SEQ. ID NO: 77, 79, 81, 83, 85, 87, 89, or 91.
Clause 40. The genetically modified feed crop of any one of clauses 1-39, wherein the HPO is a nonspecific vanadium-dependent haloperoxidase (VHPO) gene sequence selected from any one of SEQ ID NO: 77, 79, 81, 83, 85, 87, 89, or 91.
Clause 41. The genetically modified feed crop of any one of clauses 1-40, wherein the HPO is a nonspecific vanadium-dependent haloperoxidase (VHPO) protein comprising an amino acid sequence having at least 90-99% identity to any one of SEQ ID NO: 78, 80, 82, 84, 86, 88, 90, or 92.
Clause 42. The genetically modified feed crop of any one of clauses 1-41, wherein the HPO is a nonspecific vanadium-dependent haloperoxidase (VHPO) protein comprising an amino acid sequence selected from any one of SEQ ID NO: 78, 80, 82, 84, 86, 88, 90, or 92.
Clause 43. The genetically modified feed crop of any one of clauses 1-42, further comprising:
   a third transgenic polynucleotide construct comprising:
      a Wrinkled 1 (WRI1) gene sequence encoding a WRI1 protein.
Clause 44. The genetically modified feed crop of any one of clauses 1-43, wherein the WRI1 gene sequence has at least 90-99% identity to any one of SEQ ID NO: 14-15 or 17-18.
Clause 45. The genetically modified feed crop of any one of clauses 1-44, wherein the WRI1 gene sequence is selected from any one of SEQ ID NO: 14-15 or 17-18.
Clause 46. The genetically modified feed crop of any one of clauses 1-45, wherein the WRI1 protein comprises an amino acid sequence having at least 90-99% identity to SEQ ID NO: 16.
Clause 47. The genetically modified feed crop of any one of clauses 1-46, wherein the WRI1 protein comprises an amino acid sequence selected from SEQ ID NO: 16.
Clause 48. The genetically modified feed crop of any one of clauses 1-47, wherein the HPO gene sequence and WRI1 gene sequence are separated by a self-cleaving peptide gene sequence encoding for a self-cleaving peptide.
Clause 49. The genetically modified feed crop of any one of clauses 1-48, wherein the self-cleaving peptide gene sequence has at least 90-99% identity to any one of SEQ ID NO: 30, 32, 34, 36, 38, or 40.
Clause 50. The genetically modified feed crop of any one of clauses 1-49, wherein the self-cleaving peptide gene sequence is selected from any one of SEQ ID NO: 29, 31, 33, 35, 37, or 39.
Clause 51. The genetically modified feed crop of any one of clauses 1-50, wherein the self-cleaving peptide comprises an amino acid sequence having at least 90-99% identity to any one of SEQ ID NO: 30, 32, 34, 36, 38, or 40.
Clause 52. The genetically modified feed crop of any one of clauses 1-51, wherein the self-cleaving peptide comprises an amino acid sequence selected from any one of SEQ ID NO: 30, 32, 34, 36, 38, or 40.
Clause 53. A method for reducing methanogenesis in a livestock animal, the method comprising:
   feeding a livestock animal a genetically modified feed crop comprising:
   a transgenic polynucleotide construct comprising a haloperoxidase (HPO) gene sequence encoding a HPO protein;
      wherein the HPO protein enables biosynthesis of bromoform (CHBr₃), chloroform (CHCl₃), iodoform (CHI₃), or a combination thereof in the genetically modified feed crop.
Clause 54. The method of clause 53, wherein the HPO gene sequence encoding a HPO protein is a vanadium-dependent bromoperoxidase (VBPO), a vanadium-dependent iodoperoxidase (VIPO), vanadium-dependent chloroperoxidase (VCPO), or a nonspecific vanadium-dependent haloperoxidase (VHPO).
Clause 55. The method of clause 53 or 54, wherein the method reduces the level of methane produced and released from the livestock animal as compared to a livestock animal that is not fed the genetically modified feed crop.
Clause 56. The method of any one of clauses 53-55, wherein the livestock animal is a cow, a sheep, a goat, a buffalo, a camel, a deer, an elk, an antelope, a gazelle, or a giraffe.
Clause 57. A method of making a genetically modified feed crop for reducing methanogenesis in a livestock animal, the method comprising:
   transforming a monocot plant or a dicot plant with a transgenic polynucleotide construct comprising a haloperoxidase (HPO) gene sequence encoding a HPO protein,
      wherein the HPO protein enables biosynthesis of bromoform (CHBr₃), chloroform (CHCl₃), iodoform (CHI₃), or a combination thereof in the genetically modified feed crop; and
   growing the transformed monocot plant or dicot plant comprising the transgenic polynucleotide construct to generate the genetically modified feed crop.
Clause 58. The method of clause 57, wherein the HPO gene sequence encoding a HPO protein is a vanadium-dependent bromoperoxidase (VBPO), a vanadium-dependent iodoperoxidase (VIPO), vanadium-dependent chloroperoxidase (VCPO), or a nonspecific vanadium-dependent haloperoxidase (VHPO).
Clause 59. The method of clause 57 or 58, wherein the genetically modified feed crop comprises an increased level of HPO protein as compared to a plant that is not transformed with the transgenic polynucleotide construct.
Clause 60. The method of any one of clauses 57-8, wherein the livestock animal is a cow, a sheep, a goat, a buffalo, a camel, a deer, an elk, an antelope, a gazelle, or a giraffe.
Clause 61. Use of the genetically modified feed crop of any one of clauses 1-51 for reducing methanogenesis in a livestock animal.
Clause 62. The use of clause 61, wherein the livestock animal is a ruminant animal.
Clause 63. The use of clause 61 or 62, wherein the livestock animal is a cow, a sheep, a goat, a buffalo, a camel, a deer, an elk, an antelope, a gazelle, or a giraffe.

### EXAMPLES

### Example 1

Diversion of Soil-Derived Bromine from Native Methyl Bromide Biosynthesis to Bromoform Biosynthesis by Expression of VBPO

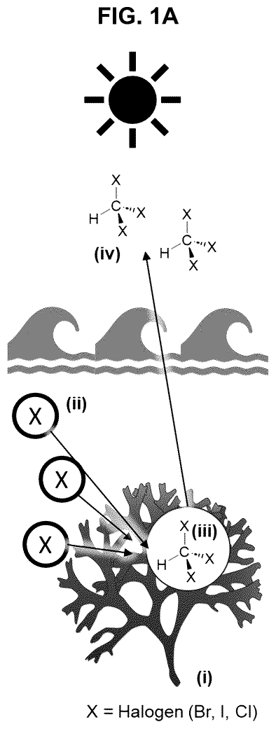

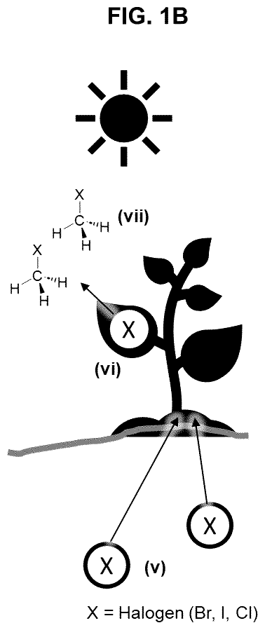

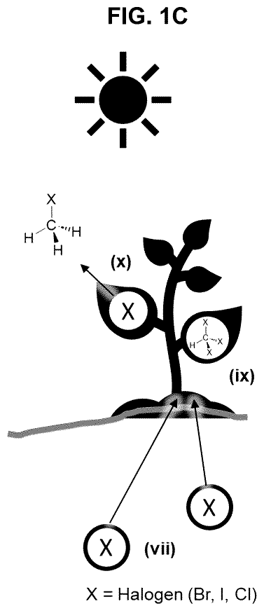

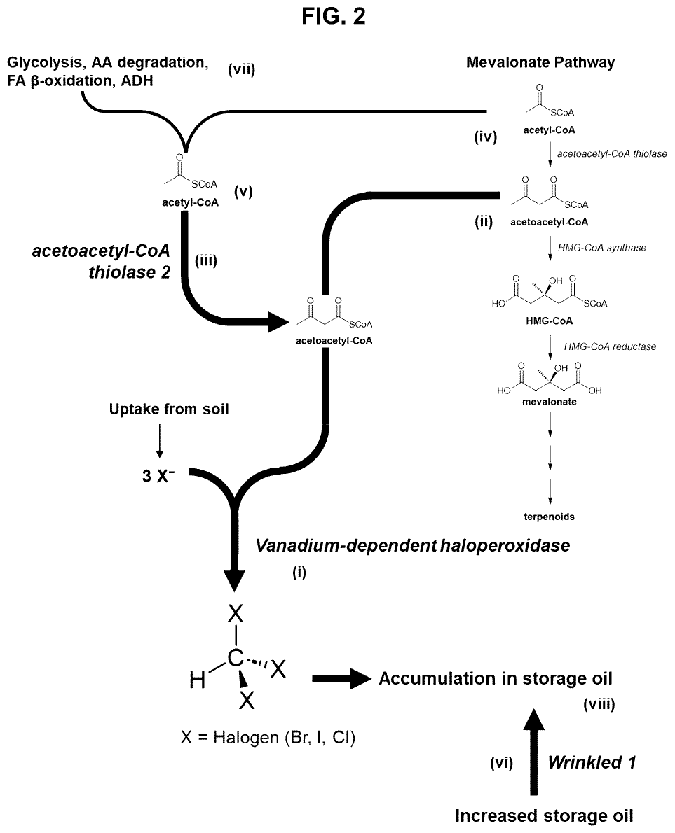

Normally, bromine taken up from soil in unmodified land plants accumulates in tissues or is converted to volatile methyl bromide (CH₃Br) via enzymes encoded by HOL (Harmless to Ozone Layer) genes, followed by release to the atmosphere (FIG. 1A-C). Expression of a VBPO gene derived from Asparagopsis taxiformis or another marine organism (SEQ ID NO: 1-8) instead diverts a portion of the naturally occurring bromine to synthesis of bromoform. VBPO accepts many organic substrates which it combines with bromide to make bromoform or other organobromine molecules. One organic substrate whose conversion to bromoform by VBPO has been individually validated is acetoacetyl or its protein conjugate AcAc-COA. AcAc-COA is naturally present in land plants as a component of the mevalonate pathway that provides precursors for terpenoid synthesis. Consequently, as a first component, expression of VBPO in land plant tissues results in synthesis of bromoform from bromide and naturally occurring AcAc-CoA (FIG. 2).

In Arabidopsis, VBPO (SEQ ID NO: 1) was transgenically expressed under control of a seed-specific Napin promoter derived from Brassica napus (SEQ ID NO: 24) and grown in commercial potting soil under typical conditions. Mature Arabidopsis seeds of 5 independent transgenic lines were manually harvested and cleaned of chaff. Seed samples weighing from 0.3951 to 0.1072 grams were frozen in liquid nitrogen and ground by mechanical shaking with steel beads. Methanol (0.8 or 1.6 mL) was added to each seed sample, and samples were blended with beads, vortexed thoroughly, allowed to incubate overnight at 4° C., and vortexed again. Samples were centrifuged to collect debris, and the supernatants were analyzed by Gas Chromatography using an Agilent 6890 instrument. GC analysis used a 30 meter long, 250 μm diameter 5% phenyl methyl siloxane column with hydrogen carrier gas running in 1:1 split mode with port temperature of 250° C. and continuous flow 0.9 mL/min. Accounting for original sample mass and extraction volume, this quantification revealed that VBPO results in expression of 159.02 micrograms of bromoform per gram of fresh weight of mature seed in one transgenic line, and 1,653.68 micrograms bromoform per gram of fresh weight of mature seed in another.

The VBPO transgene includes a protein sequence (SEQ ID NO: 2, 4-8) reverse-translated to DNA and codon-optimized for expression in dicots (represented by Arabidopsis thaliana) (SEQ ID NO: 1) or monocots (SEQ ID NO: 3) (represented by corn, Zea mays), and lacking introns.

In Arabidopsis, VBPO (SEQ ID NO: 1) was transgenically expressed under control of a seed-specific Napin promoter derived from Brassica napus (SEQ ID NO: 24), and the transgenically-modified Arabidopsis was grown in commercial potting soil under typical conditions. This resulted in the production of ˜159.02 μg bromoform per gram fresh weight of mature seed in one independent transgenic line, and ˜1,653.68 μg bromoform per gram in another transgenic line, as determined by gas chromatography with mass spectrometry (GC-MS). In comparison, the experimental controls of Arabidopsis with no transgenically expressed VBPO did not produce any bromoform.

To assess the genetic stability of seed-specific production of bromoform enabled by this invention Arabidopsis plants transgenically expressing VBPO under control of the Napin promoter were brought to homozygosity using selective marker segregation and grown for six generations. Bromoform was extracted in methanol from seed samples of the first and sixth generations of five independent transgenic lines and quantified by GC-MS analysis at Avazyme, Inc. By comparing bromoform levels in sixth-generation plant seeds to first-generation seeds of the corresponding line, this experiment is expected to reveal that bromoform production levels remain stable across generations and are not subject to genetic silencing or otherwise counteracted by native processes.

TABLE 1
Nucleotide and Amino Acid Sequences

SEQ ID NO: 1-Asparagopsis taxiformis Vanadium-dependent Bromoperoxidase #1, DNA sequence
codon-optimized for Arabidopsis thaliana
ATGGCGGAGGAGAGACGACAAAACGCACTTGAAATCAGGATCCAAGCTGCTAAGCTAGCTAAGAAGAGGGACCATC
CTACACACAAAGCCAATGGAGATGAAGACAGATATCCAGAGACACTTATAGGTAGCTTTACTAAAGGACTGCCGCA
CGAGAAGGAAACAGGTTTATTGTCTAACCCTGCTGACTTCGCTGATTTCGTTCGAGCTATTAATACCGGTGCTATC
AAGGACATGAGGAGGTTGAAATTGGGTATCGATGAAGATCCTCGTTTTATCAGTGGAATCGCGTCCAAAGGTGACA
AGCACCCTTTTGCTGATACACGAGCTTGGGAATCTATGGCAGCAGGACTGACTTACGATCTTGAGGGACCAGATGC
CCAGGCGGTCACTATGCCTCCAGCGCCTAAGCTTGATAGCGACGAACTGGTGACGGAGATCACGGAATCTTATTGG
ATGGCTTTATTGAGAGATGTTCCTTTTACCGAGTTTGAACGAGATGGGAACACTGCTGCCGCTGCAGCTAGCATAT
CCCGGACACGATGGGTTCAATATAATGAGCAACCTTCGCAAAGACCTAGCACACTTACAGACGAAGAGATAGCAAG
ACTACGAGGACCGTATACCAAAAAAAACGTATTCAGGGGTGTCACCAATGGGGAGAATGTAGGCCCTTATTTATCA
CAGTTTCTTCTGGTTGGAACAAAGGGCATTGGTGATGCCCAACAAGTTAGTGATGGTTATGTACAGTATGGAGGAA
TGAGAATGGACCAAAGAGTTCGAGTGGCTGTGCCTAAAAGAGATTACATGACCACATGGGCTTCTTGGCTCGATGT
TCAAAATGCTGGTGATCTTAGAGGTAGAGAAATCTATGACGACGACACACCATTCCGATTCATAACAACGCCACGT
GATCTGGCTACATGGGTGCATTTTGATGCATTATACCAGGCTTATTTGAATGCTTGTATAATACTATTAGATATTA
AGGCTCCGTTCGATCCTCACATCCCTTTCCAAGCGGATGATGATGTTGATAAACAGCAAGGATTCGCAACGTTTGG
TGGTCCGCATATTCTTTCTCTTTGCACTGAGGTGGCTACAAGAGCGCTGAAAGCGGTTCGTTTTCAGAAGTACAAC
TTACACAGGAGGTTGAGACCTGAGGCAATTGGTGGTCTTGTCGAGAGGTTTAAGAAAACAAATGGCGACCCAAAAT
TCGCACCTGTTAAAAAATTGGTTAATGACTTAGACGGTGATATGTTGCGGCGAGTTGAGCAACATAACTGTGAACA
AAATAAGCTCTCCGATGACGGCCATGCTAGAAGGGAAGACTATAGCCCAGAGGGAGAGTCTTCACAATCATATCTT
TTACCTATGGCATTCCCTGAGGGTTCCCCTATGCATCCTTCATACGGTGCGGGACACGCCACGGTGGCCGGTGCAT
GTGTCACTGTGCTTAAAGCCTTTTTCGATCATGAATATGAACTTGATTTTTGCTATGTCCCTACAACCGATGGAAA
GAGGCTTGAGAAAGTTAATATTAATGAAAAACTTACTGTTGAGGGCGAACTTAATAAGCTTTGTGCTAACATAAGT
ATTGGTCGTAACTGGGCGGGCGTTCATTACTACTCTGACTATTTCGAGAGTATCAAGGTTGGAGAGGAGATCGCTA
TTGGTATCTTGCAGGAACAAAAATTGACTTATGGAGAGGATTTTTTTATGACTCTACCTAAGTTTGACGGTGAGAA
AATAAGAATATAATGA
SEQ ID NO: 2-Asparagopsis taxiformis Vanadium-dependent Bromoperoxidase #1, protein sequence
MAEERRQNALEIRIQAAKLAKKRDHPTHKANGDEDRYPETLIGSFTKGLPHEKETGLLSNPADFADEVRAINTGAI
KDMRRLKLGIDEDPRFISGIASKGDKHPFADTRAWESMAAGLTYDLEGPDAQAVTMPPAPKLDSDELVTEITESYW
MALLRDVPFTEFERDGNTAAAAASISRTRWVQYNEQPSQRPSTLTDEEIARLRGPYTKKNVFRGVTNGENVGPYLS
QFLLVGTKGIGDAQQVSDGYVQYGGMRMDQRVRVAVPKRDYMTTWASWLDVQNAGDLRGREIYDDDTPFRFITTPR
DLATWVHFDALYQAYLNACIILLDIKAPFDPHIPFQADDDVDKQQGFATFGGPHILSLCTEVATRALKAVRFQKYN
LHRRLRPEAIGGLVERFKKTNGDPKFAPVKKLVNDLDGDMLRRVEQHNCEQNKLSDDGHARREDYSPEGESSQSYL
LPMAFPEGSPMHPSYGAGHATVAGACVTVLKAFFDHEYELDFCYVPTTDGKRLEKVNINEKLTVEGELNKLCANIS
IGRNWAGVHYYSDYFESIKVGEEIAIGILQEQKLTYGEDFFMTLPKFDGEKIRI
SEQ ID NO: 3-Asparagopsis taxiformis Vanadium-dependent Bromoperoxidase #1, DNA coding
sequence codon-optimized for Zea mays
ATGGCAGAAGAAAGGCGGCAGAACGCGCTAGAGATCCGCATTCAAGCGGCAAAGCTCGCAAAGAAGCGGGATCACC
CGACACATAAGGCAAACGGCGATGAGGACCGCTATCCAGAGACGCTTATCGGCTCATTCACCAAAGGCTTGCCGCA
CGAGAAGGAGACAGGGCTTCTCTCCAATCCTGCAGACTTTGCTGATTTCGTTAGGGCTATTAATACAGGCGCCATC
AAGGACATGAGGCGGCTCAAGCTGGGTATTGACGAAGACCCTCGGTTCATCTCCGGTATAGCCTCAAAAGGCGACA
AACATCCCTTCGCGGACACCAGAGCCTGGGAATCTATGGCTGCTGGCTTGACCTATGATCTGGAGGGGCCTGACGC
GCAGGCCGTTACGATGCCCCCCGCCCCGAAACTTGACAGCGACGAGTTGGTGACTGAAATTACCGAATCATACTGG
ATGGCCCTTCTCCGAGATGTTCCGTTTACTGAGTTCGAGCGCGACGGAAATACTGCAGCTGCTGCAGCGTCCATAA
GCAGAACTCGATGGGTACAGTACAACGAGCAACCTTCACAGCGGCCATCGACTTTGACGGACGAGGAAATAGCCCG
TTTGCGCGGGCCTTATACGAAGAAGAATGTTTTCAGGGGGGTGACCAACGGTGAGAACGTAGGCCCATACCTCTCC
CAGTTCCTCCTCGTGGGCACCAAAGGCATCGGCGATGCACAGCAAGTGTCGGATGGATACGTCCAGTACGGCGGCA
TGAGGATGGATCAGCGCGTAAGGGTAGCTGTGCCTAAGAGGGATTACATGACCACTTGGGCGAGCTGGCTGGACGT
TCAGAATGCGGGGGACTTGAGAGGACGCGAAATATACGACGACGACACGCCATTCAGGTTCATAACTACCCCGCGT
GATCTGGCAACTTGGGTGCACTTCGACGCCTTGTACCAAGCATACCTTAACGCGTGTATTATCCTGCTCGATATCA
AGGCTCCGTTCGATCCGCACATACCATTTCAAGCCGACGACGACGTCGACAAGCAGCAAGGATTCGCAACCTTCGG
AGGTCCGCATATCCTCAGTCTCTGCACAGAGGTCGCCACCAGGGCACTCAAAGCCGTTCGCTTCCAGAAGTATAAC
CTACACCGGCGTCTCCGCCCTGAAGCCATCGGAGGCCTGGTTGAGCGGTTCAAGAAGACCAATGGCGACCCAAAGT
TCGCTCCTGTGAAAAAGCTCGTGAATGACCTCGACGGAGACATGCTTCGCCGAGTCGAGCAACACAATTGCGAGCA
GAACAAACTTTCCGACGATGGCCACGCGAGAAGAGAGGACTACAGCCCGGAGGGAGAGTCAAGCCAGAGCTATCTT
CTTCCTATGGCGTTCCCGGAGGGTTCTCCTATGCATCCATCATATGGTGCTGGCCACGCTACCGTCGCCGGGGCGT
GCGTGACAGTGTTGAAGGCTTTTTTCGACCACGAATACGAGCTTGACTTCTGCTATGTCCCCACCACCGACGGTAA
GCGTCTCGAGAAGGTCAACATCAATGAGAAGTTGACCGTTGAGGGCGAGCTGAATAAGTTGTGTGCAAACATCAGC
ATAGGGCGCAACTGGGCAGGGGTCCACTACTACTCTGACTATTTCGAGTCCATTAAGGTGGGAGAGGAGATAGCCA
TCGGTATTCTGCAGGAGCAGAAGCTTACATACGGGGAGGACTTCTTTATGACACTACCGAAATTTGATGGCGAGAA
GATTAGGATTTAATGA
SEQ ID NO: 4-Asparagopsis taxiformis Vanadium-dependent Bromoperoxidase #2, protein sequence
MTDTQNPNRAEVAFKVRVSAAELARARGSPAHLSNNSESRFRNPDGTRSLLANFTKGLPHIKETALVESAIDYDRE
VRAIDSGDPRDFADLPLGPQGVEPRFTSGIASDPEVGTRAWESGGAGLVEDLEGPDAQAVTMPPAPELDSDELVAE
VTECYWMSLLRDVPFPTFESNSHIQAAAESINNTQWIKFKNNPPAHLTAAERSRLRGPVTTANVERGITPGDEVGP
YLSQFLLVGTTGIANGNEVGDGFIQYGGMRMDQRVRVAKPHIDYMTTFGAYLDVQNAANVSGRELYKEEEPRFRFI
HTPRDLATYVHFDALYQAYLNACIILLDIGAPFDSGIPFQLDNDIDKQQGFATFGGPHILSLVTEVATRALKAVRF
QKFNVHRRLRPEAIGARVDRYCATKAPEFAGAAKLSEALDKELLQKVHDHNKKQNLLSDRGNPRANDENPDGEVSE
GNLLMPMAFPEGSPMHPAYGAGHATVAGACVTVLKAFFDGGYRLPFCYITDEDGTGLQAVEIDEPLTVDGELNKIC
SNISIGRNWAGVHYFTDYIESIRIGEEIAIGILQEQKLTFSENFSMTLNKFDGSTIRI
SEQ ID NO: 5-Trichodesmium erythraeum (cyanobacterium) Vanadium-dependent Bromoperoxidase
protein sequence
MRERSQKSYSIRVDAAELARSRQHPIHEANGDEQRYGGDKYFMSFTKGLIHNPNTGLLEDPRDFVEFRRAIDNGFI
DPFTDRVRHGAKYKVNDQGNIVEEDNPQDLDFRQWEAPTAGVVHELEGPDAQAVTIPPAPPLLDSTRNTNPELIFE
MAEVYELAILRDVPLNNEDNKNTTDELTASIARLNNLDYVNNQNGRPRKVNQAGELDLQTVERGSSPGVEVGPYLS
QFLLIGNKGVNGVDNTTTGRIAYGVQQIDQRVPVASVQNYMTQWENYVRVQRGIKQERETYVEGNGSHRFIYTPRD
LATYVHYDALYQAYLNACFILLGMGTPFDPSFDHLSGGGEAAKDPKTRPHAGGFALYAGPHILTLVTEVATRALKA
IRFQKENNHIRLRPEALAARIDLVRRKNNGEIQESLIPQALSQYVHGFIDKLGSNGYGSLTTSTLGRIAKIAESSY
LLPMAFPEGSPMHPAYGAGHATVAGACVTILKAFFDTSTVLIKDSNNEYKFKHQEAQDQFIAFRTNSSGTDLITED
INNISEALTLEGELNKLAANISIGRNMAGVHYFSDYYDSIRMGEQIAIGILQEQALTYSTDPFVMSVPTEDGNIVR
IGY
SEQ ID NO: 6-Synechococcus (cyanobacterium) Vanadium-dependent Bromoperoxidase protein
sequence
MTDQRKLTAQRVREDANALAAGRIHPRHQANGDEQRYESANYAMSFTKGLDHNTTTGLIEQSGDFEAFRSAIDNGF
AEDFTRHVAVPRAEPRRKWEAPTAGTVYELQGPDPQAVTIPPAPALCSDELTFEMAEVYELALLRDLPFNAFVAGG
GSAALADSTARLNSLAYAQDGFNRRPRTTNSSNQLDAQTVFRGSSPGVDQGPYLSQFMLIGNASPSEGITPEQGFI
NFGAQRIDQRVLEARQQDDYMMKWDDWHRVQQGYEVRADRFDPCKSSGPGQAFTGQRRFIHTPRDLATYVHVDALY
QAYLNACLLLLGNGTAFDPGFDLLSGGGEGLLHDPAGGQKVPLNAGGFALWGGPHVLSLVTEVATRGLKAVRYQKF
NNHLRLRPEALAARIEKAQEIESRFPTICGCFSEMASDLQQVVDLIRNHNQSLAGEATALLPMAFAEGSPMHPAYG
AGHATVAGACVTILKAFFNTSALFVKINDVAGFHSKQHILARLKCGDSVEAGAYQETDCGKRLEFERCGSFHLIEG
KYATFKPDGKTNQSCCPLTLEGELNKLAANISIGRNMAGVHYFSDYYDSLRMGEEIAIGILEEQALCYKTDPFVLS
VPTFDGDVRRIGQR
SEQ ID NO: 7-Neopyropia yezoensis (red alga/seaweed) Vanadium-dependent Bromoperoxidase
protein sequence
MAGPTASGTARRNEAFDKRVAAATVARERVHPPHAANGEEYEYRATEDRHPAVAAAKAAAAGASREASNGDPGATD
GGATAAGAPPSSQSQVQGPSHIGSFTKGLQHNLSTGLLEDPAAFRAFVRAIDSGDPRDFVDVPIADRFTRPPTRGV
RSWESASAGLAFDLQGPDAQALTMPPAPRLGSAELVAEMIEVYAMALARDVPFATAEWAAAKPIAAAVRELNATRW
HGAAATDRELGLSPAAAARRRVTHFTPATVFRGVGRGVADGPYLSQFLLLGNAGVGGSSGAPTDGFITYGANRVDL
RVPVATPGVDYLTDWESFVDVQNGADTRGTETYVPAADGTKTRFIATPRDLATYVHYDALYQAYLNAALWLLAEGV
PFDAGLPWTAPDEVDHQQGFATFGGPHLLTLVTEVATRALKAVRFQKFNVHRRLRPEAVGGLLDRLPVAPGPLAPV
APLAEEAAETLRRVADANAAAAAGGSGAGAPVTHSVLLPMAFPEGSPMHPSYGAGHATVAGACVTVLKGWFDAGHA
LGTTAYEATPDGRCLREVSTGGVALTVEGELNKLAANISIGRNWAGVHYYSDYIESVQLGEAVAIGILEEQKLTYG
ENWSMTVPLTDGTTVRI
SEQ ID NO: 8-Saccharina japonica (brown alga/seaweed) Vanadium-dependent Bromoperoxidase
protein sequence
MKRVRPTQPRALYSAFSRRHVACALLCVVSCIFVSFNHQTFVGLTALLAAFPPCLGYDEPAEPTQPLLSGSVCRVR
DSLDFLDPVPRAKVTLLKRLAIAKDEFSVGPTCHINNGDEENVPLFAGQFHKTLPHDKFGQVDEDAYKKLQECIFT
SDINVCDDVPSGAKKNGAKLTNPLGGTAHQVAGADSDNIFIPTPDSLLSERLAAQQSEVYWMALARDIPFGEFATN
DLIRLAAENLQGLPAFKGLNIPRSKGGKIDPVTDLFRTTWPGVTTGPVVSQFMLSDFLIDSINVTPKADPLTPFTD
YMTSFQPWLDVQNGASDVDTTFDSENPRFIRNGRDLATISFRDLLYTEAFRAALILFTQGALGGSIGPYEEAERQQ
GFATFGAPHILTAMGSASSSTRHAWYQKWQVHRMLRPEAYGGLVHNTLMKNVITPLPDSILRNTELLNRVEVHNQK
MNGDGEKTFLLPMASAQGSPTHPAYPSGHAINVGAYITSLKAFLGFEAGQRCFPDPVVSNNEGTERIPYVPSGREI
VGECINDKGKKVDGLTYEGELNKVSANVLIGRSHLGVHWRMDGVYGALVGEVSAVRRLQQELPGLAEARPNDDLKR
KEIPPATYNFRLYSGKMLELYGANLYKLDGKLCEGAFTGDDFCDEVQEDDYESFEHIVEDLAKFSLHTEL
SEQ ID NO: 9-Arabidopsis thaliana Acetoacetyl-CoA Thiolase 2 DNA sequence with first intron
intact (intron shown in lowercase)
ATGGCCCATACATCAGAATCTGTGAATCCTAGAggtaacaaaacactttcattagtaaactgaatttgaaggatga
attttttttttgatatatgaatgttgatgaatcaGATGTTTGCATTGTGGGTGTTGCACGTACTCCAATGGGTGGC
TTTCTCGGATCTCTTTCATCTTTACCTGCCACAAAGCTTGGATCTTTAGCTATTGCAGCTGCTTTGAAGAGAGCAA
ATGTTGATCCAGCTCTTGTTCAAGAAGTTGTCTTTGGCAATGTTCTTAGTGCTAATTTGGGTCAAGCTCCTGCTCG
TCAAGCTGCTTTAGGTGCAGGAATCCCTAACTCTGTTATCTGTACTACAGTTAACAAGGTTTGTGCATCAGGCATG
AAAGCGGTAATGATTGCTGCTCAAAGTATCCAGTTAGGGATCAATGATGTAGTTGTGGCGGGTGGTATGGAAAGCA
TGTCTAATACACCAAAATATTTGGCAGAAGCAAGGAAGGGATCTCGTTTTGGTCATGATTCTTTAGTAGATGGAAT
GTTGAAGGATGGACTATGGGATGTCTATAACGACTGTGGGATGGGAAGCTGTGCAGAATTATGCGCTGAGAAGTTT
CAGATTACAAGGGAGCAGCAAGATGACTATGCAGTTCAGAGTTTTGAGCGTGGTATTGCTGCCCAGGAAGCTGGCG
CCTTCACATGGGAAATCGTCCCGGTTGAAGTTTCTGGAGGAAGAGGTAGGCCATCAACCATTGTTGACAAGGACGA
AGGTCTTGGGAAGTTTGATGCTGCAAAATTGAGGAAACTCCGTCCTAGTTTCAAAGAGAATGGAGGGACTGTTACA
GCTGGAAATGCGTCTAGCATAAGTGATGGTGCAGCTGCCCTTGTCCTAGTGAGCGGAGAGAAGGCTCTTCAGCTAG
GACTTCTAGTATTAGCAAAAATTAAAGGGTATGGTGACGCAGCTCAGGAACCAGAGTTTTTCACTACTGCTCCTGC
TCTTGCTATACCAAAAGCCATTGCACATGCTGGTTTGGAATCTTCTCAAGTTGATTACTATGAGATCAATGAAGCA
TTTGCAGTTGTAGCACTTGCAAATCAAAAGCTACTCGGGATTGCTCCAGAGAAAGTGAACGTAAATGGAGGAGCTG
TCTCCTTAGGACACCCTCTAGGCTGCAGTGGCGCCCGTATTCTAATCACGTTGCTTGGGATACTAAAGAAGAGAAA
CGGAAAGTACGGTGTGGGAGGAGTGTGCAACGGAGGAGGAGGTGCTTCTGCTCTAGTTCTTGAGCTCCTTTGATAA
SEQ ID NO: 10-Arabidopsis thaliana Acetoacetyl-CoA Thiolase 2 DNA sequence
ATGGCCCATACATCAGAATCTGTGAATCCTAGAGATGTTTGCATTGTGGGTGTTGCACGTACTCCAATGGGTGGCT
TTCTCGGATCTCTTTCATCTTTACCTGCCACAAAGCTTGGATCTTTAGCTATTGCAGCTGCTTTGAAGAGAGCAAA
TGTTGATCCAGCTCTTGTTCAAGAAGTTGTCTTTGGCAATGTTCTTAGTGCTAATTTGGGTCAAGCTCCTGCTCGT
CAAGCTGCTTTAGGTGCAGGAATCCCTAACTCTGTTATCTGTACTACAGTTAACAAGGTTTGTGCATCAGGCATGA
AAGCGGTAATGATTGCTGCTCAAAGTATCCAGTTAGGGATCAATGATGTAGTTGTGGCGGGTGGTATGGAAAGCAT
GTCTAATACACCAAAATATTTGGCAGAAGCAAGGAAGGGATCTCGTTTTGGTCATGATTCTTTAGTAGATGGAATG
TTGAAGGATGGACTATGGGATGTCTATAACGACTGTGGGATGGGAAGCTGTGCAGAATTATGCGCTGAGAAGTTTC
AGATTACAAGGGAGCAGCAAGATGACTATGCAGTTCAGAGTTTTGAGCGTGGTATTGCTGCCCAGGAAGCTGGCGC
CTTCACATGGGAAATCGTCCCGGTTGAAGTTTCTGGAGGAAGAGGTAGGCCATCAACCATTGTTGACAAGGACGAA
GGTCTTGGGAAGTTTGATGCTGCAAAATTGAGGAAACTCCGTCCTAGTTTCAAAGAGAATGGAGGGACTGTTACAG
CTGGAAATGCGTCTAGCATAAGTGATGGTGCAGCTGCCCTTGTCCTAGTGAGCGGAGAGAAGGCTCTTCAGCTAGG
ACTTCTAGTATTAGCAAAAATTAAAGGGTATGGTGACGCAGCTCAGGAACCAGAGTTTTTCACTACTGCTCCTGCT
CTTGCTATACCAAAAGCCATTGCACATGCTGGTTTGGAATCTTCTCAAGTTGATTACTATGAGATCAATGAAGCAT
TTGCAGTTGTAGCACTTGCAAATCAAAAGCTACTCGGGATTGCTCCAGAGAAAGTGAACGTAAATGGAGGAGCTGT
CTCCTTAGGACACCCTCTAGGCTGCAGTGGCGCCCGTATTCTAATCACGTTGCTTGGGATACTAAAGAAGAGAAAC
GGAAAGTACGGTGTGGGAGGAGTGTGCAACGGAGGAGGAGGTGCTTCTGCTCTAGTTCTTGAGCTCCTTTGATAA
SEQ ID NO: 11-Arabidopsis thaliana Acetoacetyl-CoA Thiolase 2 protein sequence
MAHTSESVNPRDVCIVGVARTPMGGFLGSLSSLPATKLGSLAIAAALKRANVDPALVQEVVFGNVLSANLGQAPAR
QAALGAGIPNSVICTTVNKVCASGMKAVMIAAQSIQLGINDVVVAGGMESMSNTPKYLAEARKGSRFGHDSLVDGM
LKDGLWDVYNDCGMGSCAELCAEKFQITREQQDDYAVQSFERGIAAQEAGAFTWEIVPVEVSGGRGRPSTIVDKDE
GLGKFDAAKLRKLRPSFKENGGTVTAGNASSISDGAAALVLVSGEKALQLGLLVLAKIKGYGDAAQEPEFFTTAPA
LAIPKAIAHAGLESSQVDYYEINEAFAVVALANQKLLGIAPEKVNVNGGAVSLGHPLGCSGARILITLLGILKKRN
GKYGVGGVCNGGGGASALVLELL
SEQ ID NO: 12-Arabidopsis thaliana Acetoacetyl-CoA Thiolase 2 DNA sequence codon-optimized
for Zea mays
ATGGCTCATACGTCAGAGTCAGTAAACCCACGTGACGTTTGCATCGTGGGGGTAGCCCGTACCCCAATGGGCGGTT
TCCTCGGAAGCCTCTCCTCCCTGCCAGCGACCAAGCTCGGGTCACTGGCGATCGCTGCAGCGCTGAAGCGAGCTAA
CGTGGACCCGGCCCTGGTGCAGGAGGTAGTCTTCGGAAATGTCCTGTCAGCCAATCTTGGGCAAGCTCCCGCTAGG
CAAGCTGCCCTCGGCGCGGGCATTCCGAACAGCGTCATCTGCACTACCGTCAACAAGGTGTGCGCGTCCGGAATGA
AAGCGGTTATGATTGCGGCCCAATCCATACAACTTGGCATCAATGATGTTGTAGTGGCCGGCGGTATGGAGTCAAT
GTCTAACACGCCGAAGTATCTAGCAGAGGCAAGAAAGGGAAGCAGGTTTGGCCATGATTCCCTTGTCGACGGTATG
CTCAAAGACGGGCTGTGGGACGTTTACAACGACTGCGGCATGGGGAGCTGCGCGGAGCTGTGCGCAGAAAAGTTTC
AGATTACCCGCGAACAGCAGGACGATTACGCCGTGCAGTCATTCGAACGCGGGATCGCCGCGCAGGAGGCAGGTGC
CTTCACGTGGGAAATCGTTCCCGTCGAGGTAAGTGGTGGTCGCGGCCGGCCTAGCACTATCGTTGATAAGGACGAG
GGTCTGGGCAAGTTCGACGCTGCGAAGCTCCGGAAGCTAAGGCCGTCCTTCAAGGAGAACGGCGGGACGGTCACGG
CCGGCAATGCATCTAGCATAAGCGATGGAGCGGCGGCGCTAGTCCTTGTCAGCGGGGAAAAGGCTCTACAGCTAGG
CTTGCTCGTCCTCGCCAAGATTAAGGGCTATGGCGATGCTGCGCAGGAACCAGAGTTCTTTACGACGGCGCCGGCG
CTGGCTATCCCCAAGGCTATCGCTCATGCGGGACTGGAATCCTCTCAAGTGGATTATTACGAAATTAATGAGGCGT
TCGCAGTGGTCGCCCTGGCTAACCAGAAGCTTCTCGGGATCGCGCCGGAGAAGGTTAATGTGAATGGTGGTGCCGT
GTCTCTGGGTCACCCGCTCGGATGCAGCGGTGCACGTATCTTGATCACGCTACTCGGTATCCTCAAAAAACGGAAC
GGGAAGTACGGGGTCGGCGGTGTTTGCAACGGGGGGGGAGGAGCGAGCGCCCTGGTCCTCGAGCTCTTGTGATAA
SEQ ID NO: 13-Arabidopsis thaliana Acetoacetyl-CoA Thiolase 2 DNA sequence codon-optimized
for Zea mays with addition of a 5′-UTR derived from an intron of Oryza sativa Cytosolic
Superoxide Dismutase 2 (UTR shown in lowercase)
atcttctgatgagaataagcaaatccctattttttttgttgaagaaaagaaaagcgtgtccacgtggactggactc
tgaaattttcctcctcccttctggagtcttcctcatcagaaatcagaagaggagagggtgggcaactcgcagatcg
ccttctcgtcgcgctcgcgccgcaggggtcgcctgaggtatgcagcttcacctcccccaactttctagggttctaa
tcgcctctgctcgctcggattatgcgtggtgggtatggctccgattccgccggagtagctggatctgtgtgccccg
tgctaatttaggtttggctttcgattcggccgcgcttcggtttgttctcgcgcgtgattgcttcgttcggccatag
ggctcatctcaggctcgacatgtggacggccacaaacataaaaaatccttgttaaatttacggttcatgtggtagg
ggggcttttcaggatcggaggtttaggtgatttggagtagcaaaacgattttgctgtgttagagaattcgagaatc
tgtgccgtcagatgctaattagctggttttgaacaaatgttaagacatcctgatattatttgctgcgtttaatgtt
caaactttttgcacgggtgttgctgagctataaaggatatgaggatatagtaccaagttctatcgttttgttctga
tacatacaatttgctgtagtttatggcaccatcatactgagatatatatacctgtgcacctttcggcttctgtgca
gaacacatagacaatggtgaaggATGGCTCATACGTCAGAGTCAGTAAACCCACGTGACGTTTGCATCGTGGGGGT
AGCCCGTACCCCAATGGGCGGTTTCCTCGGAAGCCTCTCCTCCCTGCCAGCGACCAAGCTCGGGTCACTGGCGATC
GCTGCAGCGCTGAAGCGAGCTAACGTGGACCCGGCCCTGGTGCAGGAGGTAGTCTTCGGAAATGTCCTGTCAGCCA
ATCTTGGGCAAGCTCCCGCTAGGCAAGCTGCCCTCGGCGCGGGCATTCCGAACAGCGTCATCTGCACTACCGTCAA
CAAGGTGTGCGCGTCCGGAATGAAAGCGGTTATGATTGCGGCCCAATCCATACAACTTGGCATCAATGATGTTGTA
GTGGCCGGCGGTATGGAGTCAATGTCTAACACGCCGAAGTATCTAGCAGAGGCAAGAAAGGGAAGCAGGTTTGGCC
ATGATTCCCTTGTCGACGGTATGCTCAAAGACGGGCTGTGGGACGTTTACAACGACTGCGGCATGGGGAGCTGCGC
GGAGCTGTGCGCAGAAAAGTTTCAGATTACCCGCGAACAGCAGGACGATTACGCCGTGCAGTCATTCGAACGCGGG
ATCGCCGCGCAGGAGGCAGGTGCCTTCACGTGGGAAATCGTTCCCGTCGAGGTAAGTGGTGGTCGCGGCCGGCCTA
GCACTATCGTTGATAAGGACGAGGGTCTGGGCAAGTTCGACGCTGCGAAGCTCCGGAAGCTAAGGCCGTCCTTCAA
GGAGAACGGCGGGACGGTCACGGCCGGCAATGCATCTAGCATAAGCGATGGAGCGGCGGCGCTAGTCCTTGTCAGC
GGGGAAAAGGCTCTACAGCTAGGCTTGCTCGTCCTCGCCAAGATTAAGGGCTATGGCGATGCTGCGCAGGAACCAG
AGTTCTTTACGACGGCGCCGGCGCTGGCTATCCCCAAGGCTATCGCTCATGCGGGACTGGAATCCTCTCAAGTGGA
TTATTACGAAATTAATGAGGCGTTCGCAGTGGTCGCCCTGGCTAACCAGAAGCTTCTCGGGATCGCGCCGGAGAAG
GTTAATGTGAATGGTGGTGCCGTGTCTCTGGGTCACCCGCTCGGATGCAGCGGTGCACGTATCTTGATCACGCTAC
TCGGTATCCTCAAAAAACGGAACGGGAAGTACGGGGTCGGCGGTGTTTGCAACGGGGGGGGAGGAGCGAGCGCCCT
GGTCCTCGAGCTCTTGTGATAA
SEQ ID NO: 14-Arabidopsis thaliana Wrinkled 1 DNA sequence with first intron intact
(intron shown in lowercase)
ATGAAGAAGCGCTTAACCACTTCCACTTGTTCTTCTTCTCCATCTTCCTCTGTTTCTTCTTCTACTACTACTTCCT
CTCCTATTCAGTCGGAGGCTCCAAGGCCTAAACGAGCCAAAAGGGCTAAGAAATCTTCTCCTTCTGGTGATAAATC
TCATAACCCGACAAGCCCTGCTTCTACCCGACGCAGCTCTATCTACAGAGGAGTCACTAGgtttttatttttttgg
aaattaaatgattggttgttgagattggatttgggttttgtcttaaaactgcatttgtaagattgcatgttgtttt
gtgggattttgcagACATAGATGGACTGGGAGATTCGAGGCTCATCTTTGGGACAAAAGCTCTTGGAATTCGATTC
AGAACAAGAAAGGCAAACAAGTTTATCTGGGAGCATATGACAGTGAAGAAGCAGCAGCACATACGTACGATCTGGC
TGCTCTCAAGTACTGGGGACCCGACACCATCTTGAATTTTCCGGCAGAGACGTACACAAAGGAATTGGAAGAAATG
CAGAGAGTGACAAAGGAAGAATATTTGGCTTCTCTCCGCCGCCAGAGCAGTGGTTTCTCCAGAGGCGTCTCTAAAT
ATCGCGGCGTCGCTAGGCATCACCACAACGGAAGATGGGAGGCTCGGATCGGAAGAGTGTTTGGGAACAAGTACTT
GTACCTCGGCACCTATAATACGCAGGAGGAAGCTGCTGCAGCATATGACATGGCTGCGATTGAGTATCGAGGCGCA
AACGCGGTTACTAATTTCGACATTAGTAATTACATTGACCGGTTAAAGAAGAAAGGTGTTTTCCCGTTCCCTGTGA
ACCAAGCTAACCATCAAGAGGGTATTCTTGTTGAAGCCAAACAAGAAGTTGAAACGAGAGAAGCGAAGGAAGAGCC
TAGAGAAGAAGTGAAACAACAGTACGTGGAAGAACCACCGCAAGAAGAAGAAGAGAAGGAAGAAGAGAAAGCAGAG
CAACAAGAAGCAGAGATTGTAGGATATTCAGAAGAAGCAGCAGTGGTCAATTGCTGCATAGACTCTTCAACCATAA
TGGAAATGGATCGTTGTGGGGACAACAATGAGCTGGCTTGGAACTTCTGTATGATGGATACAGGGTTTTCTCCGTT
TTTGACTGATCAGAATCTCGCGAATGAGAATCCCATAGAGTATCCGGAGCTATTCAATGAGTTAGCATTTGAGGAC
AACATCGACTTCATGTTCGATGATGGGAAGCACGAGTGCTTGAACTTGGAAAATCTGGATTGTTGCGTGGTGGGAA
GAGAGAGCCCACCCTCTTCTTCTTCACCATTGTCTTGCTTATCTACTGACTCTGCTTCATCAACAACAACAACAAC
AACCTCGGTTTCTTGTAACTATTTGGTCTGATAA
SEQ ID NO: 15-Arabidopsis thaliana Wrinkled 1 DNA sequence
ATGAAGAAGCGCTTAACCACTTCCACTTGTTCTTCTTCTCCATCTTCCTCTGTTTCTTCTTCTACTACTACTTCCT
CTCCTATTCAGTCGGAGGCTCCAAGGCCTAAACGAGCCAAAAGGGCTAAGAAATCTTCTCCTTCTGGTGATAAATC
TCATAACCCGACAAGCCCTGCTTCTACCCGACGCAGCTCTATCTACAGAGGAGTCACTAGACATAGATGGACTGGG
AGATTCGAGGCTCATCTTTGGGACAAAAGCTCTTGGAATTCGATTCAGAACAAGAAAGGCAAACAAGTTTATCTGG
GAGCATATGACAGTGAAGAAGCAGCAGCACATACGTACGATCTGGCTGCTCTCAAGTACTGGGGACCCGACACCAT
CTTGAATTTTCCGGCAGAGACGTACACAAAGGAATTGGAAGAAATGCAGAGAGTGACAAAGGAAGAATATTTGGCT
TCTCTCCGCCGCCAGAGCAGTGGTTTCTCCAGAGGCGTCTCTAAATATCGCGGCGTCGCTAGGCATCACCACAACG
GAAGATGGGAGGCTCGGATCGGAAGAGTGTTTGGGAACAAGTACTTGTACCTCGGCACCTATAATACGCAGGAGGA
AGCTGCTGCAGCATATGACATGGCTGCGATTGAGTATCGAGGCGCAAACGCGGTTACTAATTTCGACATTAGTAAT
TACATTGACCGGTTAAAGAAGAAAGGTGTTTTCCCGTTCCCTGTGAACCAAGCTAACCATCAAGAGGGTATTCTTG
TTGAAGCCAAACAAGAAGTTGAAACGAGAGAAGCGAAGGAAGAGCCTAGAGAAGAAGTGAAACAACAGTACGTGGA
AGAACCACCGCAAGAAGAAGAAGAGAAGGAAGAAGAGAAAGCAGAGCAACAAGAAGCAGAGATTGTAGGATATTCA
GAAGAAGCAGCAGTGGTCAATTGCTGCATAGACTCTTCAACCATAATGGAAATGGATCGTTGTGGGGACAACAATG
AGCTGGCTTGGAACTTCTGTATGATGGATACAGGGTTTTCTCCGTTTTTGACTGATCAGAATCTCGCGAATGAGAA
TCCCATAGAGTATCCGGAGCTATTCAATGAGTTAGCATTTGAGGACAACATCGACTTCATGTTCGATGATGGGAAG
CACGAGTGCTTGAACTTGGAAAATCTGGATTGTTGCGTGGTGGGAAGAGAGAGCCCACCCTCTTCTTCTTCACCAT
TGTCTTGCTTATCTACTGACTCTGCTTCATCAACAACAACAACAACAACCTCGGTTTCTTGTAACTATTTGGTCTG
ATAA
SEQ ID NO: 16-Arabidopsis thaliana Wrinkled 1 protein sequence
MKKRLTTSTCSSSPSSSVSSSTTTSSPIQSEAPRPKRAKRAKKSSPSGDKSHNPTSPASTRRSSIYRGVTRHRWTG
RFEAHLWDKSSWNSIQNKKGKQVYLGAYDSEEAAAHTYDLAALKYWGPDTILNFPAETYTKELEEMQRVTKEEYLA
SLRRQSSGFSRGVSKYRGVARHHHNGRWEARIGRVFGNKYLYLGTYNTQEEAAAAYDMAAIEYRGANAVTNFDISN
YIDRLKKKGVFPFPVNQANHQEGILVEAKQEVETREAKEEPREEVKQQYVEEPPQEEEEKEEEKAEQQEAEIVGYS
EEAAVVNCCIDSSTIMEMDRCGDNNELAWNFCMMDTGFSPFLTDONLANENPIEYPELFNELAFEDNIDFMEDDGK
HECLNLENLDCCVVGRESPPSSSSPLSCLSTDSASSTTTTTTSVSCNYLV
SEQ ID NO: 17-Arabidopsis thaliana Wrinkled 1 DNA sequence codon-optimized for Zea mays
ATGAAAAAGCGGCTCACCACTTCCACATGCTCATCCTCCCCGAGCAGCTCAGTTTCCTCCTCTACAACCACATCTA
GCCCCATCCAGTCTGAGGCGCCCCGACCAAAGAGGGCCAAACGGGCTAAGAAGTCCTCCCCGTCCGGGGATAAGTC
TCATAATCCCACGTCCCCCGCTTCTACTCGCCGGTCTTCAATATACCGCGGAGTTACCCGCCATAGGTGGACCGGT
CGTTTCGAGGCTCATTTGTGGGATAAGTCTTCGTGGAATAGTATTCAGAATAAAAAAGGTAAGCAAGTGTACCTCG
GTGCTTATGATTCGGAGGAAGCCGCCGCCCACACCTACGACCTGGCAGCCCTGAAGTATTGGGGCCCCGACACCAT
CCTAAACTTTCCAGCTGAAACGTACACCAAGGAGCTCGAGGAGATGCAAAGGGTCACGAAGGAGGAATACCTGGCC
TCTCTTAGACGTCAGAGCAGTGGCTTCTCCAGGGGTGTTTCCAAGTACCGCGGAGTCGCACGGCATCACCACAATG
GTAGATGGGAGGCCAGGATCGGGCGGGTGTTCGGCAATAAATACCTGTACCTCGGGACTTACAACACCCAGGAGGA
AGCTGCTGCAGCTTATGACATGGCGGCTATTGAGTACCGCGGCGCCAACGCAGTGACGAATTTCGACATCTCCAAC
TACATCGACCGTCTCAAGAAAAAGGGCGTCTTCCCGTTTCCAGTTAATCAAGCGAACCACCAAGAAGGTATATTGG
TCGAGGCGAAGCAGGAGGTCGAGACCCGGGAGGCTAAGGAGGAACCTCGCGAAGAAGTGAAACAGCAATACGTGGA
GGAACCGCCACAAGAAGAGGAGGAGAAGGAGGAGGAGAAGGCTGAGCAGCAAGAGGCCGAGATCGTAGGCTACTCA
GAGGAAGCCGCAGTGGTGAACTGCTGCATTGACTCGTCGACAATCATGGAGATGGATCGGTGCGGTGACAATAACG
AATTGGCTTGGAACTTCTGTATGATGGACACGGGGTTCAGTCCATTCCTAACTGACCAGAACCTAGCGAACGAGAA
CCCCATAGAATACCCCGAACTATTCAACGAGTTGGCGTTCGAAGACAATATAGACTTTATGTTCGACGACGGTAAA
CATGAGTGTCTGAACCTCGAAAATCTAGACTGCTGCGTCGTCGGCAGGGAATCACCCCCCTCCAGTTCCTCTCCGC
TGTCGTGCCTCAGTACGGACTCGGCGAGCTCTACCACGACCACTACAACTAGCGTTTCATGCAACTACTTGGTCTG
ATAA
SEQ ID NO: 18-Arabidopsis thaliana Wrinkled 1 DNA sequence codon-optimized for Zea mays
with addition of a 5′-UTR derived from an intron of Oryza sativa Cytosolic Superoxide
Dismutase 2
atcttctgatgagaataagcaaatccctattttttttgttgaagaaaagaaaagcgtgtccacgtggactggactc
tgaaattttcctcctcccttctggagtcttcctcatcagaaatcagaagaggagagggtgggcaactcgcagatcg
ccttctcgtcgcgctcgcgccgcaggggtcgcctgaggtatgcagcttcacctcccccaactttctagggttctaa
tcgcctctgctcgctcggattatgcgtggtgggtatggctccgattccgccggagtagctggatctgtgtgccccg
tgctaatttaggtttggctttcgattcggccgcgcttcggtttgttctcgcgcgtgattgcttcgttcggccatag
ggctcatctcaggctcgacatgtggacggccacaaacataaaaaatccttgttaaatttacggttcatgtggtagg
ggggcttttcaggatcggaggtttaggtgatttggagtagcaaaacgattttgctgtgttagagaattcgagaatc
tgtgccgtcagatgctaattagctggttttgaacaaatgttaagacatcctgatattatttgctgcgtttaatgtt
caaactttttgcacgggtgttgctgagctataaaggatatgaggatatagtaccaagttctatcgttttgttctga
tacatacaatttgctgtagtttatggcaccatcatactgagatatatatacctgtgcacctttcggcttctgtgca
gaacacatagacaatggtgaaggATGAAAAAGCGGCTCACCACTTCCACATGCTCATCCTCCCCGAGCAGCTCAGT
TTCCTCCTCTACAACCACATCTAGCCCCATCCAGTCTGAGGCGCCCCGACCAAAGAGGGCCAAACGGGCTAAGAAG
TCCTCCCCGTCCGGGGATAAGTCTCATAATCCCACGTCCCCCGCTTCTACTCGCCGGTCTTCAATATACCGCGGAG
TTACCCGCCATAGGTGGACCGGTCGTTTCGAGGCTCATTTGTGGGATAAGTCTTCGTGGAATAGTATTCAGAATAA
AAAAGGTAAGCAAGTGTACCTCGGTGCTTATGATTCGGAGGAAGCCGCCGCCCACACCTACGACCTGGCAGCCCTG
AAGTATTGGGGCCCCGACACCATCCTAAACTTTCCAGCTGAAACGTACACCAAGGAGCTCGAGGAGATGCAAAGGG
TCACGAAGGAGGAATACCTGGCCTCTCTTAGACGTCAGAGCAGTGGCTTCTCCAGGGGTGTTTCCAAGTACCGCGG
AGTCGCACGGCATCACCACAATGGTAGATGGGAGGCCAGGATCGGGCGGGTGTTCGGCAATAAATACCTGTACCTC
GGGACTTACAACACCCAGGAGGAAGCTGCTGCAGCTTATGACATGGCGGCTATTGAGTACCGCGGCGCCAACGCAG
TGACGAATTTCGACATCTCCAACTACATCGACCGTCTCAAGAAAAAGGGCGTCTTCCCGTTTCCAGTTAATCAAGC
GAACCACCAAGAAGGTATATTGGTCGAGGCGAAGCAGGAGGTCGAGACCCGGGAGGCTAAGGAGGAACCTCGCGAA
GAAGTGAAACAGCAATACGTGGAGGAACCGCCACAAGAAGAGGAGGAGAAGGAGGAGGAGAAGGCTGAGCAGCAAG
AGGCCGAGATCGTAGGCTACTCAGAGGAAGCCGCAGTGGTGAACTGCTGCATTGACTCGTCGACAATCATGGAGAT
GGATCGGTGCGGTGACAATAACGAATTGGCTTGGAACTTCTGTATGATGGACACGGGGTTCAGTCCATTCCTAACT
GACCAGAACCTAGCGAACGAGAACCCCATAGAATACCCCGAACTATTCAACGAGTTGGCGTTCGAAGACAATATAG
ACTTTATGTTCGACGACGGTAAACATGAGTGTCTGAACCTCGAAAATCTAGACTGCTGCGTCGTCGGCAGGGAATC
ACCCCCCTCCAGTTCCTCTCCGCTGTCGTGCCTCAGTACGGACTCGGCGAGCTCTACCACGACCACTACAACTAGC
GTTTCATGCAACTACTTGGTCTGATAA
SEQ ID NO: 19-the promoter of Arabidopsis thaliana Ubiquitin 10; the promoter additionally
contains a short linker sequence derived from the cauliflower mosaic virus 35S promoter,
denoted in lowercase
AGTCTAGCTCAACAGAGCTTTTAACCCAAATTGGTACAATAGAATACAACTTTAGATCATAATTCTCAAAAGAAAG
AGATTCCTTAGCTATTCTATCTGCCACTCCATTTCCTTCTCGGCTTGTATGCACAAGCATAAAATCCTCAAACTTG
CTAAGTAGATACTTTATGTCTTGGATAATTGGATTGAGACTTGACAAGCATAACTTTCATGTAACCAAAGACACAA
GTTGCTGAGAATCCACCTCAAAAATGATCTTCCTATAATTGAATCGGGATAATGACAGCACAGCCCATCTAAGAGC
CTCCACTTCTACTTCCAGCACGCTTCTTACTTTTACCACAGCTCTTGCACCTAACCATAACACCTTCCCTGTATGA
TCGCGAAGCACCCACCCTAAGCCACATTTTAATCCTTCTGTTGGCCATGCCCCATCAAAGTTGCACTTAACCCAAG
ATTGTGGTGGAGCTTCCCATGTTTCTCGTCTGTCCCGACGGTGTTGTGGTTGGTGCTTTCCTTACATTCTGAGCCT
CTTTCCTTCTAATCCACTCATCTGCATCTTCTTGTGTCCTTACTAATACCTCATTGGTTCCAAATTCCCTCCCTTT
AAGCACCAGCTCGTTTCTGTTCTTCCACAGCCTCCCAAGTATCCAAGGGACTAAAGCCTCCACATTCTTCAGATCA
GGATATTCTTGTTTAAGATGTTGAACTCTATGGAGGTTTGTATGAACTGATGATCTAGGACCGGATAAGTTCCCTT
CTTCATAGCGAACTTATTCAAAGAATGTTTTGTGTATCATTCTTGTTACATTGTTATTAATGAAAAAATATTATTG
GTCATTGGACTGAACACGAGTGTTAAATATGGACCAGGCCCCAAATAAGATCCATTGATATATGAATTAAATAACA
AGAATAAATCGAGTCACCAAACCACTTGCCTTTTTTAACGAGACTTGTTCACCAACTTGATACAAAAGTCATTATC
CTATGCAAATCAATAATCATACAAAAATATCCAATAACACTAAAAAATTAAAAGAAATGGATAATTTCACAATATG
TTATACGATAAAGAAGTTACTTTTCCAAGAAATTCACTGATTTTATAAGCCCACTTGCATTAGATAAATGGCAAAA
AAAAACAAAAAGGAAAAGAAATAAAGCACGAAGAATTCTAGAAAATACGAAATACGCTTCAATGCAGTGGGACCCA
CGGTTCAATTATTGCCAATTTTCAGCTCCACCGTATATTTAAAAAATAAAACGATAATGCTAAAAAAATATAAATC
GTAACGATCGTTAAATCTCAACGGCTGGATCTTATGACGACCGTTAGAAATTGTGGTTGTCGACGAGTCAGTAATA
AACGGCGTCAAAGTGGTTGCAGCCGGCACACACGAGTCGTGTTTATCAACTCAAAGCACAAATACTTTTCCTCAAC
CTAAAAATAAGGCAATTAGCCAAAAACAACTTTGCGTGTAAACAACGCTCAATACACGTGTCATTTTATTATTAGC
TATTGCTTCACCGCCTTAGCTTTCTCGTGACCTAGTCGTCCTCGTCTTTTCTTCTTCTTCTTCTATAAAACAATAC
CCAAAGAGCTCTTCTTCTTCACAATTCAGATTTCAATTTCTCAAAATCTTAAAAACTTTCTCTCAATTCTCTCTAC
CGTGATCAAGGTAAATTTCTGTGTTCCTTATTCTCTCAAAATCTTCGATTTTGTTTTCGTTCGATCCCAATTTCGT
ATATGTTCTTTGGTTTAGATTCTGTTAATCTTAGATCGAAGACGATTTTCTGGGTTTGATCGTTAGATATCATCTT
AATTCTCGATTAGGGTTTCATAGATATCATCCGATTTGTTCAAATAATTTGAGTTTTGTCGAATAATTACTCTTCG
ATTTGTGATTTCTATCTAGATCTGGTGTTAGTTTCTAGTTTGTGCGATCGAATTTGTCcccttcctctatataagg
aa
SEQ ID NO: 20-the promoter of Zea mays Ubiquitin 1
CTGCAGTGCAGCGTGACCCGGTCGTGCCCCTCTCTAGAGATAATGAGCATTGCATGTCTAAGTTATAAAAAATTAC
CACATATTTTTTTTGTCACACTTGTTTGAAGTGCAGTTTATCTATCTTTATACATATATTTAAACTTTACTCTACG
AATAATATAATCTATAGTACTACAATAATATCAGTGTTTTAGAGAATCATATAAATGAACAGTTAGACATGGTCTA
AAGGACAATTGAGTATTTTGACAACAGGACTCTACAGTTTTATCTTTTTAGTGTGCATGTGTTCTCCTTTTTTTTT
GCAAATAGCTTCACCTATATAATACTTCATCCATTTTATTAGTACATCCATTTAGGGTTTAGGGTTAATGGTTTTT
ATAGACTAATTTTTTTAGTACATCTATTTTATTCTATTTTAGCCTCTAAATTAAGAAAACTAAAACTCTATTTTAG
TTTTTTTATTTAATAATTTAGATATAAAATAGAATAAAATAAAGTGACTAAAAATTAAACAAATACCCTTTAAGAA
ATTAAAAAAACTAAGGAAACATTTTTCTTGTTTCGAGTAGATAATGCCAGCCTGTTAAACGCCGTCGACGAGTCTA
ACGGACACCAACCAGCGAACCAGCAGCGTCGCGTCGGGCCAAGCGAAGCAGACGGCACGGCATCTCTGTCGCTGCC
TCTGGACCCCTCTCGAGAGTTCCGCTCCACCGTTGGACTTGCTCCGCTGTCGGCATCCAGAAATTGCGTGGCGGAG
CGGCAGACGTGAGCCGGCACGGCAGGCGGCCTCCTCCTCCTCTCACGGCACCGGCAGCTACGGGGGATTCCTTTCC
CACCGCTCCTTCGCTTTCCCTTCCTCGCCCGCCGTAATAAATAGACACCCCCTCCACACCCTCTTTCCCCAACCTC
GTGTTGTTCGGAGCGCACACACACACAACCAGATCTCCCCCAAATCCACCCGTCGGCACCTCCGCTTCAAGGTACG
CCGCTCGTCCTCCCCCCCCCCCCCTCTCTACCTTCTCTAGATCGGCGTTCCGGTCCATGGTTAGGGCCCGGTAGTT
CTACTTCTGTTCATGTTTGTGTTAGATCCGTGTTTGTGTTAGATCCGTGCTGCTAGCGTTCGTACACGGATGCGAC
CTGTACGTCAGACACGTTCTGATTGCTAACTTGCCAGTGTTTCTCTTTGGGGAATCCTGGGATGGCTCTAGCCGTT
CCGCAGACGGGATCGATTTCATGATTTTTTTTGTTTCGTTGCATAGGGTTTGGTTTGCCCTTTTCCTTTATTTCAA
TATATGCCGTGCACTTGTTTGTCGGGTCATCTTTTCATGCTTTTTTTTGTCTTGGTTGTGATGATGTGGTCTGGTT
GGGCGGTCGTTCTAGATCGGAGTAGAATTCTGTTTCAAACTACCTGGTGGATTTATTAATTTTGGATCTGTATGTG
TGTGCCATACATATTCATAGTTACGAATTGAAGATGATGGATGGAAATATCGATCTAGGATAGGTATACATGTTGA
TGCGGGTTTTACTGATGCATATACAGAGATGCTTTTTGTTCGCTTGGTTGTGATGATGTGGTGTGGTTGGGCGGTC
GTTCATTCGTTCTAGATCGGAGTAGAATACTGTTTCAAACTACCTGGTGTATTTATTAATTTTGGAACTGTATGTG
TGTGTCATACATCTTCATAGTTACGAGTTTAAGATGGATGGAAATATCGATCTAGGATAGGTATACATGTTGATGT
GGGTTTTACTGATGCATATACATGATGGCATATGCAGCATCTATTCATATGCTCTAACCTTGAGTACCTATCTATT
ATAATAAACAAGTATGTTTTATAATTATTTTGATCTTGATATACTTGGATGATGGCATATGCAGCAGCTATATGTG
GATTTTTTTAGCCCTGCCTTCATACGCTATTTATTTGCTTGGTACTGTTTCTTTTGTCGATGCTCACCCTGTTGTT
TGGTGTTACTTCTGCAG
SEQ ID NO: 21-the promoter of Arabidopsis thaliana Ribulose-1,5,-bisphosphate
Carboxylase/Oxygenase Small Subunit 1a
GTGGTCGAGATTGTGTATTATTCTTTAGTTATTACAAGACTTTTAGCTAAAATTTGAAAGAATTTACTTTAAGAAA
ATCTTAACATCTGAGATAATTTCAGCAATAGATTATATTTTTCATTACTCTAGCAGTATTTTTGCAGATCAATCGC
AACATATATGGTTGTTAGAAAAAATGCACTATATATATATATATTATTTTTTCAATTAAAAGTGCATGATATATAA
TATATATATATATATATATATGTGTGTGTGTATATGGTCAAAGAAATTCTTATACAAATATACACGAACACATATA
TTTGACAAAATCAAAGTATTACACTAAACAATGAGTTGGTGCATGGCCAAAACAAATATGTAGATTAAAAATTCCA
GCCTCCAAAAAAAAATCCAAGTGTTGTAAAGCATTATATATATATAGTAGATCCCAAATTTTTGTACAATTCCACA
CTGATCGAATTTTTAAAGTTGAATATCTGACGTAGGATTTTTTTAATGTCTTACCTGACCATTTACTAATAACATT
CATACGTTTTCATTTGAAATATCCTCTATAATTATATTGAATTTGGCACATAATAAGAAACCTAATTGGTGATTTA
TTTTACTAGTAAATTTCTGGTGATGGGCTTTCTACTAGAAAGCTCTCGGAAAATCTTGGACCAAATCCATATTCCA
TGACTTCGATTGTTAACCCTATTAGTTTTCACAAACATACTATCAATATCATTGCAACGGAAAAGGTACAAGTAAA
ACATTCAATCCGATAGGGAAGTGATGTAGGAGGTTGGGAAGACAGGCCCAGAAAGAGATTTATCTGACTTGTTTTG
TGTATAGTTTTCAATGTTCATAAAGGAAGATGGAGACTTGAGAAGTTTTTTTTGGACTTTGTTTAGCTTTGTTGGG
CGTTTTTTTTTTTTGATCAATAACTTTGTTGGGCTTATGATTTGTAATATTTTCGTGGACTCTTTAGTTTATTTAG
ACGTGCTAACTTTGTTGGGCTTATGACTTGTTGTAACATATTGTAACAGATGACTTGATGTGCGACTAATCTTTAC
ACATTAAACATAGTTCTGTTTTTTGAAAGTTCTTATTTTCATTTTTATTTGAATGTTATATATTTTTCTATATTTA
TAATTCTAGTAAAAGGCAAATTTTGCTTTTAAATGAAAAAAATATATATTCCACAGTTTCACCTAATCTTATGCAT
TTAGCAGTACAAATTCAAAAATTTCCCATTTTTATTCATGAATCATACCATTATATATTAACTAAATCCAAGGTAA
AAAAAAGGTATGAAAGCTCTATAGTAAGTAAAATATAAATTCCCCATAAGGAAAGGGCCAAGTCCACCAGGCAAGT
AAAATGAGCAAGCACCACTCCACCATCACACAATTTCACTCATAGATAACGATAAGATTCATGGAATTATCTTCCA
CGTGGCATTATTCCAGCGGTTCAAGCCGATAAGGGTCTCAACACCTCTCCTTAGGCCTTTGTGGCCGTTACCAAGT
AAAATTAACCTCACACATATCCACACTCAAAATCCAACGGTGTAGATCCTAGTCCACTTGAATCTCATGTATCCTA
GACCCTCCGATCACTCCAAAGCTTGTTCTCATTGTTGTTATCATTATATATAGATGACCAAAGCACTAGACCAAAC
CTCAGTCACACAAAGAGTAAAGAAGAACA
SEQ ID NO: 22-the promoter of Arabidopsis thaliana Chlorophyll a/b Binding Protein 3
GAATTCATGTGTGAGGGCAATTAGTGATTGTAAAAATAAAATTGTGTTTTGTAAAAAACTTTTACTGTCGAAATTA
TTTAGGGTGATGAAAAAATCAGTAAACTACGAATGATAGCTTAAAGAGTTTCTATCAAAGTGATTGAGGAATAGTT
TGTTGCAAATTAAACCTCTAACAAAATGTTTTCTGTTGTGGTTTTTCATCTCTACAAATTTTGAATTTTATGATGA
ATTAGAAAGATAGAATGAGTTACTTTAGATTTTAAAAGGTTGTTCAAGTTTACAAAACAGATTACTAGAATCATGA
TTAAAAATTTACAAGCTACATATTGTCTAAACCAATGATGTTGAACATACCAGATGATAGTTTTTCAGTGTTTGAA
CAATCAATTGGATAGTTTTTATGTTTCTGCAAAATATGCAAATAATCAGTGTTTTTGAGTCTTTGCATTTTGATTT
AAAAGCAAAAACAACTGAGTTTCAAGGTTAAATTAATTACATTATTCATGAGATTTATCAGGTTAGTGGATAAACT
GACAATGGAATCAATGTTATTGTAAATTGGTAGTGATGTTGGACTTCTAATGTTACTCTCTATGATGTTTCGGTCA
TCAATATCACACTATCTTTACTTTTATTTAAAGGAAAGATCACACAAATAAGTTATCTCTATTCAGAACTATTAAG
CTGCTTCCAAAAGACTTGCAACATGTGGTCTCGAAATGCTTTGGCTGCAATGAAAAAATCATAGCAAAAGCTAGTG
GACTAGAGACTGCCACATAAGAATAGTAAACGTTAAAACCAAAATCTCAAAAATCCAATGAGTAAAGAGATATAGA
TTACTTCATAGATAACAAACGTTACTCGCAATTTTCCTATATAATCCAACCCTACCTAACCATTTTCAATCACTCT
CACTCACAAGTTAGTCACCAAAAAAAAAAAAAAACACAAAAAGTTTCA
SEQ ID NO: 23-the promoter of Ipomoea batatas Ribulose-1,5,-bisphosphate Carboxylase/
Oxygenase Small Subunit 1
GATCCTTATTCGTGAGATGAGTCGGGTCGGGTCAACGCACCATGCAAATGTCATACTTATATACTCAAATATAACA
CTAATCAAAAAAACAATTTTTGTTACATATAAGAGAAAAAGTAATACATTTTTCATAATAAGTAATGTTGACAAGT
GCCCCTTACTTATAAGGGCAAATATAATACTTTTGAGGAAAAATGTAATACTTTTAAATCGAAATGTAAAAGTATT
GTATTTTCCCTTAAAAGTATTACATTTACCCTTATAAGTAACAAAAATTTTATTCTTGATTAGTATTGCATTTGAG
CATATAAGTATGACATTTGTGCATATAAGTGTTACATTTACATATTGACCCGAACCGACCCGATCCGTCTCACGGT
GAGACGGTCTCACACCAATTTTTGCCAATTGTATATTATTTCCTTAATAACATTGCAACTTAACGTGCGCAATAAT
GAATTGAGCAAAAGAAGCCCGTACATGCATCTGCTCCAAAGTCCCAAATACTGGTACCCGTAAAGATCGAGCTTAG
GATTTGTTAATCGGGCTTTTATGGATTTGTGGGTTTGATAAGATATTTTGATACCGGGCTGGGTTGAATGAAAAAG
CCCAATATAGTCTAGCAGCTGCTGAGAAATGGGAATGGGACTACTGACAGAAAGGATGGCGAAGGGCAAAAATAAG
CAACCATGATCACACAGTCATATCCTCTTGAGGATGAGATAAGATTAACGAGGGCCTCCCACGTGGCACCCCTTTT
GTGGTGGCTTATATCAATACATTGCCCTGATGTGGATATTAAGGTTGTAATGTCATGCACCACATAAATCCAATGG
CCCTGCTTCCTCAAAGATTAGGTTGATTAAACGTCGCCCGTTAGATTGGACTCATGTGACACCTTATCGTTATATA
TACCAAGCAATAGCACACTATCACCACCATTACAAAGTAAAGTAGTAAGTAATACAGAGAAGAGCGGCAGCTGCTA
GCA
SEQ ID NO: 24-the promoter of Brassica napus Napin
GACGGTGGCATTCAGCTCCCAATTTATATTCCCAACGGCACTACCTCCAAAATTTATAGACTCTCATCCCCTTTTA
AACCAACTTAGTAAACGTTTTTTTTTTTAATTTTATGAAGTTAAGTTTTTACCTTGTTTTTAAAAAGAATCGTTCA
TAAGATGCCATGCCAGAACATTAGCTACACGTTACACATAGCATGCAGCCGCGGAGAATTGTTTTTCTTCGCCACT
TGTCACTCCCTTCAAACACCTAAGAGCTTCTCTCTCACAGCACACACATACAATCACATGCGTGCATGCATTATTA
CACGTGATCGCCATGCAAATCTCCTTTATAGCCTATAAATTAACTCATCCGCTTCACTCTTTACTCAAACCAAAAC
TCATCAATACAAACAAGATTAAAAACATACACCTCGAGATG
SEQ ID NO: 25-the promoter of Arabidopsis thaliana Cruciferin
GCTCATGCTAAGCTGCGCAAAATACTTCCTAATCAAAACAGTAACAACGAGTAATTAGCAAAATCCGAGCAGAAAA
CTCTCACCCACCTCCGAAATTCACGTCTTCACTAAAATTTTCGAAAGGAATCGATCAATACCAACCCATTACACAA
AATACATAATCAAAATGGCGAGAATCGTACCTGGAAACTTTGCTTCAAGTCGCAGAGAGAGGAAAAGGAAGATCGT
GGAGAAAGGGGTTTAGGGTTTAAGCTCAGACTTCTATTGGAGTAAATGGGACGGTGTCACATTTTCCGTTTTGGAA
ATGAACTTTGGGCTCACGTTATGGGCTATTAGATATTTGATGGGCTTTCTAGTAAATACAATATAAGTTATTGGGC
TTAGTTTAAATAAGCCCATGTTGGAAATATTTGACACATGTCTTGGCTACTAGTGCTAAACATGCAACCGAACAGT
TGTCGAGACAAGTCGCAGCATATACAATGGATCAAACACGCCTAGTGTCGCCGCGTCTCGCTCATGTGTCACCTTG
TTTCCTCGTTTTTTTTTAATTTTTCATAAGTTCTTTTGTTTTATCTTCAATACAAATTTTTGGCTGTATCTTGCAA
ACTCTTCGATCATATCGCCAATATACGTGAACACTGGTGATCTAATTTGTTGTGTTAATTGTTAAATTTAGATTCT
ATTCTCCGGTTTAAAAGTGAATTATATGTATCATGGTTAAAACATTGTAAGTAAGATGATAATAAAATGATAAATT
TAGTTGATGGATAACGTGAAGCAAAAAATGAGATAGATACATTTGATTTTGTCGTATTTTGACATATGCGGAGAGT
GAGCTACGCGCATGAAGATCAAGAGACACTTGCTCGAGCTCACAGAGTGACGTGTAAAAAGCTTAGACTGAAGTCC
CCATGCAAACCTAATCCTACGTGGCTCAAACCACGAGCTCACTTGACAATATATAAACTCCTCCTAAGTCCCGTTC
TCTTCATCCATCTCTCACAACAAACAAAAAGAAA
SEQ ID NO: 26-the terminator of Arabidopsis thaliana Heat Shock Protein
ATATGAAGATGAAGATGAAATATTTGGTGTGTCAAATAAAAAGCTTGTGTGCTTAAGTTTGTGTTTTTTTCTTGGC
TTGTTGTGTTATGAATTTGTGGCTTTTTCTAATATTAAATGAATGTAAGATCTCATTATAATGAATAAACAAATGT
TTCTATAATCCATTGTGAATGTTTTGTTGGATCTCTTCTGCAGCATATAACTACTGTATGTGCTATGGTATGGACT
ATGGAATATGATTAAAGATAAG
SEQ ID NO: 27-the terminator of Agrobacterium tumefaciens Nopaline Synthase
AATAAAGTTTCTTAAGATTGAATCCTGTTGCCGGTCTTGCGATGATTATCATATAATTTCTGTTGAATTACGTTAA
GCATGTAATAATTAACATGTAATGCATGACGTTATTTATGAGATGGGTTTTTATGATTAGAGTCCCGCAATTATAC
ATTTAATACGCGATAGAAAACAAAATATAGCGCGCAAACTAGGATAAATTATCGCGCGCGGTGTCATCTATGTTAC
TAGATC
SEQ ID NO: 28-DNA sequence of a polycistronic gene sequence encoding VBPO, AACT2, and WRI1
separated by the self-cleaving P2A peptide
atggcggaggagagacgacaaaacgcacttgaaatcaggatccaagctgctaagctagctaagaagagggaccatc
ctacacacaaagccaatggagatgaagacagatatccagagacacttataggtagctttactaaaggactgccgca
cgagaaggaaacaggtttattgtctaaccctgctgacttcgctgatttcgttcgagctattaataccggtgctatc
aaggacatgaggaggttgaaattgggtatcgatgaagatcctcgttttatcagtggaatcgcgtccaaaggtgaca
agcacccttttgctgatacacgagcttgggaatctatggcagcaggactgacttacgatcttgagggaccagatgc
ccaggcggtcactatgcctccagcgcctaagcttgatagcgacgaactggtgacggagatcacggaatcttattgg
atggctttattgagagatgttccttttaccgagtttgaacgagatgggaacactgctgccgctgcagctagcatat
cccggacacgatgggttcaatataatgagcaaccttcgcaaagacctagcacacttacagacgaagagatagcaag
actacgaggaccgtataccaaaaaaaacgtattcaggggtgtcaccaatggggagaatgtaggcccttatttatca
cagtttcttctggttggaacaaagggcattggtgatgcccaacaagttagtgatggttatgtacagtatggaggaa
tgagaatggaccaaagagttcgagtggctgtgcctaaaagagattacatgaccacatgggcttcttggctcgatgt
tcaaaatgctggtgatcttagaggtagagaaatctatgacgacgacacaccattccgattcataacaacgccacgt
gatctggctacatgggtgcattttgatgcattataccaggcttatttgaatgcttgtataatactattagatatta
aggctccgttcgatcctcacatccctttccaagcggatgatgatgttgataaacagcaaggattcgcaacgtttgg
tggtccgcatattctttctctttgcactgaggtggctacaagagcgctgaaagcggttcgttttcagaagtacaac
ttacacaggaggttgagacctgaggcaattggtggtcttgtcgagaggtttaagaaaacaaatggcgacccaaaat
tcgcacctgttaaaaaattggttaatgacttagacggtgatatgttgcggcgagttgagcaacataactgtgaaca
aaataagctctccgatgacggccatgctagaagggaagactatagcccagagggagagtcttcacaatcatatctt
ttacctatggcattccctgagggttcccctatgcatccttcatacggtgcgggacacgccacggtggccggtgcat
gtgtcactgtgcttaaagcctttttcgatcatgaatatgaacttgatttttgctatgtccctacaaccgatggaaa
gaggcttgagaaagttaatattaatgaaaaacttactgttgagggcgaacttaataagctttgtgctaacataagt
attggtcgtaactgggcgggcgttcattactactctgactatttcgagagtatcaaggttggagaggagatcgcta
ttggtatcttgcaggaacaaaaattgacttatggagaggatttttttatgactctacctaagtttgacggtgagaa
aataagaataGGAAGCGGAGCTACTAACTTCAGCCTGCTGAAGCAGGCTGGAGACGTGGAGGAGAACCCTGGACCT
ATGGCCCATACATCAGAATCTGTGAATCCTAGAGATGTTTGCATTGTGGGTGTTGCACGTACTCCAATGGGTGGCT
TTCTCGGATCTCTTTCATCTTTACCTGCCACAAAGCTTGGATCTTTAGCTATTGCAGCTGCTTTGAAGAGAGCAAA
TGTTGATCCAGCTCTTGTTCAAGAAGTTGTCTTTGGCAATGTTCTTAGTGCTAATTTGGGTCAAGCTCCTGCTCGT
CAAGCTGCTTTAGGTGCAGGAATCCCTAACTCTGTTATCTGTACTACAGTTAACAAGGTTTGTGCATCAGGCATGA
AAGCGGTAATGATTGCTGCTCAAAGTATCCAGTTAGGGATCAATGATGTAGTTGTGGCGGGTGGTATGGAAAGCAT
GTCTAATACACCAAAATATTTGGCAGAAGCAAGGAAGGGATCTCGTTTTGGTCATGATTCTTTAGTAGATGGAATG
TTGAAGGATGGACTATGGGATGTCTATAACGACTGTGGGATGGGAAGCTGTGCAGAATTATGCGCTGAGAAGTTTC
AGATTACAAGGGAGCAGCAAGATGACTATGCAGTTCAGAGTTTTGAGCGTGGTATTGCTGCCCAGGAAGCTGGCGC
CTTCACATGGGAAATCGTCCCGGTTGAAGTTTCTGGAGGAAGAGGTAGGCCATCAACCATTGTTGACAAGGACGAA
GGTCTTGGGAAGTTTGATGCTGCAAAATTGAGGAAACTCCGTCCTAGTTTCAAAGAGAATGGAGGGACTGTTACAG
CTGGAAATGCGTCTAGCATAAGTGATGGTGCAGCTGCCCTTGTCCTAGTGAGCGGAGAGAAGGCTCTTCAGCTAGG
ACTTCTAGTATTAGCAAAAATTAAAGGGTATGGTGACGCAGCTCAGGAACCAGAGTTTTTCACTACTGCTCCTGCT
CTTGCTATACCAAAAGCCATTGCACATGCTGGTTTGGAATCTTCTCAAGTTGATTACTATGAGATCAATGAAGCAT
TTGCAGTTGTAGCACTTGCAAATCAAAAGCTACTCGGGATTGCTCCAGAGAAAGTGAACGTAAATGGAGGAGCTGT
CTCCTTAGGACACCCTCTAGGCTGCAGTGGCGCCCGTATTCTAATCACGTTGCTTGGGATACTAAAGAAGAGAAAC
GGAAAGTACGGTGTGGGAGGAGTGTGCAACGGAGGAGGAGGTGCTTCTGCTCTAGTTCTTGAGCTCCTTGGTTCAG
GTGCCACTAATTTTTCTCTCTTGAAACAGGCCGGTGACGTTGAAGAGAACCCAGGTCCAATGAAGAAGCGCTTAAC
CACTTCCACTTGTTCTTCTTCTCCATCTTCCTCTGTTTCTTCTTCTACTACTACTTCCTCTCCTATTCAGTCGGAG
GCTCCAAGGCCTAAACGAGCCAAAAGGGCTAAGAAATCTTCTCCTTCTGGTGATAAATCTCATAACCCGACAAGCC
CTGCTTCTACCCGACGCAGCTCTATCTACAGAGGAGTCACTAGACATAGATGGACTGGGAGATTCGAGGCTCATCT
TTGGGACAAAAGCTCTTGGAATTCGATTCAGAACAAGAAAGGCAAACAAGTTTATCTGGGAGCATATGACAGTGAA
GAAGCAGCAGCACATACGTACGATCTGGCTGCTCTCAAGTACTGGGGACCCGACACCATCTTGAATTTTCCGGCAG
AGACGTACACAAAGGAATTGGAAGAAATGCAGAGAGTGACAAAGGAAGAATATTTGGCTTCTCTCCGCCGCCAGAG
CAGTGGTTTCTCCAGAGGCGTCTCTAAATATCGCGGCGTCGCTAGGCATCACCACAACGGAAGATGGGAGGCTCGG
ATCGGAAGAGTGTTTGGGAACAAGTACTTGTACCTCGGCACCTATAATACGCAGGAGGAAGCTGCTGCAGCATATG
ACATGGCTGCGATTGAGTATCGAGGCGCAAACGCGGTTACTAATTTCGACATTAGTAATTACATTGACCGGTTAAA
GAAGAAAGGTGTTTTCCCGTTCCCTGTGAACCAAGCTAACCATCAAGAGGGTATTCTTGTTGAAGCCAAACAAGAA
GTTGAAACGAGAGAAGCGAAGGAAGAGCCTAGAGAAGAAGTGAAACAACAGTACGTGGAAGAACCACCGCAAGAAG
AAGAAGAGAAGGAAGAAGAGAAAGCAGAGCAACAAGAAGCAGAGATTGTAGGATATTCAGAAGAAGCAGCAGTGGT
CAATTGCTGCATAGACTCTTCAACCATAATGGAAATGGATCGTTGTGGGGACAACAATGAGCTGGCTTGGAACTTC
TGTATGATGGATACAGGGTTTTCTCCGTTTTTGACTGATCAGAATCTCGCGAATGAGAATCCCATAGAGTATCCGG
AGCTATTCAATGAGTTAGCATTTGAGGACAACATCGACTTCATGTTCGATGATGGGAAGCACGAGTGCTTGAACTT
GGAAAATCTGGATTGTTGCGTGGTGGGAAGAGAGAGCCCACCCTCTTCTTCTTCACCATTGTCTTGCTTATCTACT
GACTCTGCTTCATCAACAACAACAACAACAACCTCGGTTTCTTGTAACTATTTGGTCTGATAA
SEQ ID NO: 29-DNA sequence encoding the P2A self-cleaving peptide
GCTACTAACTTCAGCCTGCTGAAGCAGGCTGGAGACGTGGAGGAGAACCCTGGACCT
SEQ ID NO: 30-protein sequence of the self-cleaving P2A peptide
ATNFSLLKQAGDVEENPGP
SEQ ID NO: 31-DNA sequence encoding the E2A self-cleaving peptide
CAATGCACTAATTATGCCTTACTGAAGCTAGCAGGCGATGTTGAAAGCAATCCTGGTCCTTAA
SEQ ID NO: 32-protein sequence of the self-cleaving E2A peptide
QCTNYALLKLAGDVESNPGP
SEQ ID NO: 33-DNA sequence encoding the F2A self-cleaving peptide
GGTTCAGGTGTTAAGCAAACTCTTAATTTTGATTTGCTAAAGCTAGCGGGCGATGTAGAAAGCAACCCCGGACCAT
AA
SEQ ID NO: 34-protein sequence of the self-cleaving F2A peptide
GSGVKQTLNFDLLKLAGDVESNPGP
SEQ ID NO: 35-DNA sequence encoding the T2A self-cleaving peptide
GAAGGCCGGGGGTCTCTATTGACTTGTGGAGATGTCGAGGAGAATCCGGGACCTTAG
SEQ ID NO: 36-protein sequence of the self-cleaving T2A peptide
EGRGSLLTCGDVEENPGP
SEQ ID NO: 37-DNA sequence encoding an alternative sequence of the P2A self-cleaving peptide
GGTAGTGGTGCGACTAACTTTAGTTTGCTCAAACAGGCAGGTGATGTAGAAGAGAATCCAGGGCCTTGA
SEQ ID NO: 38-alternative protein sequence of the self-cleaving P2A peptide
GSGATNFSLLKQAGDVEENPGP
SEQ ID NO: 39-DNA sequence encoding an alternative sequence of the F2A self-cleaving peptide
GTTAAGCAAACTCTGAACTTCGATTTATTAAAGCTGGCGGGTGACGTAGAATCGAATCCCGGACCATAG
SEQ ID NO: 40-alternative protein sequence of the self-cleaving F2A peptide
VKQTLNFDLLKLAGDVESNPGP
SEQ ID NO: 41-DNA sequence encoding the Vanadium-dependent iodoperoxidase of Zobellia
galactanivorans
ATGAAGAAGATTCTTATCGCACTAATATCGTTTGCTTTTGCGGTTTCGTGCAAAGCTCCACAAAAAGAAGAACCTA
TTAACATTACCCCCGAAGAGCTTGACGCTTCAATAGACAGGGTGACGGAAATTATGATCCACGATATCTTTTCCCC
TCCCGTTGCGAGTAGGATCTTTGCCTATCCCAACGTTGCCGCCTACGAAATTGTAGCCGCCACCAATGACAACTAC
AACTCCTTGGCCGGCCAATTGAACGGGCTGACCGCCATACCGGAACCCGATACCACTAAGACCATCAACTACGAGC
TTGCAGCCGTCGTCGCCCATATGGAGCTTAGCAAAAGGTTGATTTTCTCAGAAGACCGAATGGAATCCCTGCGCGA
TAGCCTATACATGGTTTGGGAAGGGAAAAATCCTGTTCTATTCTCCGATTCCAAAGCCTACGGCCTACAAGTGGCC
GACCATATAGGCGAATGGATGAACAAGGACAATTACGCCCAAACCCGCACCATGCCGAAATTTACGGTAGATGCGG
ACGACCCCGGCCGCTGGCAACCCACCCCACCTGCCTACATGGACGGTATTGAACCCCACTGGAATAAAATCAGGCC
ATTTGTATTGGATTCGGCAGCACAGTTCAAGCCCGTTCCACCTCCGGCATATTCCCTTGAAGAAGACTCCGCGTTT
TATAAAGAATTAAAAGAAGTCTATGACGTAAGGAACAAAATCACCGAGGAAGGCGATAGTTCCGAAGAAATTCAGA
TTGCCCGCTTTTGGGATTGTAACCCTTATGTATCGGTTACCCGTGGCCACTTGATGTTCGCCACCAAGAAAATAAC
CCCAGGTGCGCATTGGATGGGAATTGCCAAAATTGCCGCACGTAAAACCAACAGTGATTTTGCCAAAACCCTTTTC
GCCTATACCAAGGCCTCGGTAGCCATGGCGGATGCCTTTATCAGTTGTTGGGACGAAAAGTACAGAAGCAACCTCA
TCCGTCCGGAAACCGTAATCAACCAACATATAGACGACAGCTGGAAACCAGTGCTACAAACCCCTCCGTTTCCAGA
GTACACCAGCGGACATAGTGTAGTCTCAGGGGCGGCATCGGTTGTACTGACCGAGGTCTTTGGTGACAATTTCTCC
TTTGACGACGATACGGAAGTACCTTACGGCCTACCTATCCGAAGCTTTAAATCCTTTAAGCAAGCTGCCGACGAAG
CAGCGATCAGTCGCATGTACGGAGGCATACACTACCGTGCAGCTATTGAAGTAGGGGTAAAACAAGGCAGGGACCT
AGGTACCTTTGTCGTAAACAAACTACATATGCTATCCGATAAGAAAGTAGCCCAAAACTAG
SEQ ID NO: 42-protein sequence of the Vanadium-dependent iodoperoxidase of Zobellia
galactanivorans
MGSMKKILIALISFAFAVSCKAPQKEEPINITPEELDASIDRVTEIMIHDIFSPPVASRIFAYPNVAAYEIVAATN
DNYNSLAGQLNGLTAIPEPDTTKTINYELAAVVAHMELSKRLIFSEDRMESLRDSLYMVWEGKNPVLFSDSKAYGL
QVADHIGEWMNKDNYAQTRTMPKFTVDADDPGRWQPTPPAYMDGIEPHWNKIRPFVLDSAAQFKPVPPPAYSLEED
SAFYKELKEVYDVRNKITEEGDSSEEIQIARFWDCNPYVSVTRGHLMFATKKITPGAHWMGIAKIAARKTNSDFAK
TLFAYTKASVAMADAFISCWDEKYRSNLIRPETVINQHIDDSWKPVLQTPPFPEYTSGHSVVSGAASVVLTEVEGD
NFSFDDDTEVPYGLPIRSFKSFKQAADEAAISRMYGGIHYRAAIEVGVKQGRDLGTFVVNKLHMLSDKKVAQN
SEQ ID NO: 43-DNA sequence encoding the Vanadium-dependent iodoperoxidase of Laminaria
digitata
ATGAAGGGGCTTGCAGGACCAGCCGGTGCTATGGCCGTTGTCGCGCTCGGGCTTGTCCCCGGCGGAGCAATCGGGA
AATCTTTGCGACAAGAGCCCTCTGAACCCCGCCTAAGTGGCGGCGTGGATACGGCTGCATCGCCATCGAAGGACAC
CCTGAAAGGGAGTCTTTCGCGTAAGCTCCAAGTCGTCAACGATGATGCCCTCGATGTTAGTGGCACGCCCGCAGAA
AGAGCTGCCAACGCGCTGAACCAGCGGATCGAATTTGCGGAGACGGAGTTCACGGCATCCGAAGGCACGCTCCACC
TTAACAACGGAGACCGCTCATCCGCCGCCACGTTCCACAAGTCGCTGCCGCACGACAGCCTAGGACAGGTGAACAG
CGAGGACTTTGACCTCCTCATGGAGTGCATCGCTCAAGGTGATTTCGACACATGCGAGCTGGTGCCGGCCGGAGAC
GACGGCAGGCTGTCCAACCCCCTCGGGGGTATCGCCGTCGAGATGGCGGGAGCCGCCGGCCCCGCTTTGACCCTCC
CTCCGGCCTCAGCGATCAACTCCGAGGACTTGGCTGCTCAAATGGCGGAACAGTACTGGATGGCCTTGACCAGGGA
CGTACCTTTCTCTCAGTACGGCGAGGATGAGGCGACAGTGGCTGCAGCAGACAACTTGGCCACCATGCCTGGTTTT
GCCGACATTGTCGGGGTGGCCGTCGATCCGGAAACCAGAAGAGCGGATCCGCAGTCGCAGCTTTTCCGGTCCTCTG
CCTTCGGCGTCGAGACAGGGCCCTTCATTTCCCAGCTACTGGTGAAGGACTTCACGATTGATTCTATCACTGTGAC
GCCTATGCAGAAGACGTTTGCGCCCGGAGCAGACTACATGACCGACTACGACGAATGGCTCTCCATACAGAACGGT
GGCAGCCCTGACTCGGAAGCGGACCTAGACGACGAGGACCGGTACATCCGCAACTCCCGCGACCTCTCTAGGCTGG
TGGCTACCGACACCGTCAACACGGAGGCGTACCGAGCCGCTCTGATTCTTCTCGACCCGGACCAGGGAGCCGATGG
CCGGGCAGCCATCAGCGCTCCCGGCTTGAACGGTCCCTACGCGGACAGCAGCCGCCAGGCCGGCTTCGTCAACTAC
GGCGTGTCTCACCTTATGAGGCTCGTCGGAACCGCCGAGCTGGCCCAGAAGTCCGCGTGGTACCAAAAGTGGAACG
TGCACATGTTCGTACGTCCGGAGGCTTTCGGCGGAAGCATCCACAACGTCCTCTTGGGCAAACTCGACGTAGAGAT
CGCCCCCTCGCTTCTCAAAAACACGGATTTGCTAGACAGGGTGGCTGCACGGAACGGGGAAATCAACGGGCGGCCA
GGAGTACTCGACCGCACCTACCTCCTCTCCCAGGCCCTCCCCGAGGGGTCGCCAACTCACCCATCGTACCCCGCTG
GGCACGCCACCCAGAACGGTGCATTCGCCACGGTGCTCAAGGCCCTGGTCGGGCTGGAGCGTGGCTCTGTCTGCTT
CAATGACCCCGTGTTCCCCGACGACGAAGGGCTGACCCTTCTGCCCTACACCGGAGACGACGGAAACAACTGCCTA
ACATTCGAGGGAGAAATCAACAAGCTGGCCGTCAACGTGGCATTGGGCAGGAACATGTTGGGTGTTCACTGGAGGA
TCGACAGCGAGTTGGGTTTGCTCCTCGGCGAGACGGCAGCTGTGAGGATCCTGCAGCAGGAGGCCGTGGCATACCC
AGAGAACGCGGGATACGAGTTCCGTCTGATGTCAGGCAAGACCATCAGGCTCGAAACCGACGGGACATTCTTCATC
GACGACACGCTGTGCAGCGGGGACGCGTTCATGGGAGCTGACCTGTGCTAA
SEQ ID NO: 44-protein sequence of the Vanadium-dependent iodoperoxidase of Laminaria
digitata
MKGLAGPAGAMAVVALGLVPGGAIGKSLRQEPSEPRLSGGVDTAASPSKDTLKGSLSRKLQVVNDDALDVSGTPAE
RAANALNQRIEFAETEFTASEGTLHLNNGDRSSAATFHKSLPHDSLGQVNSEDEDLLMECIAQGDEDTCELVPAGD
DGRLSNPLGGIAVEMAGAAGPALTLPPASAINSEDLAAQMAEQYWMALTRDVPFSQYGEDEATVAAADNLATMPGF
ADIVGVAVDPETRRADPQSQLFRSSAFGVETGPFISQLLVKDFTIDSITVTPMQKTFAPGADYMTDYDEWLSIQNG
GSPDSEADLDDEDRYIRNSRDLSRLVATDTVNTEAYRAALILLDPDQGADGRAAISAPGLNGPYADSSRQAGFVNY
GVSHLMRLVGTAELAQKSAWYQKWNVHMFVRPEAFGGSIHNVLLGKLDVEIAPSLLKNTDLLDRVAARNGEINGRP
GVLDRTYLLSQALPEGSPTHPSYPAGHATQNGAFATVLKALVGLERGSVCENDPVFPDDEGLTLLPYTGDDGNNCL
TFEGEINKLAVNVALGRNMLGVHWRIDSELGLLLGETAAVRILQQEAVAYPENAGYEFRLMSGKTIRLETDGTFFI
DDTLCSGDAFMGADLC
SEQ ID NO: 45-DNA sequence encoding the Vanadium-dependent iodoperoxidase of Algoriphagus
marincola
ATGAATATGAATAGTAAGTCGCTACTACTTCTTTGTGCGCTCGTTGTGGGCACGGCTTCCTTTGCTGCTGAAAAAC
AAGAGCCTTGGAGTAGTATCTATAAATCTCAACTTTTTCACATTACAGAGGTCATGGTGACAGATGTTGCCAGCCC
ACCTGTCGCAGCTAGAATCTATGCCTATTCCTGTCTTGCCAGCTATTTGGTCATGAAGCAGCATGGAGCTGACTTT
AGAGAAAAGGACTTTACCCAAGCTTCCATTCTTGACTTTCCAGAACTTGATTCCAATCAAGAGTTGAGCTCTCCCG
AATTTGCTGCAATCTATGCCATGCTTCGTGTTGGGGAAAATGTAATGCCTTCCGGCTATCTTTTAAAAGAAAAGCA
GCAAAACTGGATAGATCAAGCCATCAAATCAAAACTCATCAAAAAGTCTAAAGTTGATTTGCACATTCAGGAAGCA
GAAAAAGTCGTGAGTCAAGTCATGAAATTAGCGAGTACGGATGGCTACCGCGAATTACCAGCCTTGACCCGCTACA
GTCCCACAGAGGCAGAAGGTCGCTGGTATCCTACTCCTCCAGCCTACATCGAGGCGATAGAACCTGAATGGCGTAC
CATTCAACCCTTTTTCCTAGTCTCCCTCGATGATTATGATCCCAGCCCGATGGCACCTTTCAGCCTGGAACCGGAA
AGTTCATTTCATCAGCAAATGATAGAGGTCTACGAGACTACTAAGTCCTTGGATGAAGAGCAAAAACTAATTGCCA
ATTTCTGGGATTGCAATCCATTTATGGTAGAGTTTTCGGGTCATATGGCTATTGGGGTAAAGAAAATTTCACCAGG
TGGACACTGGATGGGGATTACCGGAATTGCTGCTGAAAAAGCAGAATTGAATTTGGCAGAAACTGCTTATATCCAT
GCCCTTGTCGGGATGACGCTACACGATGCATTTATTTCTTGCTGGAAAACCAAATACGAAACCGATCGGATTCGCC
CTGAAACCATCATCAACAAAACCATTGACCAACGCTGGCGACCACTGCTTCAGACGCCACCTTTTCCTGAGTACAC
TTCAGGTCATTCGGTGATTTCAAGGGCAGCTGCCATTGTTTTGACGGGTTACTTTGGAGATAATTTCTCCTACATC
GATGACTCGGAAACTTATTTTGGTCTTCCTGAGCGTGCATTTGATTCATTTTTACAAGCTTCAGAAGAGGCTGCCA
TTTCCAGGCTTTACGGGGGTATTCACTTCAGAGACGCTATTGAAGAGGGAGTTCGCCAAGGAGAAAAAATCGGAAA
AATGATCTGGCAAGCTATCGCAGAAAAAGAACTACTGACCAGGCAAAATCAATAA
SEQ ID NO: 46-protein sequence of the Vanadium-dependent iodoperoxidase of Algoriphagus
marincola
MNMNSKSLLLLCALVVGTASFAAEKQEPWSSIYKSQLFHITEVMVTDVASPPVAARIYAYSCLASYLVMKQHGADE
REKDFTQASILDFPELDSNQELSSPEFAAIYAMLRVGENVMPSGYLLKEKQQNWIDQAIKSKLIKKSKVDLHIQEA
EKVVSQVMKLASTDGYRELPALTRYSPTEAEGRWYPTPPAYIEAIEPEWRTIQPFFLVSLDDYDPSPMAPFSLEPE
SSFHQQMIEVYETTKSLDEEQKLIANFWDCNPFMVEFSGHMAIGVKKISPGGHWMGITGIAAEKAELNLAETAYIH
ALVGMTLHDAFISCWKTKYETDRIRPETIINKTIDQRWRPLLQTPPFPEYTSGHSVISRAAAIVLTGYFGDNESYI
DDSETYFGLPERAFDSFLQASEEAAISRLYGGIHERDAIEEGVRQGEKIGKMIWQAIAEKELLTRQNQ
SEQ ID NO: 47-DNA sequence encoding the Vanadium-dependent iodoperoxidase of Saccharina
japonica
ATGAAGGGTACCGCAGGATCCGCCCTTGGCGTGGTCGTCATCGCCTTCGGGCTTTTACCCGGAGCTATCGGGGAAT
CGCTGACACGAGAGCATGCTGAACACTACCCAGCCGGTGGTGTAGAATCGAGCGCCTTAGACGGCAGTGCTCTGCG
TCGTCTCCAGCCTGTATCCGATGATGCCTTCGACGTCAACGGCACGCCCGCGGAAAGAGCTGCCAACGCGCTGATG
GAGCGGACCGATTGGGCGGAGACCGAGTTCTTGGCATCGGAAGACGTGTTCCACGAAAACCTTGGAGATTTTTCCT
CGGCCGCCACGTTCCACAAGTCGCTTCCACACGACGAACTCGGACAGGTGTTCAGCTTCGATTTCGAAGAACTCAT
CGAGTGTGTTGCTTCAGGAGATTTCGACACGTGCCAGGAGGTGCCGGCCGGAAACGTCGAAGGCTTCCTGGTCAAC
CCTCTTGGTGGTCTCGCCAATGACATGGCAGGACCCGCGGGGCATGCTTTAACCATCCCTGCCGCTCCAGCACTCG
ACTCCGAGGACCTGGCTGCTCAAATTGCGGAGGTGTACTGGATGGCCCTGACAAGGGAGGTGCCGTTTTCACAGTA
CGGCGAAGACCCGACAACTGTGGAGGCCGCAGCCAGCTTGGCCGCCATGCCGGGTTTCGCGGATCTCAACTTTGTG
GCGGTCGGGCCGGATGGCAGCCCCGACCCACTGACGCAGCTTTTCCGGTCTTCGGCCGTCGGTGTAGAGATAGGAC
CCCATGTTTCACAGCTACTGGTGAATAACTTCACGATTGACTCCATCACTGTGGAGGCTAAGCAAAATACGTTTGC
GCCCGGAGAAGACTACATGGCCGAGTTCGACGAATGGCTATCCATACAGAACGGTGAATTTCCTGTAGAACCGGAG
ATCCTCGACCCTGTGCCGCGGTATATCCGCAACGGACGCGACCTTTCGACAATGGCAGCAACCGACACCATCAATA
CGGAGGCGTACCGATCCGCCCTGATCCTTATCGAGCAAGACGCCATCAGCCGTTTCGGTATCAACGGCCCGTACGT
GGCAGACGGTCGTCAGAGCGGTTTCGTCAACTACGGCATCTCCCATACTATGAGACTTGTTGGAAGCGGCGAGCTG
TCCATGAGGTCCTCATGGTACCAGAAGTGGAACGTTCACCTGTATGCCCGTCCGGAGGCCATCGGTGGAACCATCC
ACAACGTCCTCAATGGCGATCTCGACATAACGTTCGCCGACTCGATTATCAACAACGACGAGTTGTTCAGTAAGGT
GGAGGCTAGGAACATGGAGATCACCGGGATACCATCCACCTTCCTTCTACCCCAGACCGTCAGGGAGGGGTCACCA
ACTCACCCATCGTACCCCTCTGGACACGCCGTCCAAAACGGTGCCTTTTCAACGATACTCAAGGCTCTTGTCGGGC
TGGAGAGGGGATTGGAGTGCTTCAACGACCCCGTGGTCCCCTCTGATGATGGATTGACCCTTCTGCCCTTCGACGG
CTGCCTGACGTTCGAGGGAGAAATCAACAAATATGCCGCCAACGTCGCTCTTGGCAGGAATTGGATGGGTGTTCAC
TGGAGGATGGACGCTCAAGAAGGGTTGCTGCTCGGAGAAACGGTCGCCGTGCGGATCCTGATGCAGGAGCTCGCGG
GGTTCCCAGAGTTGGAGCCCTACGAGTTCCGTGTCATGTCAGGCGAGAGGAACGTGACGGAGGCGGTGACGGATAG
ACGTATCGCCAACATGTACCTCAAGTACGCCGGGTTGCGGGATTGGTGGACGCATGATGTTCGATCCGGAGAATAT
CTTCTCCGGGGGGCGCTGGATATATTTTCGAGCCCCAGCCAATCAGTGATTGACCGGTTGGGCTACGGTGTCCATA
CCGTGTGCGGCGGTGTCGACGCTCGGTGTCGACGCTCGGGCCCTCGTTTTTAA
SEQ ID NO: 48-protein sequence of the Vanadium-dependent iodoperoxidase of Saccharina
japonica
MKGTAGSALGVVVIAFGLLPGAIGESLTREHAEHYPAGGVESSALDGSALRRLQPVSDDAFDVNGTPAERAANALM
ERTDWAETEFLASEDVFHENLGDFSSAATFHKSLPHDELGQVFSFDFEELIECVASGDFDTCQEVPAGNVEGFLVN
PLGGLANDMAGPAGHALTIPAAPALDSEDLAAQIAEVYWMALTREVPFSQYGEDPTTVEAAASLAAMPGFADLNFV
AVGPDGSPDPLTQLFRSSAVGVEIGPHVSQLLVNNFTIDSITVEAKQNTFAPGEDYMAEFDEWLSIQNGEFPVEPE
ILDPVPRYIRNGRDLSTMAATDTINTEAYRSALILIEQDAISRFGINGPYVADGRQSGFVNYGISHTMRLVGSGEL
SMRSSWYQKWNVHLYARPEAIGGTIHNVLNGDLDITFADSIINNDELFSKVEARNMEITGIPSTFLLPQTVREGSP
THPSYPSGHAVQNGAFSTILKALVGLERGLECENDPVVPSDDGLTLLPFDGCLTFEGEINKYAANVALGRNWMGVH
WRMDAQEGLLLGETVAVRILMQELAGFPELEPYEFRVMSGERNVTEAVTDRRIANMYLKYAGLRDWWTHDVRSGEY
LLRGALDIFSSPSQSVIDRLGYGVHTVCGGVDARCRRSGPRF
SEQ ID NO: 49-DNA sequence encoding an alternative Vanadium-dependent iodoperoxidase of
Laminaria digitata
ATGAAGGGGCTTGCAGGACCAGCCGGTGCTATGGTCGTTGTCGCGCTCGGGCTTGTCCCCGGCGGAGCAATCGGGA
AATCTTTGCGACAAGAGCCCTCTGAACCCCGCCTCAGTGGCGGCGTGGATACGGCTGTATCGCCATCGAAGGACAC
CCTGAAAGGGAGTCTTTCGCGTAATCTTCAAGTCGCCAACGATGATGCCCTCGATGTTAATGGCACGCCCGCAGAA
AGAGCTGCCAACGCGCTGGCCCAGCGGATCGAATTGGCGGAGACGGAGTTCGCGGCATCCGAAGGCTTGTTCCACG
TCAACAACGGAGACCGCTCATCCGCCGCCACGTTCCACAAGTCGCTGCCGCACGACAGCCTAGGACAGGTGGACAG
CGCGGCCTTTGAAGCCCTCACAGAGTGCATCGCTCAAGGTGATTTCGACATATGCGAGCTGGTGCCGGCCGGAGAC
GTTGGCAGGCTGTCCAACCCCCTCGCGGGTATCACCGTCGAGATGGCCGGAGCCGCCGGCTCCGCCTTGACCCTCC
CTCCGGCCTCAGCGCTCGACTCCGAGGACTTGGCTGCTCAAATGGCAGAACTGCACTGGATGGCCTTGACCAGGGA
CGTACCTTTCTCTCAGTACGGCGAAGATGAGGCGACTGTGGCTGCAGCAGACAACTTGGCCACCATGCCTGGTTTT
CAAAACATGGTCGGGGTGGCCGTCGATCGGGATGGCAGAGCGGATCCGCAGTCGCAGCTTTTCCGGACCTCTGCCT
TCGGCGTCGAGACAGGGCCCTTCATCTCCCAGCTACTGGTTCAGGACTTCACGATTGATTCCATCACTGTGGCGCC
TATCCAGAAGACGTTTGAACCCGGAGCAGACTACATGGCCGACTACGACGAATGGCTCTTCATACAGAACGGTGGC
GTGCCTGACCATGACGATGTGCTCTTTGACGACGTGAACCGGTACATCCGCAACTCCCGCGACCTCTCTAGGCTGG
TGGCTGCCGACACCGTCAACACGGAGGCGTACCGAGCCGCTCTGATTCTTCTCGAGCAGGGAGCCATCAGCGGTCC
CGGCTCCAACGGTCCCTACGCGGGCAGCAGCCGCCAGGCCGGCTTCGTCAACTACGGCGTGTCCCACCTTATGAGG
CTCGTCGGAACCGCCGAGCTGTCCCAGAAGTCCGCGTGGTACCAGAAGTGGAACGTGCACATGTTCGTACGTCCGG
AGGCTTTCGGCGGAACCATCCACAATGTCCTCTTGGGCAAACTCAACGTAGACATAAACCCCTCGCTTCTCAAAAA
CACGGAGTTGCTAGAGAGGGTGGCTGAACGGAACGGGGTAATCAACGGGCGGCCAGGAGTACTCGACCGCACCTAC
CTCCTCTCCCAGGCCGTCATCGAGGGGTCGCCAACTCACCCATCGTACCCCGCTGGGCACGCCACCCAGAACGGTG
CATTCGCCACGGTGCTCAAGGCCCTGGTCGGGCTGGAGCGTGGCTCTGACTGCTTCAGAGACCCCAAGGTCCCCGA
CGACGAAGGGCTGACCCTTCTGGACTTCACCGGGGACTGCCTAACATTCGAGGGAGAAATCAACAAGCTGGCCGTC
AACGTGGCATTCGGCAGGAACATGTGCGGTGTTCACTGGAGGATCGACAGCGAGCAGGGTTTGCTCCTCGGCGAGA
TGGCAGCTGTGAGGATCCTGCAGCAGGAGGCCGTGACATTCCCAGAGAACGCGGGATACGAGTTCAATCTGATGTC
AGGCGAGACCATCAGGCTCGAAACCGACGGGACATTCTTCATCAACGACAGGCTATGCAGCGGGGACGCGTTCATG
GGAGCTGACCTGTGCTAAT
SEQ ID NO: 50-protein sequence of an alternative Vanadium-dependent iodoperoxidase of
Laminaria digitata
MKGLAGPAGAMVVVALGLVPGGAIGKSLRQEPSEPRLSGGVDTAVSPSKDTLKGSLSRNLQVANDDALDVNGTPAE
RAANALAQRIELAETEFAASEGLFHVNNGDRSSAATFHKSLPHDSLGQVDSAAFEALTECIAQGDFDICELVPAGD
VGRLSNPLAGITVEMAGAAGSALTLPPASALDSEDLAAQMAELHWMALTRDVPFSQYGEDEATVAAADNLATMPGF
QNMVGVAVDRDGRADPQSQLFRTSAFGVETGPFISQLLVQDFTIDSITVAPIQKTFEPGADYMADYDEWLFIQNGG
VPDHDDVLFDDVNRYIRNSRDLSRLVAADTVNTEAYRAALILLEQGAISGPGSNGPYAGSSRQAGFVNYGVSHLMR
LVGTAELSQKSAWYQKWNVHMFVRPEAFGGTIHNVLLGKLNVDINPSLLKNTELLERVAERNGVINGRPGVLDRTY
LLSQAVIEGSPTHPSYPAGHATQNGAFATVLKALVGLERGSDCFRDPKVPDDEGLTLLDFTGDCLTFEGEINKLAV
NVAFGRNMCGVHWRIDSEQGLLLGEMAAVRILQQEAVTFPENAGYEFNLMSGETIRLETDGTFFINDRLCSGDAFM
GADLC
SEQ ID NO: 51-DNA sequence encoding the Vanadium-dependent iodoperoxidase of Zobellia
galactanivorans, codon-optimized for dicot plants
ATGGGTTCTATGAAAAAGATCCTGATCGCGTTGATCTCTTTCGCGTTTGCAGTATCCTGCAAAGCACCTCAAAAAG
AGGAACCGATAAACATCACTCCGGAGGAATTGGATGCGTCTATTGACCGGGTGACAGAGATCATGATCCATGATAT
CTTCAGCCCACCTGTTGCTTCCCGAATTTTCGCATATCCAAACGTGGCGGCGTACGAAATTGTCGCTGCAACTAAT
GATAATTACAACTCACTTGCTGGTCAACTTAACGGACTAACGGCAATACCAGAGCCAGATACAACCAAAACCATCA
ATTACGAGCTGGCTGCTGTCGTTGCTCATATGGAACTCTCTAAGCGTCTCATTTTCTCAGAGGACCGTATGGAATC
CCTTAGAGATAGCTTGTACATGGTTTGGGAGGGCAAAAATCCTGTTCTGTTCAGCGACTCCAAGGCCTATGGGTTA
CAAGTCGCTGACCACATCGGTGAGTGGATGAACAAGGACAACTATGCACAAACCCGTACAATGCCGAAATTCACGG
TGGATGCCGATGATCCAGGTAGGTGGCAGCCAACCCCGCCTGCTTATATGGATGGGATTGAACCACATTGGAACAA
AATCAGACCATTTGTACTTGATTCTGCCGCGCAGTTCAAGCCAGTCCCACCGCCCGCTTATTCATTGGAAGAAGAT
AGTGCTTTCTACAAGGAATTGAAGGAGGTTTATGATGTGCGGAACAAGATTACTGAGGAGGGAGATTCATCTGAGG
AGATACAGATTGCTAGATTCTGGGATTGTAATCCATACGTGTCAGTGACTAGAGGACACTTGATGTTCGCAACTAA
AAAAATCACACCAGGTGCTCATTGGATGGGAATCGCTAAAATTGCGGCCAGAAAGACAAATTCGGACTTCGCTAAA
ACTCTCTTTGCATACACTAAAGCCTCTGTGGCAATGGCTGATGCATTTATCTCTTGTTGGGATGAGAAGTACAGAA
GTAACTTGATCAGGCCAGAGACAGTAATCAACCAACACATTGATGATAGCTGGAAACCTGTTTTGCAGACACCACC
ATTCCCCGAATACACGTCTGGCCATAGCGTCGTGTCGGGTGCCGCTAGTGTCGTTCTGACAGAGGTGTTTGGAGAT
AATTTCAGTTTCGATGATGACACGGAGGTCCCATATGGGCTGCCGATTAGATCGTTTAAAAGCTTTAAGCAGGCTG
CTGACGAAGCCGCCATTAGCAGAATGTACGGGGGAATCCACTATCGTGCAGCTATTGAGGTTGGCGTTAAACAGGG
AAGGGATTTGGGGACCTTCGTAGTGAATAAATTACATATGTTATCTGATAAGAAGGTCGCGCAGAATTAATAG
SEQ ID NO: 52-DNA sequence encoding the Vanadium-dependent iodoperoxidase of Zobellia
galactanivorans, codon-optimized for monocot plants
ATGGGGTCTATGAAGAAGATCCTTATAGCTCTGATCTCGTTCGCGTTCGCTGTCTCGTGCAAGGCCCCGCAAAAGG
AGGAGCCGATAAACATAACTCCAGAAGAGCTGGATGCTTCCATTGATAGGGTGACGGAGATCATGATCCATGACAT
ATTCTCCCCGCCGGTGGCTTCCAGGATTTTCGCGTATCCAAACGTGGCAGCGTACGAGATAGTGGCAGCAACAAAT
GATAACTACAACTCGCTCGCTGGGCAACTGAACGGCCTTACAGCAATTCCAGAGCCGGATACCACCAAAACTATTA
ATTACGAGCTGGCAGCTGTTGTCGCCCACATGGAGCTGAGCAAAAGACTGATCTTCTCGGAGGATCGCATGGAGTC
ACTACGCGACAGTCTCTACATGGTCTGGGAAGGCAAGAACCCGGTGCTGTTCAGTGACTCCAAGGCATACGGCCTT
CAAGTCGCCGACCATATAGGTGAGTGGATGAACAAAGACAATTACGCGCAGACGAGAACGATGCCCAAGTTCACTG
TTGACGCGGATGATCCTGGCCGGTGGCAGCCAACCCCGCCTGCGTACATGGATGGCATCGAGCCCCATTGGAATAA
AATTAGACCGTTCGTGCTGGACTCGGCGGCACAGTTTAAACCCGTTCCGCCGCCTGCGTACAGCCTGGAGGAGGAT
TCCGCGTTCTACAAAGAGCTGAAGGAGGTCTACGACGTGCGCAATAAGATAACAGAGGAGGGAGATTCCAGCGAGG
AGATCCAAATAGCCAGATTTTGGGATTGCAATCCTTACGTGTCGGTTACCAGGGGGCACCTCATGTTCGCGACGAA
GAAGATTACGCCAGGCGCGCATTGGATGGGAATCGCGAAGATAGCGGCGAGGAAGACGAACTCGGATTTCGCCAAG
ACGCTGTTCGCGTACACAAAGGCGTCGGTTGCCATGGCAGATGCTTTTATATCCTGTTGGGATGAGAAGTACAGGT
CAAATCTGATTAGGCCCGAGACAGTCATCAATCAACACATTGACGACTCATGGAAACCGGTCCTCCAGACACCCCC
ATTCCCCGAGTACACCTCTGGACATTCTGTAGTATCTGGTGCTGCGTCAGTTGTCCTCACAGAGGTGTTCGGTGAC
AATTTCTCGTTCGATGATGACACCGAGGTGCCGTACGGCCTGCCTATTCGGTCTTTTAAGTCTTTCAAGCAAGCTG
CCGATGAAGCCGCCATATCGCGTATGTACGGTGGAATACACTACAGAGCCGCAATTGAAGTGGGCGTGAAGCAGGG
CAGGGATTTGGGTACCTTCGTCGTAAACAAGCTCCACATGTTGAGCGACAAGAAGGTAGCCCAGAACTAATAG
SEQ ID NO: 53-DNA sequence encoding the Vanadium-dependent iodoperoxidase of Laminaria
digitata, codon-optimized for dicot plants
ATGAAAGGGCTTGCGGGACCCGCTGGTGCTATGGCAGTCGTTGCACTAGGCTTGGTTCCTGGCGGAGCGATAGGGA
AGTCGCTTCGTCAGGAGCCTAGTGAGCCAAGACTTTCTGGAGGAGTTGATACCGCAGCATCTCCAAGCAAAGACAC
TCTCAAAGGGTCACTTAGTCGTAAGCTACAGGTAGTGAACGATGATGCTCTAGACGTTTCAGGTACCCCAGCAGAA
AGGGCCGCGAATGCTTTAAACCAGCGTATTGAATTCGCCGAAACAGAATTCACAGCATCTGAAGGTACTTTACATC
TTAATAATGGCGACAGATCTTCAGCTGCTACATTTCATAAGTCACTCCCACATGATAGTTTAGGCCAAGTGAATAG
TGAAGATTTCGATTTGCTTATGGAGTGCATTGCTCAGGGAGATTTTGATACTTGCGAACTTGTTCCGGCGGGAGAC
GATGGGCGACTATCTAATCCGTTGGGGGGTATCGCTGTCGAAATGGCAGGTGCGGCTGGACCTGCACTCACGCTAC
CTCCTGCATCCGCAATCAATAGTGAGGATCTAGCAGCTCAAATGGCTGAACAGTACTGGATGGCCCTAACAAGAGA
TGTGCCATTTAGTCAATACGGTGAAGATGAGGCAACCGTCGCTGCAGCTGATAACTTGGCAACTATGCCAGGTTTT
GCTGATATTGTTGGTGTGGCTGTGGATCCAGAAACTAGGCGGGCTGATCCTCAGTCTCAACTTTTCAGATCCAGCG
CTTTCGGAGTTGAGACTGGCCCTTTCATCTCACAACTTTTGGTGAAAGATTTTACTATAGATAGTATTACGGTTAC
CCCTATGCAAAAGACCTTTGCTCCTGGAGCGGATTACATGACTGATTACGATGAGTGGCTTTCCATCCAAAATGGA
GGATCCCCAGACTCCGAAGCGGATTTGGATGATGAGGACAGATATATACGAAATAGCCGAGATCTCAGTAGACTCG
TTGCGACCGACACTGTTAATACAGAGGCTTATAGGGCTGCTTTGATACTCTTAGATCCCGATCAAGGAGCCGATGG
ACGTGCTGCCATCAGCGCACCAGGCCTTAACGGACCTTACGCTGATTCGTCTAGACAGGCCGGTTTTGTCAACTAC
GGAGTCTCCCATCTGATGCGGTTGGTAGGTACTGCCGAGCTTGCTCAAAAATCTGCGTGGTATCAGAAATGGAATG
TGCATATGTTTGTTCGGCCTGAGGCATTTGGAGGAAGTATACATAACGTTTTATTGGGAAAACTCGATGTTGAGAT
AGCACCTTCCCTACTTAAAAATACTGATTTGCTTGACCGTGTTGCTGCACGTAACGGAGAGATCAACGGAAGGCCT
GGGGTCCTAGATCGAACATATCTTTTAAGTCAAGCTCTGCCTGAGGGTAGCCCAACTCATCCATCTTATCCTGCAG
GACACGCCACTCAGAACGGTGCTTTCGCTACCGTTCTTAAGGCTTTGGTTGGATTGGAAAGAGGCTCCGTTTGCTT
TAACGACCCCGTTTTCCCTGACGATGAAGGACTGACACTCCTTCCTTATACGGGAGATGATGGAAATAATTGTCTT
ACATTTGAGGGAGAAATTAATAAACTCGCTGTGAACGTTGCTTTGGGAAGGAACATGCTTGGGGTCCATTGGAGAA
TAGATAGTGAGTTGGGTCTTCTATTGGGTGAAACCGCCGCTGTTAGGATTTTACAGCAAGAAGCAGTTGCTTATCC
AGAAAATGCTGGATATGAATTTAGATTGATGTCTGGAAAAACTATAAGACTAGAAACAGATGGTACGTTTTTTATT
GATGACACTTTATGCAGTGGCGATGCTTTTATGGGCGCTGATCTTTGCTAATAG
SEQ ID NO: 54-DNA sequence encoding the Vanadium-dependent iodoperoxidase of Laminaria
digitata, codon-optimized for monocot plants
ATGAAGGGCCTGGCTGGTCCAGCAGGCGCCATGGCCGTGGTCGCGCTTGGTCTGGTTCCAGGCGGGGCAATTGGGA
AGAGCCTCCGCCAGGAGCCTTCTGAGCCTCGCCTCAGTGGGGGTGTCGACACCGCGGCTTCACCTTCCAAAGATAC
GCTCAAAGGATCACTGTCCCGCAAGCTTCAGGTGGTCAACGATGACGCTCTGGATGTGAGCGGCACACCCGCCGAG
AGGGCAGCCAATGCGTTGAACCAGAGAATAGAGTTCGCCGAAACCGAGTTCACGGCGTCTGAGGGGACGCTACACC
TGAATAACGGCGACCGGTCTTCCGCGGCTACGTTCCACAAGAGTCTCCCACACGACTCGCTGGGACAAGTGAACTC
CGAAGATTTTGACCTTTTGATGGAGTGTATCGCCCAAGGAGATTTTGACACTTGCGAGCTGGTTCCGGCCGGGGAC
GATGGCAGGTTGAGCAATCCTCTCGGCGGGATCGCCGTAGAGATGGCTGGGGCGGCAGGTCCTGCCCTCACGCTGC
CCCCTGCAAGCGCGATTAATTCCGAAGATCTCGCCGCCCAGATGGCTGAGCAATACTGGATGGCGCTGACACGGGA
CGTGCCGTTTAGTCAGTACGGGGAAGATGAGGCCACAGTTGCCGCGGCCGACAATCTTGCAACCATGCCGGGCTTC
GCAGATATTGTCGGAGTTGCTGTCGACCCGGAAACTAGGCGCGCCGACCCACAATCGCAACTGTTTAGGTCCTCAG
CATTCGGTGTCGAAACCGGGCCCTTCATCAGCCAACTCCTCGTAAAGGACTTCACCATCGATAGCATCACCGTCAC
CCCCATGCAGAAGACTTTTGCCCCTGGCGCCGACTACATGACCGACTACGACGAATGGCTGAGTATCCAGAACGGC
GGGAGCCCAGATTCCGAAGCCGATCTCGATGATGAGGATCGCTACATCAGGAATTCCCGCGACTTGAGTCGCTTGG
TGGCGACAGATACGGTTAATACAGAGGCCTATAGGGCGGCTCTCATTTTGCTGGACCCCGACCAAGGTGCTGACGG
CCGTGCCGCTATTAGTGCGCCTGGCTTGAATGGCCCCTACGCCGACAGCTCCAGGCAGGCCGGGTTTGTCAATTAC
GGCGTCTCGCATTTGATGCGACTCGTTGGAACCGCGGAGCTGGCTCAGAAGTCAGCGTGGTATCAGAAGTGGAACG
TCCACATGTTCGTCAGGCCGGAGGCCTTTGGAGGCAGTATCCACAACGTGCTCCTCGGCAAGCTCGACGTGGAAAT
CGCTCCATCTCTTCTTAAGAATACAGATCTCCTCGATCGCGTGGCCGCGCGCAATGGCGAGATCAACGGTAGGCCC
GGCGTCTTGGATCGAACCTACTTGCTCAGTCAAGCGCTGCCGGAAGGCTCACCCACACATCCGTCGTACCCGGCTG
GCCACGCGACCCAGAATGGGGCGTTCGCCACCGTGCTGAAGGCCCTAGTAGGCCTAGAGCGAGGTAGCGTGTGCTT
TAACGATCCAGTCTTCCCGGATGATGAAGGCCTGACCCTCTTGCCATACACGGGCGACGATGGTAACAACTGCCTG
ACGTTCGAGGGCGAGATAAATAAGCTCGCCGTCAACGTCGCACTGGGTCGGAACATGCTAGGCGTTCACTGGCGCA
TCGATAGTGAACTGGGACTGCTGCTCGGCGAAACCGCCGCCGTGAGGATCCTTCAGCAGGAAGCCGTGGCTTATCC
AGAGAATGCTGGCTACGAGTTCCGCCTGATGAGCGGAAAGACCATTAGACTGGAGACGGACGGCACATTCTTCATA
GATGACACGTTGTGCTCCGGAGACGCCTTTATGGGTGCGGACCTGTGCTAATAG
SEQ ID NO: 55-DNA sequence encoding the Vanadium-dependent iodoperoxidase of Algoriphagus
marincola, codon-optimized for dicot plants
ATGAACATGAATTCCAAATCACTGTTATTGCTATGCGCACTAGTAGTCGGTACTGCTTCTTTTGCTGCAGAAAAAC
AAGAGCCATGGTCTTCGATTTATAAGTCTCAACTCTTCCATATAACGGAAGTCATGGTGACTGATGTTGCCTCACC
TCCAGTAGCGGCGAGGATTTACGCTTATTCGTGTCTTGCATCGTACTTAGTTATGAAACAACATGGAGCAGATTTT
AGAGAAAAGGATTTCACGCAGGCCAGCATATTGGATTTTCCAGAGTTAGACTCTAACCAAGAACTAAGTTCACCTG
AGTTTGCGGCAATTTATGCTATGTTAAGAGTTGGTGAAAACGTGATGCCTTCCGGATACCTGCTCAAGGAAAAACA
ACAAAATTGGATAGACCAGGCAATCAAGAGCAAACTGATTAAAAAATCAAAAGTGGATTTGCATATTCAAGAAGCT
GAAAAGGTGGTATCTCAGGTGATGAAGCTCGCTAGTACCGACGGCTATCGTGAACTCCCTGCATTGACCAGATATT
CACCTACAGAAGCTGAGGGCAGGTGGTATCCTACACCGCCAGCCTACATAGAAGCTATAGAGCCCGAGTGGCGTAC
TATCCAACCATTTTTCTTGGTCTCGTTGGATGATTACGATCCTAGTCCTATGGCACCGTTCTCTCTAGAGCCTGAG
TCATCATTCCATCAGCAGATGATTGAAGTCTACGAGACGACAAAGTCATTGGATGAGGAGCAAAAACTCATAGCTA
ATTTCTGGGACTGTAACCCCTTCATGGTGGAATTTTCTGGACACATGGCCATTGGAGTTAAAAAGATTAGTCCAGG
GGGGCACTGGATGGGAATCACAGGAATCGCGGCTGAAAAGGCAGAATTAAATTTAGCAGAGACCGCTTATATTCAT
GCTCTGGTTGGTATGACTTTACATGATGCGTTTATTTCGTGCTGGAAGACTAAGTATGAAACGGATCGTATTCGTC
CGGAGACTATTATCAATAAAACCATCGATCAAAGATGGAGACCTCTTCTGCAGACTCCGCCATTCCCTGAATATAC
CTCAGGCCATTCAGTCATTTCTAGAGCTGCAGCAATAGTTTTGACCGGCTACTTCGGTGATAATTTTAGTTACATA
GATGATTCTGAGACCTACTTTGGGTTACCAGAACGGGCATTCGACTCATTCCTTCAGGCGAGCGAGGAGGCGGCGA
TTTCCAGGCTATACGGGGGTATTCATTTTAGGGACGCTATAGAGGAAGGTGTTCGTCAAGGTGAAAAGATCGGTAA
GATGATATGGCAAGCGATCGCAGAGAAGGAGCTGCTAACTCGTCAGAACCAATAATAG
SEQ ID NO: 56-DNA sequence encoding the Vanadium-dependent iodoperoxidase of Algoriphagus
marincola, codon-optimized for monocot plants
ATGAATATGAACTCTAAGAGTCTGCTGCTCTTGTGCGCCCTGGTGGTGGGCACAGCAAGCTTTGCGGCGGAGAAGC
AGGAGCCCTGGTCGTCAATTTACAAATCGCAGCTGTTCCATATCACGGAGGTCATGGTTACTGACGTCGCGTCTCC
CCCTGTGGCTGCGCGGATCTACGCATACAGCTGTCTCGCCTCCTATCTTGTTATGAAGCAGCACGGGGCTGACTTT
CGCGAGAAGGATTTCACGCAAGCCTCCATTCTGGACTTCCCAGAACTTGACAGTAACCAAGAGTTGTCTTCACCAG
AGTTCGCCGCGATATACGCTATGCTTCGCGTGGGCGAGAATGTCATGCCGTCCGGGTACCTCTTGAAAGAAAAGCA
ACAAAATTGGATCGACCAAGCCATTAAGTCAAAGCTGATCAAGAAATCAAAAGTCGACCTACACATCCAGGAGGCG
GAGAAGGTTGTGAGCCAAGTGATGAAGCTCGCCTCGACTGATGGCTACCGTGAGCTTCCGGCCCTAACTAGGTATA
GCCCGACAGAGGCCGAGGGTCGCTGGTACCCTACACCACCGGCCTACATTGAGGCGATCGAACCCGAGTGGAGAAC
AATTCAACCGTTTTTCCTCGTGTCTCTTGATGACTACGACCCTTCTCCGATGGCACCATTTTCGCTGGAACCAGAG
TCAAGCTTTCATCAGCAGATGATAGAGGTCTACGAGACTACCAAGTCGCTTGACGAAGAGCAAAAACTCATCGCAA
ACTTTTGGGACTGCAATCCTTTCATGGTCGAGTTCTCGGGCCATATGGCAATCGGTGTTAAAAAGATCTCTCCGGG
GGGTCACTGGATGGGCATAACTGGTATCGCGGCCGAGAAGGCGGAATTGAATCTGGCCGAGACAGCGTACATACAT
GCGCTCGTAGGTATGACCCTTCATGACGCTTTCATCTCTTGTTGGAAGACCAAGTACGAAACAGACCGGATCAGGC
CGGAAACGATCATAAATAAGACCATCGACCAGCGCTGGCGCCCGCTGTTGCAGACTCCTCCGTTTCCAGAATACAC
GAGTGGCCACTCCGTCATCTCCCGCGCTGCCGCCATCGTGCTTACCGGCTACTTTGGAGATAATTTCAGCTATATT
GACGATTCGGAGACGTACTTCGGCTTGCCGGAGCGAGCTTTCGACTCATTCTTGCAAGCATCCGAGGAAGCAGCCA
TCAGTCGCTTGTACGGCGGGATTCACTTCCGCGACGCCATCGAGGAAGGTGTTCGCCAAGGCGAGAAGATCGGAAA
AATGATCTGGCAGGCGATCGCCGAAAAGGAACTCCTTACCCGACAGAACCAGTAATAG
SEQ ID NO: 57-DNA sequence encoding the Vanadium-dependent iodoperoxidase of Saccharina
japonica, codon-optimized for dicot plants
ATGAAAGGAACTGCTGGATCCGCTTTGGGGGTAGTGGTCATCGCATTCGGACTTCTCCCTGGAGCAATCGGGGAAA
GCCTTACCAGGGAACATGCTGAACATTATCCCGCAGGAGGAGTTGAGTCAAGCGCTTTGGACGGGTCTGCTCTCCG
AAGACTTCAGCCAGTCAGTGATGACGCATTTGATGTGAATGGAACTCCGGCTGAGAGAGCTGCTAATGCTCTTATG
GAGCGGACCGATTGGGCAGAGACCGAATTCCTGGCTTCGGAGGATGTGTTTCACGAAAACCTGGGTGATTTCAGCT
CCGCTGCTACATTCCATAAGAGCCTACCACACGATGAACTTGGTCAGGTTTTTTCGTTTGATTTCGAAGAACTAAT
TGAATGTGTTGCTTCTGGAGATTTTGATACATGTCAGGAGGTCCCCGCAGGGAACGTTGAGGGATTTTTGGTCAAC
CCATTAGGGGGTCTGGCTAATGATATGGCCGGTCCTGCAGGTCATGCCCTTACTATTCCTGCAGCGCCGGCACTGG
ACTCAGAAGATCTGGCGGCTCAGATAGCTGAGGTTTACTGGATGGCTCTGACACGTGAAGTTCCATTCTCACAGTA
CGGTGAGGATCCGACAACCGTCGAAGCAGCTGCGAGTCTTGCGGCTATGCCTGGATTTGCAGATTTGAATTTTGTT
GCAGTAGGACCTGATGGCTCGCCTGATCCACTAACACAACTATTTAGAAGTTCTGCAGTAGGTGTTGAGATTGGCC
CTCATGTCAGTCAACTCCTTGTGAATAACTTTACGATCGATTCTATTACTGTAGAAGCGAAGCAAAACACTTTTGC
CCCTGGAGAAGATTACATGGCTGAATTTGATGAATGGTTGTCGATCCAAAACGGTGAGTTCCCTGTTGAACCTGAA
ATTCTAGACCCTGTTCCTAGATACATCAGGAACGGTCGAGATCTTAGTACTATGGCTGCGACTGATACAATTAATA
CTGAAGCCTACAGGTCAGCACTCATTTTAATAGAGCAGGACGCTATCTCACGGTTCGGAATAAACGGGCCTTACGT
GGCTGATGGAAGACAGAGTGGGTTTGTCAACTACGGTATCTCTCATACCATGAGGTTGGTTGGTAGTGGAGAACTT
TCCATGCGGTCATCTTGGTACCAAAAGTGGAATGTGCATCTTTATGCACGGCCCGAAGCCATCGGAGGAACCATAC
ACAACGTCCTTAACGGGGACTTGGATATTACCTTCGCTGACTCGATTATTAATAATGATGAGTTATTCTCTAAAGT
CGAGGCGCGTAATATGGAGATCACTGGTATCCCTTCTACATTCCTTTTGCCTCAAACTGTGAGAGAAGGCAGCCCG
ACTCACCCGTCCTATCCTTCTGGTCATGCTGTGCAAAACGGTGCCTTTTCCACAATTCTAAAAGCCTTGGTCGGAT
TGGAACGTGGATTAGAATGCTTTAACGATCCTGTTGTTCCATCAGATGATGGATTAACCTTGCTTCCGTTCGATGG
ATGCTTAACGTTTGAGGGTGAAATAAACAAGTACGCCGCCAACGTCGCGCTAGGACGGAATTGGATGGGTGTTCAT
TGGAGAATGGATGCTCAGGAGGGTCTCTTGCTAGGAGAGACAGTTGCTGTCAGAATATTAATGCAGGAGTTAGCTG
GTTTTCCGGAATTAGAACCATACGAATTCAGGGTTATGAGCGGAGAAAGGAATGTGACCGAGGCTGTGACCGACAG
GAGGATCGCGAACATGTATCTGAAATACGCTGGATTGAGAGACTGGTGGACACATGATGTCCGTTCCGGAGAGTAC
CTTCTGAGAGGGGCACTCGACATTTTCTCGAGTCCGTCACAATCAGTCATCGACCGACTTGGTTACGGTGTTCATA
CCGTTTGCGGTGGTGTCGACGCTCGATGCAGGAGGTCAGGTCCACGTTTCTAATAG
SEQ ID NO: 58-DNA sequence encoding the Vanadium-dependent iodoperoxidase of Saccharina
japonica, codon-optimized for monocot plants
ATGAAGGGGACCGCCGGGTCAGCGCTGGGGGTCGTAGTAATTGCGTTTGGCCTGCTACCTGGGGCGATCGGTGAAT
CACTGACTCGCGAGCACGCCGAGCACTACCCCGCCGGCGGCGTCGAAAGTTCTGCACTGGACGGGTCCGCGCTCCG
GAGACTCCAGCCAGTCTCCGATGACGCCTTCGATGTTAACGGTACGCCCGCCGAACGGGCGGCCAATGCTCTTATG
GAGCGCACAGACTGGGCTGAAACGGAGTTCCTCGCCTCCGAGGACGTCTTTCACGAAAATCTGGGCGACTTTAGCA
GCGCAGCCACGTTCCACAAGAGCTTGCCCCACGATGAGCTCGGTCAAGTGTTCTCGTTCGATTTCGAAGAACTGAT
CGAATGCGTTGCCTCCGGTGACTTCGACACGTGTCAGGAGGTTCCGGCCGGCAATGTGGAGGGCTTCCTGGTTAAC
CCTTTGGGGGGCTTGGCCAACGATATGGCAGGACCAGCAGGCCATGCACTTACGATCCCCGCCGCGCCAGCGCTAG
ATTCCGAGGACCTCGCCGCTCAGATTGCCGAGGTCTACTGGATGGCCCTGACCCGCGAAGTACCGTTCTCACAATA
CGGGGAAGACCCGACAACAGTTGAAGCAGCAGCCAGTCTCGCCGCTATGCCGGGGTTTGCAGATTTGAACTTCGTT
GCCGTCGGCCCGGACGGCAGCCCGGACCCACTCACCCAGCTATTTCGGTCTAGTGCTGTAGGAGTTGAGATCGGCC
CTCACGTAAGCCAGCTGCTCGTTAATAACTTCACAATTGACTCAATAACGGTGGAGGCAAAGCAGAACACATTCGC
CCCCGGCGAGGACTATATGGCGGAGTTCGACGAGTGGCTAAGCATCCAGAACGGTGAATTCCCTGTTGAGCCGGAG
ATCCTTGATCCCGTACCAAGATATATCCGGAATGGCCGGGACCTTTCTACCATGGCGGCCACCGATACGATCAACA
CGGAGGCCTACCGCTCTGCCCTCATCCTCATTGAACAAGATGCCATCTCAAGGTTTGGCATTAATGGGCCATATGT
TGCGGACGGGCGCCAGAGTGGATTCGTCAACTACGGCATCTCTCACACAATGCGTCTGGTGGGATCCGGCGAGCTG
AGTATGCGGTCCTCATGGTATCAGAAGTGGAACGTCCATCTCTACGCCAGGCCTGAGGCTATTGGCGGTACTATTC
ACAACGTACTCAACGGCGATTTGGACATCACCTTCGCGGACTCCATCATCAACAACGACGAACTTTTCTCCAAAGT
GGAAGCCAGGAACATGGAGATTACTGGCATTCCCTCCACTTTCCTGCTCCCGCAGACGGTTCGTGAGGGAAGCCCT
ACACATCCTAGCTATCCGTCCGGCCACGCAGTTCAGAACGGCGCGTTCAGTACCATTTTGAAAGCACTGGTGGGCT
TGGAGAGGGGACTCGAATGCTTCAATGATCCAGTTGTCCCTAGCGATGATGGCCTGACCCTCCTGCCTTTCGACGG
CTGCCTCACCTTCGAAGGCGAAATTAATAAGTATGCCGCCAACGTGGCTCTCGGCCGCAACTGGATGGGTGTCCAC
TGGCGAATGGATGCCCAAGAAGGGCTCCTCCTGGGGGAGACGGTGGCCGTTCGGATCCTAATGCAGGAACTGGCAG
GATTTCCTGAGCTGGAGCCCTATGAGTTCCGGGTCATGTCCGGGGAACGCAACGTGACTGAGGCGGTCACTGACCG
TCGGATTGCCAATATGTATCTCAAGTACGCCGGGCTCCGGGACTGGTGGACTCACGATGTGAGGTCCGGAGAGTAC
CTCCTCAGGGGCGCGCTGGATATTTTTTCAAGTCCCAGCCAGTCCGTCATCGACAGACTTGGATACGGCGTCCACA
CTGTCTGTGGGGGGGTGGATGCCAGATGCCGAAGAAGTGGCCCGCGCTTTTAATAG
SEQ ID NO: 59-DNA sequence encoding an alternative Vanadium-dependent iodoperoxidase of
Laminaria digitata, codon-optimized for dicot plants
ATGAAGGGTTTGGCCGGACCAGCGGGAGCTATGGTCGTGGTTGCGCTAGGACTTGTTCCGGGAGGAGCAATCGGAA
AAAGCTTAAGACAAGAGCCAAGCGAGCCACGGCTATCAGGCGGAGTCGATACTGCTGTAAGCCCTTCCAAAGATAC
TCTGAAGGGTTCACTTTCAAGAAACTTGCAGGTCGCAAACGATGATGCTTTGGACGTTAACGGAACACCGGCAGAA
AGGGCGGCAAATGCTCTAGCTCAAAGGATCGAGCTTGCTGAAACCGAGTTCGCTGCCTCCGAGGGACTCTTCCACG
TCAATAACGGCGACAGGTCTAGCGCCGCTACTTTTCATAAATCCTTACCACACGATTCTTTGGGTCAGGTCGATTC
TGCTGCTTTCGAGGCTTTGACTGAGTGCATTGCCCAGGGTGATTTTGATATCTGCGAACTCGTACCTGCCGGGGAT
GTAGGTAGGTTGTCGAATCCCCTAGCAGGTATAACTGTTGAGATGGCTGGTGCGGCTGGTTCTGCCCTTACACTCC
CTCCCGCAAGTGCTCTGGATTCCGAGGATTTAGCGGCTCAGATGGCTGAGCTACACTGGATGGCTCTTACAAGAGA
TGTACCTTTTTCACAGTATGGGGAGGATGAAGCCACAGTCGCTGCTGCAGATAATCTTGCGACGATGCCAGGATTC
CAAAACATGGTTGGCGTGGCAGTCGATCGGGATGGACGAGCGGACCCCCAATCACAACTATTCAGAACTTCCGCTT
TTGGTGTCGAGACGGGTCCTTTCATCTCACAACTTTTGGTCCAAGATTTCACTATAGACAGCATTACTGTTGCTCC
TATTCAAAAAACCTTTGAACCAGGGGCTGATTATATGGCTGACTACGATGAATGGCTGTTCATACAGAACGGAGGA
GTACCGGACCATGATGACGTTCTTTTCGACGACGTTAATAGATATATTAGGAACTCTAGAGATCTTAGTAGACTTG
TCGCTGCTGATACGGTGAATACGGAGGCTTATAGAGCCGCACTTATTCTTCTTGAGCAAGGAGCAATATCTGGTCC
AGGATCGAATGGTCCTTACGCAGGATCTTCAAGACAAGCCGGTTTTGTGAACTACGGAGTTTCTCATCTTATGCGA
TTGGTGGGTACCGCGGAGTTATCACAAAAAAGTGCGTGGTATCAAAAATGGAATGTGCACATGTTCGTAAGACCTG
AGGCGTTTGGTGGTACCATTCACAACGTGCTCCTTGGAAAACTTAATGTTGATATAAATCCCTCATTGCTAAAAAA
TACAGAACTGTTGGAAAGAGTTGCAGAGCGGAATGGTGTTATAAACGGGCGGCCAGGTGTGCTCGATCGAACATAC
CTCTTATCTCAAGCCGTAATTGAGGGATCACCTACTCACCCAAGTTATCCGGCGGGCCACGCAACCCAAAATGGAG
CTTTTGCTACTGTGCTGAAAGCATTGGTAGGCCTAGAGAGGGGCTCCGATTGTTTTAGAGATCCTAAGGTTCCAGA
TGATGAAGGACTTACACTCCTTGACTTTACTGGAGATTGTCTGACATTTGAGGGAGAAATTAACAAACTCGCGGTT
AATGTTGCATTTGGTCGAAATATGTGTGGAGTGCACTGGAGAATAGATTCAGAACAAGGCCTCCTCTTGGGTGAAA
TGGCTGCCGTTCGTATCTTACAACAAGAAGCTGTAACATTTCCAGAAAATGCCGGCTATGAATTCAATCTTATGAG
CGGTGAGACTATCAGGCTTGAGACCGACGGAACTTTCTTCATCAATGATCGTCTGTGCAGTGGTGATGCTTTCATG
GGAGCCGATTTATGCTAATAG
SEQ ID NO: 60-DNA sequence encoding the Vanadium-dependent iodoperoxidase of Laminaria
digitata, codon-optimized for monocot plants
ATGAAAGGCCTAGCAGGCCCCGCCGGGGCGATGGTTGTGGTGGCCTTGGGTCTCGTGCCGGGCGGCGCGATCGGGA
AATCCCTCCGGCAGGAACCTAGTGAGCCACGGCTTAGCGGAGGCGTGGACACCGCAGTCAGTCCATCAAAGGACAC
CCTAAAGGGCTCCCTGTCCAGAAATCTGCAAGTCGCGAACGATGACGCCCTCGATGTTAACGGTACACCGGCGGAA
CGGGCCGCCAACGCTCTGGCACAGCGTATAGAGCTTGCAGAAACTGAGTTCGCGGCGAGCGAGGGTCTTTTCCATG
TGAACAACGGCGACAGGTCTTCTGCCGCTACCTTTCACAAATCTCTCCCTCACGATTCCCTCGGTCAGGTCGACTC
GGCGGCGTTCGAGGCTCTAACCGAGTGTATTGCACAGGGCGACTTCGACATATGTGAGTTGGTACCAGCCGGGGAC
GTTGGGAGGCTCAGCAACCCACTGGCCGGTATAACGGTCGAGATGGCCGGCGCCGCAGGCAGCGCTCTTACTCTGC
CCCCAGCCAGCGCCCTCGACTCAGAAGACCTCGCAGCTCAAATGGCTGAACTGCACTGGATGGCTCTCACAAGGGA
CGTGCCTTTCTCGCAGTATGGCGAGGACGAGGCGACGGTCGCTGCAGCAGACAACCTGGCAACGATGCCAGGCTTT
CAGAACATGGTCGGCGTTGCGGTCGATAGAGATGGTAGGGCCGATCCGCAATCTCAACTCTTCAGGACTTCAGCGT
TCGGCGTTGAAACAGGCCCTTTTATCTCCCAGCTGCTTGTGCAGGACTTCACTATAGACTCCATCACGGTCGCGCC
TATTCAGAAAACCTTCGAGCCGGGAGCCGATTACATGGCCGACTACGACGAGTGGCTCTTCATACAGAACGGGGGG
GTTCCAGACCACGATGACGTATTGTTTGATGACGTTAACCGCTACATTCGCAACTCGAGGGATCTCTCGCGCCTGG
TCGCTGCCGATACGGTCAATACCGAGGCATACAGGGCCGCGCTGATCCTCCTGGAACAGGGCGCCATTTCTGGGCC
TGGCTCCAACGGACCTTACGCGGGCTCTAGCAGGCAAGCCGGATTCGTCAACTATGGTGTCTCACACCTCATGAGA
CTGGTTGGGACGGCTGAGCTTTCCCAGAAATCGGCCTGGTATCAGAAGTGGAACGTTCACATGTTCGTCAGACCAG
AGGCTTTTGGTGGGACGATACACAACGTGCTGCTAGGCAAACTTAATGTGGACATTAACCCTAGCCTGCTGAAAAA
CACCGAGCTGCTTGAGCGGGTCGCCGAGCGCAATGGAGTTATTAACGGGAGACCAGGTGTGCTGGACCGGACTTAC
CTGCTGAGTCAGGCGGTCATTGAGGGTTCCCCCACACACCCATCCTACCCGGCGGGGCATGCCACACAGAATGGGG
CATTTGCGACTGTTCTGAAGGCGCTCGTCGGGCTGGAGCGAGGGAGCGACTGCTTCCGGGACCCCAAGGTGCCTGA
CGATGAGGGTCTCACACTGCTCGACTTCACAGGCGACTGCCTGACCTTCGAGGGTGAGATCAACAAGTTGGCCGTC
AATGTGGCATTCGGTCGTAATATGTGTGGAGTGCACTGGCGCATAGACTCGGAGCAGGGCCTTTTGCTGGGTGAAA
TGGCTGCGGTCAGAATTCTCCAGCAGGAGGCAGTGACGTTCCCGGAGAATGCGGGGTACGAGTTCAACCTAATGTC
CGGGGAGACTATTCGACTCGAAACTGATGGGACCTTCTTCATAAATGATAGATTGTGCAGTGGCGACGCATTCATG
GGCGCCGATCTTTGC
SEQ ID NO: 61-DNA sequence encoding the Vanadium-dependent chloroperoxidase of Alternaria
didymospora
ATGACGATTGACTTCACTCCTGTCGAGCTTCCTGTGGTCGAGGAAGACGCCGAGTACAACTGGAACTACATACTCT
TCTGGAACAATGTCGGCTTGGAACTCAATCGCGTTACACACACCTTTGGGGCCCTCAAGGCCGGTCCACCACTTTC
GCCCAGAGCTCTAGGCATGCTTCAGTTGGCTGTGCACGATGCCTACTTTGCCATCCATCCCTCTGCTGGCTTCACG
ACCTTCTTGACACCAGGCGCTGAAGACGGTGCATACCGTCTGCCTGATCCAAGTTATGCAAAGGACGCTCGCCAGG
CAGTAGCTGGAGCAGCAATCGCGATGCTGTCCAAGCTTTACATGAAGCCCAAGGTCGTCCCCCGCAGCCCTATCTC
TCACAACGCCTATGCCCAGCTCCAGCACGTCCTGGACATCTCGGTAACCAAAGCCCCTGCGGCTTGCGATCCGGCA
TCTAGCAGTTTCATCTTCGGCAAAGCAGTCGCTACTGCTGTCTTCGACCTGCTTTTCCACAAGGAAGGTGCCGACC
AATCTGGCTACAGTCCCAAGCCCGGCCCTTTCAAGTTCAACGATGAGCCCACTCACCCTGTTGAACTCATTCCCGT
TGATGCCAACATACCCGATGGTGATAAGATGCCGCGCCGCCAGTACCACGCCCCCTACTATGGCGAGACGGCCAAG
CGCTTTGGCACCCAGACTGAGCACATGCTTGCCGACCCACCAGGCATACGTTGCGCTGGCGAGGTTGCCGAATATG
ACGATGCTATCCGTGAAGTCTACGCCATGGGCGGAGCACCCGGTCTCAACACCACCAAGCGCACTCCTCACCAGAC
AGTCCAAGGCATGTTTTGGGCCTACGACGGCCCGAAACTGATCGGTACGCCACCAAGGCTCTACAACCAGATCGTT
CGCAAAATCGCAGTGACGTACAAGAAGGACAATGACCTGGTCAACAGCGAGGTCAACAACGCCGACTTCGCCCGTC
TCCTTGCTCTGGTGAACGTGGCTATGACCGACGCAGGCATCTTCGCCTGGAAAGAGAAGTGGGAGTTTGAGTTCTG
GCGCCCGCTTTCTGGTGTCCGTGACGATGTTCTCCGTGACCCTGAGGGTAAAGCGTCGACCGCGGCGATCCATTCT
GGCTTAGCCTCGGCGCCCCAGCTACAAAACTCAGACGAAGCTCCTTTCAAGCCCCCCTTCCCCGCCTACCCCTCCG
GCCACGCTACATTCGGCGCTGCGGCCTTCCAGATGGTCCGCAAGTACTACAACGGGCGTCTTGGAAAATGGGCGAC
GACGAGCCGAGACACCATCGCCGTCGAAATGTTTGTATCTGAGGAGCTGAACGGAGTGAGCAGGGACCTCAGCAAC
CCCTACGACCCCAAACGCCCCATCACAGATCAACCTGGTATCGTGCCGACCCGGATGCCACGCCGATTCTCTTCCT
GTTGGGAGATGATGTTCGAAAACGCCGTCTCACGTATCTTCCTCGGTGTGCACTGGCGCTTCGACGCCGCAGCCGG
ACAGGACATTTTGATCCCTACGACGAAGAAGGACGTCTATGCTGTCGACGACAAGGGCGCTGCTCTGTTCAAGAAT
GTTGAGGATATTCGTTATAAGACGAAGGGCACGAGGAAGGGACACAAGGGTTTGTTGCCCATTGGCGGTGTGCCCC
TTGGTATTGAAATTGCGAATGAGATTTACAACAACAAACTCAGCCCTACGCCACCGGGGGAGCAGCCAATGCCGCA
GCCTCCGCAGCACCAGGGGCCACCAAGGAAGAAGGGGGAGTTGGCAGAGGCGAAGGATGAGGAACAGGCTCCTATG
ATGGATGTAGCGCCCTAG
SEQ ID NO: 62-Protein sequence of the Vanadium-dependent chloroperoxidase of Alternaria
didymospora
MTIDFTPVELPVVEEDAEYNWNYILFWNNVGLELNRVTHTFGALKAGPPLSPRALGMLQLAVHDAYFAIHPSAGFT
TFLTPGAEDGAYRLPDPSYAKDARQAVAGAAIAMLSKLYMKPKVVPRSPISHNAYAQLQHVLDISVTKAPAACDPA
SSSFIFGKAVATAVFDLLFHKEGADQSGYSPKPGPFKENDEPTHPVELIPVDANIPDGDKMPRRQYHAPYYGETAK
RFGTQTEHMLADPPGIRCAGEVAEYDDAIREVYAMGGAPGLNTTKRTPHQTVQGMFWAYDGPKLIGTPPRLYNQIV
RKIAVTYKKDNDLVNSEVNNADFARLLALVNVAMTDAGIFAWKEKWEFEFWRPLSGVRDDVLRDPEGKASTAAIHS
GLASAPQLQNSDEAPFKPPFPAYPSGHATFGAAAFQMVRKYYNGRLGKWATTSRDTIAVEMFVSEELNGVSRDLSN
PYDPKRPITDQPGIVPTRMPRRFSSCWEMMFENAVSRIFLGVHWRFDAAAGQDILIPTTKKDVYAVDDKGAALFKN
VEDIRYKTKGTRKGHKGLLPIGGVPLGIEIANEIYNNKLSPTPPGEQPMPQPPQHQGPPRKKGELAEAKDEEQAPM
MDVAP
SEQ ID NO: 63-DNA sequence encoding the Vanadium-dependent chloroperoxidase of Curvularia
inaequalis
ATGGGGTCCGTTACACCCATCCCACTCCCTAAGATCGATGAACCCGAAGAGTACAACACCAACTACATACTATTCT
GGAACCATGTCGGTTTGGAACTCAACCGCGTAACTCACACTGTTGGAGGCCCCCTGACGGGACCACCTCTCTCTGC
CAGGGCTCTGGGTATGCTGCACTTGGCTATTCACGACGCCTACTTTTCTATCTGCCCTCCGACCGACTTCACCACC
TTCCTCTCACCTGATACTGAGAATGCCGCGTACCGTCTACCTAGCCCTAATGGTGCAAATGATGCTCGCCAAGCAG
TCGCTGGAGCTGCCCTCAAGATGCTGTCTTCACTGTACATGAAGCCCGTCGAGCAGCCTAACCCTAACCCCGGCGC
CAACATCTCCGACAACGCTTATGCTCAGCTTGGCTTGGTTCTCGACCGATCAGTTCTGGAGGCACCTGGTGGCGTG
GACCGAGAGTCAGCCAGTTTCATGTTTGGTGAGGATGTAGCCGATGTCTTCTTCGCACTCCTCAACGATCCTCGAG
GTGCTTCGCAGGAGGGCTACCACCCTACACCCGGCCGCTATAAATTTGACGATGAACCTACTCACCCTGTCGTCCT
CATTCCAGTAGACCCCAACAACCCTAATGGTCCCAAGATGCCTTTCCGTCAGTACCACGCCCCATTCTACGGCAAG
ACCACGAAGCGTTTTGCTACGCAGAGCGAGCACTTCCTGGCCGACCCACCGGGCCTGCGTTCTAATGCGGACGAGA
CCGCGGAGTATGACGACGCCGTCCGCGTCGCTATCGCCATGGGTGGTGCTCAGGCTCTCAACTCCACCAAGCGTAG
CCCATGGCAGACAGCACAGGGCCTATACTGGGCCTACGATGGGTCAAACCTCATTGGCACACCACCTCGCTTTTAC
AACCAGATCGTACGTCGCATCGCAGTTACGTACAAGAAGGAAGAGGACCTTGCCAACAGCGAAGTCAACAATGCGG
ATTTCGCCCGCCTCTTCGCCCTCGTCGACGTCGCTTGCACAGACGCTGGTATCTTTTCCTGGAAGGAGAAATGGGA
GTTCGAATTCTGGCGCCCACTATCTGGTGTGCGAGACGACGGCCGTCCAGACCATGGAGATCCTTTCTGGCTCACT
CTCGGTGCCCCAGCTACTAACACCAACGACATTCCATTCAAGCCTCCTTTCCCAGCTTACCCATCTGGTCACGCGA
CCTTTGGCGGTGCTGTGTTCCAAATGGTGCGTCGATACTACAACGGCCGCGTAGGTACATGGAAGGACGACGAACC
CGACAACATTGCCATCGATATGATGATCTCGGAGGAGCTCAACGGCGTGAACCGCGACCTACGCCAGCCTTATGAC
CCCACGGCCCCAATCGAAGACCAACCCGGTATCGTGCGCACCCGCATTGTTCGCCACTTCGACTCGGCCTGGGAAC
TCATGTTCGAAAACGCCATTTCGCGCATCTTCCTCGGTGTCCACTGGCGTTTCGATGCCGCCGCCGCCCGCGACAT
TCTCATTCCCACGACGACAAAGGACGTCTACGCTGTCGACAACAATGGCGCCACCGTGTTCCAGAACGTAGAGGAC
ATTAGGTACACAACCAGGGGTACGCGTGAGGACCCCGAGGGCCTCTTCCCTATCGGTGGTGTGCCACTGGGTATCG
AGATTGCGGATGAGATTTTTAATAATGGACTTAAGCCTACGCCCCCGGAGATCCAGCCTATGCCGCAGGAGACACC
GGTGCAGAAGCCGGTGGGACAGCAGCCGGTTAAGGGCATGTGGGAGGAAGAGCAGGCGCCGGTAGTCAAGGAGGCG
CCGTAG
SEQ ID NO: 64-Protein sequence of the Vanadium-dependent chloroperoxidase of Curvularia
inaequalis
MGSVTPIPLPKIDEPEEYNTNYILFWNHVGLELNRVTHTVGGPLTGPPLSARALGMLHLAIHDAYFSICPPTDFTT
FLSPDTENAAYRLPSPNGANDARQAVAGAALKMLSSLYMKPVEQPNPNPGANISDNAYAQLGLVLDRSVLEAPGGV
DRESASFMFGEDVADVFFALLNDPRGASQEGYHPTPGRYKFDDEPTHPVVLIPVDPNNPNGPKMPFRQYHAPFYGK
TTKRFATQSEHFLADPPGLRSNADETAEYDDAVRVAIAMGGAQALNSTKRSPWQTAQGLYWAYDGSNLIGTPPRFY
NQIVRRIAVTYKKEEDLANSEVNNADFARLFALVDVACTDAGIFSWKEKWEFEFWRPLSGVRDDGRPDHGDPFWLT
LGAPATNTNDIPFKPPFPAYPSGHATFGGAVFQMVRRYYNGRVGTWKDDEPDNIAIDMMISEELNGVNRDLRQPYD
PTAPIEDQPGIVRTRIVRHFDSAWELMFENAISRIFLGVHWRFDAAAARDILIPTTTKDVYAVDNNGATVFQNVED
IRYTTRGTREDPEGLFPIGGVPLGIEIADEIFNNGLKPTPPEIQPMPQETPVQKPVGQQPVKGMWEEEQAPVVKEA
P
SEQ ID NO: 65-DNA sequence encoding the Vanadium-dependent chloroperoxidase of Pyrenophora
tritici-repentis
ATGGCAAATTTCACTCCTGTTCGGCTTCCTAAGGTCGAGGAAGATGAGGTGTACAATAAGAACTACATCCTCTACT
GGAATAATGTCGGCTTAGACCTCAACCGTGTTACACACACCTTGGGAGGCCCTCAGACCGGTCCACCCATCTCCGC
CAGAGCTCTAGGCATGCTTCAATTGGCTATCCACGATGCCTACTTTACTATCAAGCCCTCTGCCGACTTCACAACC
TTCTTGACACCCAACGCCGAAATCGACGCATACCGTCTGCCCGATCCGACTCATTCGGACGATGCCCGCCAGGCGG
TAGCGGGAGCAGCAGTCACGATGCTGTCGATGCTTTACATGAGGCCTGCGGAAATGCCCAGACCCTCCCCCATATC
CAATGACACCTATGCCCAGCTCGAGTATATTATAGAATCCTCGATGACCAACGCACCCGGCGGCTGCAATACGGTA
TCCTACAGTTTCAACTTCGGCAAGGCAGTAGCCACTAAATTCTTCGACCTGCTTTTCCACGAGGAAGGTGCCAATC
AACGTGGCTACACCCCCACATTCGCTCCTTTCAAGTTCAACGATGAACCCACTCATCCTGTTGACCTTGTCCCCGA
GGACGCCAACGAGCCCGGTGGTAACATAGTTCCTAGACGCCAGTATCATGCCCCCTTCTATGGTACGAGAGCCAAG
CGCTTTGCGACACAGACTGATCACATTATTGCCGATCCACCAGGCATTCGTTCCGGCGCTGGTGAGGTCACCGAAT
ATGACGACGCTATCCGTGAAGTTTACGCCATGGGTGGAGCAGCCAGTCTCAACACTACCAAGCGCACTCCCCACCA
GACAGTCCAAGGCATGTTCTGGGCTTACGATGGCGCAAAGTTGATCGGTACACCACCGCGACTATACAACCAGATC
ATACGCAAAATCGCAGTGGCGTACAAGCAAGAAGATAACCTGGCCGAAAGCGAGATCAACAACGCCGACTTTGCTC
GTCTCCTCGCTCTGGTGAACGTGGCTATGGCTGATGCAGGCATCTTCTCCTGGAAGGAAAAGTGGCAGTTTGAGTT
CTGGCGCCCGCTTTCCGGTGTCCGCGCCGATAGTCTTCGTGACCCTAAACTTGTAGACCGTGGCGACCCATTCTGG
CTTACGCTCGGGGCTCCAGCTACAAACTCAGATTCACTTCCCTTCAAGCCGCCCTTCCCCGCCTACCCCTCTGGTC
ACGCCACGTTCGGTGGTGCAGCCTTCCAGATGGTACGCAAGTACTACAACGGGCGTCCTGGACTTGGTTCCTGGGC
AGACGACGAGCCAGACAACATCGCCGTCGAATTTGTCTCTGAGGAACTGAATGGAATAAGCAGAGATCTCCGCCAA
CCCCACGACCCCAAACGCGACATTACCGACCAACCCGGTACCGTGCGTACCCGTCTCCCCCGCCACTTCTCTTCGT
GCTGGGAGATGATGTTCGAAAACGCCGTCTCACGCATCTTCCTAGGTGTGCACTGGCGCTTCGACGCTGCGGCCGC
CAAGGACATCTTGGTTCCCACGACGAAGAAGGATGTCTACGCTGTAGACGACAAGGGCGCTTCTCTGTTCAAGAAT
GTTGAGGATATTCGTTATAGGACGAAGAGTACAAGGGAGGGATTTGAGGGTAAATACCCCATTGGTGGTGTACCCC
TGGGTATTGAGATCGCGAATGAGATTTTCGACAACGGACTCGTGCCTACGCCGCCGGAATTGCAGCCTGTGGTGCA
GGGTATGCCGCAGCCTACACCGCAGCCTCCGCAGCATCAGGGGCCACCAAGGAAGATGGAGAAGTTGCCAAAGCCG
AAGGATGAGGATCAGGTTCCTATGATGGATGTGGAGCCCTAG
SEQ ID NO: 66-Protein sequence of the Vanadium-dependent chloroperoxidase of Pyrenophora
tritici-repentis
MANFTPVRLPKVEEDEVYNKNYILYWNNVGLDLNRVTHTLGGPQTGPPISARALGMLQLAIHDAYFTIKPSADFTT
FLTPNAEIDAYRLPDPTHSDDARQAVAGAAVTMLSMLYMRPAEMPRPSPISNDTYAQLEYIIESSMTNAPGGCNTV
SYSFNFGKAVATKFFDLLFHEEGANQRGYTPTFAPFKFNDEPTHPVDLVPEDANEPGGNIVPRRQYHAPFYGTRAK
RFATQTDHIIADPPGIRSGAGEVTEYDDAIREVYAMGGAASLNTTKRTPHQTVQGMFWAYDGAKLIGTPPRLYNQI
IRKIAVAYKQEDNLAESEINNADFARLLALVNVAMADAGIFSWKEKWQFEFWRPLSGVRADSLRDPKLVDRGDPFW
LTLGAPATNSDSLPFKPPFPAYPSGHATFGGAAFQMVRKYYNGRPGLGSWADDEPDNIAVEFVSEELNGISRDLRQ
PHDPKRDITDQPGTVRTRLPRHFSSCWEMMFENAVSRIFLGVHWRFDAAAAKDILVPTTKKDVYAVDDKGASLEKN
VEDIRYRTKSTREGFEGKYPIGGVPLGIEIANEIFDNGLVPTPPELQPVVQGMPQPTPQPPQHQGPPRKMEKLPKP
KDEDQVPMMDVEP
SEQ ID NO: 67-DNA sequence encoding the Vanadium-dependent chloroperoxidase of
Graciliariopsis chorda
ATGGTCGCTTCATTTTCCATTCTGCTCCTGCTTTCTGCCGTGCTTCTCCACGTCCACGCAATCTCTCTCAGCAACC
CATCTTTGCAAACGCTTCCAAATGTGTCCCGTCAAACAACCTGTTCATCCTCTGAAGACTGTCCAGACACAAACCC
CTTCGTAAGATGCGTTCGCAATCAGTGCATCGATCTTCGAGACACAGTTCCAGATTTACCAACCCGCCCACCCACA
ACAACATCAGCCACACAACCCAACGCACCCAACACACCAGCAGAGGGCGGTTCCTGTCTAACCATAACAGAATGTC
CATCCGGTCTTATCTGCGTCGCCACCAATCCCGGTGGCAGATGTGAACTGTCCAGAGGAATGTTCGGAGTTGCAAC
CTTCGTCGTTAACTTGATTCAGCAAGACTATACACCCGCACCTGAACTATCACCGGATCAAGGTGGCCCTACAGTC
ACTTCTAGAGCAATGGCCATCATCTTTCTCGCCGCTCATGACACCTTTGCAGTCGTCTCTGGAGAGTTCTCGGCTA
ACACAAATCTCGGAGTGCTTCGATCCATCCCGATTCGAGTCGCCGCACGCCGTTTAGACGACGAGGAAAGGGAGCG
ACAAGCGGAGGCTGCCATGCAGGCCGCCGCTTTAACCGCAGCAAAAAGACTGTACCCGTCTTTAACACGAATTATA
GACCCCGTGTTCGAACTCGTTACGGCCCGTGCTCAATCGCAATTTGTTGAGTTTGGAGAGGATGTTGGAAATGACT
TGTTCGAGTCTAGACTCAATGATGGATCTCAACGCGATCAACTTGACGATGAGTTTGAGATTGGGGAGCTTTTGAG
ACATCAACCCGACCCTAATTTCCCCATCACTGCAGGCGATCCGACTGCCGTGCAGAGGAACTTTGGTCGATTTTGG
GGTGAAGTCACACCGTTTGCCATTCGTGACGTCGAGCGTGATGCATTCTTAGGTCCATTCCCGGAACTTCCCTCTG
CCGAATATCGTGCAAATCTTGCGGAGGTTGATGTAATGGGCGAATGTAACGACTTTGTCAATGACGATGGTATCCT
TCTTCAGGACATTGGAATCTTTTGGGGCTATGACGGGGCATCCGAAATAGGAGTTCCTCCTCGTCTGTTCTTGCAG
ACTGTGCTTGCTGTCGATGAAGTTCAGGAGCTTTCTTTGCCAGATAGTGTTCGAGCTCTCACTGGTGTGGGGGTTG
CCATGGCTGACGCCGGTATTGCAGCTTGGTACTGGAAGTTTTTCTACGATCTTTGGAGGCCTACTATCGGGGTGCG
AAACGACACAATCTCTCCTGATCCAGAGTGGAATCCGCGTGGAATTCCGTTAAGTAACCGCAATGACATACCGAGA
CCACCGGAGTGTGTCGGAGTCAATCCTAACTTCCCTGCATATCCATCCGGACACGCTTCATTCGGGGCAGCTTGTT
TTGTCACCCTTGCCAATATCCTTGGCAAAGAACCCAAGGATGTGGTTGCCAGAATTACTTCGGATGAATTTAACGG
GGTCACTACGGAGGGCACTACGGGAGAGGTGCGAAGAGTGTTCACCCAAACTATCAACTTTCAGGAAGCTATTGAA
CAGAATAATGCAGGCCGCGTGTACTTGGGCGTTCATTGGAGATTTGATTCCGACGGTGGGGAGGATGTCGGTAGAC
AGATTGCTGAAATTGCAGCTAACCAATTCGACATTTAA
SEQ ID NO: 68-Protein sequence of the Vanadium-dependent chloroperoxidase of Graciliariopsis
chorda
MVASFSILLLLSAVLLHVHAISLSNPSLQTLPNVSRQTTCSSSEDCPDTNPFVRCVRNQCIDLRDTVPDLPTRPPT
TTSATQPNAPNTPAEGGSCLTITECPSGLICVATNPGGRCELSRGMFGVATFVVNLIQQDYTPAPELSPDQGGPTV
TSRAMAIIFLAAHDTFAVVSGEFSANTNLGVLRSIPIRVAARRLDDEERERQAEAAMQAAALTAAKRLYPSLTRII
DPVFELVTARAQSQFVEFGEDVGNDLFESRLNDGSQRDQLDDEFEIGELLRHQPDPNFPITAGDPTAVQRNFGREW
GEVTPFAIRDVERDAFLGPFPELPSAEYRANLAEVDVMGECNDFVNDDGILLQDIGIFWGYDGASEIGVPPRLFLQ
TVLAVDEVQELSLPDSVRALTGVGVAMADAGIAAWYWKFFYDLWRPTIGVRNDTISPDPEWNPRGIPLSNRNDIPR
PPECVGVNPNFPAYPSGHASFGAACFVTLANILGKEPKDVVARITSDEFNGVTTEGTTGEVRRVFTQTINFQEAIE
QNNAGRVYLGVHWRFDSDGGEDVGRQIAEIAANQFDI
SEQ ID NO: 69-DNA sequence encoding the Vanadium-dependent chloroperoxidase of
Rhodopirellula baltica
ATGTTTCGTCGCGATTTGCAACGATTCGAAAGGCCCGTCGCTTCTCCTTTTCTCAATCGCCGCGCGGGGAATACCA
TGCCACTCAGTTGGAATCGTTGGATCGGAACGTCCAAGTCAGCTCGATCTCGCAAACAACAACGAAAATCGACTCG
CCGCAAGTTGATGTCAGAGAATCTGGAGACCAGGAACCTTCTCGCCGCCAATCTGTTCCACAACGAGGCGATGCCG
GAAGACGTCAATGAAGATGATCAGGTCACGGCTTTGGATGCTCTCACCATCATCAATCGGATGAACCGCGAACAAG
CTGGTGAATCCGCGGGTGATGTCAGACGCGGACAGATGACGGACGTCAACAACGACGGTCGCAACAGTGCCCTGGA
TGCGTTGATGGTGATCAACCGTTTGAACCGCGACCAAGGTGGACCACGAGATTCAGGATCGCCCGACGCGGGATCG
GGAAACCAAGACCAAACAGATTCCGAAACGAGCGGAGACAATTCGGATGTTGAGGGCACCAATGGCGATGATCCAT
CCACCGACGGAACAACGTCGGGCGATCAAACAGATGCTGATTCAGCGATTGAGTTGACCGGGGATGCTGTGCTTGA
TTGGAACAACCTGTTCAACGAGTTGACCTCCAACAGCGAGGATTATCAAAACCCGGGGTACGCTTCTCGAGCGATG
GCGATGTTGAACGTGGCGATCTATGACTCCGTCGCGATAGCTTCTGGCGATTCTGAAATTGCATTCTACGAATACG
ATTCATCATTGACGAACTCTTCCGGTGACGTTTCATCGCAAGCTGCCGCATCGAGTGCCGCCTACTCCGTCCTGAG
TTCCCTGTACTCGGACCAGCAAGAAACGATCGATTCGTTCTATGGAGATGTGCTAGCAACTTACAGCCAAGATCCA
GAAACCGAGGCGGGGTTGATCCTCGGCGCGGAAGTTAACAGTACCGTGATCGCGAATCGTGCAAATGATGGATCGG
ACTCGGTCGTGGAGTACACCTACACCGATGAGATAGGTTCTTTTCATTCGGATCCGCTGAATCCGGACGTCCCAGT
ATGGGGACCGGGTTGGGGTGATGTGGACACGTTCGCAATCTCCGATGCGGACGCCTTCACACCGGAGTCGCCTCCG
GATTTGACCAGCGAAGAATACGCGGCGTCTTACAACGAAGTGAAGGAATTGGGGGCGGTCGACAGCACCACACGAA
CGGCCGACCAAACCGAAGCGGGGATCTTTTGGGCTTACGACCGAGATGGTTTGGGCACGCCGCTCACGCTGTTCAA
CGACATTCTAGAAACAGTCGCCGTGCAAGAAGGCAATACGTTTGAGGAGAATGCGGCTTTGTTCGCCCAGGCATCA
GTCGCGATGGCTGATGCGGGGGTTGTGGCGTGGACGACCAAGTTCGGAGAAGAACTTTGGCGTCCGGTCACCGCAA
TCCAAGAAGGCGATTTCGATGGAAACACGCTGACCGAAGGCGATGCTGATTGGACCGCTTTGGGGGCGCCTGATGG
AGGTGATGACATTGTTGGCTTCACGCCGCAATTTCCGACCTACATCTCCGGCCACGCGACCTTTGGCGGTGCCTTG
TTTGGGACCCTGCAAGAATTCTATGGCACCGATGACATCTCGTTCACTGTCGCGTCGGAAGAACTTGAGATTCTGT
TGGACAATCCTGAATTGCAGGAAGCCTACGGTTTGAATCTTGATGACGCCGAGCGAACTTTCAGTTCCTTCAGTGA
GGCCATGGCCGAGAATGGACGCAGCCGAGTTTACTTGGGCATTCACTTCGACTTCGATGATTTGGTCGGTCAGGAA
GTCGGGCAATCCATCGCCGCAGCGGTCGCATCGGAATTCAGCGTGGCGACGCCCGAAGATGACTCCACTGGCAAAG
GCGTCGACGATGGCCAACTCGCAACAATGGACCGTGACGACCAGCCAAACCGAGACGGCGTCGACGGTTCGAGGCA
GCGGAATCGCGAACCGGTCGCTCGGGACACGACTCAAGTGCCTCAGCAACAAACTTCATCGCGGGATGAGTCGAAT
CGACGATCAGAGGACGATCAGCGAATCGCAGCCATTGACCAGATTTTCGCAGAGAACCGCTTGATTTGA
SEQ ID NO: 70-Protein sequence of the Vanadium-dependent chloroperoxidase of Rhodopirellula
baltica
MFRRDLQRFERPVASPFLNRRAGNTMPLSWNRWIGTSKSARSRKQQRKSTRRKLMSENLETRNLLAANLFHNEAMP
EDVNEDDQVTALDALTIINRMNREQAGESAGDVRRGQMTDVNNDGRNSALDALMVINRLNRDQGGPRDSGSPDAGS
GNQDQTDSETSGDNSDVEGTNGDDPSTDGTTSGDQTDADSAIELTGDAVLDWNNLFNELTSNSEDYQNPGYASRAM
AMLNVAIYDSVAIASGDSEIAFYEYDSSLTNSSGDVSSQAAASSAAYSVLSSLYSDQQETIDSFYGDVLATYSQDP
ETEAGLILGAEVNSTVIANRANDGSDSVVEYTYTDEIGSFHSDPLNPDVPVWGPGWGDVDTFAISDADAFTPESPP
DLTSEEYAASYNEVKELGAVDSTTRTADQTEAGIFWAYDRDGLGTPLTLENDILETVAVQEGNTFEENAALFAQAS
VAMADAGVVAWTTKFGEELWRPVTAIQEGDFDGNTLTEGDADWTALGAPDGGDDIVGFTPQFPTYISGHATFGGAL
FGTLQEFYGTDDISFTVASEELEILLDNPELQEAYGLNLDDAERTESSFSEAMAENGRSRVYLGIHFDFDDLVGQE
VGQSIAAAVASEFSVATPEDDSTGKGVDDGQLATMDRDDQPNRDGVDGSRQRNREPVARDTTQVPQQQTSSRDESN
RRSEDDQRIAAIDQIFAENRLI
SEQ ID NO: 71-DNA sequence encoding the Vanadium-dependent chloroperoxidase of
Nitrosomonas sp. Nm84
ATGGATCACATCTTATATTGGAATAATGTCGCACTCGAGGCCAATCGCCGCGACTTCAGTAATGTACCTGGCACCG
ATAATCCCAATCCTAAACCTGAACAGGGCGGCCCGACACTCAGTTCCCGTGCCTTGGCCATTGTACATTTGGCAAT
GTATGACGCGCATGCTGGTGTCATCAGCAATCCGGCCACGCTGCCACGCTACCTCGCTTCACCATCCGCACCGGTT
GGTGGCGCCAGCGCGGCCGCATCAGTAGCAGCTGCAGCACATGCTTGCTTGAGCGCTTTGTACCCGCGCCAGAAGC
ATCATTTCGATGCAGCGCACCAAGCTGCAGGCCTATCCGGTGCGGGACTTGCTGAGGGTCATGGCTTCGGACTGGC
CGTAGCTACAGCGATACTGGCCGACCGTGCCAAAGATCCAGGTGCAGGCGATGACGGCTATGCTGCTTCGATGCAT
CCCGGCGGCCACCGTCCCGACCCGGCCAATCCGAAGCAGGGTTTTCATGCACCTTTCTACGGCGCACGCAGCAAGG
GTTTCGCAATCACGAAACGGCATGAGCTTGACAAACCACCTGCTCTTGGAAGTGGGGAGTATAACAAGGCACTTAA
ACAGGTTCGAGGCAAGGGCATTGCGCCTGAATGGATAGGCACACTGCCAGACCACCTCGAGAAACGCACACCGGAA
GAAACATTAATCGGCCTATACTGGGCCTACGACGGTCCACGCGAAATCGGAACACCACCACGTTTCTTCAACCAAA
TCATTCGCAAACTTGTGACAACTGCAATAAATCCGGCTACGGGCATGATCAACACCGTCGATGACAATGCGCGCTT
ATTCGCGTTCGTAAACGTGACGATGGGTGATGCCGGCATTTTAGCTTGGGATCAGAAGTATATTCACGACTTCTGG
CGACCAGTGCTGGGTATCCGTGAACACAGTGCTTCGATGGGACCTGCCGCACCCACACCTTCGCACGCCATCGGTG
ATGACTGCGATCCCTGCTGGCTGCCGCTGGGTGCCCCCGCAAGCAACAGCAACGATCGCAATTTCACGCCCCCGTT
CCCGGCTTACCCATCGGGACACGCTACCTTCGGAGCTGCTGCGCTGCATGTAACTCGGTTATTCTATGGTATTGCT
CACAACAATCGCGCCACCGACAAGCTATTCAAGCGTTCCTTGGTTTCCGATGAACTCAACGGTATAACACACGACA
ATGCTGGTAATGTTCGATCGCGACATGCGCGACACTTTCCGGGTGGACTCTGGCAGATGATCGTGGAGAATGGCTT
TTCTCGCGTGTTCCTTGGCGTGCACTGGTCGTTTGATGCGTTCGCACTCGACAACGCCAACAAGCCCGATCTGACG
CGCAATATCGGCGGTGTGCCGCTGGGATTAGCAATTGCAGAAGATATTTTTGCTGCAGGTGGAGGCAAGGCGCCCG
CTAAATCTTGTGTTGAACCACGACCCTAA
SEQ ID NO: 72-Protein sequence of the Vanadium-dependent chloroperoxidase of Nitrosomonas
sp. Nm84
MDHILYWNNVALEANRRDFSNVPGTDNPNPKPEQGGPTLSSRALAIVHLAMYDAHAGVISNPATLPRYLASPSAPV
GGASAAASVAAAAHACLSALYPRQKHHFDAAHQAAGLSGAGLAEGHGFGLAVATAILADRAKDPGAGDDGYAASMH
PGGHRPDPANPKQGFHAPFYGARSKGFAITKRHELDKPPALGSGEYNKALKQVRGKGIAPEWIGTLPDHLEKRTPE
ETLIGLYWAYDGPREIGTPPRFFNQIIRKLVTTAINPATGMINTVDDNARLFAFVNVTMGDAGILAWDQKYIHDFW
RPVLGIREHSASMGPAAPTPSHAIGDDCDPCWLPLGAPASNSNDRNFTPPFPAYPSGHATFGAAALHVTRLFYGIA
HNNRATDKLFKRSLVSDELNGITHDNAGNVRSRHARHFPGGLWQMIVENGFSRVFLGVHWSFDAFALDNANKPDLT
RNIGGVPLGLAIAEDIFAAGGGKAPAKSCVEPRP
SEQ ID NO: 73-DNA sequence encoding the Vanadium-dependent chloroperoxidase of
Agrobacterium species RAC06
ATGCTTGTTCTACCTGATGTCGACGAACCTGCCGGGCTTAACGCATTTCCGGTCCTGTACTGGAATCATGCCGGCC
TCCAAATGAACCGTATCACGCATTCGCTTCGCGGCCCGCAAGGTGGCCCTACCATGAGCTCTAGAGCTCTGGGACT
TCTTCATCTTGCCATGCACGACGCCTTCTTCGGCGCTCTCGGTCATGACGAACACTCGTCTCCCGCAACCTGGTTG
CGAGACGTCAATCGTCCGCCACTCGATCCTTCCATCCAGGCAACGGAAGCAAATGCCAATGCAGCCCTCACCGCTG
CCGCCATTACAATCCTTGAGCGACTATATTGTGCTCCGGGCGAGGTCTGTGACGCGGCAAGCAATGCGCTGCGCGA
CGCGATGCGCAACTTCAAGAGCCCATTCGGTGTGGCAATTGCAGCAGGATCGCCGGCGCATCACTATGGCGTGACC
ATTGCCGAAAGAGTCCACGCAGCACTTGCGGTATTGCCAGGCGAGCCGGGCGCCGATCAGGGCGACTACCGGCCCC
GGGGAGGTCGCTATCACTTCGATGACGAACCACTCAACCCTGTCCGCCGCGTGGACATTGATCCTCGAGACCCTTC
AAAAGGACAAAAGGCACAGCGTGTCTATCACGCGCCCTTTTACGGTGAGACCGTGAGAACCTTTGCAGCGACCGAT
CCGAATGGCCATCGGATCGCCGACTGGCCAAGACCACCGGAGCCAGAATACGTGGCAGCACTTGAGGAGGTCCAGC
GGCTTGGCGGCGCACCGCTCCTGAACACAACGCATCGGACGGCAGACGAGACGGTGGCGGCCTATTTCTGGGCTTA
TGATGGAGCCAATCTCATTGGAACACCTCCGCGTCTTTACAATCAGATCCTGCGCAATATCGCCTGGTCGAGGAGC
GATGCCTCGCTGAGCGACTTCGACAGAAGCAAAGAGTTCGTCCGGCTGTTTGCGCTTGCCAATGTGGCGATGGCGG
ATGCAGGCAAATTCGCCTGGGCCGAAAAATACCGGCACGATCTCTGGCGCCCATTGTCTGGCATTCGCCAACACGA
CGCAAGCGCATTCGCCTCCGAGACCGGAAGCACAATGGTGACGCCGCCTGCCGATCCGTTCTGGCTCGCACTTGGT
GCGCCAGAAACAAACTCCAACCGTCTGAGCTTCAAACCACCCTTCCCCGCCTATCCCTCAGGCCATGCAACCTTTG
GCGCAGCATGCTTTCAGATGGCAAGGCTCTACTACGCCTCGAACGGGACGGCCGCTGTTGACAGAAATGGGGTCGA
CGACATCGGTTTCACATTTGTGTCGGAGGAGATGAATGGCGTAAGCCGCGACTTGCATCAGCCTTACGATCCTTCA
TCGCCGATCGAGGACCAGCCAGGGCTTGTTCGGACCTATGTGAAGCGCAACTTCCCGTCCCTCTGGCATGCAATAT
GGGAAAATGCGTTCAGCCGGATTTGGCTGGGTGTCCACTGGCGTTTCGACGCCTTTGACTATCGCGATACCGGAAG
CGGTCGCGATGCCGACGGGCGCGAGCGCTACAAGGACCCTGCCGATGTCGGCTATTCGCATGTCTGGACTGCAAAG
CGGAACGCCGGCGACCCACTGCCGATCGGCGGCGTCCCCTTGGGTCTCGGCATCGCCAATGACATTTTTGACAGCG
GCATGCGACAGCAAGCCGACGAAATGCCGAAAGCGCGGTCGACACCTCAGCAAAGCAAGATCACGGACACGACCTA
CTAA
SEQ ID NO: 74-Protein sequence of the Vanadium-dependent chloroperoxidase of Agrobacterium
sp. RAC06
MLVLPDVDEPAGLNAFPVLYWNHAGLQMNRITHSLRGPQGGPTMSSRALGLLHLAMHDAFFGALGHDEHSSPATWL
RDVNRPPLDPSIQATEANANAALTAAAITILERLYCAPGEVCDAASNALRDAMRNFKSPFGVAIAAGSPAHHYGVT
IAERVHAALAVLPGEPGADQGDYRPRGGRYHEDDEPLNPVRRVDIDPRDPSKGQKAQRVYHAPFYGETVRTFAATD
PNGHRIADWPRPPEPEYVAALEEVQRLGGAPLLNTTHRTADETVAAYFWAYDGANLIGTPPRLYNQILRNIAWSRS
DASLSDEDRSKEFVRLFALANVAMADAGKFAWAEKYRHDLWRPLSGIRQHDASAFASETGSTMVTPPADPFWLALG
APETNSNRLSFKPPFPAYPSGHATFGAACFQMARLYYASNGTAAVDRNGVDDIGFTFVSEEMNGVSRDLHQPYDPS
SPIEDQPGLVRTYVKRNFPSLWHAIWENAFSRIWLGVHWRFDAFDYRDTGSGRDADGRERYKDPADVGYSHVWTAK
RNAGDPLPIGGVPLGLGIANDIFDSGMRQQADEMPKARSTPQQSKITDTTY
SEQ ID NO: 75-DNA sequence encoding the Vanadium-dependent chloroperoxidase of
Bradyrhizobium yuanmingense
ATGCAGTGGCAGGTCAACGGAGCGTCGCAAATCGGTGTTCCGCCGCGGCTGTATAATCAAATCGTTCGTGAGATAA
TTGGGAACAATGTGAACGGAGCTGAGCGGGCGGCGGCCTCGGCGCGTCTTTTAGCGCTCGTCAACGTCGCGATGGC
GGACGCAGGTATCGCTTCTTGGTACTACAAGTACACGTACCAGCTTTGGCGCCCTGTATTAGGAATTAGAGAATAC
GACGATAGTTATTGGTACAACGGCACCGCGGTTTCTCACGCGCTTCACAAACGGTGCGATCCTTGGTGGATACCCC
TAGGCTCCCCAAGAACGAACGAGTCTGGTCGACATAGCTTTACTCCTCCCTTCCCCGCCTATCCTTCGGGGCACGC
TACGTTCGGTGCCGCTGCCTTCGAAATAACTCGGCGTTTTTTCGGGGTCGCACCCGGTGCCCAAGACAATCTATTC
TTTAACACAATATCGGATGAGTGCGACGGCCGAGCCATTGCCGAAGATGGCTCTTTCAGGGGGCGTCAGCGGCGGC
ACCACGATTCCTTGCTCCGTGGAATGTTCGACAATGCGGTGAGTCGCGTGTATCTCGGTGTTCACCGGCGCTTTGA
CGGCATTGGAGACAACGTTACCACTCACCAAGATATCTTGAATGATAACTCGAATATTGGCGGCGTACCCTTGGGA
CGCGCTCTAGCCCACGATATTTTCAACAATGGCCTTGCAAAGAGCGCTGCAGCGAGAGCGGTAATCACTCCCAAAA
ATCTCGCTCCGGTACCTTGA
SEQ ID NO: 76-Protein sequence of the Vanadium-dependent chloroperoxidase of Bradyrhizobium
yuanmingense
MQWQVNGASQIGVPPRLYNQIVREIIGNNVNGAERAAASARLLALVNVAMADAGIASWYYKYTYQLWRPVLGIREY
DDSYWYNGTAVSHALHKRCDPWWIPLGSPRTNESGRHSFTPPFPAYPSGHATFGAAAFEITRRFFGVAPGAQDNLF
FNTISDECDGRAIAEDGSFRGRQRRHHDSLLRGMEDNAVSRVYLGVHRREDGIGDNVTTHQDILNDNSNIGGVPLG
RALAHDIFNNGLAKSAAARAVITPKNLAPVP
SEQ ID NO: 77-DNA sequence encoding a nonspecific or partially specific Bromine-lodine
haloperoxidase of Ascophyllum nodosum codon-optimized for plant expression
ATGCAAACTTGCTCAACCTCAGATGATGCTGATGACCCTACACCACCTAACGAGCGGGATGACGAGGCATTCGCTA
GCAGAGTAGCAGCTGCCAAGAGAGAGCTCGAGGGTACTGGCACAGTCTGCCAGATCAATAATGGAGAAACTGACCT
GGCTGCGAAGTTCCACAAGTCACTCCCTCACGATGATTTGGGACAGGTTGACGCTGATGCTTTTGCCGCTCTGGAA
GATTGTATCCTTAATGGAGATTTGTCTATCTGTGAGGATGTGCCGGTGGGGAACTCTGAGGGAGATCCTGTAGGTA
GATTGGTAAACCCTACAGCTGCATTTGCAATTGACATTTCAGGGCCGGCATTCAGCGCCACCACAATCCCCCCTGT
TCCTACCCTTCCGTCGCCTGAATTGGCGGCTCAACTGGCTGAAGTCTATTGGATGGCACTGGCAAGAGATGTACCG
TTCATGCAATATGGAACAGATGATATTACCGTGACCGCCGCTGCTAACTTGGCTGGAATGGAGGGTTTCCCAAACC
TCGATGCTGTTTCTATTGGTTCGGACGGCACTGTCGACCCCCTCTCCCAATTATTCCGAGCTACCTTTGTAGGTGT
GGAGACTGGACCTTTTATTAGTCAATTGCTGGTCAATTCATTTACGATAGATAGTATTACTGTTGAACCTAAGCAG
GAGACCTTTGCTCCAGATGTGAACTACATGGTTGACTTCGATGAATGGCTCAACATTCAGAACGGTGGCCCCCCAG
CCGGACCGGAGCTTCTCGATGACGAGCTCCGGTTCGTCCGGAACGCCCGGGATTTGGCAAGAGTTACCTTTACCGA
TAACATCAACACTGAAGCTTACCGTGGGGCTCTCATTCTGCTTGGGTTGGATGCTTTCAACAGGGCGGGTGTTAAC
GGACCTTTCATTGACATTGACCGGCAGGCTGGCTTCGTGAATTTCGGGATTTCTCACTACTTTCGTCTGATCGGAG
CAGCCGAGTTAGCACAAAGGTCTTCTTGGTACCAAAAGTGGCAAGTACACAGGTTTGCTAGGCCTGAAGCGTTAGG
AGGTACTCTCCATCTAACCATAAAAGGAGAGCTCAATGCGGACTTTGACTTGAGTTTGTTGGAGAACGCTGAGCTG
TTAAAACGTGTTGCTGCGATTAACGCAGCACAAAATCCAAACAACGAAGTCACGTATCTTTTACCTCAGGCGATTC
AGGAGGGTTCTCCCACTCATCCATCTTATCCTTCTGGACACGCAACTCAAAACGGAGCATTTGCTACTGTTCTTAA
GGCCCTCATTGGCTTAGATAGGGGTGGCGATTGTTACCCTGATCCTGTTTACCCAGACGATGACGGTTTGAAACTA
ATCGATTTTAGGGGGAGTTGTCTTACTTTCGAGGGTGAGATAAACAAATTAGCCGTTAACGTAGCATTTGGCAGAC
AGATGCTCGGGATCCATTATCGTTTCGATGGGATCCAAGGACTTTTGTTAGGGGAGACGATCACGGTTCGTACTCT
CCACCAGGAATTAATGACATTTGCTGAGGAGTCAACGTTCGAATTTAGACTCTTCACTGGTGAGGTCATTAAGTTG
TTCCAGGATGGAACATTTACCATCGATGGCTTTAAGTGCCCTGGCCTTGTGTATACTGGGGTCGAGAACTGTGTTA
GCTAATAG
SEQ ID NO: 78-Protein sequence of a nonspecific or partially specific Bromine-lodine
haloperoxidase of Ascophyllum nodosum
MQTCSTSDDADDPTPPNERDDEAFASRVAAAKRELEGTGTVCQINNGETDLAAKFHKSLPHDDLGQVDADAFAALE
DCILNGDLSICEDVPVGNSEGDPVGRLVNPTAAFAIDISGPAFSATTIPPVPTLPSPELAAQLAEVYWMALARDVP
FMQYGTDDITVTAAANLAGMEGFPNLDAVSIGSDGTVDPLSQLFRATFVGVETGPFISQLLVNSFTIDSITVEPKQ
ETFAPDVNYMVDFDEWLNIQNGGPPAGPELLDDELRFVRNARDLARVTFTDNINTEAYRGALILLGLDAFNRAGVN
GPFIDIDRQAGFVNFGISHYFRLIGAAELAQRSSWYQKWQVHRFARPEALGGTLHLTIKGELNADFDLSLLENAEL
LKRVAAINAAQNPNNEVTYLLPQAIQEGSPTHPSYPSGHATQNGAFATVLKALIGLDRGGDCYPDPVYPDDDGLKL
IDFRGSCLTFEGEINKLAVNVAFGRQMLGIHYRFDGIQGLLLGETITVRTLHQELMTFAEESTFEFRLFTGEVIKL
FQDGTFTIDGFKCPGLVYTGVENCVS
SEQ ID NO: 79-DNA sequence encoding a nonspecific or partially specific Bromine-lodine
haloperoxidase of Bradyrhizobium sp. B039
ATGAGCTACGGACTTCGGCACGTCCAACTGGCAGCTTGCGCCGACTATAGCCGACTGCACAATGCGGCGCGAACGG
TAAGCCAAGCCTACCCATTCGGGACACCACTGTTCTGCCGGCCGAGAAAAATCTGCTTCTCGGTTGAACCGCGTGC
GGCAAGTGAAGCAGTTCACAATCAAGCCACATCAAGATCGCTAGAGTCCGCGGGCAGCGCGACGCGACGTCGGCCA
GAGCCGCCTCGCGAAACGTCCTTCAGATCGAAAGGGCACGCCATGAAATGCTCCTCGTGGACCATCTCCCGCTTAT
CGCTGATTGTAGCGCTGATCGCGGCCTCGGCGTCGCTGGCACGCGCCGACGTGATCATGGACTGGAATGCAAAGGC
CGACGCGATCGCTGCCGAGAAGCAAATTCTTCCAGCGCCCCACAGCCGCGTCTTGTCCATGATGCACGTCGCGATG
TTCGAGGCGGTCAACGCGATCGACCGCCGGTACGCGCCCTACAAGCTCTCCTTGCCCGCGGATCGTTCGACGTCGC
GGGAAGCTGCGGCGGCAGTTGCCGCCCACGACGTGCTGCTGTCGGTCTATCCGGACCTCAAGCCGGACCTGGATGC
GACGCTGACGAATTCTCTGGCGCCGATTGCCGACGGCGAGTCCAAGATCGCCGGCATCAGCCTCGGGAAAGAGGCC
GCAATGCAGATCATCGAGCTTCGTGCGAACGATGGCAGCGCCGCTCCGGAGACCTACCGCCCCCTGACGACACCGG
GCGCGTACGTTCCGACCACCGTTCCGCTGTTCTCAACCACGGGCGCGACGACGCCCTGGGTCATGGTTTCCGGATC
GCAGTTTCGCCCCGGCCCGCCGCCGGCGCTTGGTTCAGAGGTGTGGACCCGGGATGTCAACGAAATCCGCGAGGTC
GGCAGCCGAAGCAGTACAACCCGGACGCCGGAGCAAACGACGATCGGCCGATTCTGGTTCTTTGTCGGTGCCCGCA
CCTACAATCCGATCGTGAGGCAAGCTGCAATGGCCAAGGGCATGGATCTCATCGACTGCGCCCGGCTGTTCGCGCT
GACGTCAATTGCCGGCAACGACGCCCTCGTTGCGGTGTTCGATGCAAAATACCATTACAATCTCTGGCGACCGATC
ACCGCCATACGCAACGCCGACCTGACGTCGAATCCGGCGACGCCACGCGACCCGTCCTGGCTGCCACTCGGCGAAA
CGCCGATGCATCCGGAATATCCCTGTGCTCACTGCATCACGTCGGCGGCGATTTCGACCGTGCTCCAGAGCGTCGT
TGGAGATTTCGGCGAGTTCTCGCTGACCAGCTCCACGGCACCAGGTGTCACGCGCAAATGGTCGCGGCTCCAGGAC
TACAGCGACGAAGTCTCCAACGCCCGCATCTGGGCCGGCTTTCACTACCGGTTCTCGACCGAGGTCGGCAAGGACA
TGGGTCGGAAGATCGGCGCACTGACCGTCGCGACCCAGCTTCGCGGTGTGGAAGCGATGGCGGAGCCGAAGCGTTA
A
SEQ ID NO: 80-Protein sequence of a nonspecific or partially specific haloperoxidase of
Bradyrhizobium sp. B039
MSYGLRHVQLAACADYSRLHNAARTVSQAYPFGTPLFCRPRKICFSVEPRAASEAVHNQATSRSLESAGSATRRRP
EPPRETSFRSKGHAMKCSSWTISRLSLIVALIAASASLARADVIMDWNAKADAIAAEKQILPAPHSRVLSMMHVAM
FEAVNAIDRRYAPYKLSLPADRSTSREAAAAVAAHDVLLSVYPDLKPDLDATLTNSLAPIADGESKIAGISLGKEA
AMQIIELRANDGSAAPETYRPLTTPGAYVPTTVPLFSTTGATTPWVMVSGSQFRPGPPPALGSEVWTRDVNEIREV
GSRSSTTRTPEQTTIGRFWFFVGARTYNPIVRQAAMAKGMDLIDCARLFALTSIAGNDALVAVFDAKYHYNLWRPI
TAIRNADLTSNPATPRDPSWLPLGETPMHPEYPCAHCITSAAISTVLQSVVGDFGEFSLTSSTAPGVTRKWSRLQD
YSDEVSNARIWAGFHYRFSTEVGKDMGRKIGALTVATQLRGVEAMAEPKR
SEQ ID NO: 81-DNA sequence encoding a nonspecific or partially specific haloperoxidase of
Fucus distichus
ATGCAGTGGCAGGTCAACGGAGCGTCGCAAATCGGTGTTCCGCCGCGGCTGTATAATCAAATCGTTCGTGAGATAA
TTGGGAACAATGTGAACGGAGCTGAGCGGGCGGCGGCCTCGGCGCGTCTTTTAGCGCTCGTCAACGTCGCGATGGC
GGACGCAGGTATCGCTTCTTGGTACTACAAGTACACGTACCAGCTTTGGCGCCCTGTATTAGGAATTAGAGAATAC
GACGATAGTTATTGGTACAACGGCACCGCGGTTTCTCACGCGCTTCACAAACGGTGCGATCCTTGGTGGATACCCC
TAGGCTCCCCAAGAACGAACGAGTCTGGTCGACATAGCTTTACTCCTCCCTTCCCCGCCTATCCTTCGGGGCACGC
TACGTTCGGTGCCGCTGCCTTCGAAATAACTCGGCGTTTTTTCGGGGTCGCACCCGGTGCCCAAGACAATCTATTC
TTTAACACAATATCGGATGAGTGCGACGGCCGAGCCATTGCCGAAGATGGCTCTTTCAGGGGGCGTCAGCGGCGGC
ACCACGATTCCTTGCTCCGTGGAATGTTCGACAATGCGGTGAGTCGCGTGTATCTCGGTGTTCACCGGCGCTTTGA
CGGCATTGGAGACAACGTTACCACTCACCAAGATATCTTGAATGATAACTCGAATATTGGCGGCGTACCCTTGGGA
CGCGCTCTAGCCCACGATATTTTCAACAATGGCCTTGCAAAGAGCGCTGCAGCGAGAGCGGTAATCACTCCCAAAA
ATCTCGCTCCGGTACCTTGA
SEQ ID NO: 82-Protein sequence of a nonspecific or partially specific haloperoxidase of
Fucus distichus
MLCHAADTTRGSPMPDTGVLRLLTSEQRAKGWRRQLEGEKSLGFHPSETPYIKYLEGSETWKKVKLPTDGISASKI
LGKIMARVRIATALAVVLAAPCLAFDEVTASGVFPEEHKHTGEGRHLQTCTNSDDALDPTAPNRRDNVAFASRRDA
ARRERDGTGTVCQITNGETDLATMFHKSLPHDELGQVTADDFAILEDCILNGDFSICEDVPAGDPAGRLVNPTAAF
AIDISGPAFSATTIPPVPTLSSPELAAQLAELYWMALARDVPFMQYGTDEITTTAAANLAGMGGFPNLDAVSIGSD
GTVDPFSQLFRATFVGVETGPFVSQLLVNSFTIDAITVEPKQETFAPDLNYMVDFDEWLNIQNGGPPAGPEELDEE
LRFIRNARDLARVSFVDNINTEAYRGSLILLELGAFSRPGINGPFIDSDRQAGFVNFGTSHYFRLIGAAELAQRAS
CYQKWQVHRFARPEALGGTLHNTIAGDLDADFDISLLENDELLKRVAEINAAQNPNNEVTYLLPQAIQVGSPTHPS
YPSGHATQNGAFATVLKALIGLDRGGECFPNPVFPSDDGLELINFEGACLTYEGEINKLAVNVAFGRQMLGIHYRF
DGIQGLLLGETITVRTLHQELMTFAEEATFEFRLFTGEVIKLFQDGTFSIDGDMCSGLVYTGVADCQA
SEQ ID NO: 83-DNA sequence encoding a nonspecific or partially specific haloperoxidase of
Phytomonospora endophytica
ATGCACAAGACGTCCCGCAGGCGACTCATCGTCGCCGCCGCCCTGACCCCGGCGGCGCTCGCGCTGCCCACCCCCG
CCCACGCCGACACGATCGACCCGGTCGCCGAGTGGTACGACCTGACCGCCGACGCGGTCACCACGTCCGGACCGTC
CGCGCAGGTCAGCGGCAGCCGGATCTGGGCCCTGGCCTGGCTTTCGGCCGCCCGCGCGCTGCACGGCGACTCGCAC
CCCGACCGGCAGAGCGCCGCGCTGGTCTCCGCCGTCCACGCCGTGCTGCGCGACCAGATCCCGTCCCGCGCGACGG
ATCTCGATGCCGCGCTGGAGGCGTCGCTGGCCCGCGTTCCCGACGGCTCCCCGAAACGACGCGGAACGGCGGCGGG
CCGTGCCGAAGCCCGGTCGCTCCTCACCGGCCGGGCCGGTGACGGTCTCGACCCCGCGTCGGTCAACGCGCCCTAC
ACCCCCGACCCGCCGACACCGGGACGATGGCAGCCGACGCCACCGGCCTTCGGTCCTGGACAGCAGTCCGGAACCC
GTCACGCCAGGCCGTTCCTGCTCGGCCGCGCCGACCGCTTCCGCCCACCACCGCCGCCCGCGCTCGACTCCGCCCG
CGATGCCCGCGACCTCGCCGAGGTCCGTGCCTTCGGCGCGCTGGACAGCACGGTGCGAACCCAGGCCCAGACCGAC
ACCGCGCAGTTCTGGTACGGCTCCTCCCTGGTGCTGTACAACGGGATCCTGCGGGCCGCGCTACTCCAGTCCCGGC
GGTCGGCGTACCGGCGCGCGCGGCTGGTCGCGCTGTTCCACGTCGCCCTCGTCGACACGCAGATCGCGACCTCCGA
CGCCAAGTACCACCACCGCTCGTGGCGGCCGGTCACGGCGATCCGGGCGCTGCACGATCCACAGTGGACGCCGTAC
CACGTCACGCCCTCGCACCCCGACTACGTCTCGGGCCACAACACCTACTCCGGCGCCGCCGAGGCGGTGCTCGCCG
AACTGGCGGGCCCCGCGCGGCCTTATACGATCGGCAGCCCCAGCGCGCCGGGTGTCACCCGCACCTACACCGACTG
GTCGACCCCGTCGCGCGAGAACGTCGACGCTCGCGTCTGGTCGGGGATCCACACCAGGACCGCCGACGAGGCCGGG
ATCATCCTCGGCCGGAAGGTCGCCCGCCACGCCCTGAGCCGCGCCGAACGGCTCTTCGACTAG
SEQ ID NO: 84-Protein sequence of a nonspecific or partially specific haloperoxidase of
Phytomonospora endophytica
MHKTSRRRLIVAAALTPAALALPTPAHADTIDPVAEWYDLTADAVTTSGPSAQVSGSRIWALAWLSAARALHGDSH
PDRQSAALVSAVHAVLRDQIPSRATDLDAALEASLARVPDGSPKRRGTAAGRAEARSLLTGRAGDGLDPASVNAPY
TPDPPTPGRWQPTPPAFGPGQQSGTRHARPFLLGRADRFRPPPPPALDSARDARDLAEVRAFGALDSTVRTQAQTD
TAQFWYGSSLVLYNGILRAALLQSRRSAYRRARLVALFHVALVDTQIATSDAKYHHRSWRPVTAIRALHDPQWTPY
HVTPSHPDYVSGHNTYSGAAEAVLAELAGPARPYTIGSPSAPGVTRTYTDWSTPSRENVDARVWSGIHTRTADEAG
IILGRKVARHALSRAERLED
SEQ ID NO: 85-DNA sequence encoding a nonspecific or partially specific haloperoxidase of
Cellulomonas xylanylitca
ATGCACTCACGTCGTCGCATCGTCGCGGCTGCCGCTGCGGTTCTCCTGGCACCCATGATCGCGGTGCCGGTCGTCG
GATCGGCGGCGAGCGCCAAACCGCTCGGATCATCGATCGCCGTCGACTGGCAGCGGACGGCGAACCGCACGATCTA
CACCGAGGCCGGCAGTGCGCCGCCGCTCGGCGCGCTCTACCTCGCCTTCACGTCGCTCGCTGTCCACGACGCTGCG
ACGAAGGCACAGCGGCACGGCATGCACGCAGCCGCGGCAGCGGTGGCGACGGCCGCGCACGACGTGCTCGTCGAGT
ACTTCCCGGCATCCGGGGCGGCACTCGACACCGACCTCGCCAACAGCCTGGCGAGGGTCCGCGACGGCGCGAAGCA
GGACGCCGGTGTGGCCATCGGTGCCGCCGCCGCGGACAGGATGATCGCCTCCCGCGTCGACGACGGCCGCTTCGAC
CCCGCGTACGTCTACAGCAAGCCGCCCGCTCCTGGGGTGTGGCAGCCTGCGCTGGGGGGCGCGATGGCCCTGCCCT
GGCTGGGCTACGTCGACCCGGTCGTGCACATCCGCCCCGTTTTCCTCGACGGGCCGGACCCCATCACCAGTGCCGC
CTACGCGCGGGACTACGACGAGGTCCGCGCGGTCGGCGAGGTCGGCTCGACGACCCGCACCAGCGACCAGACGGCG
ATCGCGCAGTTCTTCGCGAACAACCCGATGGTCATGTACCGCACGGCAGTGTGCGACCTGCTCGCCGCCGAGCCGC
TGGGTCTGCTGCCGACGACCCGCCTCTTCGCCCGGATCGACGCGGCCGTCGTGACCTCGTTCATCGCGAGCTGGCG
GCTCAAGTACGAGATCGGGTATTGGCGGCCCTTCCAGGCCATCGCTGGCGCAGACACGGACGACAACTCCGCGACC
ACGCCCCAGGACAACTGGGTGCCCTTGGTGACCAACCCGGCGTACGCCGACTACACGAGCGGGCACGCCAACGCGA
CGTCGCCGTTTGCACAGGTGCTGCGCCGAACGCTCGGTGACGACACCGGGCTGGTCCTCAGGGCCGGGCCCATCAC
CCGCAGCTATGCCACCTTGACCGCGCTGGAGCACGACGCCCTCAACGCCCGGATCTGGGGTGGCCTCCACTTCCGG
GACGCCATGGACGACGGCTACTACCTCGGCCACACGACTGCGGACCGCGTGATGGCGGCCGTCCACTGA
SEQ ID NO: 86-Protein sequence of a nonspecific or partially specific haloperoxidase of
Cellulomonas xylanylitca
MHSRRRIVAAAAAVLLAPMIAVPVVGSAASAKPLGSSIAVDWQRTANRTIYTEAGSAPPLGALYLAFTSLAVHDAA
TKAQRHGMHAAAAAVATAAHDVLVEYFPASGAALDTDLANSLARVRDGAKQDAGVAIGAAAADRMIASRVDDGRED
PAYVYSKPPAPGVWQPALGGAMALPWLGYVDPVVHIRPVFLDGPDPITSAAYARDYDEVRAVGEVGSTTRTSDQTA
IAQFFANNPMVMYRTAVCDLLAAEPLGLLPTTRLFARIDAAVVTSFIASWRLKYEIGYWRPFQAIAGADTDDNSAT
TPQDNWVPLVTNPAYADYTSGHANATSPFAQVLRRTLGDDTGLVLRAGPITRSYATLTALEHDALNARIWGGLHFR
DAMDDGYYLGHTTADRVMAAVH
SEQ ID NO: 87-DNA sequence encoding a nonspecific or partially specific haloperoxidase of
Nostoc sp. P10264
ATGCAAGTAGATCCTAATTCTGATCAAGGTTTCGAGCAAAATGCTCAGGCTAATAACGAAATAGATAAACTGAAAC
AACCCCGTGTAGCTAGCAGACGTTTTTTTGGTACTCACGCCAGTAGACGCTCATTTCTCGGTCGCGCTGGTTTGTT
TAGTGCCGCTAGCGTTGTTGCAGGCGTTTTAGATTCACCTTTCTCCTCAAAAAAGGGAGAAGATATTGTACAAGCC
CAAGACATCAACAGGCTCCCTAATCGACCTTTCGACTACAAGAGCTTTGTTAATAGAGCTTATCGAGTACGTGTTG
AAGCAGCCAGACTTCAACAGAGTATTCCGATTCCGCCGCATCCGACCAACGGGGATGAGGCGAAGTATGCTAACAA
AATTGCGACGGATACACGAGGATTACCCCATAACAAGCTGGGCGAAGTGGATCTGAAGGCTTATCAATCTTTTATT
AATGCGTTGAACTCCGAAGATCCCAATGAGTATGAAAAGATCATCCTGGGTGGCAAACGGAAGCTGCTACAACCGC
TCGGTCCATTAGCGGTTAGCTTGAGTGGGCTGAATGCGACCCAATTGACAATTCCAGTGCCACCTACCCTCGATAG
TGCAGAGCAGGCGGCTGAATTCCTCGAAATTGCTTGGCAACAACTGTTGCAGGATATACCCTTTAGCGAGTTCCAC
AATAATACAACTAACCCGCTGATTCTGGCAGCAGTTCAGGATCTGAATCGGTTGTCAGCATTCAAAGGGCCTAAAC
AGAACGGACGGGTTACACCCGAACTCCTATTTCGAGGCACCGCGATTTACGTTAATGCCTCTGGTGCAACGAGTTA
CTACACGTTACCAGGGGTAGATGTGGGCCCATACGTCTCCCAGTTTTTACTGCGTCCTGTGCCCGCAGGAACACAA
AGTTATCTTCAGCTTAACCGTGTTCCCCTAGCTATTCCCGAAAACAGTTTTCAAACCAACTATAACGAGTGGTTGC
TGGTGCAAAATGGTGGCGATTCTGGTCGAACGATTAAGTTTGACCCAACCCCTCGCTACTTTATCAACGGGCGCGA
CCAGTCAGAGATTGCCCACACGCCACCACCCGTCTATACAAATGCTGCTCTAATTCTACTCGCCAAGCCAGCCCTT
GATAACCTCCTAGCTGGTGGCGTTGGCTCACCTTACAATCCCGGTAATCCTTATAACAACTCGAAGACCCAAGCGA
GTGGTTCTGCGACATTTGGTCCAGGGTATTCACAATCTATCATCTCTTGGGTTAGTCCACATGCAATCAGAGCAGC
TTATTGGCAGAAGTACTATGTTCACCGTCGTCTGCGAGCAGAGGCTTATGGTGGGTTGGTTCACAACAACAAGGTG
AATAAAACAAACTATCCGATTCACCCCGATGTTTTCCAGTCAGAAGCACTTGATCGCTTCTTCAGCAAAAACAAAT
CCTATCTGCTACCGCAAGCTTTTCCTGAAGGGGCACCATTCCACTCTTCTTATCCAGGGGGCGCTTCGGTGTCCGC
AGGTGCCAGTGTCACGATTTTGAAAGCACTATTTGATGAGAATTATGTGATTCCGAATCCGGTGGTACCCAATCCC
AAAGATCCCACCCAGTTGATTCCCTATCAAGGCCCACTGCTGACCGTGGGCGGCGAATTGAACAAACTGGCGGCAA
ATATTGGCTTGGGTCGTGATAGTGCTGGGATTCACTGGCGCACAGATGCAGCAGCGTCGTTGGCCCTTGGCGAAGC
GATCGCCATCAGCATTCTTAAGGATGAAAAGCTAACGTTCCGCGAAAAATTTCAAGGTTTCACGTTCACAAAATTT
AATGGTACAAGAATCACCATTTGA
SEQ ID NO: 88-Protein sequence of a nonspecific or partially specific haloperoxidase of
Nostoc sp. P10264
MQVDPNSDQGFEQNAQANNEIDKLKQPRVASRRFFGTHASRRSFLGRAGLFSAASVVAGVLDSPFSSKKGEDIVQA
QDINRLPNRPFDYKSFVNRAYRVRVEAARLQQSIPIPPHPTNGDEAKYANKIATDTRGLPHNKLGEVDLKAYQSFI
NALNSEDPNEYEKIILGGKRKLLQPLGPLAVSLSGLNATQLTIPVPPTLDSAEQAAEFLEIAWQQLLQDIPFSEFH
NNTTNPLILAAVQDLNRLSAFKGPKQNGRVTPELLFRGTAIYVNASGATSYYTLPGVDVGPYVSQFLLRPVPAGTQ
SYLQLNRVPLAIPENSFQTNYNEWLLVQNGGDSGRTIKFDPTPRYFINGRDQSEIAHTPPPVYTNAALILLAKPAL
DNLLAGGVGSPYNPGNPYNNSKTQASGSATFGPGYSQSIISWVSPHAIRAAYWQKYYVHRRLRAEAYGGLVHNNKV
NKTNYPIHPDVFQSEALDRFFSKNKSYLLPQAFPEGAPFHSSYPGGASVSAGASVTILKALFDENYVIPNPVVPNP
KDPTQLIPYQGPLLTVGGELNKLAANIGLGRDSAGIHWRTDAAASLALGEAIAISILKDEKLTFREKFQGFTFTKE
NGTRITI
SEQ ID NO: 89-DNA sequence encoding a nonspecific or partially specific haloperoxidase of
Skeletonema marinoi
ATGACCGACCCACCACCACCACCACCACTGTCAACGTCTCCCTTCACATTCTCCACCACTGACAACAGCGATGGAC
GCACCAACTTTGAACGCCCAGTCGCGTATGCCGAGGCATATGTCGAGGAAATAGAGACGTATTATCATGTGAACAA
CCGCAATGTTGATTCCGATTATGCACCACCAGACGTGAACAATCTATCATCCAATGATGATGATGATGATGTGAAC
TCGAACTCGAAGCCGAAGAAAGTTTACTATCAAATAGCAGGGATGGTGCTGCTGCCGTTGCTTCTTTACGCCGGAT
ACACACTTGGTGCTAAAACCAGCAGTCAAAAGATTGCCAGCGCCGTCGCTGTAGCAAATGTTACAATAAGCAATGA
TCCCATGTCAAAAACACCCAAGGCAAAAACACCCAAGACAACAGGAGCAGGAGGACCCATAGCCAAGCAAGATAAG
AAAAGGCGCGATAGCAACGATCGGGATGTTTGTCTAGAGTGGAATGCTATCGCAATCACATCCAACATCTTCGATC
ACAAAGGAAAGGCAAAGTCAGCGCAAGACCTTTCCTTGACGATGGGGCCAGTTGCATCAGCTCGCGCATTTGCAAT
GATGCACACGGCCATGTTTGAGGCATACAATTGTGAAAAAAAGACTCATGATAACTCTTACCCAGCTGGTCATCCC
ATTCATAAAATCAATTGCACTGGAGTTTCGACATTATGTACAGTTGCGCAAGCCGTCCATGACGTAATGTGTGGAG
TAAAAGGTGAAGATGACATCATTGGCCTTTTCGAGGAAGATCAGGATGACCAGGATATTTGTGACATGGCCGACAC
CGCTCTTCGAAATGCACTCTCAAGAGAAGAAGACAAAGATAAGTGTAAAAAAGGCACCGAGGCAGGCCGTGATATC
GCTAAATATGTACTTATGGAAAGGGCAAATGATGGCTACGGAACAGAAAATCGGACCTTGAAAGATGAAATGTATG
TCCCCCCAAGTGGAAACAACCCTGGATGTCACATGCCAGATGCCGAGAACCCCGAACAGGGCGTCCTTGGGGGGGG
GTTTGGTAAGGTGAAAGCATTCTTTCTCACGAGGGAGGAGCTCATGCAATTCTCAGGGGAGGATGCGCCTGGTGTC
ACCGTCAATGAAAGGACAGGTGAGGTCAAATTTGACTTAAATGATACAGAGTATCTCGCCGCTTTGATGGAAGTAA
GAGAGTTGGGAGTATTCCGTGGTGGAACATCTGGAGAATATGCTCCCACGGATGACGAAACAGTGGTTATTGGACA
GTATTGGTCTGCTGACGGAAGTCCACATATAGGTACGCCAATCGCTTTGTTAAATGACATGACACGGAAAATTGTC
ATGGAGGTCGATATTGACGAGAAACAAAGCTGTTACTTATTTCCATTGCTAAGTGTTGTTATGGGTAATGTTGCAA
TTGCTGTGTGGCATAAGAAGTTCAATAACAATCTTTGGCGGCCACTTTTGGCAATCAGCTCATCGTTTCCTGAATC
AGCATGGGCGAGAATGGGGGCCTCACGAGCGAATCCGTTCGACAACGAAACTAATTTTGACCCTCCCTTCCCATCA
TTCCCATCTGGTCATGCAGCCTTTTGCTGTGCGACGTTCCAGACGATTGCTAACGTCTACCAAACGCATAACATCA
CATTCAATTACACATCACCCGAGGTGAATGGTGAAACTTATGACCAGTTCAACCGAAAACGCAAGTGTATCATACG
TGAATTTCCCACGCTGAAGCATGCAATGGCTGAGTGCGCCGCCTCTCGTGTTTTCAATGGAGTTCATTACTCCTTT
GATGGGGATGGGGGTTGTAAAGTTGGCATCGATATTGCTAACTACGTGTACGAGAACAAGTTCCTGCCTCTGGACG
GAAGCCAAAAGACTTCGATGCCGGATATGAGGGAAGAAATTTTTGATGAAATGAAGAAATACTTGAACAATACTGC
AACAAGTGGCTATTCTCCAAACTTTTGCAGTAAAGCTCCTGAATATCCCTTGCCCCAACCTTAA
SEQ ID NO: 90-Protein sequence of a nonspecific or partially specific haloperoxidase of
Skeletonema marinoi
MTDPPPPPPLSTSPFTFSTTDNSDGRTNFERPVAYAEAYVEEIETYYHVNNRNVDSDYAPPDVNNLSSNDDDDDVN
SNSKPKKVYYQIAGMVLLPLLLYAGYTLGAKTSSQKIASAVAVANVTISNDPMSKTPKAKTPKTTGAGGPIAKQDK
KRRDSNDRDVCLEWNAIAITSNIFDHKGKAKSAQDLSLTMGPVASARAFAMMHTAMFEAYNCEKKTHDNSYPAGHP
IHKINCTGVSTLCTVAQAVHDVMCGVKGEDDIIGLFEEDQDDQDICDMADTALRNALSREEDKDKCKKGTEAGRDI
AKYVLMERANDGYGTENRTLKDEMYVPPSGNNPGCHMPDAENPEQGVLGAGFGKVKAFFLTREELMQFSGEDAPGV
TVNERTGEVKFDLNDTEYLAALMEVRELGVFRGGTSGEYAPTDDETVVIGQYWSADGSPHIGTPIALLNDMTRKIV
MEVDIDEKQSCYLFPLLSVVMGNVAIAVWHKKENNNLWRPLLAISSSFPESAWARMGASRANPEDNETNFDPPFPS
FPSGHAAFCCATFQTIANVYQTHNITFNYTSPEVNGETYDQFNRKRKCIIREFPTLKHAMAECAASRVENGVHYSF
DGDGGCKVGIDIANYVYENKFLPLDGSQKTSMPDMREEIFDEMKKYLNNTATSGYSPNFCSKAPEYPLPQP
SEQ ID NO: 91-DNA sequence encoding a nonspecific or partially specific haloperoxidase of
Actinarchaeum halophilum
ATGAGCAGAGAAAACGATACAACTCGATCAGATCCAGATCTATCGTCACCGGAAGAGTCGAAATCGAACGTGCCGA
TGATCGACCGACGGAAGCTGCTACGCGGGATCGCAGTCGCGGGTGGGTTCGCTGCTGGTGGCCCACAGTTGTTCGC
CCAGGAGGCGGATGCCTATGATGGTGAGTACGACGACGTGTATGAGCTCCAGGATCGCCTCGAAGAGGCAATTGCC
AGTCGGCAGGAGACGATTCAGCAGTTGCTCGTCAATCTAACGACAATCGAATCGGATTACGACAGCACGTATTTCG
ATCTTACGAAGAAGATGCACGGAATCGAAATGTATCCATCGTTGTTCATGAAGGGAATGTCGATGGAGACGAACAG
TAACGATGACCCATATCCAGAAGGCTTCGTTTCCGTCGGGGCCCACGAGGCCGTCATGAGTCCGATTCTTCGGAAT
CGGGATGAATATAATAATATCCCACAGGGTGACGAACGCCGGTTTATTAGTCCCCAGTCAGTCCATTCTTTCCCAA
CGGAGGGGATGGATTCGTGGTCCGGAACGATGCCACCCGCACCATCTCCCGGCAGCGACCATATGGCCGGGGAGAT
GGTCGATCTCTATTGGATGGCGGCCCTTCGTGATCAGCCGTTCGATGCCTATAGTGGGACGCTCGACGGTGCGATC
ACGAAATCGGATATCAATGCAGATATCACCACCATAGCGGACGCGATACAAACCCCGTGGTGGCACAATACTGGCT
ATCTGTTCGTGGAAGCAGCGACCGACGAACTTGATTATGGACCGTACCTCTCTCAGTTTCTGATTCAGGATGTAAT
GTTCGGTGCATTCCCGATTAGTCCAGAGATCAAGACGTTCCAACAGGGTCAAGATTATCGTACAACACGCCAGGAA
TGGTTACGACTGCTAGCAGGAGAAGGCCAGGGAGCAGAGTCCTCACCGCCTGCATCAAGGTTGACTGAAGGGCCAC
GCTACATTGCTACAGGACGCGATCTCGCGGCGTTGGTGAATTTCGATCCGTCATACCACGTTTATGTCATGGCCGC
ACTCACGCTCCTGGATCAGGGGATCTCAATGAACCCAGCACTCCAATACATGACAGACGCAGGAAACAAGATATTC
GATTACATCGACGGTGGACCTGTAGCGCTGTTGGATCTCATTGGTCGAGCTGCACGTAACGCTCTTCTCACAGCGT
GGTATCACAAGTGGCGCGTCCATCTCCGCCTTCGTCCCGAAACGTTCGCCGGTCGAATTCACTCCCAACGGATAGA
TAGTCGATATTATGGCATTTCAGATCTCGTTTTCGAATCGAATGCGCTCACCGAAATCGAGGAGAACACCGGGACT
GCCTTTCTCCCGCTGGCGTACAAGGAGGGGGCACCGGTACATCCGGCGTATCCCAGTGGACACTCAACTATCGCAG
GAGCCTGTGGAACCGTTCTGAAAACCTTTTTCAAGAACGAAGACTGGCCTGGAGAGCTGTTCGTTCCAGTCGATCA
TGGAGCCAGCCGAACGACCGTTCCCGTCCCAGCGAACCATCAGGGGGTCCATCAGGAGATTGATAAACTGATGTCG
AATATGGGTCTCGGACGCTTATTCGCTGGCGTTCACTACTACAGCGATCACTACTGGGGAATCAAACTCGGAGAAC
AAACAGCCGTTGCCATGTTACGAGACGTCCTCGATCAAGCATACACTGACGGTCGAGATGTCACTCCAACCTTTAC
CGAATATTTCGGAGATTACAGCAACCCACTGGAGATCTCGATCGAGGAATTAGAGAATCTTCGCGAGAGTGCCACA
CACGGTCTTACATATCCAAAATCGTAA
SEQ ID NO: 92-Protein sequence of a nonspecific or partially specific haloperoxidase of
Actinarchaeum halophilum
MSRENDTTRSDPDLSSPEESKSNVPMIDRRKLLRGIAVAGGFAAGGPQLFAQEADAYDGEYDDVYELQDRLEEAIA
SRQETIQQLLVNLTTIESDYDSTYFDLTKKMHGIEMYPSLFMKGMSMETNSNDDPYPEGFVSVGAHEAVMSPILRN
RDEYNNIPQGDERRFISPQSVHSFPTEGMDSWSGTMPPAPSPGSDHMAGEMVDLYWMAALRDQPFDAYSGTLDGAI
TKSDINADITTIADAIQTPWWHNTGYLFVEAATDELDYGPYLSQFLIQDVMFGAFPISPEIKTFQQGQDYRTTRQE
WLRLLAGEGQGAESSPPASRLTEGPRYIATGRDLAALVNFDPSYHVYVMAALTLLDQGISMNPALQYMTDAGNKIF
DYIDGGPVALLDLIGRAARNALLTAWYHKWRVHLRLRPETFAGRIHSQRIDSRYYGISDLVFESNALTEIEENTGT
AFLPLAYKEGAPVHPAYPSGHSTIAGACGTVLKTFFKNEDWPGELFVPVDHGASRTTVPVPANHQGVHQEIDKLMS
NMGLGRLFAGVHYYSDHYWGIKLGEQTAVAMLRDVLDQAYTDGRDVTPTFTEYFGDYSNPLEISIEELENLRESAT
HGLTYPKS
SEQ ID NO: 93-a double-35S promoter sequence used to provide constitutive plant expression
TCAGAAGACCAAAGGGCTATTGAGACTTTTCAACAAAGGGTAATATCGGGAAACCTCCTCGGATTCCATTGCCCAG
CTATCTGTCACTTCATCAAAAGGACAGTAGAAAAGGAAGGTGGCACCTACAAATGCCATCATTGCGATAAAGGAAA
GGCTATCGTTCAAGATGCCTCTGCCGACAGTGGTCCCAAAGATGGACCCCCACCCACGAGGAGCATCGTGGAAAAA
GAAGACGTTCCAACCACGTCTTCAAAGCAAGTGGATTGATGTGATAACATGGTGGAGCACGACACTCTCGTCTACT
CCAAGAATATCAAAGATACAGTCTCAGAAGACCAAAGGGCTATTGAGACTTTTCAACAAAGGGTAATATCGGGAAA
CCTCCTCGGATTCCATTGCCCAGCTATCTGTCACTTCATCAAAAGGACAGTAGAAAAGGAAGGTGGCACCTACAAA
TGCCATCATTGCGATAAAGGAAAGGCTATCGTTCAAGATGCCTCTGCCGACAGTGGTCCCAAAGATGGACCCCCAC
CCACGAGGAGCATCGTGGAAAAAGAAGACGTTCCAACCACGTCTTCAAAGCAAGTGGATTGATGTGATATCTCCAC
TGACGTAAGGGATGACGCACAATCCCACTATCCTTCGCAAGACCTTCCTCTATATAAGGAAGTTCATTTCATTTGG
AGAGGACACGCTGAAATCACCAGTCTCTCTCTACAAATCTATCTCTCTCGAGCTTTCGCAGATCTGTCGATCGACC
SEQ ID NO: 94-the 19 kDa Zein promoter used to provide seed-specific expression in monocots
TGCCAAGTGCCCGATAAAAAGCACTTGGCAAAGCGCCGAGCACTCGGAAAGAGCCGGATTCCGGTGTGGTTGTTTC
ATGGAAGTTGTAAGTAGACCATCTTAACAAGGTCAACAATAGAAGTACAACTCACAACATTAGATACTGCCAAAGT
TAATGTTGAGAGGCTTCGTGAACTCCTTATGGATTTACTAGTAGTTGAAAAACCAATACTGGTTATCTATATGAGT
TATAATAATTAAACATTGGTAATCAAAGTGAACAATTCTTAGGTTAACATGAAGACTACAAAGCATGTCAAGAACG
GTTGAAACTGTTAAAACACTAAGAAACTCTAGATTAATTGTGTTGGACTATGTCCATACTTCTAAGAATTTGGTAT
GCCAGTTTATAGAAGATTTGTCACATAATGTGATAGATGGTGCATCGAGAAAGATGGACTTGAGACCCACTTAATG
CCATTCCATAGTAGTAGCATTTTCTATGTGATTGGAGATCTCATGAAGTAAAATGGTGAAATAAGCTAGAGAATGA
CTGATGGAGAGACTCCTTAAATAAAATCTAGGTAATTTCATATGAACATTTCTCCTTCGATGTACGATAGGCTAAC
TAAAATCTTAATATGATTTGAGTGACATATCTAAGCAGGGATGTTATCTTATAGAACATCCTATAAGGAACACACC
TATATGACTTTGACTACTAATCACAGTCTATGATATGTGGGAAATATTTTTTTACTCATGAAAGGCCTCGAAGTTT
GACTTATATGCTTCAAACCAATGGATAACTGATTGAATCTGTTTCAGACAGCAGCATAGTTAACTTCAAAATTCTA
TATCTCCCAAACTTCTCAACCAAAAATTATTAAATTATGCTGACAGTAAGAACTTTTATCCCTCTACAAAAGTCTC
CATAGTTGGAAGAGCCAATTCGGCCATTTACTAGATGAAACCTACCAGTTAACAGACCCGCTCATTTTTGTCAGGC
AAACAAGAACCCTTATATATCAAAGAGTAGTGGATTTGAATTTGTCTAATATGAAGTTTATGGGCTTGGATCATTT
AAAGTATTTTATAATTCTGAAAGACAAGATTAAACTAACTATGTATGATCCTTTAGCACCGTACAGATGGTCGATG
GGACAGTTACATGGTTTATATAGATGGACCTATCTATATTCCTTCAGTGGTTGAGAACAAGGTAAAGGTGTGTCAC
ACTCACGCGTGCGCCGCCTCCGCCGAGACGAGGCATGGCGTTGCAGGGTGAGGCATGGCATGGTGCAGCACGATCG
CAGTCATGGTCGTGGCACAGTGTAGCACTGTTGTGGATAGATAGATAAACTATTGTTGTAGTTAGAGTGCATGAGT
TTTTGAATGCAACACTAGAATTTTCACAATTTAACTTAATTAAGAAGTTATATCTTGATGGCACAGAGTTACCTGA
CCATTGATCTCACATGCCCTTTTAGCTCTCTCGTACTCTAGACTTACCCTGACTATTTAGGTCTCCTGACTCGCCA
ATATAAACACCATCCTATCATTTGCTGTCGAATACTTCTGTGTCACAACTCAACTGTCAGATGAGTACTCCATTAC
AGCACTGAGCAGGCCTCCGAAATGAATATCTACTCAGATGAAGGACGACAATCGTGTTCTAGGGAAATATCAGCGA
CACAACTCCTTACTTCCTCCGCTTCTTCCTAGTGTTTTTTGTTGTGATTGAGTCGACATAGCAACAACACTGCACT
ATTACAACCAGTACGACTATATCAACTAGGCAATGTCTTCCTTATATGTTACTATTTATTTTGCTCATATTCATTA
TGTTTAAATCACATAGGCGCCTTTATGTTGGCATCAAAAAATCAGTATCAACTTTATAGATTAAAATGAAACTAAA
AGTACCTAAATTTCTATCGGTGGGGATCGAGTGATTCTTTAAACCGATTATTACACAAGTTAACCACACTAAAATT
AACAATGGTGAATCGTGCCATGATTTTTTTCTAGTGGAAAATAGCCAAACCAAGCAACACATATGTGGCTATCGTT
ACACATGTGTAAAGGTATTGCATCACACCATTGTCACCCATGTATGTGGACAATACCGAGAGGAAAAACCACTTAT
TTATTGTATTTTATCAAGTTTGTCTTGCTTACGTATAAATTATAACCCAACAAAGTAATCACTAAATGTCAAAACC
AACTAGATACCATGTCATCTCTACCTTATCTTACTAATATTCTTTTTGCAAAATCCAAAATTAATCTTGCACAAGC
ACAAGGACTGAGATGTGTATAAATATCTCTTAAATTAGTAGCTAATATATCGCACATATTATTGAGACCAACTAGC
GACATAGAAAGCACAATAGTGTACCAACC
SEQ ID NO: 95-a tricistronic DNA sequence providing expression of VBPO, AACT2, and WRI1 in
monocot seeds under the control of the 19 kDa Zein promoter, and in which the VBPO sequence
is codon-optimized for monocots
tgccaagtgcccgataaaaagcacttggcaaagcgccgagcactcggaaagagccggattccggtgtggttgtttc
atggaagttgtaagtagaccatcttaacaaggtcaacaatagaagtacaactcacaacattagatactgccaaagt
taatgttgagaggcttcgtgaactccttatggatttactagtagttgaaaaaccaatactggttatctatatgagt
tataataattaaacattggtaatcaaagtgaacaattcttaggttaacatgaagactacaaagcatgtcaagaacg
gttgaaactgttaaaacactaagaaactctagattaattgtgttggactatgtccatacttctaagaatttggtat
gccagtttatagaagatttgtcacataatgtgatagatggtgcatcgagaaagatggacttgagacccacttaatg
ccattccatagtagtagcattttctatgtgattggagatctcatgaagtaaaatggtgaaataagctagagaatga
ctgatggagagactccttaaataaaatctaggtaatttcatatgaacatttctccttcgatgtacgataggctaac
taaaatcttaatatgatttgagtgacatatctaagcagggatgttatcttatagaacatcctataaggaacacacc
tatatgactttgactactaatcacagtctatgatatgtgggaaatatttttttactcatgaaaggcctcgaagttt
gacttatatgcttcaaaccaatggataactgattgaatctgtttcagacagcagcatagttaacttcaaaattcta
tatctcccaaacttctcaaccaaaaattattaaattatgctgacagtaagaacttttatccctctacaaaagtctc
catagttggaagagccaattcggccatttactagatgaaacctaccagttaacagacccgctcatttttgtcaggc
aaacaagaacccttatatatcaaagagtagtggatttgaatttgtctaatatgaagtttatgggcttggatcattt
aaagtattttataattctgaaagacaagattaaactaactatgtatgatcctttagcaccgtacagatggtcgatg
ggacagttacatggtttatatagatggacctatctatattccttcagtggttgagaacaaggtaaaggtgtgtcac
actcacgcgtgcgccgcctccgccgagacgaggcatggcgttgcagggtgaggcatggcatggtgcagcacgatcg
cagtcatggtcgtggcacagtgtagcactgttgtggatagatagataaactattgttgtagttagagtgcatgagt
ttttgaatgcaacactagaattttcacaatttaacttaattaagaagttatatcttgatggcacagagttacctga
ccattgatctcacatgcccttttagctctctcgtactctagacttaccctgactatttaggtctcctgactcgcca
atataaacaccatcctatcatttgctgtcgaatacttctgtgtcacaactcaactgtcagatgagtactccattac
agcactgagcaggcctccgaaatgaatatctactcagatgaaggacgacaatcgtgttctagggaaatatcagcga
cacaactccttacttcctccgcttcttcctagtgttttttgttgtgattgagtcgacatagcaacaacactgcact
attacaaccagtacgactatatcaactaggcaatgtcttccttatatgttactatttattttgctcatattcatta
tgtttaaatcacataggcgcctttatgttggcatcaaaaaatcagtatcaactttatagattaaaatgaaactaaa
agtacctaaatttctatcggtggggatcgagtgattctttaaaccgattattacacaagttaaccacactaaaatt
aacaatggtgaatcgtgccatgatttttttctagtggaaaatagccaaaccaagcaacacatatgtggctatcgtt
acacatgtgtaaaggtattgcatcacaccattgtcacccatgtatgtggacaataccgagaggaaaaaccacttat
ttattgtattttatcaagtttgtcttgcttacgtataaattataacccaacaaagtaatcactaaatgtcaaaacc
aactagataccatgtcatctctaccttatcttactaatattctttttgcaaaatccaaaattaatcttgcacaagc
acaaggactgagatgtgtataaatatctcttaaattagtagctaatatatcgcacatattattgagaccaactagc
gacatagaaagcacaatagtgtaccaaccATGGCAGAAGAAAGGCGGCAGAACGCGCTAGAGATCCGCATTCAAGC
GGCAAAGCTCGCAAAGAAGCGGGATCACCCGACACATAAGGCAAACGGCGATGAGGACCGCTATCCAGAGACGCTT
ATCGGCTCATTCACCAAAGGCTTGCCGCACGAGAAGGAGACAGGGCTTCTCTCCAATCCTGCAGACTTTGCTGATT
TCGTTAGGGCTATTAATACAGGCGCCATCAAGGACATGAGGCGGCTCAAGCTGGGTATTGACGAAGACCCTCGGTT
CATCTCCGGTATAGCCTCAAAAGGCGACAAACATCCCTTCGCGGACACCAGAGCCTGGGAATCTATGGCTGCTGGC
TTGACCTATGATCTGGAGGGGCCTGACGCGCAGGCCGTTACGATGCCCCCCGCCCCGAAACTTGACAGCGACGAGT
TGGTGACTGAAATTACCGAATCATACTGGATGGCCCTTCTCCGAGATGTTCCGTTTACTGAGTTCGAGCGCGACGG
AAATACTGCAGCTGCTGCAGCGTCCATAAGCAGAACTCGATGGGTACAGTACAACGAGCAACCTTCACAGCGGCCA
TCGACTTTGACGGACGAGGAAATAGCCCGTTTGCGCGGGCCTTATACGAAGAAGAATGTTTTCAGGGGGGTGACCA
ACGGTGAGAACGTAGGCCCATACCTCTCCCAGTTCCTCCTCGTGGGCACCAAAGGCATCGGCGATGCACAGCAAGT
GTCGGATGGATACGTCCAGTACGGCGGCATGAGGATGGATCAGCGCGTAAGGGTAGCTGTGCCTAAGAGGGATTAC
ATGACCACTTGGGCGAGCTGGCTGGACGTTCAGAATGCGGGGGACTTGAGAGGACGCGAAATATACGACGACGACA
CGCCATTCAGGTTCATAACTACCCCGCGTGATCTGGCAACTTGGGTGCACTTCGACGCCTTGTACCAAGCATACCT
TAACGCGTGTATTATCCTGCTCGATATCAAGGCTCCGTTCGATCCGCACATACCATTTCAAGCCGACGACGACGTC
GACAAGCAGCAAGGATTCGCAACCTTCGGAGGTCCGCATATCCTCAGTCTCTGCACAGAGGTCGCCACCAGGGCAC
TCAAAGCCGTTCGCTTCCAGAAGTATAACCTACACCGGCGTCTCCGCCCTGAAGCCATCGGAGGCCTGGTTGAGCG
GTTCAAGAAGACCAATGGCGACCCAAAGTTCGCTCCTGTGAAAAAGCTCGTGAATGACCTCGACGGAGACATGCTT
CGCCGAGTCGAGCAACACAATTGCGAGCAGAACAAACTTTCCGACGATGGCCACGCGAGAAGAGAGGACTACAGCC
CGGAGGGAGAGTCAAGCCAGAGCTATCTTCTTCCTATGGCGTTCCCGGAGGGTTCTCCTATGCATCCATCATATGG
TGCTGGCCACGCTACCGTCGCCGGGGCGTGCGTGACAGTGTTGAAGGCTTTTTTCGACCACGAATACGAGCTTGAC
TTCTGCTATGTCCCCACCACCGACGGTAAGCGTCTCGAGAAGGTCAACATCAATGAGAAGTTGACCGTTGAGGGCG
AGCTGAATAAGTTGTGTGCAAACATCAGCATAGGGCGCAACTGGGCAGGGGTCCACTACTACTCTGACTATTTCGA
GTCCATTAAGGTGGGAGAGGAGATAGCCATCGGTATTCTGCAGGAGCAGAAGCTTACATACGGGGAGGACTTCTTT
ATGACACTACCGAAATTTGATGGCGAGAAGATTAGGATTGGAAGCGGAGCTACTAACTTCAGCCTGCTGAAGCAGG
CTGGAGACGTGGAGGAGAACCCTGGACCTATGGCCCATACATCAGAATCTGTGAATCCTAGAGATGTTTGCATTGT
GGGTGTTGCACGTACTCCAATGGGTGGCTTTCTCGGATCTCTTTCATCTTTACCTGCCACAAAGCTTGGATCTTTA
GCTATTGCAGCTGCTTTGAAGAGAGCAAATGTTGATCCAGCTCTTGTTCAAGAAGTTGTCTTTGGCAATGTTCTTA
GTGCTAATTTGGGTCAAGCTCCTGCTCGTCAAGCTGCTTTAGGTGCAGGAATCCCTAACTCTGTTATCTGTACTAC
AGTTAACAAGGTTTGTGCATCAGGCATGAAAGCGGTAATGATTGCTGCTCAAAGTATCCAGTTAGGGATCAATGAT
GTAGTTGTGGCGGGTGGTATGGAAAGCATGTCTAATACACCAAAATATTTGGCAGAAGCAAGGAAGGGATCTCGTT
TTGGTCATGATTCTTTAGTAGATGGAATGTTGAAGGATGGACTATGGGATGTCTATAACGACTGTGGGATGGGAAG
CTGTGCAGAATTATGCGCTGAGAAGTTTCAGATTACAAGGGAGCAGCAAGATGACTATGCAGTTCAGAGTTTTGAG
CGTGGTATTGCTGCCCAGGAAGCTGGCGCCTTCACATGGGAAATCGTCCCGGTTGAAGTTTCTGGAGGAAGAGGTA
GGCCATCAACCATTGTTGACAAGGACGAAGGTCTTGGGAAGTTTGATGCTGCAAAATTGAGGAAACTCCGTCCTAG
TTTCAAAGAGAATGGAGGGACTGTTACAGCTGGAAATGCGTCTAGCATAAGTGATGGTGCAGCTGCCCTTGTCCTA
GTGAGCGGAGAGAAGGCTCTTCAGCTAGGACTTCTAGTATTAGCAAAAATTAAAGGGTATGGTGACGCAGCTCAGG
AACCAGAGTTTTTCACTACTGCTCCTGCTCTTGCTATACCAAAAGCCATTGCACATGCTGGTTTGGAATCTTCTCA
AGTTGATTACTATGAGATCAATGAAGCATTTGCAGTTGTAGCACTTGCAAATCAAAAGCTACTCGGGATTGCTCCA
GAGAAAGTGAACGTAAATGGAGGAGCTGTCTCCTTAGGACACCCTCTAGGCTGCAGTGGCGCCCGTATTCTAATCA
CGTTGCTTGGGATACTAAAGAAGAGAAACGGAAAGTACGGTGTGGGAGGAGTGTGCAACGGAGGAGGAGGTGCTTC
TGCTCTAGTTCTTGAGCTCCTTGGTTCAGGTGCCACTAATTTTTCTCTCTTGAAACAGGCCGGTGACGTTGAAGAG
AACCCAGGTCCAATGAAGAAGCGCTTAACCACTTCCACTTGTTCTTCTTCTCCATCTTCCTCTGTTTCTTCTTCTA
CTACTACTTCCTCTCCTATTCAGTCGGAGGCTCCAAGGCCTAAACGAGCCAAAAGGGCTAAGAAATCTTCTCCTTC
TGGTGATAAATCTCATAACCCGACAAGCCCTGCTTCTACCCGACGCAGCTCTATCTACAGAGGAGTCACTAGACAT
AGATGGACTGGGAGATTCGAGGCTCATCTTTGGGACAAAAGCTCTTGGAATTCGATTCAGAACAAGAAAGGCAAAC
AAGTTTATCTGGGAGCATATGACAGTGAAGAAGCAGCAGCACATACGTACGATCTGGCTGCTCTCAAGTACTGGGG
ACCCGACACCATCTTGAATTTTCCGGCAGAGACGTACACAAAGGAATTGGAAGAAATGCAGAGAGTGACAAAGGAA
GAATATTTGGCTTCTCTCCGCCGCCAGAGCAGTGGTTTCTCCAGAGGCGTCTCTAAATATCGCGGCGTCGCTAGGC
ATCACCACAACGGAAGATGGGAGGCTCGGATCGGAAGAGTGTTTGGGAACAAGTACTTGTACCTCGGCACCTATAA
TACGCAGGAGGAAGCTGCTGCAGCATATGACATGGCTGCGATTGAGTATCGAGGCGCAAACGCGGTTACTAATTTC
GACATTAGTAATTACATTGACCGGTTAAAGAAGAAAGGTGTTTTCCCGTTCCCTGTGAACCAAGCTAACCATCAAG
AGGGTATTCTTGTTGAAGCCAAACAAGAAGTTGAAACGAGAGAAGCGAAGGAAGAGCCTAGAGAAGAAGTGAAACA
ACAGTACGTGGAAGAACCACCGCAAGAAGAAGAAGAGAAGGAAGAAGAGAAAGCAGAGCAACAAGAAGCAGAGATT
GTAGGATATTCAGAAGAAGCAGCAGTGGTCAATTGCTGCATAGACTCTTCAACCATAATGGAAATGGATCGTTGTG
GGGACAACAATGAGCTGGCTTGGAACTTCTGTATGATGGATACAGGGTTTTCTCCGTTTTTGACTGATCAGAATCT
CGCGAATGAGAATCCCATAGAGTATCCGGAGCTATTCAATGAGTTAGCATTTGAGGACAACATCGACTTCATGTTC
GATGATGGGAAGCACGAGTGCTTGAACTTGGAAAATCTGGATTGTTGCGTGGTGGGAAGAGAGAGCCCACCCTCTT
CTTCTTCACCATTGTCTTGCTTATCTACTGACTCTGCTTCATCAACAACAACAACAACAACCTCGGTTTCTTGTAA
CTATTTGGTCTGATAAcgatcgttcaaacatttggcaataaagtttcttaagattgaatcctgttgccggtcttgc
gatgattatcatataatttctgttgaattacgttaagcatgtaataattaacatgtaatgcatgacgttatttatg
agatgggtttttatgattagagtcccgcaattatacatttaatacgcgatagaaaacaaaatatagcgcgcaaact
aggataaattatcgcgcgcggtgtcatctatgttactagatcatatgaagatgaagatgaaatatttggtgtgtca
aataaaaagcttgtgtgcttaagtttgtgtttttttcttggcttgttgtgttatgaatttgtggctttttctaata
ttaaatgaatgtaagatctcattataatgaataaacaaatgtttctataatccattgtgaatgttttgttggatct
cttctgcagcatataactactgtatgtgctatggtatggactatggaatatgattaaagataagggaa

### Example 2

### Enhanced Synthesis of AcAc-COA in Target Tissues to Provide Increased Organic Substrate for Bromoform Synthesis by VBPO

Another embodiment described herein is the transgenic expression of acetoacetyl-CoA thiolase 2 (AACT2) of Arabidopsis thaliana (“Arabidopsis”) or other organisms, which provides increased AcAc-COA availability (SEQ ID NO: 9-15). AcAc-COA is present throughout the plant due to the universal importance and wide-ranging functions of its derivatives. However, its local abundance is a limiting factor for bromoform synthesis which varies temporally and with tissue type. The AACT2 enzyme converts 2 acetyl-S-CoA (Ac-COA) molecules to AcAc-COA (FIG. 2). Transgenic expression of AACT2 therefore results in relative increase of Ac-COA directed to AcAc-CoA in the cytosol vs alternate metabolic pathways.

AACT2 may be expressed under constitutive or targeted promoters as described herein. The AACT2 transgene for use in dicots consists of either the native Arabidopsis protein coding sequence sans introns (e.g., the cDNA), or the native coding sequence in which the first intron is maintained but other introns are deleted (SEQ ID NO: 9-11). The variation in intron composition, in combination with promoter selection, provides a means of tuning the transgene expression level and resultant stoichiometry of AcAc-CoA synthesis in combination with other aspects described herein to achieve the desired level of bromoform across variation in target species and plant tissues. The AACT2 transgene for use in monocots consists of the Arabidopsis protein coding sequence codon-optimized for Zea mays with or without the addition of the 5′-untranslated region (UTR) of Oryza sativa Superoxide Dismutase (SOD) (SEQ ID NO: 12-13).

### Example 3

### Increased Storage Oil and Sink Strength

Another embodiment described herein is broadly increased lipid biosynthesis conferred by overexpression of Wrinkled 1 (WRI1) of Arabidopsis. WRI1 is a transcription factor that positively regulates fatty acid synthesis at multiple points in the metabolic pathway, and in its native setting controls storage oil accumulation during seed filling. Applied in seeds, transgenic expression of WRI1 increases storage oil accumulation. Applied in other tissues, WRI1 transgenic expression can cause plant parts that do not typically synthesize storage oil to do so. The accumulation of oil increases the holding capacity of plants for synthesized bromoform, which is lipid-soluble, and reduces loss to the atmosphere through volatilization. In plants where starch is the primary carbon storage molecule in endosperm, including corn and other monocots, the role of WRI1 to alter carbon allocation towards oil synthesis is more central, and this mechanism of increased oil accumulation differs from conventional high-oil corn produced through breeding for increased embryo (germ) size.

WRI1 overexpression also results in increased Ac-COA as a substrate for AACT2, and increased medium to long chain fatty acid synthesis provides increased material for formation of cuticular polymers, which provides a barrier to VOC emission and further sink material to which bromoform is adsorbed. A significant benefit is the containment provided by synthesis of bromoform in self-contained, oil-bearing plant organs. This benefit is compounded by the fact that engineered plant parts used for animal feed are living tissue (e.g., seeds or forage plants) in which bromoform biosynthesis remains active up to the moment of consumption. This maximizes the portion of synthesized bromoform that is received by animals rather than lost during harvest, shipping, and storage of dead material.

As is the case with AACT2, The WRI1 transgene may exist as several versions with varying intron composition to adjust expression level depending on target system and application. The WRI1 transgene for use in dicots consists of either the native Arabidopsis protein coding sequence sans introns (e.g., the cDNA), or the native coding sequence in which the first intron is maintained but other introns are deleted (SEQ ID NO: 14-16). WRI1 transgenes for use in monocots consist of a Zea mays codon-optimized coding sequence, with or without an appended 5′-untranslated region (UTR) derived from the first intron of rice (Oryza sativa) Cytosolic Superoxide Dismutase 2 (OsSODCc2) (SEQ ID NO: 17-18).

AACT2, WRI1, and VBPO were assembled into a tricistronic sequence joined by the P2A self-cleaving peptide (SEQ ID NO: 28) and transiently expressed under control of the Ubiquitin 10 promoter (SEQ ID NO: 19) in Nicotiana tabacum. Another construct containing VBPO alone under control of the Ubiquitin 10 promoter was also transiently expressed in Nicotiana tabacum. Leaf tissue samples from the transformed area were taken 3 days after transformation, frozen in liquid nitrogen and ground using steel or cubic zirconia beads, then methanol was added to each tissue sample, and samples were blended with beads, vortexed thoroughly, allowed to incubate overnight at 4° C., and vortexed again. Samples were centrifuged to collect debris, and the supernatants were analyzed for absolute bromoform concentration (accounting for original sample mass and extraction volume) using GC-MS at Avazyme, Inc. Comparison of bromoform content in this experiment between leaves expressing VBPO alone, and leaves expressing VBPO in combination with AACT2 and WRI1, is expected to quantify the percentage by which expression of the additional genes enhances total bromoform synthesis and/or accumulation.

### Example 4

### Targeted Expression via Promoter Combinations

Another embodiment described herein is the use of different promoters to direct expression of the transgenic enzymes to different parts of the plant according to the specifics of its use as animal feed. As with the coding sequences themselves, the use of promoters is combinatorial and varies with target species and application.

For any of the listed transgenes in dicots, expression covering most plant tissues is conferred by the Ubiquitin 10 promoter of Arabidopsis (pAtUBQ10) (SEQ ID NO: 19). In monocots constitutive expression can be conferred by the Ubiquitin 1 promoter of corn (pZmUbi1) or another constitutive promoter such as that of Alcohol Dehydrogenase 1 (pZmAdh1) (SEQ ID NO: 20).

Expression that is primarily in photosynthetic tissues consumed by animals in forage applications (e.g., leaves, stems) and which is low in root, seed, and reproductive tissue is conferred in dicots and monocots by a ribulose-1,5-bisphosphate carboxylase/oxygenase small subunit promoter derived from Arabidopsis (pAtRbcS1a) or another plant such as sweet potato (Ipomoea batatas) (pIbRbcS1), or a chlorophyll a/b binding protein promoter derived from Arabidopsis (pAtCAB3) or another plant such as black pine (Pinus thunbergii) (pPtCAB6) (SEQ ID NO: 21-23).

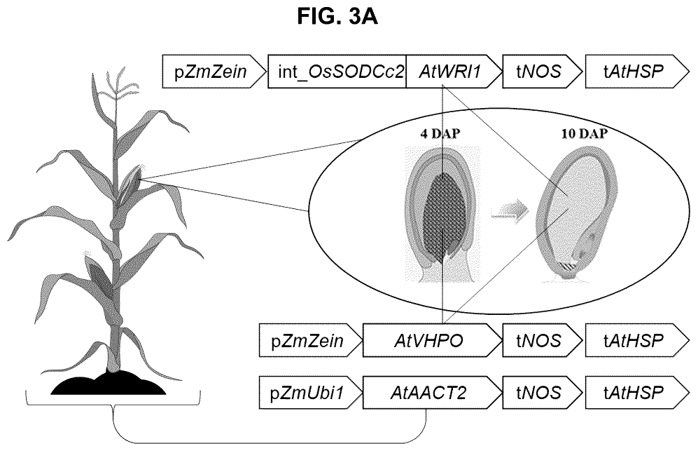

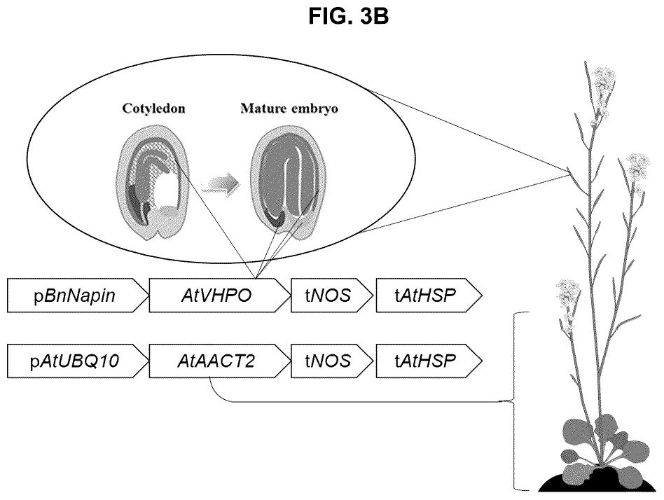

Expression primarily within the internal seed tissues of monocots and dicots, including the endosperm and/or embryo, is conferred by promoters of seed storage proteins derived from Napin of rapeseed (Brassica napus) (pBnNapin) or Cruciferin of Arabidopsis thaliana (pAtCruciferin) in dicots, and additionally in monocots from an appropriate α-zein gene of corn (pZmZein) or other appropriate variety-specific zein (SEQ ID NO: 24-25). Promoters and coding sequences are used in combination with the terminators of Arabidopsis heat-shock protein (tAtHSP), Agrobacterium tumefaciens nopaline synthase (tNOS), or other appropriate terminators (SEQ ID NO: 26-27). FIG. 3A-B show how combinations of some or all protein-coding sequences under specific and constitutive promoters provides endosperm-specific expression in monocots and dicots.

Leaves of Nicotiana tabacum and elite corn variety Zea mays LH244 were transiently transformed with two separate constructs expressing AACT2, WRI1, and VBPO assembled into a tricistron (SEQ ID NO: 28) under control of the 35S promoter (SEQ ID NO: 93) and Arabidopsis thaliana Ubiquitin 10 promoter (SEQ ID NO: 19). Bromoform was extracted from transformed leaf tissue as described in Example 3 and analyzed for absolute bromoform concentration at Avazyme, Inc. This experiment is expected to reveal the percentage by which expression of VBPO under the Ubiquitin promoter enhanced bromoform production and/or accumulation relative to expression under the 35S promoter.

A tricistronic sequence containing AACT2, WRI1, and VBPO as described in Example 3 was transgenically expressed in Arabidopsis and Setaria italica variety ‘Red Siberian’ in three different constructs. At least five Arabidopsis plants were transformed for each of 2 genetic constructs carrying the tricistronic transgene (SEQ ID NO: 28), in one of which the transgene was under control of the Napin promoter (SEQ ID NO: 24) and in the other of which the gene was under control of the 35S promoter (SEQ ID NO: 93). At least five Setaria plants were transformed for each of 2 genetic constructs, in one of which the transgene (SEQ ID NO: 28) was under control of the 35S promoter (SEQ ID NO: 93) and in the other of which the transgene was under control of the 19 kDa Zein promoter (SEQ ID NO: 94) and the VBPO gene sequence within the tricistron was codon-optimized for monocot expression using the maize genetic code (SEQ ID NO: 3 or 95). Mature seed of each species of transformed plants was harvested and screened for transformants using resistance to kanamycin, and transformant viability was assessed as the frequency of kanamycin resistant plants surviving to produce the first true leaf. These results revealed that in Arabidopsis, expression of the bromoform biosynthesis construct under the 35S promoter yielded no viable transformants, while seed-tailored expression under the Napin promoter yielded ˜1% viable transformants in the first generation. In Setaria, seed-tailored expression of the bromoform biosynthesis construct under the Zein promoter yielded ˜0.2% viable transformants, while expression under the 35S promoter yielded a single viable transformant from ˜8,000 seeds, or ˜0.01% viability; the surviving transformant of the 35S construct also had stunted growth.

### Example 5

Assessment of Methane Emissions from Livestock

Bromoform-bearing plants are used to reduce methane emissions from livestock by incorporation into feed. In this example, bromoform is delivered to livestock in any fresh, dried, or processed plant tissue. Efficacy of this method can be demonstrated by methane reduction from rumen fluid upon exposure to engineered Arabidopsis, crop plants, or other model plants such as Setaria. Rumen fluid studies are a widely accepted analyses for assessing animal nutrition. Typically, a small amount of fresh rumen material is extracted from living cows or other animals that are exposed to a given treatment and monitored via gas chromatography in a sealed system for detailed effects on emission of methane, hydrogen, volatile fatty acids, and other components of digestion.

### Example 6

### Modulation of Bromoform Levels in Transgenically Modified Plants

Tailored expression of bromoform biosynthesis is used to create a common animal feed grain such as corn (Zea mays) that is calibrated according to its inclusion rate in a specific livestock diet to provide an overall effective dose to eliminate maximum methane emissions. For example, an effective dosage rate of bromoform for methane elimination in cows is ˜25 μg per gram total feed, and corn commonly makes up ˜1-80% of cattle diets. Thus, using the means provided herein to adjust multiple components of bromoform biosynthesis, corn seed is specifically engineered to produce bromoform at a rate of from ˜2500 μg per gram, when included at ˜1% of feed, to ˜31.25 μg per gram, when included at ˜80% of feed. The same tailored methods are applied for other feed components making up a large portion of livestock diets such as soybean grain, barley grain, silages, or hays.

### Example 7

### Methane-Reducing Forage Crops

Bromoform biosynthesis is used to provide a methane-reducing forage crop directly consumed by animals grazing in a pasture rather than mixed into a farmer-provided ration with other feed components. In this experimental example, the crop is a plant species commonly planted in a managed pasture including monocots such as ryegrass (Lolium sp.), sorghum (Sorghum sp.) and fescue (Festuca sp.), or dicots such as alfalfa (Medicago sp.), clover (Trifolium), and pea (Pisum sp.). The pasture crop may have a perennial or an annual cultivation cycle. Using the regulatory genetic components as described herein, bromoform biosynthesis may be constitutive, limited spatially to aboveground plant parts that grazing animals primarily eat, or limited temporally to daytime when grazing primarily takes place. Bromoform is made at a level corresponding to the proportion of pasture in the animal diet and the proportion of bromoform-bearing species in the pasture.

For example, if ˜25 μg per gram dry matter in total feed is an effective dosage rate for methane elimination in cows, and the forage crop is applied to cattle eating a 100% forage diet at a particular point in time, the forage crop may include a mixture of red clover (Trifolium pratense), perennial ryegrass (Lolium perenne), meadow brome (Bromus commutatus), tall fescue (Festuca arundinacea), and alfalfa (Medicago sativa), where each plant species is engineered to produce ˜25 μg bromoform per gram dry matter in aboveground plant parts.

Alternatively, the forage crop may include a single engineered pasture plant providing a higher level of bromoform that is consumed in a mixture with other unmodified food components. For example, if cattle are eating a diet of 50% forage and 50% farmer-provided rations, and the forage pasture is made up of ˜20% perennial ryegrass, the forage crop may include perennial ryegrass containing ˜250 μg bromoform per gram dry matter such that, when consumed alongside other forage plants and farmer-supplied rations, a sufficient dose of bromoform is provided to achieve maximum methane elimination from the total diet. In this experimental example, the bromoform-bearing plant may be consumed with spatial or temporal separation from other feed sources, while still providing sufficient total bromoform to achieve maximum methane reduction. The efficacy of a methane-reducing forage crop may be demonstrated in a model system comprising feeding engineered forage species to methane-generating insects such as termites, cockroaches, and beetles.

Leaf disks of the grass Setaria viridis A10.1 were transiently transformed with a genetic construct expressing a tricistronic, partially monocot-optimized sequence of AACT2, WRI1, and VBPO (SEQ ID NO: 95) under control of the Arabidopsis thaliana Ubiquitin 10 promoter (SEQ ID NO: 19). Three days after transformation, to simulate methane reduction in grazing livestock consuming a forage plant, transgenic Setaria leaf disks were fed to a highly methanogenic, leaf litter-feeding species of cockroach, Gromphadorhina portentosa, which is able to consume similar foods to cattle and in which methane production occurs in the gut via the same groups of microbes and through the same fermentation process as in cattle. Cockroaches were transferred into chambers containing a methane sensor. The chamber containing the sensor, cockroaches, and food source was sealed for a period of two hours, and methane accumulation at the end of this period was measured. The chamber as unsealed and cockroaches were maintained in the chamber for an additional 44 hours with the transgenic Setaria leaf disks as a sole food source, after which the chamber was again sealed for another 2-hour accumulation period and endpoint methane measurement. Cockroaches consuming untransformed Setaria leaf disks were used as a control, and each treatment group consisted of 3-4 cockroaches weighing ˜20 grams. Accumulated methane in cockroaches consuming the transgenic Setaria leaf disks decreased by 24% over 48 hours, from 3.95 parts per million methane per gram biomass to 3 parts per million methane per gram biomass. In contrast, accumulated methane in cockroaches consuming the control nontransgenic Setaria leaf disks increased by 2% over 48 hours, from 4.7 parts per million methane per gram biomass to 4.8 parts per million methane per gram biomass. The results of this experiment indicate that leaf tissues of a forage crop can be successfully engineered to reduce enteric methane emissions in animals that consume them.

### Example 8

### Animal Feed Containing High Levels of Bromoform

A plant containing bromoform at a very high level is used as a supplement or small fraction of an animal diet to deliver a sufficient dose of bromoform to the animal to eliminate total methane emissions. In this experimental example, the plant may be an existing component of animal feed such as corn, ryegrass, beets, or canola/rapeseed. The plant may also be a species not commonly incorporated into animal feed, but nevertheless is suitable as a component of animal diets, such as duckweed (family Lemnoideae), coral bean (Erythrina beteroana), seepweed (Suaeda sp.), crambe (Crambe sp.), seashore mallow (Kosteletzkya virginica), or saltgrass (Distichlis sp.).

This experimental example may also use a processed bromoform-bearing plant product, for example, freeze-dried plant tissue or oil pressed from seeds. The plant product is included in animal diets according to the amount of bromoform synthesized. For example, if engineered canola seeds contain ˜2,500 μg bromoform per gram plant matter, they may be supplemented to livestock diets at ˜1% of total feed to provide an overall effective dose of ˜25 μg per gram bromoform in total feed.

To determine the methane-suppressing effect of a feed prepared in this manner, transgenic Arabidopsis seed of a variety documented to produce ˜1,653.68 μg per gram bromoform as described in Example 1 was incorporated into a synthetic cattle diet, The synthetic cattle diet consisted of (by mass) 25% ground corn; 40% Setaria hay; 10% ground soybeans; 10% fresh carrot; 5% vegetable oil; and 10% ground Arabidopsis seed made up of transgenic and nontransgenic portions calculated to achieve a specific dosage of bromoform. A version of this diet was prepared to include ˜25 μg per gram total bromoform as 1.5% transgenic Arabidopsis and 8.5% nontransgenic Arabidopsis and was compared to a control containing no bromoform (as 10% nontransgenic Arabidopsis). Each synthetic cattle diet was fed to a highly methanogenic, leaf litter-feeding species of cockroach, Gromphadorhina portentosa, which is able to consume similar foods to cattle and in which methane production occurs in the gut via the same groups of microbes and through the same fermentation process as in cattle. After consuming the control diet for at least 1 week, cockroaches were transferred into chambers containing a methane sensor assembly pre-calibrated against known concentrations of methane, with a restricted gas exchange rate to outside air. This system allowed enteric methane emitted by the cockroaches to accumulate in the chamber, where it was measured by the sensor. ˜ 20 grams of cockroach biomass, consisting of 3-5 individuals, was used as each experimental unit. Cockroaches were maintained in the methane measurement chamber for 1 week with constant access to the either the control or experimental diet, and methane was measured ˜5 times per minute for the duration. Results of this experiment revealed that methane emissions from cockroaches consuming the control diet accumulated to ˜97 parts per million, while emissions from cockroaches consuming the control diet began dropping within 6 hours after introduction of the experimental diet and continued dropping for 5 days until reaching a level of ˜18 parts per million. Accounting for the fact that ambient air contains ˜2 parts per million methane, this represented an 83% reduction in methane emissions from use of the invention described here.

### Example 9

### Improvements to Effective Dose of Bromoform

Delivery via plant material within which the compound was natively biosynthesized provides for a novel means of incorporating and distributing bromoform within a livestock diet. Inclusion within plant tissues, as opposed to mixing or supplementation, may alter the way in which bromoform moves through the digestive system and comes in contact with methane-producing microbes during feed digestion. This in turn may reduce the required dosage of bromoform needed to achieve a given level of methane reduction. These factors may also improve the safety profile of bromoform by reducing side effects to the livestock animal, excretion of excess, or accumulation within animal tissue. They may also improve the utility of bromoform by improving its palatability as a component of animal feed, because the total dosage is reduced or because the total amount of bromoform consumed is distributed into the interior of food particles rather than mixed with or coated on the outside.

Experimental versions of the cattle diet as described in Example 8 were prepared as follows. Diet A included ˜25 μg per gram total bromoform (as 1.5% transgenic Arabidopsis and 8.5% nontransgenic Arabidopsis); Diet B included ˜50 μg per gram total bromoform (as 3% transgenic Arabidopsis and 7% nontransgenic Arabidopsis); and Diet C included ˜100 μg per gram total bromoform (as 6% transgenic Arabidopsis and 4% nontransgenic Arabidopsis). Four control diets were additionally prepared as follows. Diet D included no bromoform (as 10% nontransgenic Arabidopsis); Diet E included ˜25 μg per gram total bromoform (as 0.0025% synthetic bromoform diluted into the vegetable oil portion of the diet and 10% nontransgenic Arabidopsis); Diet F included ˜50 μg per gram total bromoform (as 0.005% synthetic bromoform diluted into the vegetable oil portion of the diet and 10% nontransgenic Arabidopsis); and Diet G included ˜100 μg per gram total bromoform (as 0.01% synthetic bromoform diluted into the vegetable oil portion of the diet and 10% nontransgenic Arabidopsis).

One experimental unit (as described in Example 8) per diets A-F was fed on control diet D for at least one week, then transferred to a methane chamber as described in Example 8 and fed on the respective experimental diet for an additional week. Methane accumulation was measured at 0, 6, 48, 96, and 168 hours after introduction of the experimental diet. This experiment was repeated three times.

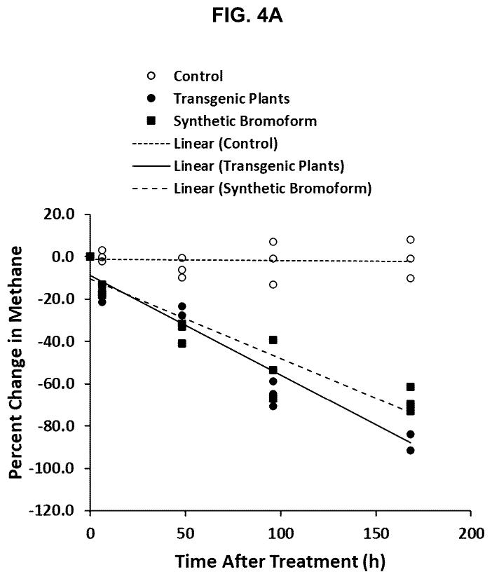

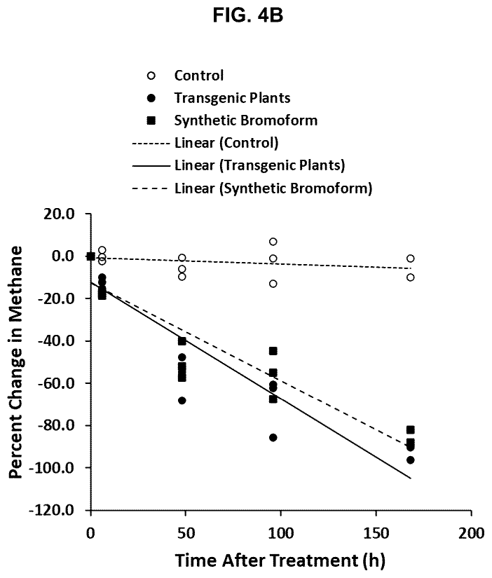

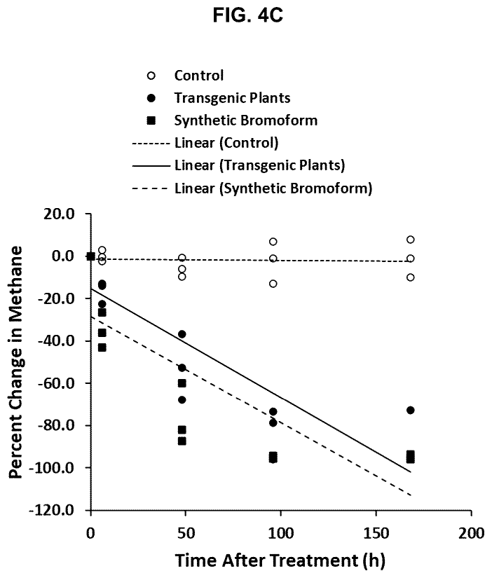

As shown in FIG. 4A-C, all experimental diets strongly reduced methane emissions, while the control diet did not. The results of this experiment also show that a given dosage of bromoform delivered through transgenic plants is more effective at reducing methane than the same amount of purified bromoform, and this effect as especially pronounced at lower concentrations. As shown in FIG. 4A, diets A and E delivered 25 μg per gram bromoform through transgenic plants and synthetic bromoform respectively. At the final time point of 196 hours (one week), the average reduction in methane emission due to inclusion of 1.5% transgenic plants in diet A over three repetitions of the experiment was 81.7%, while the average reduction in methane due to inclusion of 0.0025% synthetic bromoform was 68.0%. This indicates that the present invention may be at least 20.1% more effective at methane reduction than other means of delivering an equivalent amount of bromoform at around this dosage level.

FIG. 4B shows that diets B and F delivered 50 μg per gram bromoform through transgenic plants and synthetic bromoform respectively. At the final time point, the average reduction in methane emission due to inclusion of 3% transgenic plants in diet B over three repetitions of the experiment was 91.6%, while the average reduction in methane due to inclusion of 0.005% synthetic bromoform was 84.4%. This indicates that the present invention may be at least 8.5% more effective at methane reduction than other means of delivering an equivalent amount of bromoform at around this dosage level.

FIG. 4C shows that diets C and G delivered 100 μg per gram bromoform through transgenic plants and synthetic bromoform respectively. At the final time point, the average reduction in methane emission due to inclusion of 6% transgenic plants in diet C over three repetitions of the experiment was 87.8%, while the average reduction in methane due to inclusion of 0.01% synthetic bromoform was 95.1%. In this portion of the results, synthetic bromoform was 8.3% more effective than transgenic plants at the relevant dosage level. However, comparison of the overall dose-response results for both forms of bromoform shown in FIG. 4A-C establishes a general pattern showing that delivery of bromoform through transgenic plants is nearly as effective at reducing methane as a dose of synthetic bromoform twice as large: 25 μg per gram plant-delivered bromoform reduced enteric methane emissions 96.8% as well as 50 μg per gram synthetic bromoform, and 50 μg per gram plant-delivered bromoform reduced enteric methane emissions 96.3% as well as 100 μg per gram synthetic bromoform.

### Example 10

### Improvements to Storage Stability of Bromoform

Use of synthetic or Asparagopsis-derived bromoform is significantly hampered by instability of bromoform in these forms, requiring specialized storage conditions and frequent precise dosage. The same limitation extends to non-bromoform based additives as well, including microbial products and synthetic chemicals, which must be delivered, stored, and mixed into cattle feed using special procedures. The ability to store and process grains and plant parts modified with the present invention through the existing agricultural supply chain and feed machinery as a convenient source of bromoform for methane reduction is a significant improvement.

Seed of transgenic Arabidopsis plants expressing the VBPO gene under control of the Napin promoter was subjected to a preliminary stability experiment mimicking supply chain conditions. Freshly harvested mature transgenic seed was divided into four parts. Part A was immediately ground and extracted in methanol as described in Example 1 for quantification of bromoform. Part B was immediately stored at −80° C. in an airtight container. Part C stored for 4 weeks at 25° C. and 70% ambient humidity, with air circulation, and then ground and extracted in methanol for quantification of bromoform as described in Example 1. Part D was stored for 4 weeks at 25° C. and 70% ambient humidity, with air circulation, and then used to prepare a simulated cattle diet containing 5% transgenic plant material as described in Example 9. At the same time as Part D, Part B was removed from cold storage and used to prepare an identical simulated cattle diet. Diets composed using Parts B and D were each fed to a single experimental unit of cockroaches as described in Example 8, and methane was measured at 0 and 72 hours. In cockroaches consuming diets containing 5% ambiently stored transgenic plant parts in Diet D, methane emissions were reduced by 72% after 72 hours.

Oil extracts of Asparagopsis seaweed have been reported to decrease in bromoform content by 44% after four weeks of exposure to air at the environmental conditions used in this experiment, and freeze-dried Asparagopsis decreases in bromoform content by 16% even inside of a sealed bag at the same conditions. Extracted bromoform in samples of Part A and C was quantified by GC-MS at Avazyme, Inc. The results of this experiment are expected to show the amount of bromoform present in transgenic seeds at the time of harvest sampled in Part A, and after a month of storage at ambient conditions sampled in Part C. By comparison of concentrations in Part C and part A, the results of the experiment are expected to reveal the extent to which bromoform is lost over time from transgenic seeds at ambient conditions, and the extent to which delivery of bromoform via plant tissues as described In the present invention offers practical advantages in bromoform stability relative to current alternatives including, for example, oil preparations of seaweed, oil or aqueous preparations of synthetic bromoform, or freeze-dried seaweed.

### Example 11

In some applications, production of particular haloforms is preferred. For example, iodoform may be preferred in some livestock feed applications for having lower volatility than bromoform, an improved safety profile, and/or a wider dosage range. The production of a particular haloform may also be preferred to enable increased accumulation and/or production of the haloform in plants due to lower phytotoxicity or differences in halogen substrate availability within plant tissues or improved species-specific suitability for a desired plant species. Certain haloforms are preferred due to co-benefits in agricultural production, such as antifungal activity during plant growth or storage, or silage quality. To produce iodoform in somatic tissues, Vanadium-dependent iodoperoxidase (VIPO; SEQ ID NO: 41, 43, 45, 47, 49 or 51-60) is expressed under a constitutive or photosynthetic promoter (SEQ ID NO: 19-23, 93) in a monocot or dicot plant. Iodoform is extracted as described for bromoform in Example 3 and quantified by GC-MS. To produce iodoform in seeds, VIPO is expressed under a seed-specific promoter (SEQ ID NO: 24-25, or 94) and quantified as described in Example 1. Seeds or transiently or stably transformed leaf tissue is fed to cockroaches or added to rumen fluid as described in Examples 5, 7, or 8 and used to assess the methane-reducing effect of iodoform biosynthesized within plant tissues.

### Example 12

The ability to synthesize a mixture of haloforms are desirable as a means to maximize usage of all available substrates within plants, improve safety to both plants and animals, improve palatability to animals, and/or improve efficacy of the methane-reducing properties of the transgenic plant. Production of a mixture of haloforms is enabled by expression of a nonspecific haloperoxidase (VHPO; SEQ ID NO: 77, 79, 81, 83, 85, 87, 89, or 91) which accommodates incorporation of any or all of bromine, chlorine, or iodine into haloforms. The VHPO enzyme enables transgenic plants to maintain their methane-reducing properties in diverse settings by using whichever halogen is in greatest abundance at a particular time, agricultural setting, or part of the plant. Production of a mixture of haloforms is enabled by expression of any combination of Vanadium-dependent bromoperoxidase (VBPO, SEQ ID NO: 1 or 3); Vanadium-dependent iodoperoxidase (VIPO; SEQ ID NO: 41, 43, 45, 47, 49, or 51-60); Vanadium-dependent chloroperoxidase (VCPO; SEQ ID NO: 61, 63, 65, 67, 69, 71, 73, or 75); or nonspecific haloperoxidase (VHPO; SEQ ID NO: 77, 79, 81, 83, 85, 87, 89, or 91), and the selection of a particular combination of transgenes may be used to determine the profile of haloforms produced. To assess the production of haloform mixtures by particular combinations of haloperoxidase transgenes, tobacco (Nicotiana tabacum) leaves are transiently transformed with VBPO; VIPO; VCPO, and/or VHPO under control of a constitutive promoter and combined via assembly into a single construct or via co-transformation of multiple constructs. Haloforms may be extracted in methanol as described for bromoform in Example 3 and identified and quantified by GC-MS. To assess the methane-reducing effects of haloform mixtures biosynthesized by particular combinations of haloperoxidase transgenes, transiently or stably transformed leaf tissue or stably transformed seeds of Arabidopsis, Setaria, or corn are fed to cockroaches or added to rumen fluid as described in Examples 5, 7, or 8.

1. A genetically modified feed crop for reducing methanogenesis in a livestock animal, the genetically modified feed crop comprising:
a transgenic polynucleotide construct comprising a haloperoxidase (HPO) gene sequence encoding a HPO protein,
wherein the HPO protein enables biosynthesis of bromoform (CHBr₃), chloroform (CHCl₃), iodoform (CHI₃), or a combination thereof in the genetically modified feed crop.

2. The genetically modified feed crop of claim 1, wherein the HPO gene sequence encoding a HPO protein is a vanadium-dependent bromoperoxidase (VBPO), a vanadium-dependent iodoperoxidase (VIPO), vanadium-dependent chloroperoxidase (VCPO), or a nonspecific vanadium-dependent haloperoxidase (VHPO).

3. The genetically modified feed crop of claim 1, wherein the genetically modified feed crop is a monocot plant or a dicot plant.

4. The genetically modified feed crop of claim 1, wherein the HPO gene sequence has at least 90-99% identity to any one of SEQ ID NO: 1, 3, 41, 43, 45, 47, 49, 51, 52, 53, 54, 55, 56, 57, 58, 59, 60, 61, 63, 65, 67, 69, 71, 73, 75, 77, 79, 81, 83, 85, 87, 89, or 91 or the HPO protein comprises an amino acid sequence having at least 90-99% identity to any one of SEQ ID NO: 2, 4-8, 42, 44, 46, 48, 50, 62, 64, 66, 68, 70, 72, 74, 76, 78, 80, 82, 84, 86, 88, 90, or 92.

5-7. (canceled)

8. The genetically modified feed crop of claim 1, wherein the HPO gene sequence comprises a whole-plant constitutive promoter and the HPO gene sequence is expressed under the whole-plant constitutive promoter.

9. The genetically modified feed crop of claim 8, wherein the whole-plant constitutive promoter comprises a polynucleotide sequence having at least 90-99% identity to any one of SEQ ID NO: 19-20, or 93.

10. (canceled)

11. The genetically modified feed crop of claim 1, wherein the HPO gene sequence comprises an endosperm-specific promoter or photosynthetic-tissue promoter and is expressed under the endosperm-specific promoter or photosynthetic-tissue promoter.

12. The genetically modified feed crop of claim 11, wherein the endosperm-specific promoter comprises a polynucleotide sequence having at least 90-99% identity to any one of SEQ ID NO: 24-25, or 94; and the photosynthetic-tissue promoter comprises a polynucleotide sequence having at least 90-99% identity to any one of SEQ ID NO: 21-23.

13. (canceled)

14. The genetically modified feed crop of claim 1, further comprising:
a second transgenic polynucleotide construct comprising:
an acetoacetyl-CoA thiolase 2 (AACT2) gene sequence encoding an AACT2 protein.

15. The genetically modified feed crop of claim 14, wherein the AACT2 gene sequence has at least 90-99% identity to any one of SEQ ID NO: 9-10 or 12-13_or the AACT2 comprises an amino acid sequence having at least 90-99% identity to SEQ ID NO: 11.

16-18. (canceled)

19. The genetically modified feed crop of claim 14, wherein the AACT2 gene sequence comprises a whole-plant constitutive promoter and the AACT2 gene sequence is expressed under the whole-plant constitutive promoter.

20. The genetically modified feed crop of claim 19, wherein the whole-plant constitutive promoter comprises a polynucleotide sequence having at least 90-99% identity to any one of SEQ ID NO: 19-20, or 93.

21. (canceled)

22. The genetically modified feed crop of claim 14, wherein the HPO gene sequence and AACT2 gene sequence are separated by a self-cleaving peptide gene sequence encoding for a self-cleaving peptide.

23. The genetically modified feed crop of claim 22, wherein the self-cleaving peptide gene sequence has at least 90-99% identity to any one of SEQ ID NO: 29, 31, 33, 35, 37, or 39 or the self-cleaving peptide comprises an amino acid sequence having at least 90-99% identity to any one of SEQ ID NO: 30, 32, 34, 36, 38, or 40.

24-38. (canceled)

39. The genetically modified feed crop of claim 14, wherein the HPO is a nonspecific vanadium-dependent haloperoxidase (VHPO) gene sequence having at least 90-99% identity to any one of SEQ. ID NO: 77, 79, 81, 83, 85, 87, 89, or 91 or the HPO comprises an amino acid sequence having at least 90-99% identity to any one of SEQ ID NO: 78, 80, 82, 84, 86, 88, 90, or 92.

40-42. (canceled)

43. The genetically modified feed crop of claim 14, further comprising:
a third transgenic polynucleotide construct comprising:
a Wrinkled 1 (WRI1) gene sequence encoding a WRI1 protein.

44. The genetically modified feed crop of claim 43, wherein the WRI1 gene sequence has at least 90-99% identity to any one of SEQ ID NO: 14-15 or 17-18 or WRI1 protein comprises an amino acid sequence having at least 90-99% identity to SEQ ID NO: 16.

45-47. (canceled)

48. The genetically modified feed crop of claim 43, wherein the HPO gene sequence and WRI1 gene sequence are separated by a self-cleaving peptide gene sequence encoding for a self-cleaving peptide.

49. The genetically modified feed crop of claim 43, wherein the self-cleaving peptide gene sequence has at least 90-99% identity to any one of SEQ ID NO: 30, 32, 34, 36, 38, or 40 or the self-cleaving peptide comprises an amino acid sequence having at least 90-99% identity to any one of SEQ ID NO: 30, 32, 34, 36, 38, or 40.

50-52. (canceled)

53. A method for reducing methanogenesis in a livestock animal, the method comprising:
feeding a livestock animal the genetically modified feed crop of claim 1.

54-63. (canceled)
